(19) United States
(12) Patent Application Publication
(10) Pub. No.: US 2025/0236653 A1
(43) Pub. Date: Jul. 24, 2025

(54) TARGETED LOW POTENCY IL-12 FC FUSION PROTEINS AND USES THEREOF

(71) Applicant: oNKo-Innate Pty Ltd, Moonee Ponds (AU)

(72) Inventors: Richard BERRY, Mordialloc (AU); Nicholas David HUNTINGTON, Melbourne (AU); Imran Geoffrey HOUSE, Melbourne (AU); Joseph CURSONS, Melbourne (AU); Momeneh FOROUTAN, Allston, MA (US); Iva NIKOLIC, Melbourne (AU)

(21) Appl. No.: 19/033,268

(22) Filed: Jan. 21, 2025

Related U.S. Application Data
(60) Provisional application No. 63/623,458, filed on Jan. 22, 2024.
(60) Provisional application No. 63/560,957, filed on Mar. 4, 2024.
(60) Provisional application No. 63/711,820, filed on Oct. 25, 2024.

(51) Int. Cl. C07K 14/54; A61K 38/00; A61P 35/00

(52) U.S. Cl. CPC C07K 14/5434; A61P 35/00; A61K 38/00; C07K 2319/30

(57) ABSTRACT

The present invention is directed to compositions of novel, targeted, low potency IL-12 Fc fusion proteins, as well as methods of making and using such compositions.

### CROSS-REFERENCE TO RELATED APPLICATIONS

This application claims priority to U.S. Provisional Application No. 63/623,458, filed Jan. 22, 2024, U.S. Provisional Application No. 63/560,957, filed Mar. 4, 2024, and to U.S. Provisional Application No. 63/711,820, filed Oct. 25, 2024, each of which are entirely incorporated herein by reference.

### SEQUENCE LISTING

The instant application contains a Sequence Listing which is being submitted electronically in XML format and is hereby incorporated by reference in its entirety. The XML copy, created on Jan. 21, 2025, is named 51400_4004_US_SL.xml and is 196,608 bytes in size.

### BACKGROUND OF THE INVENTION

Interleukin-12 (IL-12) is a potent, pro-inflammatory cytokine that is produced by antigen presenting cells, such as, for example, dendritic cells, macrophages, and neutrophils. IL-12 belongs to the IL-12 family of cytokines. The IL-12 family of cytokines is unique in that they comprise heterodimeric cytokines. IL-12 is comprised of an alpha- (α-) subunit (encoded by the IL12A gene; also referred to herein as an “IL-12 p35 subunit”; Precursor Sequence—SEQ ID NO: 1 (as shown in FIG. 1); Mature Sequence—SEQ ID NO: 2 (as shown in FIG. 1)) and a beta-(β-) subunit (encoded by the IL12B gene; also referred to herein as an “IL-12 p40 subunit”; Precursor Sequence—SEQ ID NO: 3 (as shown in FIG. 1); Mature Sequence—SEQ ID NO: 4 (as shown in FIG. 1) that assemble to form a 70,000 Dalton (70 kDa) disulfide-linked heterodimer. Upon assembly of the IL-12 p35 and p40 subunits, a biologically active IL-12 heterodimer is formed. The receptor for IL-12, IL-12R, is a type I cytokine receptor comprising a beta-1 subunit (IL-12Rβ1—SEQ ID NO: 5 (as shown in FIG. 2); Extracellular Domain—SEQ ID NO: 6 (as shown in FIG. 2)) and a beta-2 subunit (IL-12Rβ2—SEQ ID NO: 7 (as shown in FIG. 2); Extracellular Domain—SEQ ID NO: 8 (as shown in FIG. 2)). The IL-12 p40 subunit has a binding affinity for IL-12Rβ1, and the IL-12 p35 subunit has a binding affinity for IL-12Rβ2.

The binding of IL-12 to IL-12R results in the phosphorylation of intracellular Signal Transducer And Activator Of Transcription 4 (STAT4) and triggers signaling pathways that: (i) induce TH1 cell differentiation, (ii) increase the activation and cytotoxic capacities of T- and natural killer (NK) cells, (iii) inhibit or reprogram immunosuppressive cells, such as, for example, tumor-associated macrophages (TAMs) and myeloid-derived suppressor cells (MDSCs), and (iv) induce the production of large amounts of interferon gamma (IFNγ), which is cytostatic/cytotoxic, anti-angiogenic, and can upregulate major histocompatibility complex (MHC) class 1 and MHC class 2 molecules on tumor cells to enable immune recognition.

However, the targeting of wild-type IL-12 to a target cell using an antigen binding domain (ABD) has generally not been successful due to the high affinity of IL-12 for its receptor. Instead of trafficking the “immunocytokine” to an on-target cell expressing the antigen bound by the ABD, the immunocytokine is instead trafficked to an off-target cell that expresses high levels of IL-12R. As such, there remains an unmet need for the develop of a fusion protein comprising IL-12 and an ABD capable of directing IL-12 to an on-target cell expressing an antigen bound by the ABD.

### BRIEF SUMMARY OF THE INVENTION

In one aspect, the present disclosure provides an IL-12 Fc fusion protein, comprising: (a) a first monomer comprising, from N-terminus to C-terminus: (i) a first IL-12 subunit domain, (ii) a first domain linker, (iii) a second IL-12 subunit domain, (iv) a second domain linker, and (v) a first Fc domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; (b) a second monomer comprising, from N-terminus to C-terminus: (i) a variable heavy (VH) domain, (ii) a constant heavy (CH1) domain, and (iii) a second Fc domain; and (c) a third monomer comprising, from N-terminus to C-terminus: (i) a variable light (VL) domain, and (ii) a constant light (CL) domain, wherein the VH domain and VL domain form an antigen binding domain (ABD) that binds a target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the ABD binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the ABD binds an extracellular domain (ECD) of the target antigen.

In a further embodiment and in accordance with any of the above, the first domain linker and/or the second domain linker comprises any one of SEQ ID NOs: 31-40. In some other embodiments, the first domain linker and/or the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a VH domain, (ii) a CH1 domain, and (iii) a first Fc domain; (b) a second monomer comprising, from N-terminus to C-terminus: (i) a VL domain, and (ii) a CL domain, wherein the VH domain and VL domain form an ABD that binds a first target antigen; (c) a third monomer comprising, from N-terminus to C-terminus: (i) a second Fc domain, (ii) a first domain linker, (iii) a first IL-12 subunit domain, (iv) a second domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the ABD binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the ABD binds an extracellular domain (ECD) of the target antigen.

In a further embodiment and in accordance with any of the above, the first domain linker and/or the second domain linker comprises any one of SEQ ID NOs: 31-40. In some other embodiments, the first domain linker and/or the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a first IL-12 subunit domain, (ii) a first domain linker, and (iii) a first Fc domain; (b) a second monomer comprising, from N-terminus to C-terminus: (i) a second IL-12 subunit domain, (ii) a second domain linker, (iii) a second Fc domain, (iv) a third domain linker, (v) a VH domain, and (vi) a CH1 domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (c) a third monomer comprising, from N-terminus to C-terminus: (i) a VL domain, and (ii) a CL domain, wherein the VH domain and VL domain form an ABD that binds a target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the ABD binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the ABD binds an extracellular domain (ECD) of the target antigen.

In a further embodiment and in accordance with any of the above, the first domain linker, the second domain linker, and/or the third domain linker comprises any one of SEQ ID NOs: 31-40. In some other embodiments, the first domain linker, the second domain linker, and/or the third domain linker comprises a covalent bond. In some other embodiments, at least one domain linker comprises any one of SEQ ID NOs: 31-40, and at least one domain linker comprises a covalent bond. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a VH domain, (ii) a CH1 domain, (iii) a first Fc domain, (iv) a first domain linker, and (v) a first IL-12 subunit domain; and (b) a second monomer comprising, from N-terminus to C-terminus: (i) a VL domain, (ii) a CL domain, (iii) a second Fc domain, (iv) a second domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the VH domain and VL domain form an ABD that binds a target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the ABD binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the ABD binds an extracellular domain (ECD) of the target antigen.

In a further embodiment and in accordance with any of the above, the first domain linker and/or the second domain linker comprises any one of SEQ ID NOs: 31-40. In some other embodiments, the first domain linker and/or the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a VH domain, (ii) a CH1 domain, and (iii) a first Fc domain; and (b) a second monomer comprising, from N-terminus to C-terminus: (i) a VL domain, (ii) a CL domain, (iii) a second Fc domain, (iv) a first domain linker, (v) a first IL-12 subunit domain, (vi) a second domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the VH domain and VL domain form an ABD that binds a target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the ABD binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the ABD binds an extracellular domain (ECD) of the target antigen.

In a further embodiment and in accordance with any of the above, the first domain linker and/or the second domain linker comprises any one of SEQ ID NOs: 31-40. In some other embodiments, the first domain linker and/or the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising a first Fc domain; (b) a second monomer comprising, from N-terminus to C-terminus: (i) a VL domain, and (ii) a CL domain; and (c) a third monomer comprising, from N-terminus to C-terminus: (i) a first IL-12 subunit domain, (ii) a first domain linker, (iii) a second IL-12 subunit domain, (iv) a second domain linker, (v) a second Fc domain, (vi) a VH domain, and (vii) a CH1 domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the VH domain and VL domain form an ABD that binds a target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the ABD binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the ABD binds an extracellular domain (ECD) of the target antigen.

In a further embodiment and in accordance with any of the above, the first domain linker and/or the second domain linker comprises any one of SEQ ID NOs: 31-40. In some other embodiments, the first domain linker and/or the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a first VH (VH1) domain, (ii) a first CH1 domain, and (iii) a first Fc domain; (b) a second monomer comprising, from N-terminus to C-terminus: (i) a first VL (VL1) domain, and (ii) a first CL domain, wherein the VH1 domain and VL1 domain form a first ABD that binds a first target antigen; (c) a third monomer comprising, from N-terminus to C-terminus: (i) a second VH (VH2) domain, (ii) a second CH1 domain, (iii) a second Fc domain, (iv) a first domain linker, (v) a first IL-12 subunit domain, (vi) a second domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (d) a fourth monomer comprising, from N-terminus to C-terminus: (i) a second VL (VL2) domain, and (ii) a second CL domain, wherein the VH2 domain and VL2 domain form a second ABD that binds a second target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first ABD binds a first target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the second ABD binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first ABD and the second ABD both bind a target antigen selected from a group including: (i) human OX40, (ii) human LAG3, (iii) human PD1, (iv) human ICOS, (v) human 41BB, (vi) human CCR7, and (vii) human CCR8. In other embodiments, the first ABD binds a first target antigen selected from a group including: (i) human OX40, (ii) human LAG3, (iii) human PD1, (iv) human ICOS, (v) human 41BB, (vi) human CCR7, and (vii) human CCR8; and the second ABD binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first target antigen and/or the second target antigen comprises human PD1, and the first ABD and/or the second ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first ABD and/or the second ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first ABD and/or the second ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first ABD and/or the second ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first ABD and/or the second ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first ABD and/or the second ABD comprises a VH/VL domain pair selected from a group including: (i) SEQ ID NOs: 78 and 79, respectively. In other embodiments, the first target antigen and/or the second target antigen comprises human CCR8, and the first ABD and/or the second ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the first ABD binds an ECD of the first target antigen.

In a further embodiment and in accordance with any of the above, the second ABD binds an ECD of the second target antigen.

In a further embodiment and in accordance with any of the above, the first ABD binds an ECD of the first target antigen, and the second ABD binds an ECD of the second target antigen.

In a further embodiment and in accordance with any of the above, the first domain linker and/or the second domain linker comprises any one of SEQ ID NOs: 31-40. In some other embodiments, the first domain linker and/or the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a first VH (VH1) domain, (ii) a first CH1 domain, (iii) a first Fc domain, (iv) a first domain linker, and (v) a first IL-12 subunit domain; (b) a second monomer comprising, from N-terminus to C-terminus: (i) a first VL (VL1) domain, and (ii) a first CL domain, wherein the VH1 domain and VL1 domain form a first ABD that binds a first target antigen; (c) a third monomer comprising, from N-terminus to C-terminus: (i) a second VH (VH2) domain, (ii) a second CH1 domain, (iii) a second Fc domain, (iv) a second domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (d) a fourth monomer comprising, from N-terminus to C-terminus: (i) a second VL (VL2) domain, and (ii) a second CL domain, wherein the VH2 domain and VL2 domain form a second ABD that binds a second target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first ABD binds a first target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the second ABD binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first ABD and the second ABD both bind a target antigen selected from a group including: (i) human OX40, (ii) human LAG3, (iii) human PD1, (iv) human ICOS, (v) human 41BB, (vi) human CCR7, and (vii) human CCR8. In other embodiments, the first ABD binds a first target antigen selected from a group including: (i) human OX40, (ii) human LAG3, (iii) human PD1, (iv) human ICOS, (v) human 41BB, (vi) human CCR7, and (vii) human CCR8; and the second ABD binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first target antigen and/or the second target antigen comprises human PD1, and the first ABD and/or the second ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first ABD and/or the second ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first ABD and/or the second ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first ABD and/or the second ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first ABD and/or the second ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first ABD and/or the second ABD comprises a VH/VL domain pair selected from a group including: (i) SEQ ID NOs: 78 and 79, respectively. In other embodiments, the first target antigen and/or the second target antigen comprises human CCR8, and the first ABD and/or the second ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the first ABD binds an ECD of the first target antigen.

In a further embodiment and in accordance with any of the above, the second ABD binds an ECD of the second target antigen.

In a further embodiment and in accordance with any of the above, the first ABD binds an ECD of the first target antigen, and the second ABD binds an ECD of the second target antigen.

In a further embodiment and in accordance with any of the above, the first domain linker and/or the second domain linker comprises any one of SEQ ID NOs: 31-40. In some other embodiments, the first domain linker and/or the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a first IL-12 subunit domain, (ii) a first domain linker, (iii) a second IL-12 subunit domain, and (iv) a first Fc domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising: (i) an anti-target antigen scFv, wherein the anti-target antigen scFv comprises a VH domain, a second domain linker, and a VL domain, (ii) a third domain linker, and (iii) a second Fc domain, wherein the anti-target antigen scFv is covalently attached to the third domain linker at the N-terminus of the second Fc domain.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the anti-target antigen scFv binds an extracellular domain (ECD) of the target antigen.

In a further embodiment and in accordance with any of the above, the VH domain of the anti-target antigen scFv is covalently attached to the third domain linker at the N-terminus of the second Fc domain. In other embodiments, the VL domain of the anti-target antigen scFv is covalently attached to the third domain linker at the N-terminus of the second Fc domain.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the second domain linker comprises any one of SEQ ID NOs: 31-40.

In a further embodiment and in accordance with any of the above, the first domain linker and/or the third domain linker comprises any one of SEQ ID NOs: 31-40. In some other embodiments, the first domain linker and/or the third domain linker comprises a covalent bond. In some other embodiments, the first domain linker comprises any one of SEQ ID NOs: 31-40, and the third domain linker comprises a covalent bond. In some other embodiments, the first domain linker comprises a covalent bond, and the third domain linker comprises any one of SEQ ID NOs: 31-40. In some embodiments, at least one of the domain linkers comprises a hinge region of a Fc domain.

In another aspect, the present disclosure provides an IL-12 Fc fusion protein, comprising: (a) a first monomer comprising: (i) an anti-target antigen scFv, wherein the anti-target antigen scFv comprises a VH domain, a first domain linker, and a VL domain, (ii) a second domain linker, and (iii) a first Fc domain, wherein the anti-target antigen scFv is covalently attached to the second domain linker at the N-terminus of the first Fc domain; and (b) a second monomer comprising, from N-terminus to C-terminus: (i) a second Fc domain, (ii) a third domain linker, (iii) a first IL-12 subunit domain, (iv) a fourth domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the anti-target antigen scFv binds an extracellular domain (ECD) of the target antigen.

In a further embodiment and in accordance with any of the above, the VH domain of the anti-target antigen scFv is covalently attached to the second domain linker at the N-terminus of the first Fc domain. In other embodiments, the VL domain of the anti-target antigen scFv is covalently attached to the second domain linker at the N-terminus of the first Fc domain.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40.

In a further embodiment and in accordance with any of the above, the second domain linker, the third domain linker, and/or the fourth domain linker comprises any one of SEQ ID NOs: 31-40. In some other embodiments, the second domain linker, the third domain linker, and/or the fourth domain linker comprises a covalent bond. In some other embodiments, at least one domain linker comprises any one of SEQ ID NOs: 31-40, and at least one domain linker comprises a covalent bond. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a first IL-12 subunit domain, (ii) a first domain linker, and (iii) a first Fc domain; and (b) a second monomer comprising: (i) a second IL-12 subunit domain, (ii) a second domain linker, (iii) a second Fc domain, wherein the second IL-12 subunit domain is covalently attached to the second domain linker at the N-terminus of the second Fc domain, (iv) a third domain linker, and (v) an anti-target antigen scFv, wherein: (1) the second Fc domain is covalently attached to the third domain linker at the N-terminus of the anti-target antigen scFv, (2) the anti-target antigen scFv comprises a VH domain, a fourth domain linker, and a VL domain, (3) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (4) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the anti-target antigen scFv binds an extracellular domain (ECD) of the target antigen.

In a further embodiment and in accordance with any of the above, the VH domain of the anti-target antigen scFv is covalently attached to the third domain linker at the N-terminus of the second Fc domain. In other embodiments, the VL domain of the anti-target antigen scFv is covalently attached to the third domain linker at the N-terminus of the second Fc domain.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the fourth domain linker comprises any one of SEQ ID NOs: 31-40.

In a further embodiment and in accordance with any of the above, the first domain linker, the second domain linker, and/or the third domain linker comprises any one of SEQ ID NOs: 31-40. In some other embodiments, the first domain linker, the second domain linker, and/or the third domain linker comprises a covalent bond. In some other embodiments, at least one domain linker comprises any one of SEQ ID NOs: 31-40, and at least one domain linker comprises a covalent bond. In some embodiments, at least one of the domain linkers comprises a hinge region of a Fc domain.

In another aspect, the present disclosure provides an IL-12 Fc fusion protein, comprising: (a) a first monomer comprising: (i) a first IL-12 subunit domain, (ii) a first domain linker, (iii) a second IL-12 subunit domain, wherein the first IL-12 subunit domain is covalently attached to the first domain linker at the N-terminus of the second IL-12 subunit domain, (iv) a second domain linker, (v) a first Fc domain, wherein the second IL-12 subunit domain is covalently attached to the second domain linker at the N-terminus of the first Fc domain, (vi) a third domain linker, and (vii) an anti-target antigen scFv, wherein: (1) the first Fc domain is covalently attached to the third domain linker at the N-terminus of the anti-target antigen scFv, (2) the anti-target antigen scFv comprises a VH domain, a fourth domain linker, and a VL domain, (3) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (4) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising a second Fc domain.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In a further embodiment and in accordance with any of the above, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the anti-target antigen scFv binds an extracellular domain (ECD) of the target antigen.

In a further embodiment and in accordance with any of the above, the VH domain of the anti-target antigen scFv is covalently attached to the third domain linker at the N-terminus of the second Fc domain. In other embodiments, the VL domain of the anti-target antigen scFv is covalently attached to the third domain linker at the N-terminus of the second Fc domain.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the fourth domain linker comprises any one of SEQ ID NOs: 31-40.

In a further embodiment and in accordance with any of the above, the first domain linker, the second domain linker, and/or the third domain linker comprises any one of SEQ ID NOs: 31-40. In some other embodiments, the first domain linker, the second domain linker, and/or the third domain linker comprises a covalent bond. In some other embodiments, at least one domain linker comprises any one of SEQ ID NOs: 31-40, and at least one domain linker comprises a covalent bond. In some embodiments, at least one of the domain linkers comprises a hinge region of a Fc domain.

In another aspect, the present disclosure provides an IL-12 Fc fusion protein, comprising: (a) a first monomer comprising: (i) a first anti-target antigen scFv, wherein the first anti-target antigen scFv comprises a first VH (VH1) domain, a first domain linker, and a first VL (VL1) domain, (ii) a second domain linker, and (iii) a first Fc domain, wherein the first anti-target antigen scFv is covalently attached to the second domain linker at the N-terminus of the first Fc domain; and (b) a second monomer comprising: (i) a second anti-target antigen scFv, wherein the second anti-target antigen scFv comprises a second VH (VH2) domain, a third domain linker, and a second VL (VL2) domain, (ii) a fourth domain linker, (iii) a second Fc domain, wherein the second anti-target antigen scFv is covalently attached to the fourth domain linker at the N-terminus of the second Fc domain, (iv) a fifth domain linker, (v) a first IL-12 subunit domain, (vi) a sixth domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, (3) the second Fc domain is covalently attached to the fifth domain linker at the N-terminus of the first IL-12 subunit domain, and (4) the first IL-12 subunit domain is covalently attached to the sixth domain linker at the N-terminus of the second IL-12 subunit domain.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first anti-target antigen scFv and the second anti-target antigen scFv both bind a target antigen selected from a group including: (i) human OX40, (ii) human LAG3, (iii) human PD1, (iv) human ICOS, (v) human 41BB, (vi) human CCR7, and (vii) human CCR8. In other embodiments, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8, and the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first target antigen and/or the second target antigen comprises human PD1, and the first anti-target antigen scFv and/or the second anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first anti-target antigen scFv and/or the second anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first anti-target antigen scFv and/or the second anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first anti-target antigen scFv and/or the second anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first anti-target antigen scFv and/or the second anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first anti-target antigen scFv and/or the second anti-target antigen scFv comprises a VH/VL domain pair selected from a group including: (i) SEQ ID NOs: 78 and 79, respectively. In other embodiments, the first target antigen and/or the second target antigen comprises human CCR8, and the first anti-target antigen scFv and/or the second anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the first anti-target antigen scFv binds an ECD of the first target antigen.

In a further embodiment and in accordance with any of the above, the second anti-target antigen scFv binds an ECD of the second target antigen.

In a further embodiment and in accordance with any of the above, the first anti-target antigen scFv binds an ECD of the first target antigen, and the second anti-target antigen scFv binds an ECD of the second target antigen.

In a further embodiment and in accordance with any of the above, the VH1 domain of the first anti-target antigen scFv is covalently attached to the second domain linker at the N-terminus of the first Fc domain. In other embodiments, the VL1 domain of the anti-target antigen scFv is covalently attached to the second domain linker at the N-terminus of the first Fc domain.

In a further embodiment and in accordance with any of the above, the VH2 domain of the second anti-target antigen scFv is covalently attached to the fourth domain linker at the N-terminus of the second Fc domain. In other embodiments, the VL2 domain of the second anti-target antigen scFv is covalently attached to the fourth domain linker at the N-terminus of the second Fc domain.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, and the third domain linker comprises any one of SEQ ID NOs: 31-40.

In a further embodiment and in accordance with any of the above, the second domain linker, the fourth domain linker, the fifth domain linker, and/or the sixth domain linker comprises any one of SEQ ID NOs: 31-40. In some other embodiments, the second domain linker, the fourth domain linker, the fifth domain linker, and/or the sixth domain linker comprises a covalent bond. In some other embodiments, at least one domain linker comprises any one of SEQ ID NOs: 31-40, and at least one domain linker comprises a covalent bond. In some embodiments, at least one of the domain linkers comprises a hinge region of a Fc domain.

In another aspect, the present disclosure provides an IL-12 Fc fusion protein, comprising: the IL-12 Fc fusion protein comprises: (a) a first monomer comprising: (i) a first anti-target antigen scFv, wherein the first anti-target antigen scFv comprises a first VH (VH1) domain, a first domain linker, and a first VL (VL1) domain, (ii) a second domain linker, (iii) a first Fc domain, wherein the first anti-target antigen scFv is covalently attached to the second domain linker at the N-terminus of the first Fc domain, (iv) a third domain linker, and (v) a first IL-12 subunit domain, wherein the first Fc domain is covalently attached to the third domain linker at the N-terminus of the first IL-12 subunit domain; and (b) a second monomer comprising: (i) a second anti-target antigen scFv, wherein the second anti-target antigen scFv comprises a second VH (VH2) domain, a fourth domain linker, and a second VL (VL2) domain, (ii) a fifth domain linker, (iii) a second Fc domain, wherein the second anti-target antigen scFv is covalently attached to the fifth domain linker at the N-terminus of the second Fc domain, (iv) a sixth domain linker, and (v) a second IL-12 subunit domain, wherein: (1) the second Fc domain is covalently attached to the sixth domain linker at the N-terminus of the second IL-12 subunit domain, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first anti-target antigen scFv and the second anti-target antigen scFv both bind a target antigen selected from a group including: (i) human OX40, (ii) human LAG3, (iii) human PD1, (iv) human ICOS, (v) human 41BB, (vi) human CCR7, and (vii) human CCR8. In other embodiments, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8, and the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In a further embodiment and in accordance with any of the above, the first target antigen and/or the second target antigen comprises human PD1, and the first anti-target antigen scFv and/or the second anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first anti-target antigen scFv and/or the second anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first anti-target antigen scFv and/or the second anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first anti-target antigen scFv and/or the second anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first anti-target antigen scFv and/or the second anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first anti-target antigen scFv and/or the second anti-target antigen scFv comprises a VH/VL domain pair selected from a group including: (i) SEQ ID NOs: 78 and 79, respectively. In other embodiments, the first target antigen and/or the second target antigen comprises human CCR8, and the first anti-target antigen scFv and/or the second anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively.

In a further embodiment and in accordance with any of the above, the first anti-target antigen scFv binds an ECD of the first target antigen.

In a further embodiment and in accordance with any of the above, the second anti-target antigen scFv binds an ECD of the second target antigen.

In a further embodiment and in accordance with any of the above, the first anti-target antigen scFv binds an ECD of the first target antigen, and the second anti-target antigen scFv binds an ECD of the second target antigen.

In a further embodiment and in accordance with any of the above, the VH1 domain of the first anti-target antigen scFv is covalently attached to the second domain linker at the N-terminus of the first Fc domain. In other embodiments, the VL1 domain of the anti-target antigen scFv is covalently attached to the second domain linker at the N-terminus of the first Fc domain.

In a further embodiment and in accordance with any of the above, the VH2 domain of the second anti-target antigen scFv is covalently attached to the fifth domain linker at the N-terminus of the second Fc domain. In other embodiments, the VL2 domain of the second anti-target antigen scFv is covalently attached to the fifth domain linker at the N-terminus of the second Fc domain.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, and the fourth domain linker comprises any one of SEQ ID NOs: 31-40.

In a further embodiment and in accordance with any of the above, the second domain linker, the third domain linker, the fifth domain linker, and/or the sixth domain linker comprises any one of SEQ ID NOs: 31-40. In some other embodiments, the second domain linker, the third domain linker, the fifth domain linker, and/or the sixth domain linker comprises a covalent bond. In some other embodiments, at least one domain linker comprises any one of SEQ ID NOs: 31-40, and at least one domain linker comprises a covalent bond. In some embodiments, at least one of the domain linkers comprises a hinge region of a Fc domain.

In another aspect, the present disclosure provides an IL-12 Fc fusion protein, comprising: (a) a first monomer comprising, from N-terminus to C-terminus: (i) a first IL-12 subunit domain, (ii) a first domain linker, (iii) a second IL-12 subunit domain, and (iv) a first Fc domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising, from N-terminus to C-terminus: (i) a VHH domain, (ii) a second domain linker, and (iii) a second Fc domain, wherein the VHH domain binds a target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, and the second domain linker comprises any one of SEQ ID NOs: 31-40. In some other embodiments, the first domain linker comprises a covalent bond, and the second domain linker comprises a covalent bond. In some other embodiments, the first domain linker comprises any one of SEQ ID NOs: 31-40, and the second domain linker comprises a covalent bond. In some other embodiments, the first domain linker comprises a covalent bond, and the second domain linker comprises any one of SEQ ID NOs: 31-40. In some embodiments, at least one of the domain linkers comprises a hinge region of a Fc domain.

In a further embodiment and in accordance with any of the above, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In a further embodiment and in accordance with any of the above, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)).

In a further embodiment and in accordance with any of the above, the target antigen comprises 41BB, and the VHH domain comprises: SEQ ID NO: 129. In other embodiments, the target antigen comprises CCR8, and the VHH domain comprises: SEQ ID NO: 86.

In another aspect, the present disclosure provides an IL-12 Fc fusion protein, comprising: (a) a first monomer comprising, from N-terminus to C-terminus: (i) a VHH domain, (ii) a first domain linker, and (iii) a first Fc domain; and (b) a second monomer comprising, from N-terminus to C-terminus: (i) a second Fc domain, (ii) a second domain linker, (iii) a first IL-12 subunit domain, (iv) a third domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, the second domain linker comprises any one of SEQ ID NOs: 31-40, and the third domain linker comprises any one of SEQ ID NOs: 31-40. In some other embodiments, the first domain linker, the second domain linker, and/or the third domain linker comprises a covalent bond. In some other embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

In a further embodiment and in accordance with any of the above, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In a further embodiment and in accordance with any of the above, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)).

In a further embodiment and in accordance with any of the above, the target antigen comprises 41BB, and the VHH domain comprises: SEQ ID NO: 129. In other embodiments, the target antigen comprises CCR8, and the VHH domain comprises: SEQ ID NO: 86.

In another aspect, the present disclosure provides an IL-12 Fc fusion protein, comprising: (a) a first monomer comprising, from N-terminus to C-terminus: (i) a first IL-12 subunit domain, (ii) a first domain linker, and (iii) a first Fc domain; and (b) a second monomer comprising, from N-terminus to C-terminus: (i) a second IL-12 subunit domain, (ii) a second domain linker, (iii) a second Fc domain, (iv) a third domain linker, and (v) a VHH domain, wherein: (1) the VHH domain binds a target antigen, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, the second domain linker comprises any one of SEQ ID NOs: 31-40, and the third domain linker comprises any one of SEQ ID NOs: 31-40. In some other embodiments, the first domain linker, the second domain linker, and/or the third domain linker comprises a covalent bond. In some other embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

In a further embodiment and in accordance with any of the above, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In a further embodiment and in accordance with any of the above, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)).

In a further embodiment and in accordance with any of the above, the target antigen comprises 41BB, and the VHH domain comprises: SEQ ID NO: 129. In other embodiments, the target antigen comprises CCR8, and the VHH domain comprises: SEQ ID NO: 86.

In another aspect, the present disclosure provides an IL-12 Fc fusion protein, comprising: (a) a first monomer comprising, from N-terminus to C-terminus: (i) a first IL-12 subunit domain, (ii) a first domain linker, (iii) a second IL-12 subunit domain, (iv) a second domain linker, (v) a first Fc domain, (vi) a third domain linker, and (vii) a VHH domain, wherein: (1) the VHH domain binds a target antigen, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising a second Fc domain.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, the second domain linker comprises any one of SEQ ID NOs: 31-40, and the third domain linker comprises any one of SEQ ID NOs: 31-40. In some other embodiments, the first domain linker, the second domain linker, and/or the third domain linker comprises a covalent bond. In some other embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

In a further embodiment and in accordance with any of the above, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In a further embodiment and in accordance with any of the above, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)).

In a further embodiment and in accordance with any of the above, the target antigen comprises 41BB, and the VHH domain comprises: SEQ ID NO: 129. In other embodiments, the target antigen comprises CCR8, and the VHH domain comprises: SEQ ID NO: 86.

In another aspect, the present disclosure provides an IL-12 Fc fusion protein, comprising: (a) a first monomer comprising, from N-terminus to C-terminus: (i) a first VHH domain, (ii) a first domain linker, (iii) a first Fc domain, (iv) a second domain linker, (v) a first IL-12 subunit domain, (vi) a third domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the first VHH domain binds a first target antigen; and (b) a second monomer comprising, from N-terminus to C-terminus: (i) a second VHH domain, (ii) a fourth domain linker, and (iii) a second Fc domain, wherein the second VHH domain binds a second target antigen.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, the second domain linker comprises any one of SEQ ID NOs: 31-40, the third domain linker comprises any one of SEQ ID NOs: 31-40, and the fourth domain linker comprises any one of SEQ ID NOs: 31-40. In some other embodiments, the first domain linker, the second domain linker, the third domain linker, and/or the fourth domain linker comprises a covalent bond. In some other embodiments, at least one domain linker comprises any one of SEQ ID NOs: 31-40, and at least one domain linker comprises a covalent bond. In some embodiments, at least one of the domain linkers comprises a hinge region of a Fc domain.

In a further embodiment and in accordance with any of the above, the first VHH domain binds a first target antigen selected from a group including: (i) OX40, (ii) LAG3, (iii) PD1, (iv) ICOS, (v) 41BB, (vi) CCR7, and (vii) CCR8.

In a further embodiment and in accordance with any of the above, the second VHH domain binds a second target antigen selected from a group including: (i) OX40, (ii) LAG3, (iii) PD1, (iv) ICOS, (v) 41BB, (vi) CCR7, and (vii) CCR8.

In a further embodiment and in accordance with any of the above, the first VHH domain and the second VHH domain both bind a target antigen selected from a group including: (i) OX40, (ii) LAG3, (iii) PD1, (iv) ICOS, (v) 41BB, (vi) CCR7, and (vii) CCR8. In other embodiments, the VHH domain binds a first target antigen selected from a group including: (i) OX40, (ii) LAG3, (iii) PD1, (iv) ICOS, (v) 41BB, (vi) CCR7, and (vii) CCR8, and the second VHH domain binds a second target antigen selected from a group including: (i) OX40, (ii) LAG3, (iii) PD1, (iv) ICOS, (v) 41BB, (vi) CCR7, and (vii) CCR8.

In a further embodiment and in accordance with any of the above, the first target antigen and/or the second target antigen comprises PD1, and the first VHH domain and/or the second VHH domain comprises a VHH domain described herein (see, e.g., Section (III)(A)(2)(a)). In other embodiments, the first target antigen and/or the second target antigen comprises LAG3, and the first VHH domain and/or the second VHH domain comprises a VHH domain described herein (see, e.g., Section (III)(A)(2)(b)). In other embodiments, the first target antigen and/or the second target antigen comprises OX40, and the first VHH domain and/or the second VHH domain comprises a VHH domain described herein (see, e.g., Section (III)(A)(2)(c)). In other embodiments, the first target antigen and/or the second target antigen comprises 41BB, and the first VHH domain and/or the second VHH domain comprises a VHH domain described herein (see, e.g., Section (III)(A)(2)(d)). In other embodiments, the first target antigen and/or the second target antigen comprises ICOS, and the first VHH domain and/or the second VHH domain comprises a VHH domain described herein (see, e.g., Section (III)(A)(2)(e)). In other embodiments, the first target antigen and/or the second target antigen comprises CCR7, and the first VHH domain and/or the second VHH domain comprises a VHH domain described herein (see, e.g., Section (III)(A)(2)(f)). In other embodiments, the first target antigen and/or the second target antigen comprises CCR8, and the first VHH domain and/or the second VHH domain comprises a VHH domain described herein (see, e.g., Section (III)(A)(2)(g)).

In a further embodiment and in accordance with any of the above, the first VHH domain is humanized.

In a further embodiment and in accordance with any of the above, the second VHH domain is humanized.

In a further embodiment and in accordance with any of the above, the first VHH domain is humanized, and the second VHH domain is humanized.

In a further embodiment and in accordance with any of the above, the first target antigen and/or the second target antigen comprises 41BB, and the first VHH domain and/or the second VHH domain comprises: SEQ ID NO: 129. In other embodiments, the first target antigen and/or the second target antigen comprises CCR8, and the first VHH domain and/or the second VHH domain comprises: SEQ ID NO: 86.

In another aspect, the present disclosure provides an IL-12 Fc fusion protein, comprising: (a) a first monomer comprising, from N-terminus to C-terminus: (i) a first VHH domain, (ii) a first domain linker, (iii) a first Fc domain, (iv) a second domain linker, and (v) a first IL-12 subunit domain, wherein the first VHH domain binds a first target antigen; and (b) a second monomer comprising, from N-terminus to C-terminus: (i) a second VHH domain, (ii) a third domain linker, (iii) a second Fc domain, (iv) a fourth domain linker, and (v) a second IL-12 subunit domain, wherein: (1) the second VHH domain binds a second target antigen, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In a further embodiment and in accordance with the above, the variant IL-12 p35 subunit domain comprises any one of SEQ ID NOs: 17-21.

In a further embodiment and in accordance with any of the above, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In a further embodiment and in accordance with any of the above, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In a further embodiment and in accordance with any of the above, the first domain linker comprises any one of SEQ ID NOs: 31-40, the second domain linker comprises any one of SEQ ID NOs: 31-40, the third domain linker comprises any one of SEQ ID NOs: 31-40, and the fourth domain linker comprises any one of SEQ ID NOs: 31-40. In some other embodiments, the first domain linker, the second domain linker, the third domain linker, and/or the fourth domain linker comprises a covalent bond. In some other embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

In a further embodiment and in accordance with any of the above, the first VHH domain binds a first target antigen selected from a group including: (i) OX40, (ii) LAG3, (iii) PD1, (iv) ICOS, (v) 41BB, (vi) CCR7, and (vii) CCR8.

In a further embodiment and in accordance with any of the above, the second VHH domain binds a second target antigen selected from a group including: (i) OX40, (ii) LAG3, (iii) PD1, (iv) ICOS, (v) 41BB, (vi) CCR7, and (vii) CCR8.

In a further embodiment and in accordance with any of the above, the first VHH domain and the second VHH domain both bind a target antigen selected from a group including: (i) OX40, (ii) LAG3, (iii) PD1, (iv) ICOS, (v) 41BB, (vi) CCR7, and (vii) CCR8. In other embodiments, the first VHH domain binds a first target antigen selected from a group including: (i) OX40, (ii) LAG3, (iii) PD1, (iv) ICOS, (v) 41BB, (vi) CCR7, and (vii) CCR8, and the second VHH domain binds a second target antigen selected from a group including: (i) OX40, (ii) LAG3, (iii) PD1, (iv) ICOS, (v) 41BB, (vi) CCR7, and (vii) CCR8.

In a further embodiment and in accordance with any of the above, the first target antigen and/or the second target antigen comprises PD1, and the first VHH domain and/or the second VHH domain comprises a VHH domain described herein (see, e.g., Section (III)(A)(2)(a)). In other embodiments, the first target antigen and/or the second target antigen comprises LAG3, and the first VHH domain and/or the second VHH domain comprises a VHH domain described herein (see, e.g., Section (III)(A)(2)(b)). In other embodiments, the first target antigen and/or the second target antigen comprises OX40, and the first VHH domain and/or the second VHH domain comprises a VHH domain described herein (see, e.g., Section (III)(A)(2)(c)). In other embodiments, the first target antigen and/or the second target antigen comprises 41BB, and the first VHH domain and/or the second VHH domain comprises a VHH domain described herein (see, e.g., Section (III)(A)(2)(d)). In other embodiments, the first target antigen and/or the second target antigen comprises ICOS, and the first VHH domain and/or the second VHH domain comprises a VHH domain described herein (see, e.g., Section (III)(A)(2)(e)). In other embodiments, the first target antigen and/or the second target antigen comprises CCR7, and the first VHH domain and/or the second VHH domain comprises a VHH domain described herein (see, e.g., Section (III)(A)(2)(f)). In other embodiments, the first target antigen and/or the second target antigen comprises CCR8, and the first VHH domain and/or the second VHH domain comprises a VHH domain described herein (see, e.g., Section (III)(A)(2)(g)).

In a further embodiment and in accordance with any of the above, the first VHH domain is humanized.

In a further embodiment and in accordance with any of the above, the second VHH domain is humanized.

In a further embodiment and in accordance with any of the above, the first VHH domain is humanized, and the second VHH domain is humanized.

In a further embodiment and in accordance with any of the above, the first target antigen and/or the second target antigen comprises 41BB, and the first VHH domain and/or the second VHH domain comprises: SEQ ID NO: 129. In other embodiments, the first target antigen and/or the second target antigen comprises CCR8, and the first VHH domain and/or the second VHH domain comprises: SEQ ID NO: 86.

In another aspect, the present disclosure provides a nucleic acid composition, comprising: one or more nucleic acids encoding an IL-12 Fc fusion protein, as described herein.

In another aspect, the present disclosure provides an expression vector, comprising: one or more nucleic acids encoding an IL-12 Fc fusion protein, as described herein.

In another aspect, the present disclosure provides a host cell, wherein the host cell is transformed with an expression vector, as described herein.

In another aspect, the present disclosure provides a method of making an IL-12 Fc fusion protein, comprising: (a) culturing a host cell transformed with an expression vector comprising one or more nucleic acids encoding an IL-12 Fc fusion protein, as described herein; and (b) isolating and/or purifying the IL-12 Fc fusion protein.

In another aspect, the present disclosure provides an IL-12 Fc fusion protein, comprising: (a) a first monomer; (b) a second monomer; and (c) a third monomer, wherein the first monomer, the second monomer, and the third monomer comprise: (i) SEQ ID NOs: 49, 153, and 145, respectively, (ii) SEQ ID NOs: 48, 146, and 145, respectively, (iii) SEQ ID NOs: 48, 151, and 150, respectively, (iv) SEQ ID NOs: 48, 153, and 145, respectively, (v) SEQ ID NOs: 48, 155, and 145, respectively, (vi) SEQ ID NOs: 48, 157, and 145, respectively, (vii) SEQ ID NOs: 48, 159, and 150, respectively, (viii) SEQ ID NOs: 48, 161, and 150, respectively, (ix) SEQ ID NOs: 48, 163, and 150, respectively, (x) SEQ ID NOs: 48, 165, and 150, respectively, (xi) SEQ ID NOs: 48, 167, and 150, respectively, (xii) SEQ ID NOs: 48, 169, and 150, respectively, (xiii) SEQ ID NOs: 49, 141, and 140, respectively, (xiv) SEQ ID NOs: 49, 146, and 145, respectively, (xv) SEQ ID NOs: 49, 151, and 150, respectively, (xvi) SEQ ID NOs: 48, 141, and 140, respectively, (xvii) SEQ ID NOs: 49, 155, and 145, respectively, (xviii) SEQ ID NOs: 49, 157, and 145, respectively, (xix) SEQ ID NOs: 49, 159, and 150, respectively, (xx) SEQ ID NOs: 49, 161, and 150, respectively, (xxi) SEQ ID NOs: 49, 163, and 150, respectively, (xxii) SEQ ID NOs: 49, 165, and 150, respectively, (xxiii) SEQ ID NOs: 49, 167, and 150, respectively, (xxiv) SEQ ID NOs: 49, 169, and 150, respectively, (xxv) SEQ ID NOs: 50, 141, and 140, respectively, (xxvi) SEQ ID NOs: 50, 146, and 145, respectively, (xxvii) SEQ ID NOs: 50, 151, and 150, respectively, (xxviii) SEQ ID NOs: 50, 153, and 145, respectively, (xxix) SEQ ID NOs: 50, 155, and 145, respectively, (xxx) SEQ ID NOs: 50, 157, and 145, respectively, (xxxi) SEQ ID NOs: 50, 159, and 150, respectively, (xxxii) SEQ ID NOs: 50, 161, and 150, respectively, (xxxiii) SEQ ID NOs: 50, 163, and 150, respectively, (xxxiv) SEQ ID NOs: 50, 165, and 150, respectively, (xxxv) SEQ ID NOs: 50, 167, and 150, respectively, (xxxvi) SEQ ID NOs: 50, 169, and 150, respectively, (xxxvii) SEQ ID NOs: 51, 141, and 140, respectively, (xxxviii) SEQ ID NOs: 51, 146, and 145, respectively, (xxxix) SEQ ID NOs: 51, 151, and 150, respectively, (xl) SEQ ID NOs: 51, 153, and 145, respectively, (xli) SEQ ID NOs: 51, 155, and 145, respectively, (xlii) SEQ ID NOs: 51, 157, and 145, respectively, (xliii) SEQ ID NOs: 51, 159, and 150, respectively, (xliv) SEQ ID NOs: 51, 161, and 150, respectively, (xlv) SEQ ID NOs: 51, 163, and 150, respectively, (xlvi) SEQ ID NOs: 51, 165, and 150, respectively, (xlvii) SEQ ID NOs: 51, 167, and 150, respectively, (xlviii) SEQ ID NOs: 51, 169, and 150, respectively, (xlix) SEQ ID NOs: 52, 141, and 140, respectively, (1) SEQ ID NOs: 52, 146, and 145, respectively, (li) SEQ ID NOs: 52, 151, and 150, respectively, (lii) SEQ ID NOs: 52, 153, and 145, respectively, (liii) SEQ ID NOs: 52, 155, and 145, respectively, (liv) SEQ ID NOs: 52, 157, and 145, respectively, (lv) SEQ ID NOs: 52, 159, and 150, respectively, (lvi) SEQ ID NOs: 52, 161, and 150, respectively, (lvii) SEQ ID NOs: 52, 163, and 150, respectively, (lviii) SEQ ID NOs: 52, 165, and 150, respectively, (lix) SEQ ID NOs: 52, 167, and 150, respectively, (lx) SEQ ID NOs: 52, 169, and 150, respectively, (lxi) SEQ ID NOs: 48, 174, and 173, respectively, (lxii) SEQ ID NOs: 49, 174, and 173, respectively, (lxiii) SEQ ID NOs: 50, 174, and 173, respectively, (lxiv) SEQ ID NOs: 51, 174, and 173, respectively, or (lxv) SEQ ID NOs: 52, 174, and 173, respectively.

In a further embodiment and in accordance with the above, the first monomer comprises the amino acid sequence of SEQ ID NO: 49, the second monomer comprises the amino acid sequence of SEQ ID NO: 153, and the third monomer comprises the amino acid sequence of SEQ ID NO: 145. In some other further embodiments, the first monomer comprises the amino acid sequence of SEQ ID NO: 48, the second monomer comprises the amino acid sequence of SEQ ID NO: 174, and the third monomer comprises the amino acid sequence of SEQ ID NO: 173. In some other further embodiments, the first monomer comprises the amino acid sequence of SEQ ID NO: 49, the second monomer comprises the amino acid sequence of SEQ ID NO: 174, and the third monomer comprises the amino acid sequence of SEQ ID NO: 173. In some other further embodiments, the first monomer comprises the amino acid sequence of SEQ ID NO: 50, the second monomer comprises the amino acid sequence of SEQ ID NO: 174, and the third monomer comprises the amino acid sequence of SEQ ID NO: 173. In some other further embodiments, the first monomer comprises the amino acid sequence of SEQ ID NO: 51, the second monomer comprises the amino acid sequence of SEQ ID NO: 174, and the third monomer comprises the amino acid sequence of SEQ ID NO: 173. In some other further embodiments, the first monomer comprises the amino acid sequence of SEQ ID NO: 52, the second monomer comprises the amino acid sequence of SEQ ID NO: 174, and the third monomer comprises the amino acid sequence of SEQ ID NO: 173

In another aspect, the present disclosure provides a method of treating a cancer in a human subject, comprising administering an IL-12 Fc fusion protein, as described herein, to a human subject in need thereof, wherein the IL-12 Fc fusion protein binds to a target cell.

In a further embodiment and in accordance with the above, the target cell expresses: (1) IL-12Rβ2, and (2) a cell surface antigen selected from the group consisting of: (a) human OX40, (b) human LAG3, (c) human PD1, (d) human ICOS, (e) human 41BB, (f) human CCR7, and (g) human CCR8.

In a further embodiment and in accordance with the above, the cell surface antigen is human PD1, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human LAG3, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human OX40, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human ICOS, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human 41BB, and the target cell comprises: (i) activated and/or exhausted CD4+ T cell(s), (ii) activated and/or exhausted NK cell(s), (iii) activated and/or exhausted CD8+ T cell(s), or (iv) activated and/or exhausted regulatory T cell(s). In other embodiments, the cell surface antigen is human CCR7, and the target cell comprises: (i) naïve CD4+ T cell(s), (ii) naïve CD8+ T cell(s), or (iii) naïve regulatory T cell(s). In other embodiments, the cell surface antigen is human CCR8, and the target cell comprises: (i) activated and/or exhausted CD4+ T cell(s), or (ii) activated and/or exhausted regulatory T cell(s).

In another aspect, the present disclosure provides a method of activating a target cell in a human subject, comprising administering an IL-12 Fc fusion protein, as described herein, to a human subject in need thereof, wherein the IL-12 Fc fusion protein binds to the target cell.

In a further embodiment and in accordance with the above, the target cell expresses: (1) IL-12Rβ2, and (2) a cell surface antigen selected from the group consisting of: (a) human OX40, (b) human LAG3, (c) human PD1, (d) human ICOS, (e) human 41BB, (f) human CCR7, and (g) human CCR8.

In a further embodiment and in accordance with the above, the cell surface antigen is human PD1, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human LAG3, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human OX40, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human ICOS, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human 41BB, and the target cell comprises: (i) activated and/or exhausted CD4+ T cell(s), (ii) activated and/or exhausted NK cell(s), (iii) activated and/or exhausted CD8+ T cell(s), or (iv) activated and/or exhausted regulatory T cell(s). In other embodiments, the cell surface antigen is human CCR7, and the target cell comprises: (i) naïve CD4+ T cell(s), (ii) naïve CD8+ T cell(s), or (iii) naïve regulatory T cell(s). In other embodiments, the cell surface antigen is human CCR8, and the target cell comprises: (i) activated and/or exhausted CD4+ T cell(s), or (ii) activated and/or exhausted regulatory T cell(s).

In another aspect, the present disclosure provides a method of stimulating IL-12 signaling in a target cell comprising administering an IL-12 Fc fusion protein, as described herein, to a human subject in need thereof, wherein the IL-12 Fc fusion protein binds to the target cell.

In a further embodiment and in accordance with the above, the target cell expresses: (1) IL-12Rβ2, and (2) a cell surface antigen selected from the group consisting of: (a) human OX40, (b) human LAG3, (c) human PD1, (d) human ICOS, (e) human 41BB, (f) human CCR7, and (g) human CCR8.

In a further embodiment and in accordance with the above, the cell surface antigen is human PD1, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human LAG3, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human OX40, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human ICOS, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human 41BB, and the target cell comprises: (i) activated and/or exhausted CD4+ T cell(s), (ii) activated and/or exhausted NK cell(s), (iii) activated and/or exhausted CD8+ T cell(s), or (iv) activated and/or exhausted regulatory T cell(s). In other embodiments, the cell surface antigen is human CCR7, and the target cell comprises: (i) naïve CD4+ T cell(s), (ii) naïve CD8+ T cell(s), or (iii) naïve regulatory T cell(s). In other embodiments, the cell surface antigen is human CCR8, and the target cell comprises: (i) activated and/or exhausted CD4+ T cell(s), or (ii) activated and/or exhausted regulatory T cell(s).

In another aspect, the present disclosure provides a method of inducing the production and/or secretion of one or more cytokines in a target cell comprising administering an IL-12 Fc fusion protein, as described herein, to a human subject in need thereof, wherein the IL-12 Fc fusion protein binds to the target cell.

In a further embodiment and in accordance with the above, the target cell expresses: (1) IL-12Rβ2, and (2) a cell surface antigen selected from the group consisting of: (a) human OX40, (b) human LAG3, (c) human PD1, (d) human ICOS, (e) human 41BB, (f) human CCR7, and (g) human CCR8.

In a further embodiment and in accordance with the above, the cell surface antigen is human PD1, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human LAG3, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human OX40, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human ICOS, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human 41BB, and the target cell comprises: (i) activated and/or exhausted CD4+ T cell(s), (ii) activated and/or exhausted NK cell(s), (iii) activated and/or exhausted CD8+ T cell(s), or (iv) activated and/or exhausted regulatory T cell(s). In other embodiments, the cell surface antigen is human CCR7, and the target cell comprises: (i) naïve CD4+ T cell(s), (ii) naïve CD8+ T cell(s), or (iii) naïve regulatory T cell(s). In other embodiments, the cell surface antigen is human CCR8, and the target cell comprises: (i) activated and/or exhausted CD4+ T cell(s), or (ii) activated and/or exhausted regulatory T cell(s).

In another aspect, the present disclosure provides a method of reprogramming and/or altering a differentiation state of a target cell comprising administering an IL-12 Fc fusion protein, as described herein, wherein the IL-12 Fc fusion protein binds to the target cell.

In a further embodiment and in accordance with the above, the target cell expresses: (1) IL-12Rβ2, and (2) a cell surface antigen selected from the group consisting of: (a) human OX40, (b) human LAG3, (c) human PD1, (d) human ICOS, (e) human 41BB, (f) human CCR7, and (g) human CCR8.

In a further embodiment and in accordance with the above, the cell surface antigen is human PD1, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human LAG3, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human OX40, and the target cell comprises: (i) activated and/or exhausted CD8+ T cell(s), (ii) activated and/or exhausted CD4+ T cell(s), (iii) activated and/or exhausted NK cell(s), or (iv) activated and/or exhausted regulatory T cell(s). In other embodiments, the cell surface antigen is human ICOS, and the target cell comprises: (i) activated and/or exhausted CD4+ T cell(s), (ii) activated and/or exhausted CD8+ T cell(s), (iii) activated and/or exhausted NK cell(s), or (iv) activated and/or exhausted regulatory T cell(s). In other embodiments, the cell surface antigen is human 41BB, and the target cell comprises: (i) activated and/or exhausted CD4+ T cell(s), (ii) activated and/or exhausted NK cell(s), (iii) activated and/or exhausted CD8+ T cell(s), or (iv) activated and/or exhausted regulatory T cell(s). In other embodiments, the cell surface antigen is human CCR7, and the target cell comprises: (i) naïve CD4+ T cell(s), (ii) naïve CD8+ T cell(s), or (iii) naïve regulatory T cell(s). In other embodiments, the cell surface antigen is human CCR8, and the target cell comprises: (i) activated and/or exhausted CD4+ T cell(s), or (ii) activated and/or exhausted regulatory T cell(s).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.,” “FIG.,” “Figure,” “Figures,” “FIGS.,” and “FIGS.” herein) of which:

FIG. 1 shows a list of sequences for human, wild-type IL-12 subunits alpha and beta (precursor and mature form sequences).

FIG. 2 shows a list of sequences for human, wild-type IL-12Rβ1 (+ the sequence for the extracellular domain) and IL-12Rβ2 (+ the sequence for the extracellular domain).

FIG. 3 shows a list of sequences for mouse, wild-type IL-12 subunits alpha and beta (precursor and mature form sequences).

FIG. 4 shows a list of sequences for mouse, wild-type IL-12Rβ1 (+ the sequence for the extracellular domain) and IL-12Rβ2 (+ the sequence for the extracellular domain).

FIG. 5 shows a list of variant IL-12 p35 subunit sequences (both human and mouse) comprising one or more substitution mutations.

FIG. 6 shows a list of variant IL-12 p40 subunit sequences.

FIG. 7 shows exemplary sequences for human IgG1 Fc domains (G1m allotype, as well as Fc domains comprising one or more substitutions).

FIG. 8 shows a list of exemplary domain linker sequences.

FIG. 9 shows a list of amino acid sequences for exemplary antigen binding domains (ABDs) for hen egg-white lysozyme, as well as exemplary heavy chain and light chain amino acid sequences (mouse and human) for use in control IL-12 fusion proteins.

FIG. 10 (FIGS. 10A-10D) shows a list of amino acid sequences for exemplary IL-12 Fc fusion protein monomers for use in subject IL-12 Fc fusion proteins.

FIG. 11 (FIGS. 11A and 111B) shows a list of sequences for exemplary antigen binding domains (ABDs) for PD1 (human and mouse), as well as exemplary heavy chain and light chain amino acid sequences for use in a subject IL-12 Fc fusion protein. vhCDR1-3 and vlCDR1-3 sequences underlined for select VH/VL ABD pairs.

FIG. 12 (FIGS. 12A and 12B) shows a list of sequences for exemplary ABDs that bind to LAG3 (human/mouse cross-reactive and human), as well as exemplary heavy chain and light chain amino acid sequences for use in a subject IL-12 Fc fusion protein. vhCDR1-3 and vlCDR1-3 sequences underlined for select VH/VL ABD pairs.

FIG. 13 shows a list of sequences for exemplary ABDs that bind to OX40 (human and mouse), as well as exemplary heavy chain and light chain amino acid sequences for use in a subject IL-12 Fc fusion protein. vhCDR1-3 and vlCDR1-3 sequences underlined for select VH/VL ABD pairs.

FIG. 14 (FIGS. 14A and 14B) shows a list of sequences for exemplary ABDs that bind to 41BB (human/mouse cross-reactive and human), as well as exemplary heavy chain and light chain amino acid sequences for use in a subject IL-12 Fc fusion protein. vhCDR1-3 and vlCDR1-3 sequences underlined for select VH/VL ABD pairs.

FIG. 15 (FIGS. 15A and 15B) shows a list of sequences for exemplary ABDs that bind to ICOS (human/mouse cross-reactive and human), as well as exemplary heavy chain and light chain amino acid sequences for use in a subject IL-12 Fc fusion protein. vhCDR1-3 and vlCDR1-3 sequences underlined for select VH/VL ABD pairs.

FIG. 16 shows a list of sequences for exemplary ABDs that bind to CCR7 (human), as well as exemplary heavy chain and light chain amino acid sequences for use in a subject IL-12 Fc fusion protein.

FIG. 17 (FIGS. 17A and 17B) shows a list of sequences for exemplary ABDs that bind to CCR8 (human and human/mouse cross-reactive), as well as exemplary heavy chain and light chain amino acid sequences for use in subject IL-12 Fc fusion proteins. vhCDR1-3 and vlCDR1-3 sequences underlined for select VH/VL ABD pairs.

FIG. 18 (FIGS. 18A-18H) shows schematic representations of exemplary IL-12-ABD fusion protein formats. In some exemplary configurations, the subject IL-12-ABD fusion protein is configured such that it comprises one antigen binding domain (i.e., a monovalent, monospecific format). In other exemplary configurations, the subject IL-12-ABD fusion protein is configured such that it comprises two antigen binding domains. In such configurations, the subject IL-12-ABD fusion protein may be configured such that the ABD's bind the same target antigen (i.e., a bivalent, monospecific format), whereas in other configurations the subject IL-12-ABD fusion protein is configured such that the first ABD and the second ABD bind distinct target antigens (i.e., a monovalent, bispecific format). It is expressly contemplated that the one or more ABD depicted herein can take on different configurations, as appreciated by those skilled in the art. Suitable ABD formats include, but are not limited to, a Fab format, a scFv format, and a nanobody format (VHH domain). Further, in some exemplary configurations, the subject IL-12-ABD fusion protein is configured such that the first IL-12 subunit domain and the second IL-12 subunit domain are on the same monomer of a subject IL-12 fusion protein (generally, covalently linked to each other using a domain linker), whereas in other exemplary configurations the first IL-12 subunit domain and the second IL-12 subunit domain are on separate monomers comprising a subject IL-12 fusion protein. Further, while the schematics generally show single chain IL-12 arranged, from N-terminus to C-terminus, as (variant) IL-12 p40 subunit domain-domain linker-variant IL-12 p35 subunit domain, it is also contemplated that the IL-12 subunit domains can be arranged, from N-terminus to C-terminus, as variant IL-12 p35 subunit domain-domain linker-(variant) IL-12 p40 subunit domain. As will be appreciated by those skilled in the relevant art, while all exemplary configurations show the first IL-12 subunit domain and the second IL-12 subunit domain covalently linked to one monomer or covalently linked to a first monomer and a second monomer of a subject IL-12-ABD fusion protein, it is also contemplated that a subject IL-12-ABD fusion protein can be configured such that only one of the IL-12 subunit domains (e.g., the variant IL-12 p35 subunit domain, or the (variant) IL-12 p40 subunit domain) is covalently attached to a monomer of the subject IL-12-ABD fusion protein and the other IL-12 subunit domain (e.g., the (variant) IL-12 p40 subunit domain, or the variant IL-12 p35 subunit domain) is expressed as a soluble protein that is not covalently attached to the monomers of the subject IL-12-ABD fusion protein.

FIG. 19 shows a list of exemplary IL-12 Fc fusion proteins, including ABD specificity (human), IL-12 p35 subunit domain mutations, and the amino acid sequence identifiers for such exemplary IL-12 Fc fusion proteins, as well as exemplary control fusion proteins (ABD specificity for hen egg-white lysozyme, or lacking an ABD).

FIG. 20 (FIGS. 20A and 20B) shows reducing (+DTT) and non-reducing (−DTT) SDS-PAGE analysis of exemplary purified IL-12-ABD fusion proteins.

FIG. 21 shows a representative flow cytometry analysis of PD-1 expression on purified naïve human NK cells.

FIG. 22 (FIGS. 22A and 22B) shows AlphaLISA measurements of IFNγ in the medium of naïve NK cells that were incubated with commercially available recombinant IL-12 (rIL-12) or the indicated IL-12-ABD fusion proteins. Symbols and error bars indicate the mean and SD, respectively.

FIG. 23 shows flow cytometry analysis of PD-1 on healthy donor-derived human PBMC that were activated with 600 U/mL IL-2 and 32 ng/mL OKT3.

FIG. 24 (FIGS. 24A and 24B) shows AlphaLISA measurements of IFNγ in the medium of IL-2/OKT3 activated human PBMC that were incubated with commercially available recombinant IL-12 (rIL-12) or the indicated IL-12-ABD fusion proteins, in the presence of 50 U/mL human IL-2. Symbols and error bars indicate the mean and SD, respectively.

FIG. 25 shows selected volcano plots that display differential gene expression (log-transformed fold change (log FC) on the x-axis; negative log-transformed adjusted p-value (-log(adj. p)) on the y-axis) within the listed data sets and comparisons (FIGS. 25A-25I), as well as a summary table (FIG. 25J) with the upregulation rank of each gene within each data set, and the rank of each gene after taking a product-of-ranks across the listed data sets and comparisons.

FIG. 26 shows the percentage of each listed cell type (x-axis) that is positive for each cell surface target across a range of tissues and tumor samples (defined in legend). For RNA/transcript abundance data, a cell was considered positive if at least 1 transcript was detected. For ADT/cell surface antibody labelling, a cell was considered positive if the transformed abundance is above 1 (an arbitrary threshold that was selected to exclude ‘background staining’ across a variety of markers within the associated data set).

FIG. 27 shows the average transcript abundance, as measured by normalized transcript per kilobase million (nTPM), for selected genes (y-axis) across the listed cell type clusters (x-axis) from a range of tissues in the human protein atlas. Individual cell types have been clustered in related groups (at bottom).

FIG. 28 shows the percentage of CCR8-positive cells across different cell types (x-axis) and a range of tissues (see legend) (FIG. 28A); the average transcript abundance, as measured by normalized transcript per kilobase million (nTPM), across the listed cell type clusters (x-axis) from a range of tissues in the human protein atlas, with individual cell types clustered into related groups (at bottom) (FIG. 28B); and the average transcript abundance for the Treg marker FOXP3 (x-axis) and CCR8 (y-axis) for T cell clusters within the human protein atlas (FIG. 28C).

FIG. 29 shows selected volcano plots that display differential gene expression (log-transformed fold change (log FC) on the x-axis; negative log-transformed adjusted p-value (log(adj. p)) on the y-axis) within the listed data sets and comparisons (FIGS. 29A-29F). FIGS. 29A, 29B, and 29C contain comparisons between tumor-resident naïve CD8 T cells and other tumor-resident CD8 T cell subsets. FIGS. 29D, 29E, and 29F contain comparisons between tumor-resident CD8 T cells and CD8 T cells from matched heathy tissue. FIG. 29G shows the normalized transcript abundance for CCR7 within CD8 T cells from the peripheral blood or tumor of colorectal cancer patients, with cells subset by their tissue source and co-expression of TCF7 and/or PDCD1 to identify naïve (TCF7⁺/PDCD1⁻) T cells and putative central memory T cells (T_{scm}; TCF7⁺/PDCD1⁺).

FIG. 30 (FIGS. 30A and 30B) shows a summary of flow cytometry analysis performed on immune cells that were harvested from different organs of MC38-bearing C57/BL6 mice 14 days following IL-12 treatment.

FIG. 31 shows flow cytometry plots analyzing the expression of potential ABD targets on mouse immune cell subsets that were harvested from tumors of MC38-bearing C57/BL6 mice 14 days following IL-12 treatment.

FIG. 32 shows flow cytometry plots analyzing the expression of potential ABD targets on mouse immune cell subsets that were harvested from spleens of MC38-bearing C57/BL6 mice 14 days following IL-12 treatment.

FIG. 33 shows a list of exemplary IL-12 Fc fusion proteins, including ABD specificity (mouse), IL-12 p35 subunit domain mutations, and the amino acid sequence identifiers for such exemplary IL-12 Fc fusion proteins, as well as exemplary control fusion proteins (ABD specificity for hen egg-white lysozyme, or lacking an ABD).

FIG. 34 shows reducing (+DTT) and non-reducing (−DTT) SDS-PAGE analysis of exemplary purified IL-12-ABD fusion proteins.

FIG. 35 shows flow cytometry analysis of purified C57/BL6 mouse T cells that were activated with 1 μg/mL anti-CD3 and 600 U/mL human IL-2.

FIG. 36 (FIGS. 36A and 36B) show AlphaLISA measurements of IFNγ in the medium of anti-CD3/IL-2 activated purified mouse T cells that were incubated with commercially available recombinant mouse IL-12 (rmIL-12) or the indicated IL-12-ABD fusion proteins, in the presence of 50 U/mL human IL-2. Symbols and error bars indicate the mean and SD, respectively.

FIG. 37 shows flow cytometry analysis of LAG-3 on healthy donor-derived human PBMC that were activated with 600 U/mL IL-2 and 32 ng/mL OKT3 and expanded in for 5 days in 600 U/mL IL-2.

FIG. 38 shows AlphaLISA measurements of IFNγ in the medium of activated/expanded human PBMC that were incubated with commercially available recombinant mouse IL-12 (rmIL-12) or the indicated IL-12-ABD fusion proteins, in the presence of 50 U/mL human IL-2. Symbols and error bars indicate the mean and SD, respectively.

FIG. 39 shows a histogram representing CCR8 expression as determined by flow cytometry on HEK-Blue IL-12 reporter cells (untransduced) and CCR8-HEK-Blue IL-12 reporter cells (transduced).

FIG. 40 shows SEAP measurements (represented as absorbance at 650 nm) of CCR8-HEK-Blue IL-12 reporter cells that were incubated with commercially available recombinant IL-12 (rIL-12) or the indicated IL-12 Fc or IL-12-ABD fusion proteins. Symbols and error bars indicate the mean and SD, respectively.

FIG. 41 shows flow cytometry analysis of CCR7 on healthy donor-derived human PBMC that were activated with 600 U/mL IL-2 and 32 ng/mL OKT3.

FIG. 42 shows AlphaLISA measurements of IFNγ in the medium of IL-2/OKT3 activated human PBMC that were incubated with commercially available recombinant IL-12 (rIL-12) or the indicated IL-12-ABD fusion proteins, in the presence of 50 U/mL human IL-2. Symbols and error bars indicate the mean and SD, respectively.

FIG. 43 shows flow cytometry analysis of CCR7 on purified naïve human NK cells.

FIG. 44 shows AlphaLISA measurements of IFNγ in the medium of naïve NK cells that were incubated with commercially available recombinant IL-12 (rIL-12) or the indicated IL-12-ABD fusion proteins. Symbols and error bars indicate the mean and SD, respectively.

FIG. 45 shows the amino acid sequence of full-length, human CCR8 protein.

FIG. 46 shows flow cytometry analysis of OX40 on purified C57/BL6 mouse T cells that were activated with 0.5 ug/mL anti-CD3, 0.5 ug/mL anti-CD28, and 600 U/mL human IL-2.

FIG. 47 shows AlphaLISA measurements of IFNγ in the medium of anti-CD3/anti-CD28/IL-2 activated purified mouse T cells that were incubated with commercially available recombinant mouse IL-12 (rmIL-12) or the indicated IL-12-ABD fusion proteins, in the presence of 50 U/mL human IL-2. Symbols and error bars indicate the mean and SD, respectively.

FIG. 48 shows the growth of MC38 tumor cells following subcutaneous implantation into C57/BL6 mice. Mice were treated on days 0, 7 and 14 with the indicated test articles at a dose that was the molar equivalent to 0.03 mg/kg of IL-12 Fc Y36E unless otherwise indicated. Mice that were euthanized due to ulceration are indicated by an ‘x’.

FIG. 49 (FIGS. 49A-49C) shows measurements of IFNγ in the plasma of MC38-bearing C57/BL6 mice that were treated with either PBS, IL-12 Fc Y36E, or the indicated IL-12 ABD on days 0, 7, and 14. Measurements were performed on plasma that was sampled 3 days after each treatment (days 3 (FIG. 49A), 10 (FIG. 49B), and 17 (FIG. 49C)). Symbols represent measurements for individual mice and horizontal lines indicate the median for each treatment group.

FIG. 50 (FIGS. 50A-50C) shows measurements of IL-12-Fc/IL-12 ABD fusion proteins in the plasma of MC38-bearing C57/BL6 mice that were treated with either PBS, IL-12 Fc Y36E, or the indicated IL-12 ABD on days 0, 7, and 14. Measurements were performed on plasma that was sampled 3 days after each treatment (days 3 (FIG. 50A), 10 (FIG. 50B), and 17 (FIG. 50C)). Symbols represent measurements for individual mice and horizontal lines indicate the median for each treatment group. Data points that were below the detection limit of the assay were assigned an arbitrary value of 1 pg/mL.

FIG. 51 shows the weight of MC38 tumors that were harvested from C57BL/6 mice 5 days after treatment with either PBS, 0.03 mg/kg of wild type (WT) IL-12 Fc or an equimolar dose of the indicated IL-12 ABD fusion proteins. ‘10×’ indicates where mice were treated with a 10-fold higher dose of the indicated test article. Symbols represent measurements for individual mice and horizontal lines indicate the median for each treatment group.

FIG. 52 shows the weight of spleens harvested from MC38 tumor bearing C57BL/6 mice 5 days after treatment with either PBS, 0.03 mg/kg of wild type (WT) IL-12 Fc or an equimolar dose of the indicated IL-12 ABD fusion proteins. ‘10×’ indicates where mice were treated with a 10-fold higher dose of the indicated test article. Symbols represent measurements for individual mice and horizontal lines indicate the median for each treatment group.

FIG. 53 shows the frequency of CD45+ cells in spleens that were harvested from MC38 tumor bearing C57BL/6 mice 5 days after treatment with either PBS, 0.03 mg/kg of wild type (WT) IL-12 Fc or an equimolar dose of the indicated IL-12 ABD fusion proteins (n=6/group). ‘10×’ indicates where mice were treated with a 10-fold higher dose of the indicated test article. Data is plotted in box-and-whisker format, where the whiskers represent the minimum and maximum values in each treatment group.

FIG. 54 (FIGS. 54A-54E) shows quantification of immune cell types (as indicated) in MC38 tumors that were harvested from C57BL/6 mice 5 days after treatment with either PBS, 0.03 mg/kg of wild type (WT) IL-12 Fc or an equimolar dose of the indicated IL-12 ABD fusion proteins (n=6/group). ‘10×’ indicates where mice were treated with a 10-fold higher dose of the indicated test article. Data is plotted in box-and-whisker format, where the whiskers represent the minimum and maximum values in each treatment group.

FIG. 55 shows quantification of IFNγ in plasma, spleen and MC38 tumor tissue of C57BL/6 mice 5 days after treatment with either PBS, 0.03 mg/kg of wild type (WT) IL-12 Fc or an equimolar dose of the indicated IL-12 ABD fusion proteins (n=6/group). ‘10×’ indicates where mice were treated with a 10-fold higher dose of the indicated test article. Symbols represent measurements for individual mice and horizontal lines indicate the median for each treatment group.

FIG. 56 shows the ratio of IFNγ in MC38 tumors relative to spleens of C57BL/6 mice 5 days after treatment with either 0.03 mg/kg of wild type (WT) IL-12 Fc or an equimolar dose of the indicated IL-12 ABD fusion proteins. ‘10×’ indicates where mice were treated with a 10-fold higher dose of the indicated test article. Tumor:Spleen IFNγ ratio was calculated by dividing the mean tumor IFNγ value by the mean spleen IFNγ value (from data depicted in FIG. 55).

FIG. 57 shows the growth of A20 tumor cells following subcutaneous implantation into BALB/C mice. Unless otherwise indicated, mice were treated on days, 0, 7 and 14 with 0.05 mg/kg wild type (WT) IL-12 Fc, or IL-12 ABD fusion protein at a dose that was molar equivalent to 0.03 mg/kg of IL-12 Fc. ‘10×’ indicates where mice were treated with a 10-fold higher dose of the indicated test article (molar equivalent to 0.3 mg/kg of IL-12 Fc). ‘Single’ indicates groups of mice that were treated once (on day 0).

FIG. 58 (FIGS. 58A-58C) shows measurements of IFNγ in the plasma of A20-bearing BALB/C mice that were treated on days, 0, 7 and 14 with 0.05 mg/kg wild type (WT) IL-12 Fc, or IL-12 ABD fusion protein at a dose that was molar equivalent to 0.03 mg/kg of IL-12 Fc. ‘10×’ indicates where mice were treated with a 10-fold higher dose of the indicated test article (molar equivalent to 0.3 mg/kg of IL-12 Fc). ‘Single’ indicates groups of mice that were treated once (on day 0). Plasma IFNγ levels were measured on day 3 (D3; FIG. 58A), day 10 (D10; FIG. 58B) and day 17 (D17; FIG. 58C). Symbols represent measurements for individual mice and horizontal lines indicate the median for each treatment group. The lower limit of quantification (LLoQ) is indicated by a dashed line.

FIG. 59 depicts the welfare scoring system employed for assessing toxicity in mice receiving IL-12 based treatments. In addition to the parameters outlined in FIG. 59, mice could undergo unscheduled euthanasia due to tumor size and/or ulceration of the tumor/skin overlying the tumor.

FIG. 60 shows the growth of MC38 tumor cells following subcutaneous implantation into C57BL/6 mice treated on days, 0, 7 and 14 with PBS or the indicated dosage of IL-12 ABD fusion protein.

FIG. 61 (FIGS. 61A and 61B) shows heatmaps depicting welfare scores for MC38 tumor-bearing C57BL/6 mice that were treated on days, 0, 7 and 14 with PBS or the indicated dosage of IL-12 ABD fusion protein. Individual mice are represented in separate rows. Each ‘block’ is colored according to the welfare score received by each mouse (as indicated in the legend) on that given day. *indicates mice that were euthanized due to treatment related toxicity.

FIG. 62 shows quantification of welfare scores for MC38 tumor-bearing C57BL/6 mice that were treated on days, 0, 7 and 14 with IL-12 ABD fusion protein. Symbols represent mean welfare score (measured by quantification of area-under-the-curve (AUC)), and error bars indicate the standard deviation of the measurements. The mean welfares score for mice that received PBS treatment is indicated by a solid horizontal line.

FIG. 63 (FIGS. 63A-63C) shows measurements of IFNγ in the plasma of MC38-bearing C57BL/6 mice that were treated on days, 0, 7 and 14 with PBS or the indicated IL-12 ABD fusion protein. Numbers in parathesis indicate the dose of each test article in units of mg/kg. Plasma IFNγ levels were measured on day 3 (D3; FIG. 63A), day 10 (D10; FIG. 63B) and day 17 (D17; FIG. 63C). Symbols represent measurements for individual mice and horizontal lines indicate the median for each treatment group.

FIG. 64 shows quantification of IFNγ levels in the plasma of MC38-bearing C57BL/6 mice 3 days after treatment with PBS or IL-12 ABD fusion protein (n=6 per group). Symbols and error bars represent the mean and standard deviation, respectively.

FIG. 65 (FIGS. 65A and 65B) shows the growth of AT3 tumor cells following subcutaneous implantation into C57BL/6 mice that were treated with PBS, anti-PD-1 and/or IL-12-based test articles as indicated.

FIG. 66 (FIGS. 66A and 66B) shows the cumulative welfare score for AT3 tumor-bearing C57BL/6 mice that were treated with PBS, anti-PD-1 and/or IL-12-based test articles as indicated. Solid lines indicate the cumulative welfare score (according to the table in FIG. 59) for individual mice. Horizontal dashed lines represent extrapolation of welfare curves from the point at which mice were euthanized. ‘Avg AUC’ indicates the average area under the curve for each treatment group.

FIG. 67 shows measurements of IFNγ in the plasma of AT3-bearing C57BL/6 mice four days after treatment with PBS, anti-PD-1 and/or IL-12-based test articles as indicated. Numbers in parathesis indicate the dose of each test article in units of mg/kg. Symbols represent measurements for individual mice and horizontal lines indicate the median for each treatment group.

FIG. 68 shows measurement of IFNγ in the plasma of healthy C57BL/6 mice (No tumor) or C57BL/6 mice that were subcutaneously injected with MC-38 tumors (Tumor) after treatment with a single dose of mono-Y36E-OX86 (as indicated in the legend) on day 0. Symbols and error bars represent the mean and standard deviation of measurements from 3 mice per group. The horizontal dashed line indicated the lower limit of quantification (LLoQ) of the assay.

FIG. 69 shows measurement of mono-Y36E-OX86 in the plasma of healthy C57BL/6 mice (No tumor) or C57BL/6 mice that were subcutaneously injected with MC-38 tumors (Tumor) after treatment with a single dose of mono-Y36E-OX86 (as indicated in the legend) on day 0. Symbols and error bars represent the mean and standard deviation of measurements from 3 mice per group. The horizonal dashed line indicated the lower limit of quantification (LLoQ) of the assay.

FIG. 70 shows a summary of pharmacokinetic properties of mono-Y36E-OX86 in C57BL/6 mice that were subcutaneously injected with MC-38 tumors. Pharmacokinetic properties were modelled in PK Solver software using the data shown in FIG. 69.

FIG. 71 shows histograms representing the expression of mouse OX40 (mOX40; top panel), human IL-12Rβ1 (hIL-12Rb1; middle panel) and human IL-12Rβ2 (hIL-12Rb2; bottom panel) on Jurkat NF-κB reporter cells and mOX40/hIL-12R/Jurkat NF-κB reporter cells.

FIG. 72 shows the percentage of mOX40/hIL-12R/Jurkat NF-κB reporter cells that expressed GFP following incubation with anti-OX40 mAb (OX86) or IL-12 ABD fusion proteins in the presence or absence of A20 cells that expressed mouse FcγRI.

FIG. 73 shows flow cytometry analysis of OT-II splenocytes that were activated for three days in the presence of 40 nM OVA³²³⁻³³⁹ peptide and 100 U/mL recombinant human IL-2. FMO (fluorescence minus one; top right panel) indicates staining with all antibodies in the panel (full stain; bottom right panel) except that recognizing OX40 (anti-mouse CD134 BV605, BD Biosciences).

FIG. 74 shows AlphaLISA measurements of IFNγ in the medium of OVA³²³⁻³³⁹ peptide/IL-2 activated OT-II splenocytes that were incubated with commercially available recombinant mouse IL-12 (rmIL-12) or the indicated IL-12-ABD fusion proteins, in the presence of 50 U/mL human IL-2. Symbols and error bars indicate the mean and SD, respectively.

FIG. 75 (FIGS. 75A-75C) shows a list of sequences for exemplary ABDs that bind to human OX40, as well as exemplary heavy chain and light chain amino acid sequences for use in anti-OX40 mAbs or subject IL-12 Fc fusion proteins. vhCDR1-3 and vlCDR1-3 sequences underlined for select VH/VL ABD pairs.

FIG. 76 shows a list of exemplary anti-human OX40 antibodies in traditional IgG1 bivalent format, including amino acid sequence identifiers.

FIG. 77 shows a list of exemplary IL-12 Fc fusion proteins, including ABD specificity (human), IL-12 p35 subunit domain mutations, and the amino acid sequence identifiers for such exemplary IL-12 Fc fusion proteins.

FIG. 78 shows the binding of anti-human OX40 antibodies in traditional IgG1 bivalent format (solid symbols) and OX40-targeted IL-12 Fc fusion proteins (open symbols) to hOX40-Jurkat-NF-κB cells as determined by flow cytometry.

FIG. 79 shows the binding of anti-human OX40 antibodies in traditional IgG1 bivalent format (solid symbols) and OX40-targeted IL-12 Fc fusion proteins (open symbols) to cOX40-Jurkat-NF-κB cells as determined by flow cytometry.

FIG. 80 shows EC50 values for the binding of anti-human OX40 antibodies in traditional IgG1 bivalent format and OX40-targeted IL-12 Fc fusion proteins to hOX40-Jurkat-NF-κB cells and cOX40-Jurkat-NF-κB cells as determined by flow cytometry.

FIG. 81 (FIGS. 81A and 81B) shows a list of sequences for exemplary ABDs that bind to human OX40, as well as exemplary heavy chain and light chain amino acid sequences for use in a subject IL-12 Fc fusion protein. vhCDR1-3 and vlCDR1-3 sequences underlined for select VH/VL ABD pairs.

FIG. 82 shows a list of exemplary IL-12 Fc fusion proteins, including ABD specificity (human), IL-12 p35 subunit domain mutations, and the amino acid sequence identifiers for such exemplary IL-12 Fc fusion proteins.

FIG. 83 shows the binding of OX40-targeted IL-12 Fc fusion proteins to hOX40-Jurkat-NF-κB cells as determined by flow cytometry.

FIG. 84 shows EC50 values for the binding of OX40-targeted IL-12 Fc fusion proteins to hOX40-Jurkat-NF-κB cells as determined by flow cytometry.

FIG. 85 (FIGS. 85A-85C) shows a list of sequences for exemplary ABDs that bind to human OX40, as well as exemplary heavy chain and light chain amino acid sequences for use in a subject IL-12 Fc fusion protein. vhCDR1-3 and vlCDR1-3 sequences underlined for select VH/VL ABD pairs.

FIG. 86 shows a list of exemplary IL-12 Fc fusion proteins, including ABD specificity, IL-12 p35 subunit domain mutations, and the amino acid sequence identifiers for such exemplary IL-12 Fc fusion proteins.

FIG. 87 shows the binding of an anti-human OX40 antibodies in traditional IgG1 bivalent format and OX40-targeted IL-12 Fc fusion proteins to hOX40-Jurkat-NF-κB cells as determined by flow cytometry.

FIG. 88 shows EC50 values for the binding of an anti-human OX40 antibody in traditional IgG1 bivalent format and OX40-targeted IL-12 Fc fusion proteins to hOX40-Jurkat-NF-κB cells as determined by flow cytometry.

FIG. 89 shows binding of OX40L-Fc to OX40 positive hOX40-Jurkat-NF-κB cells in the presence of anti-human OX40 antibodies in traditional IgG1 bivalent format or OX40-targeted IL-12 Fc fusion proteins.

FIG. 90 (FIGS. 90A and 90B) shows expression of OX40 (on the left y-axis) and the percentage of GFP⁺ hOX40-Jurkat-NF-κB cells (on the right y-axis) following incubation with 10 nM OX40L-Fc and/or various concentrations of the indicated IL-12 ABD fusion protein for either 1 hour (1 h) or overnight (o/n). GFP measurements were only performed following overnight incubation. Horizonal dashed lines indicate the percentage of GFP⁺ cells following incubation with OX40L-Fc alone. Horizonal dotted lines indicate the OX40 MFI following overnight incubation with OX40L-Fc alone.

FIG. 91 (FIGS. 91A and 91B) shows expression of OX40 (on the left y-axis) and the percentage of GFP⁺ hOX40-Jurkat-NF-κB cells (on the right y-axis) following incubation with 10 nM OX40L-Fc and/or various concentrations of the indicated IL-12 ABD fusion protein for either 1 hour (1 h) or overnight (o/n). GFP measurements were only performed following overnight incubation. Only cells that expressed high levels of OX40 were included in the analysis. Horizonal dashed lines indicate the percentage of GFP⁺ cells following incubation with OX40L-Fc alone.

FIG. 92 (FIGS. 92A and 92B) shows flow cytometry analysis of PBMC derived from two healthy donors (B113 (FIG. 92A) and B121 (FIG. 92B)) that were activated with 600 U/mL IL-2 and 32 ng/mL OKT3. OX40 positive cells are indicated by horizontal lines.

FIG. 93 shows AlphaLISA measurements of IFNγ in the medium of IL-2/OKT3 activated human PBMC that were incubated with wild type IL-12 Fc (WT IL-12 Fc) or the indicated IL-12-ABD fusion proteins, in the presence of 50 U/mL human IL-2. Data is shown for two PBMC donors (B113 (top panel) and B121 (bottom panel)). Symbols and error bars indicate the mean and SD, respectively.

FIG. 94 shows a graph of EC50 values for wild type IL-12 Fc (WT IL-12 Fc) and various IL-12-ABD fusion proteins determined from a PBMC IFNγ release assay. Symbols represent individual measurements and horizontal lines indicate the median value for each treatment group.

FIG. 95 shows a summary of EC50 values for wild type IL-12 Fc (WT IL-12 Fc) and various IL-12-ABD fusion proteins determined by PBMC IFNγ release assays.

FIG. 96 (FIGS. 96A-96C) shows AlphaLISA measurements of IFNγ in the medium of purified CD4+ T cells (top row), CD8+ T cells (middle row), and NK cells (bottom row) from 3 different PBMC donors (B130 (FIG. 96A), B134 (FIG. 96B), and B177 (FIG. 96C)) that were either freshly isolated (D0, left column), activated for 2 days (D2, middle column), or maintained in culture for 5 days post activation (D7, right column) and treated with WT IL-12 Fc or the indicated IL-12 ABD fusion protein in the presence of 50 U/mL IL2 (for T cells) and 1 ng/mL IL-15 (for NK cells). Symbols and error bars indicate the mean and SD, respectively.

FIG. 97 shows the expression of OX40 (left column for each panel), IL-12Rβ1 (middle column for each panel), and IL-12Rβ2 (right column for each panel) on purified CD4+ T cells, CD8+ T cells and NK cells from 3 different PBMC donors (B130 (top left panel), B134 (top right panel), and B177 (bottom panel)) that were either freshly isolated (DO; top row for each panel), activated for 2 days (D2; middle row for each panel), or maintained in culture for 5 days post activation (D7; bottom row for each panel).

FIG. 98 (FIGS. 98A and 98B) shows expression of IL-12Rβ2 (FIG. 98A) and OX40 (FIG. 98B) on purified NK cells (left column), CD4+ T cells (middle column) and CD8+ T cells (right column) that were either freshly isolated (DO, top row), activated for 2 days (D2, middle row) or maintained in culture for 5 days post activation (D7, bottom row).

FIG. 99 shows AlphaLISA measurements of IFNγ in the medium of purified CD4+ T cells (CD4, top row), CD8+ T cells (CD8, middle row) and NK cells (NK, bottom row) that were either freshly isolated (D0, left column), activated for 2 days (D2, middle column) or maintained in culture for 5 days post activation (D7, right column) and treated with WT IL-12 Fc or the indicated IL-12 ABD fusion protein in the presence of 50 U/mL IL2 (for T cells) and 1 ng/mL IL-15 (for NK cells). Symbols and error bars indicate the mean and SD, respectively.

FIG. 100 shows the maximum amount of IFNγ produced by purified CD4+ T cells, CD8+ T cells and NK cells (as indicated) in response to treatment with WT IL-12 Fc (as depicted in FIG. 99). Cells were either freshly isolated (DO), activated for 2 days (D2) or maintained in culture for 5 days post activation (D7). IFNγ measurements for CD4+ T cells and CD8+ T cells on DO were below the lower limit of quantification of the assay and were assigned an arbitrary value of 0 μg/ml.

FIG. 101 shows AlphaLISA measurements of IFNγ in the medium of freshly isolated purified CD4+ T cells that were treated with WT IL-12 Fc or the indicated IL-12 ABD fusion protein in the absence of IL-15. Symbols and error bars indicate the mean and SD, respectively. The horizontal dotted line indicated the lower limit of quantification of the assay.

FIG. 102 shows the phenotype of conventional CD4⁺ cells (ConCD4; top row and third row) that had been maintained in culture for 7 days post CD3/28 activation, and induced Treg cells (Treg; second row and fourth row) that had been activated/differentiated for 10 days in the presence of CD3/28 Dynabeads, 500 U/mL of human IL-2, 5 ng/mL TGF-β and 10 nM retinoic acid.

FIG. 103 shows AlphaLISA measurements of IFNγ in the medium of conventional CD4+ T cells (CD4⁺_{conv}; top left panel and bottom left panel) and induced Treg cells (Treg; top right panel and bottom right panel) from 2 different PBMC donors (B363 (top left panel and bottom left panel) and B364 (top right panel and bottom right panel)) that were activated with CD3/28 Dynabeads and maintained in culture for 10 days in the absence or presence of 5 ng/mL TGF-β and 10 nM retinoic acid, respectively. Symbols and error bars indicate the mean and SD of 2 replicates, respectively.

FIG. 104 shows a summary of EC50 values and fold change (relative to WT IL-12 Fc) for wild type IL-12 Fc (WT IL-12 Fc) and various IL-12-ABD fusion proteins determined by IFNγ release assays performed in conventional CD4+ T cells (CD4⁺_{conv}) and induced Treg cells (Treg) from 2 different PBMC donors (B363 and B364).

FIG. 105 shows the percentage of OX40⁺/CD4⁺ cells (top left panel and bottom left panel) and OX40⁻/CD4⁺ cells (top right panel and bottom right panel) derived from two independent PBMC donors (B130 (top left panel and top right panel) and B323 (bottom left panel and bottom right panel)) that were positive for phosphorylated STAT4 (pStat4⁺) after a 30-minute incubation with wild type IL-12 Fc (WT IL-12 Fc) or IL-12-ABD fusion proteins.

FIG. 106 shows flow cytometry analysis of purified CD4⁺ T cells (containing both conventional CD4⁺ T cells (ConCD4) and Treg cells, as depicted in the left panel) that were activated for 2 days with CD3/28 Dynabeads and 100 U/mL IL-2 and maintained in culture for 5 days post activation. The middle and right panels show OX40 expression on conventional CD4⁺ T cells (ConCD4) and Treg cells, respectively.

FIG. 107 shows the percentage of OX40⁺/Treg cells (top left panel), OX40⁻/Treg cells (bottom left panel), OX40⁺/conventional CD4 cells (top right panel) and OX40⁻/conventional CD4 cells (bottom right panel) that were positive for phosphorylated STAT4 (pStat4⁺) after a 30-minute incubation with wild type IL-12 Fc (WT IL-12 Fc) or IL-12-ABD fusion proteins.

FIG. 108 shows histograms representing the expression of human OX40 (top panel), human IL-12Rβ1 (middle panel), and human IL-12Rβ2 (bottom panel) on Jurkat NF-κB reporter cells (parental Jurkat) and hOX40/hIL-12R/Jurkat NF-κB reporter cells (hIL-12Rβ1/hIL-12Rb2/hOX40. Jurkat).

FIG. 109 (FIGS. 109A and 109B) shows the percentage of GFP positive hOX40/hIL-12R/Jurkat NF-κB reporter cells following overnight incubation with anti-human OX40 antibodies in traditional IgG1 bivalent format and OX40-targeted IL-12 Fc fusion proteins in the presence (FIG. 109A) or absence (FIG. 109B) of A20 cells that expressed human FcγRI (A20-FcγRI). Data were normalized such that unstimulated cells were attributed a value of 0% GFP positive.

FIG. 110 shows AlphaLISA measurements of IFNγ in the medium of activated PBMC that were stimulated with plate bound anti-CD3 and plate bound anti-human OX40 in traditional IgG1 bivalent format (hG1-BGB-A445 (PB)), or plate bound anti-CD3 and either WT IL-12 Fc or IL-12 ABD fusion proteins. Prior to stimulation, genes encoding IL-12Rβ2 (top right panel), STAT4 (bottom left panel), or OX40 (bottom right panel) were knocked out using CRISPR/Cas9 genome editing, as indicated. AAVS1 (top left panel) indicates CRISPR/Cas9 edited cells whereby the targeting ribonucleoproteins were specific for a gene desert (used as a negative control). Symbols and error bars indicate the mean and SD, respectively.

FIG. 111 shows the growth of MC38 tumor cells following subcutaneous implantation into naïve C57BL/6 mice (treatment naïve; top panel) and C57BL/6 mice that had previously been inoculated with MC38 cells and treated with a single dose of 0.143 mg/kg mono-Y36E-OX86 (previously treated; bottom panel). Solid lines indicate tumor growth in individual mice (n=5 per group).

FIG. 112 shows a list of sequences for exemplary ABDs that bind to human OX40, as well as exemplary heavy chain and light chain amino acid sequences for use in a subject IL-12 Fc fusion protein. vhCDR1-3 and vlCDR1-3 sequences are underlined for select VH/VL ABD pairs.

FIG. 113 shows the binding of anti-human OX40 antibodies in traditional IgG1 bivalent format to hOX40-Jurkat-NF-κB cells as determined by flow cytometry.

FIG. 114 shows the binding of anti-human OX40 antibodies in traditional IgG1 bivalent format to cOX40-Jurkat-NF-κB cells as determined by flow cytometry.

FIG. 115 shows EC50 values for the binding of an anti-human OX40 antibody in traditional IgG1 bivalent format and OX40-targeted IL-12 Fc fusion proteins to hOX40-Jurkat-NF-κB cells as determined by flow cytometry.

FIG. 116 shows binding of OX40L-Fc to OX40 positive hOX40-Jurkat-NF-κB cells in the presence of anti-human OX40 antibodies in traditional IgG1 bivalent format.

FIG. 117 shows expression of OX40 (on the left y-axis) and the percentage of GFP+ hOX40-Jurkat-NF-κB cells (on the right y-axis) following incubation with 10 nM OX40L-Fc and/or various concentrations of the indicated anti-OX40 antibodies for either 10 minutes (10 min) or overnight (o/n). GFP measurements were only performed following overnight incubation. Horizonal dashed lines indicate the percentage of GFP+ cells following incubation with OX40L-Fc alone. Horizonal dotted lines indicate the OX40 MFI following overnight incubation with OX40L-Fc alone.

FIG. 118 (FIG. 118A and FIG. 118B) shows the percentage of GFP positive hOX40-Jurkat NF-κB reporter cells following overnight incubation with anti-human OX40 antibodies in traditional IgG1 bivalent format in the presence (FIG. 118A) or absence (FIG. 118B) of A20 cells that expressed human FcγRI (A20-FcγRI). Data were normalized such that unstimulated cells were attributed a value of 0% GFP positive.

### DETAILED DESCRIPTION OF THE INVENTION

The description is presented to enable one of ordinary skill in the art to make and use the invention and is provided in the context of a patent application and its requirements. The section headings used herein are for organization purposes only and are not to be construed as limiting the subject matter described. While various embodiments of the invention(s) of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the invention(s) described herein may be employed in practicing any one of the inventions(s) set forth herein.

All patents, published patent applications, other publications, sequences from GenBank, sequences from UniProt (including, but not limited to, UniProt accession numbers: P43431, P43432, Q60837, and P97378) and other databases referred to herein are incorporated by reference in their entirety respective to the related technology.

### I. Definitions

Unless defined otherwise, technical, and scientific terms used herein have the same meaning as commonly understood by one of ordinary skill in the art to which the present disclosure belongs. For purposes of the present disclosure, the following terms are defined below. The definitions provided are intended to apply to a given term, as well as other derivative linguistic re-phrasings and grammatical equivalents of the term.

As used herein, the term “protein” refers to at least two, three, four, five, six, seven, eight, nine, 10, 11, 12, 13, 14, 15, 16, 17, 18, 19, 20, 30, 40, 50, 60, 70, 80, 90, 100 or more covalently attached amino acids, which includes proteins, polypeptides, oligopeptides, and peptides. When a “biologically functional molecule” comprises two or more proteins, such as, for example, IL-12 (which comprises an IL-12 p35 polypeptide and an IL-12 p40 polypeptide), the individual proteins comprising the two or more proteins may each be referred to as a “subunit,” “monomer,” or “domain,” and the biologically functional molecule may be referred to as a “complex.” In some embodiments, the two or more proteins of a functional complex are non-covalently attached. In some embodiments, the two or more proteins of a functional complex are covalently attached, such as, for example, through a disulfide bond.

As used herein, the term “cytokine” refers to a broad category of proteins, such as, for example, chemokines, interferons, interleukins, lymphokines, tumor necrosis factors, and the like, that are secreted by a first cell to cause an effect on one or more cells through a binding of the secreted cytokine to a receptor on the one or more cells. Cytokines may be involved in autocrine, paracrine, juxtacrine, and/or endocrine signaling.

As used herein, the term “immunocytokine” generally refers to a fusion protein comprising a cytokine (e.g., IL-12) and at least one antibody domain (e.g., a variable heavy domain, a variable light domain, a constant heavy domain, a constant light domain, a VHH domain, and the like).

As used herein, the term “wild-type” refers to an amino acid sequence or a nucleotide sequence that is found in nature, including allelic variations. A wild-type protein has an amino acid sequence (or a nucleotide sequence encoding the amino acid sequence) that has not been intentionally modified.

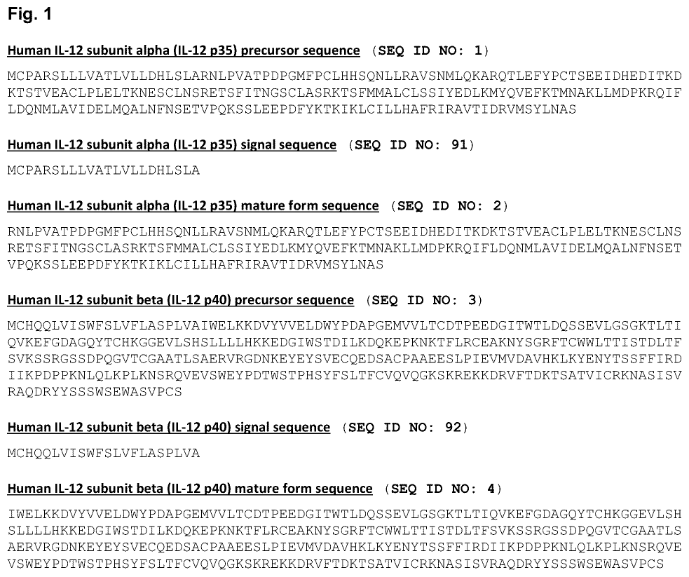

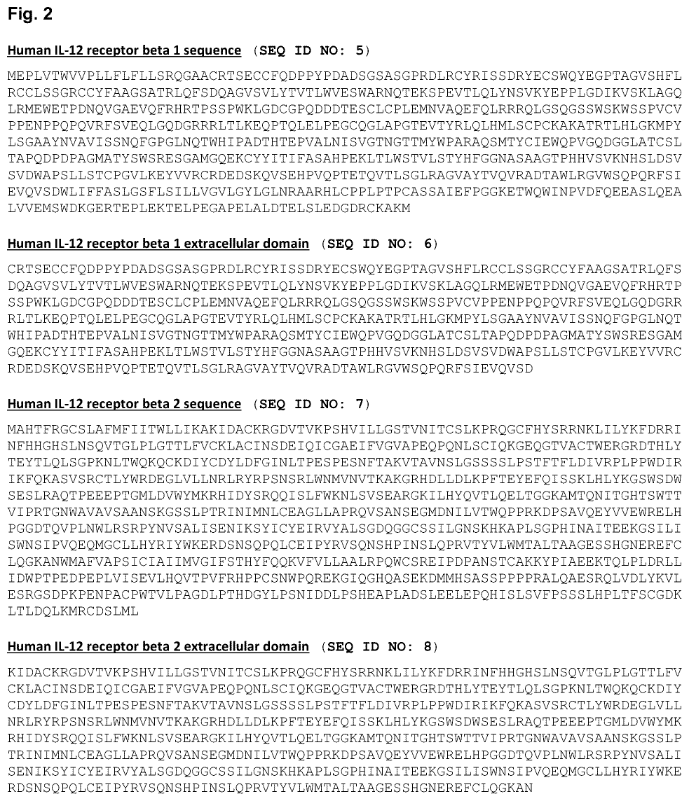

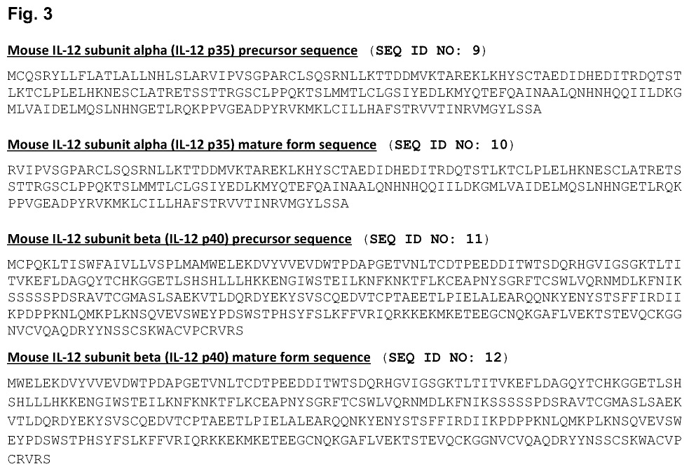

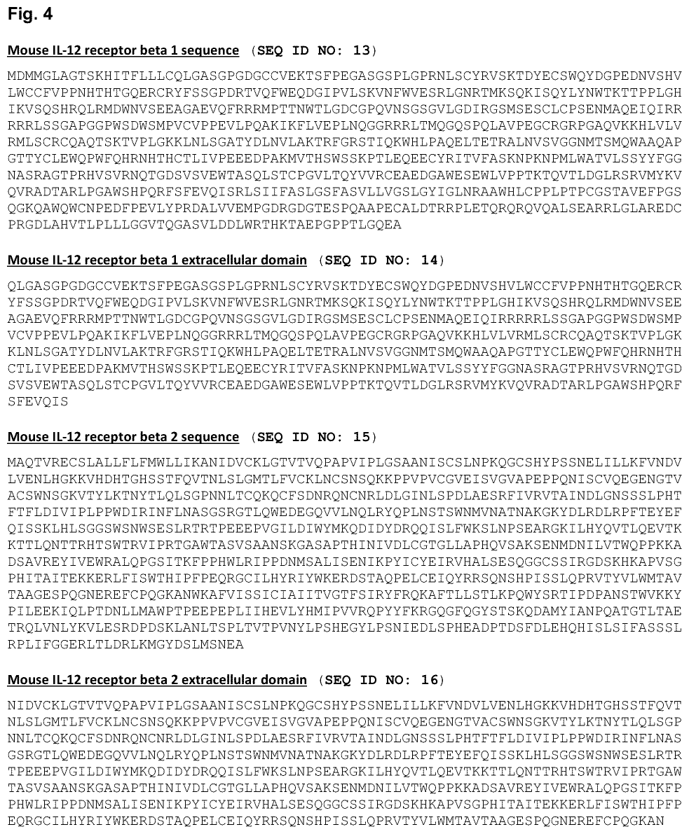

As used herein, the term “IL-12 p35 subunit” refers to the human wild-type IL-12 p35 polypeptide, whether native or recombinant, unless the context clearly dictates otherwise. As such, an IL-12 p35 subunit refers to recombinantly produced IL-12 p35 polypeptide, synthetically produced IL-12 p35 polypeptide, as well as IL-12 p35 extracted from cells or tissues. An amino acid sequence of human wild-type IL-12 p35 subunit is depicted in FIG. 1 (Precursor: SEQ ID NO: 1; Mature: SEQ ID NO: 2). An amino acid sequence of mouse wild-type IL-12 p35 subunit is depicted in FIG. 3 (Precursor: SEQ ID NO: 9; Mature: SEQ ID NO: 10). In the context of a fusion protein, the IL-12 p35 subunit may also be referred to as an “IL-12 p35 subunit domain,” wherein the IL-12 p35 subunit domain comprises at least part of the amino acid sequence encoding the IL-12 p35 subunit. In many of these instances, the IL-12 p35 subunit domain comprises one or more mutations to the IL-12 p35 subunit “parent protein.” In such instances, the IL-12 p35 subunit domain is referred to herein as a “variant IL-12 p35 subunit domain.”

As used herein, the term “IL-12 p40 subunit” refers to the human wild-type IL-12 p40 polypeptide, whether native or recombinant, unless the context clearly dictates otherwise. As such, an IL-12 p40 subunit refers to recombinantly produced IL-12 p40 polypeptide, synthetically produced IL-12 p40 polypeptide, as well as IL-12 p40 extracted from cells or tissues. An amino acid sequence of human wild-type IL-12 p40 subunit is depicted in FIG. 1 (Precursor: SEQ ID NO: 3; Mature: SEQ ID NO: 4). An amino acid sequence of mouse wild-type IL-12 p40 subunit is depicted in FIG. 3 (Precursor: SEQ ID NO: 11; Mature: SEQ ID NO: 12). In the context of a fusion protein, the IL-12 p40 subunit may also be referred to as an “IL-12 p40 subunit domain,” wherein the IL-12 p40 subunit domain comprises at least part of the amino acid sequence encoding the IL-12 p40 subunit. In some of these instances, the IL-12 p40 subunit domain comprises one or more mutations to the IL-12 p40 subunit “parent sequence.” In such instances, the IL-12 p40 subunit domain is referred to herein as a “variant IL-12 p40 subunit domain.” For the sake of brevity, as used herein, the phrase “(variant) IL-12 p40 subunit” is used to disclose embodiments wherein the IL-12 p40 subunit (or, in the case of fusion proteins, IL-12 p40 subunit domain) may comprise a wildtype IL-12 p40 polypeptide or a variant IL-12 p40 subunit (domain) as if each embodiment had been set forth individually.

As used herein, the term “single-chain” refers to a molecule comprising two or more protein domains linearly linked by peptide bonds. In some embodiments, the biologically functional IL-12 is a single chain IL-12 complex (sc-IL-12) (i.e., the IL-12 p35 subunit (or IL-12 p35 subunit domain) and the IL-12 p40 subunit (or IL-12 p40 subunit domain) are fused to form a single peptide chain). In further embodiments, the C-terminus of the IL-12 p35 subunit (domain) is connected to the N-terminus of the IL-12 p40 subunit (domain) (sc-IL-12(p35/p40)). In yet further embodiments, the sc-IL-12(p35/p40) further comprises a “linker,” wherein the C-terminus of the IL-12 p35 subunit is linked to the N-terminus of the linker and the C-terminus of the linker is linked to the N-terminus of the IL-12 p40 subunit. In other embodiments, the C-terminus of the IL-12 p40 subunit is connected to the N-terminus of the IL-12 p35 subunit (sc-IL-12(p40/p35)). In yet further embodiments, the sc-IL-12(p40/p35) further comprises a linker, wherein the C-terminus of the IL-12 p40 subunit is linked to the N-terminus of the linker and the C-terminus of the linker is linked to the N-terminus of the IL-12 p35 subunit. In many of these embodiments, the IL-12 p35 subunit (domain) comprises a variant IL-12 p35 subunit (domain). Further, in some of these embodiments, the IL-12 p40 subunit (domain) comprises a variant IL-12 p40 subunit (domain).

As used herein, the terms “linker” and “domain linker,” which can be used interchangeably, generally refer to a structure (such as, for example, a polymer (e.g., any one of the amino acid sequences comprising SEQ ID NOs: 31-40)) or a bond (such as, for example, a covalent bond) that “links” a first domain to a second domain (such as, for example, a CH1 domain and a Fc domain, a VH domain and a VL domain, a VL domain and a CL domain, a subunit of a cytokine (e.g., the p35 subunit domain of IL-12, the p40 subunit domain of IL-12, etc.) and a subunit of an antibody (e.g., a VH domain, a VL domain, a CH1 domain, a Fc domain, etc.), a first subunit of a cytokine and a second subunit of the cytokine, and the like). Such a linkage may be “direct” (i.e., in instances where the domain linker comprises a covalent bond) or “indirect” (i.e., in instances where the domain linker comprises a structure, such as, for example, any one of the amino acid sequences of SEQ ID NOs: 31-40). In many embodiments, a plurality of domain linkers is incorporated into a subject “low potency IL-12 Fc fusion proteins,” as is described in further detail herein. In some instances, a subject low potency IL-12 Fc fusion protein may comprise one or more domain linkers. In some further instances, the one or more domain linkers comprise one or more of the amino acid sequences of SEQ ID NOs: 31-40, and may optionally comprise one or more covalent bonds. In some other further instances, the one or more domain linkers comprise one or more covalent bonds, and may optionally comprise one or more of the amino acid sequences of SEQ ID NOs: 31-40. In some instances, at least one domain linker comprises a hinge region of a Fc domain.

As used herein, the term “residue” refers to a position in a protein and its associated amino acid identity. For example, Cysteine 252 (also referred to as Cys252 or C252) is a residue at position 252.

As used herein, the term “parent protein” refers to a “reference” protein, the amino acid sequence that encodes the reference protein, or the DNA sequence that encodes the amino acid sequence that encodes the reference protein. In some embodiments, the reference protein comprises a wild-type protein, the amino acid sequence encoding the wild-type protein, and/or the nucleic acid sequence encoding the amino acid sequence encoding the wild-type protein. In some embodiments, the reference protein comprises a human wild-type protein, the amino acid sequence encoding the human wild-type protein, and/or the nucleic acid sequence encoding the amino acid sequence encoding the human wild-type protein. In some embodiments, the reference protein comprises a (human) wild-type protein conjugated to an Fc domain and/or an antigen binding domain (such as, for example, an ABD comprising a VH/VL domain pair, wherein the VH domain comprises the amino acid sequence of SEQ ID NO: 41 and the VL domain comprises the amino acid sequence of SEQ ID NO: 42).

As used herein, the terms “variant protein,” “protein variant,” or “variant” refer to a protein that differs from that of a parent protein by virtue of at least one, two, three, four, five, six, seven, eight, nine, 10, 11, 12, 13, 14, 15, 16, 17, 18, 19, 20, 25, 30, 40, 50, 60, 70, 80, 90, 100 or more modifications. The terms may refer to the protein itself, a composition comprising the protein, the amino acid sequence that encodes it, or the DNA sequence that encodes it. In some embodiments, the parent protein refers to a wild-type sequence. In some embodiments, the parent protein refers to a human wild-type sequence. Thus, a “variant” of an IL-12 p35 subunit (or “variant” of an IL-12 p35 subunit domain) refers to a polypeptide in which one, two, three, four, five, six, seven, eight, nine, 10, 11, 12, 13, 14, 15, 16, 17, 18, 19, 20, 25, 30, 40, 50, 60, 70, 80, 90, 100 or more amino acid substitutions, deletions, and/or insertions are present as compared to the amino acid sequence of a reference IL-12 p35 subunit, such as, for example, a (human) wild-type IL-12 p35 subunit. Further, a “variant” of an IL-12 p40 subunit (or “variant” of an IL-12 p40 subunit domain) refers to a polypeptide in which one, two, three, four, five, six, seven, eight, nine, 10, 11, 12, 13, 14, 15, 16, 17, 18, 19, 20, 25, 30, 40, 50, 60, 70, 80, 90, 100 or more amino acid substitutions, deletions, and/or insertions are present as compared to the amino acid sequence of a reference IL-12 p40 subunit, such as, for example, a (human) wild-type IL-12 p40 subunit.

As used herein, the term “modification” refers to an amino acid substitution, insertion, and/or deletion in a polypeptide sequence or an alteration to a moiety chemically linked to a protein. As used herein, the term “amino acid modification” refers to an amino acid substitution, insertion, and/or deletion in a polypeptide sequence. The position(s) where the amino acid(s) are modified and the number of amino acid(s) that may be modified in the amino acid sequence are not particularly limited.

As will be appreciated by those skilled in the relevant art, the modifications to the IL-12 p35 subunit (domain) described throughout refer to modifications made to the mature form of the sequence (SEQ ID NO: 2) and/or variants thereof, as opposed to the precursor sequence (SEQ ID NO: 1). The precursor sequence for the IL-12 p35 subunit comprises an additional 22 amino acid residues on the N-terminus comprising the sequence: MCPARSLLLVATLVLLDHLSLA (SEQ ID NO: 91). However, the listed modifications to the mature form of the IL-12 p35 subunit can be made to the precursor form of the IL-12 p35 subunit after correcting the position of the stated modification in view of the additional leading amino acid residues in the precursor sequence. For example, the substitution modification Y40A is one of many potential modifications to the IL-12 p35 subunit mature sequence but can also refer to a substitution modification of the IL-12 p35 subunit precursor sequence comprising Y62A.

Similarly, modifications to the IL-12 p40 subunit (domain) described throughout are intended to refer to modifications made to the mature form of the sequence (SEQ ID NO: 4) and/or variants thereof, as opposed to the precursor sequence (SEQ ID NO: 3). The precursor sequence for the IL-12 p40 subunit comprises an additional 22 amino acid residues on the N-terminus comprising the sequence: MCHQQLVISWFSLVFLASPLVA (SEQ ID NO: 92).

However, it will also be appreciated that the listed modifications to the mature form of the IL-12 p40 subunit can be made to the precursor form of the IL-12 p40 subunit after correcting the position of the stated modification in view of the additional leading amino acid residues in the precursor sequence. For example, the substitution modification C177S is disclosed herein as a potential modification to the IL-12 p40 subunit mature sequence but can also refer to a substitution modification of the IL-12 p40 subunit precursor sequence comprising C199S.

As used herein, the terms “amino acid substitution” or “substitution” refer to the replacement of an amino acid at a particular position in a parent polypeptide sequence with a different amino acid. For example, Y40A designates a substitution of tyrosine at position 40 with an alanine at the same position. In some embodiments, the substitution is to an amino acid that is not naturally occurring at the particular position, either not naturally occurring within the organism or in any organism. For clarity, a protein that has been engineered to change the nucleic acid coding sequence but not to change the resulting amino acid (for example, exchanging CCU (encoding proline) to CCC (still encoding proline)) is not an “amino acid substitution.” Phrased differently, despite the creation of a new gene encoding the same protein, if the protein has the same amino acid at the particular position that it started with, it is not an amino acid substitution.

As used herein, the terms “amino acid insertion” or “insertion” refer to the addition of an amino acid residue or sequence at a particular position in a parent polypeptide sequence. For example, −40A designates an insertion of alanine after position 40 and before position 41. As a separate example, D8EPKSS or −8EPKSS designates an insertion of the sequence Glu-Pro-Lys-Ser-Ser after position 8 and before position 9.

As used herein, the terms “amino acid deletion” or “deletion” refer to the removal of an amino acid or sequence at a particular position in a parent polypeptide sequence. For example, Y40−, Y40 #, Y40( ), or Y40del designates a deletion of tyrosine at position 40. As a separate example, EPKSS8−, EPKSS8 #, EPKSS8del designates a deletion of the sequence Glu-Pro-Lys-Ser-Ser that begins at position 8.

As used herein, the terms “non-naturally occurring protein” or “non-naturally occurring protein variant” refer to a variant protein that differs from that of a parent protein by virtue of at least one, two, three, four, five, six, seven, eight, nine, 10, 11, 12, 13, 14, 15, 16, 17, 18, 19, 20, 25, 30, 40, 50, 60, 70, 80, 90, 100 or more modifications that are not isotypic. For example, because the IL-12 p35 subunit of IL-12 does not comprise an alanine at position 40, the substitution Y40A is considered a non-naturally occurring IL-12 p35 variant (or, more generally, a non-naturally occurring IL-12 variant). Modifications to a protein that are not isotypic can be referred to as “non-naturally occurring modifications.”

As used herein, the terms “percent (%) identity” and “percent (%) sequence identity,” when used in the context of two or more proteins or nucleic acids, refer to a percentage of amino acid residues (or nucleic acids encoding the amino acid residues) in a candidate sequence that are identical with the amino acid residues (or nucleic acids encoding the amino acid residues) in a specific sequence, such as, for example, the amino acid sequence of a parent protein, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill of the art, such as, for example, using publicly available computer software (e.g., BLAST, BLAST-2, ALIGN, or Megalign (DNASTAR) software).

Those skilled in the art can determine appropriate parameters for measuring alignment, including any algorithms needed to achieve maximal alignment over the full length of the sequences being compared. In some embodiments, two or more amino acid sequences are at least about 50%, 60%, 70%, 80%, 90%, 91%, 92%, 93%, 94%, 95%, 96%, 97%, 98%, 99%, or even 100% identical.

As used herein, the term “half-life” of an agent can refer to the time it takes for the agent to lose half of its pharmacologic, physiologic, or other activity, relative to such activity at the time of administration into the serum or tissue of an organism or subject, or relative to any other defined time-point. “Half-life” can also refer to the time it takes for the amount or concentration of an agent to be reduced by half of a starting amount administered into the serum or tissue of an organism or subject, relative to such amount or concentration at the time of administration into the serum or tissue of an organism or subject, or to any other defined time-point. The half-life can be measured in serum and/or any one or more selected tissues.

As used herein, the terms “host cell” and “recombinant cell” refer to an individual cell or a cell culture that can be or has been a recipient of any recombinant vector(s) or isolated polynucleotide(s). A host cell can be a transfected, transformed, transduced, or infected cell of any origin, including, but not limited to, prokaryotic, eukaryotic, mammalian, avian, insect, plant, or bacteria cells, or it can be of any origin that can be used to propagate a nucleic acid described herein. A host cell includes cells transfected or infected in vivo or in vitro with a recombinant vector or a polynucleotide of the invention. A host cell that comprises a recombinant vector of the invention may be called a “recombinant host cell.”

Host cells can include, without limitation, the cells of mammals, plants, insects, fungi, and bacteria. Bacterial cells include, without limitation, the cells of Gram-positive bacteria, such as, for example, species of the genus Bacillus, Streptomyces, and Staphylococcus, and cells of Gram-negative bacteria, such as, for example, cells of the genus Escherichia and Pseudomonas. Fungal cells can include, without limitation, yeast cells, such as, for example, Saccharomyces, Pichia pastoris, and Hansenula polymorpha. Insect cells can include, without limitation, cells of Drosophila and Sf9 cells. Plant cells include, without limitations, cells from crop plants, medicinal or ornamental plants or bulbs. Suitable mammal cells for the present invention include, but are not limited to, epithelial cell lines (e.g., porcine epithelial cells), osteosarcoma cell lines, neuroblastoma cell lines, epithelial carcinomas, glial cells, liver cell lines, Chinese hamster ovary (CHO) cells, COS cells, BHK cells, HeLa cells, D3 cells of the line of murine embryonic stem cells (mESCs), human embryonic stem cells (e.g., HS293 cells and BG01V cells), NIH 3T3 cells, human embryonic kidney (HEK) 293T cells, human mesenchymal stem cells (hMSCSs), and the like.

As used herein, the terms “cell,” “cell culture,” “cell line,” and “host cell” refer not only to the particular cell, cell culture, cell line, or host cell, but also to the progeny or potential progeny of such a cell, cell culture, cell line, or host cell, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the terms as used herein, so long as the progeny retain the same functionality or substantially the same functionality as that of the original cell, cell culture, cell line, or host cell.

As used herein, the terms “medium” or “culture medium” include any culture medium, solution, solid, semi-solid, or rigid support that may support or contain any host cells, including, but not limited to, bacterial host cells, yeast or fungal host cells, insect host cells, plant host cells, eukaryotic host cells, mammalian host cells, CHO cells, prokaryotic host cells, E. coli host cells, Pseudomonas host cells, and the like, and cell contents. Thus, the term may encompass medium in which the host cell has been grown, (e.g., medium into which a polypeptide has been secreted, including medium either before, during, or after a proliferation step). The term also may encompass buffers or other reagents that contain host cell lysates, such as, for example, in the case where a polynucleotide is produced intracellularly, and the host cells are lysed or disrupted to release the polypeptide.

As used herein, the term “fusion protein” refers to a covalent joining of at least two proteins or protein domains. Fusion proteins may comprise artificial sequences, such as, for example, a domain linker, Fc domains, variant Fc domains, a variant IL-12 p35 subunit domain, a(n) (variant) IL-12 p40 subunit domain, and the like, as described herein. As used herein, the term “Fc fusion protein” refers to a protein comprising a (variant) Fc domain, generally linked (optionally through a domain linker, as described herein) to one or more different protein domains. In some embodiments, the C-terminus of the Fc domain is linked to the N-terminus of one or more different protein domains (optionally through a domain linker, wherein the C-terminus of the Fc domain is linked to the N-terminus of the domain linker and the C-terminus of the domain linker is linked to the N-terminus of the one or more different protein domains). In some embodiments, the N-terminus of the Fc domain is linked to the C-terminus of one or more different protein domains (optionally through a domain linker, wherein the C-terminus of the one or more protein domains is linked to the N-terminus of the domain linker and the C-terminus of the domain linker is linked to the N-terminus of the Fc domain).

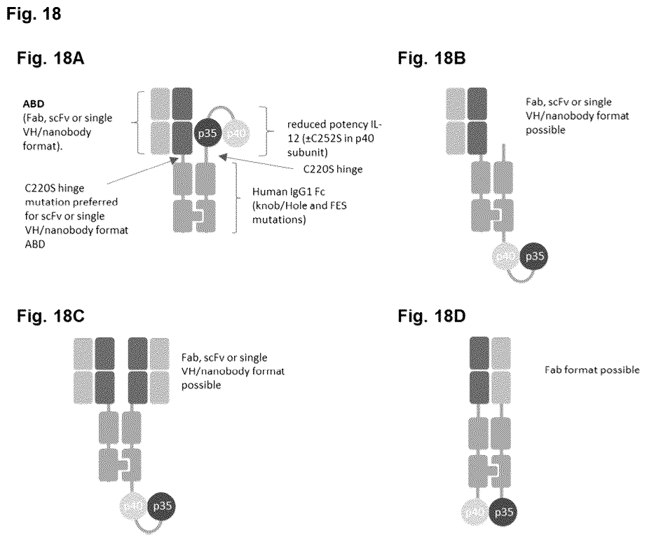

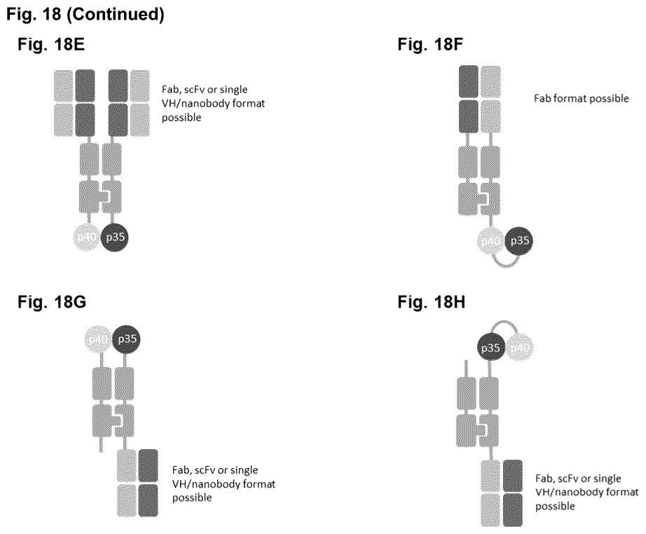

As used herein, the terms “low potency IL-12 Fc fusion proteins,” “IL-12-ABD fusion proteins,” “IL-12 Fc fusion proteins,” or “targeted IL-12 Fc fusion proteins,” which can be used interchangeably, generally refer to a fusion protein comprising: (1) a first IL-12 subunit domain and a second IL-12 subunit domain, wherein (i) the first IL-12 subunit domain comprises a variant IL-12 p35 subunit domain and the second IL-12 subunit domain comprises a (variant) IL-12 p40 subunit domain, or the first IL-12 subunit domain comprises a (variant) IL-12 p40 subunit domain and the second IL-12 subunit domain comprises a variant IL-12 p35 subunit domain, and (ii) the variant IL-12 p35 subunit domain comprises a set of amino acid substitutions that reduces binding affinity of the resultant IL-12 Fc fusion protein for IL-12Rβ2 as compared to a reference IL-12, wherein the set of amino acid substitutions is selected from a group including: (a) Y40S/K170A, (b) Y40S/K170T, (c) Y40P/K168A, (d) Y40P/K170A, and (e) Y40E/K170A; (2) at least one antigen binding domain (ABD; i.e., one ABD, two ABDs, three ABDs, etc.), wherein the ABD binds a target antigen selected from a group including: (a) OX40, (b) LAG3, (c) PD1, (d) ICOS, (e) 41BB, (f) CCR7, and (g) CCR8; (3) one or more domain linkers; and (4) a first Fc domain and a second Fc domain. As will be appreciated by those skilled in the art, a subject IL-12 Fc fusion protein can take on one of several different configurations (including, but not limited to, those described herein and shown in the figures (see, e.g., FIGS. 18A-18H)). By way of a first example, a subject IL-12 Fc fusion protein can be configured such that the ABD binds a human target antigen, a mouse target antigen, a camelid target antigen, a cynomolgus monkey target antigen, or the ABD is a humanized ABD. In some embodiments, the target antigen comprises a human target antigen (e.g., human PD1, human LAG3, human OX40, human ICOS, human 41BB, human CCR7, or human CCR8). In some embodiments, the target antigen comprises a mouse target antigen. In some embodiments, the target antigen comprises a camelid target antigen. In some embodiments, the target antigen comprises a cynomolgus monkey target antigen. In some further embodiments, the ABD is a humanized ABD. As another example, a subject IL-12 Fc fusion protein can be configured such that the ABD is in a Fab format, a scFv format, a nanobody format, as well as other formats known in the relevant art (including, but not limited to: a diabody format, a triabody format, a minibody format, a single-chain Fab (scFab) format, a F(ab)₂ format, a scFv-Fc format, a BiTE format, an intrabody format, a tandem scFv format, a DART format, and the like). In some instances, the IL-12 Fc fusion protein is configured such that the ABD is in a Fab format (i.e., the IL-12 Fc fusion protein comprises a variable heavy (VH) domain, a variable light (VL) domain, a constant heavy (CH1) domain, and a constant light (CL) domain). In some other instances, the IL-12 Fc fusion protein is configured such that the ABD is in an scFv format (i.e., the IL-12 Fc fusion protein comprises, from N-terminus to C-terminus, a VH domain, a linker, and a VL domain; or the reverse where the IL-12 Fc fusion protein comprises, from N-terminus to C-terminus, a VL domain, a linker, and a VH domain). In still other instances, the IL-12 Fc fusion protein is configured such that the ABD is in a nanobody format (i.e., the IL-12 Fc fusion protein comprises a VHH domain, wherein the VHH domain comprises a complementarity determining region (CDR) 1, a CDR2, and a CDR3, wherein the CDR1, CDR2, and CDR3 bind to a target antigen). As another example, a subject IL-12 Fc fusion protein can be configured such that the fusion protein comprises one ABD (i.e., a monovalent, monospecific format), two ABDs (e.g., a bivalent, monospecific format, or a monovalent, bispecific format), or three or more ABDs (e.g., a monovalent, multispecific format, a multivalent, monospecific format, a multivalent, multispecific format, and the like). In instances where the subject IL-12 Fc fusion protein comprises two ABDs, the first ABD and the second ABD may bind the same target antigen (i.e., a bivalent, monospecific format), or the first ABD may bind a first target antigen and the second ABD may bind a second, distinct target antigen (i.e., a monovalent, bispecific format). As will be appreciated by those skilled in the relevant art, a subject IL-12 Fc fusion protein comprising three or more ABDs can be configured in similar manners depending on the total number of ABDs and the specificity of such ABDs (e.g., monovalent, multispecific formats; multivalent, monospecific formats; multivalent, multispecific formats; and the like). As another example, a subject IL-12 Fc fusion protein can be configured such that the first Fc domain and/or the second Fc domain comprises a wild-type Fc domain or a Fc domain that comprises one or more substitution mutations. In some embodiments, at least one of the first Fc domain and second Fc domain comprises a variant Fc domain (e.g., the first Fc domain comprises a variant Fc domain and the second Fc domain either comprises a Fc domain or a variant Fc domain, the second Fc domain comprises a variant Fc domain and the first Fc domain either comprises a Fc domain or a variant Fc domain). In some embodiments, the first Fc domain and/or the second Fc domain comprise modifications that promote heterodimerization of the first and second Fc domains. In some embodiments, the first Fc domain and/or the second Fc domain comprises one or more modifications that alter Fc binding. In some embodiments, the first Fc domain and/or the second Fc domain comprises one or more modifications that alter Fc half-life. In some embodiments, the first Fc domain and/or the second Fc domain comprises one or more modifications that alter the binding of the first Fc domain and/or the second Fc domain to the neonatal Fc receptor (FcRn). In certain further embodiments, the first Fc domain and/or the second Fc domain comprise one or more modifications that increase half-life and/or binding to FcRn. As another example, a subject IL-12 Fc fusion protein can be configured such that: (1) the first IL-12 subunit domain and the second IL-12 subunit domain are on the same monomer of the subject IL-12 Fc fusion protein (covalently linked using a domain linker in several embodiments (e.g., from N-terminus to C-terminus, variant IL-12 p35 subunit domain-domain linker-(variant) IL-12 p40 subunit domain or (variant) IL-12 p40 subunit domain-domain linker-variant IL-12 p35 subunit domain)), (2) the first IL-12 subunit domain and the second IL-12 subunit domain are on separate monomers of the subject IL-12 Fc fusion protein (e.g., a first monomer and a second monomer, respectively, or the reverse), or (3) the first IL-12 subunit domain is covalently attached to a monomer of the subject IL-12 Fc fusion protein and the second IL-12 subunit domain is expressed as a soluble protein, wherein the soluble IL-12 subunit interacts with/binds the IL-12 subunit covalently attached to a monomer of the subject IL-12 Fc fusion protein. Exemplary configurations wherein one IL-12 subunit is covalently attached to a subject IL-12 Fc fusion protein and one IL-12 subunit is expressed as a soluble fragment is described in PCT Publication No. WO 2023/242769 A1, which is incorporated by reference in its entirety. In some embodiments, the first IL-12 subunit domain comprises a variant IL-12 p35 subunit domain, and the second IL-12 subunit domain comprises an IL-12 p40 subunit domain or a variant IL-12 p40 subunit domain. In some embodiments, the first IL-12 subunit domain comprises an IL-12 p40 subunit domain or a variant IL-12 p40 subunit domain, and the second IL-12 subunit domain comprises a variant IL-12 p35 subunit domain. In some embodiments, the C-terminus of the first IL-12 subunit domain is linked to the N-terminus of the first Fc domain (optionally through a domain linker), and the C-terminus of the second IL-12 subunit domain is linked to the N-terminus of the second Fc domain (optionally through a domain linker). In some embodiments, the C-terminus of the first Fc domain is linked to the N-terminus of the first IL-12 subunit domain (optionally through a domain linker), and the C-terminus of the second Fc domain is linked to the N-terminus of the second IL-12 subunit domain (optionally through a domain linker).

As used herein, the term “isolated,” when used to describe the various polypeptides disclosed herein, refers to a polypeptide that has been identified and separated and/or recovered from a cell (e.g., a host cell and/or a cell line) or cell culture from which it was expressed. Generally, an isolated polypeptide will be prepared by at least one purification step. As used herein, the term “isolated protein” refers to a protein which is substantially free of other proteins from a cell culture, such as, for example, host cell proteins.

As used herein, the terms “Fc,” “Fc region,” or “Fc domain,” which can be used interchangeably, refer to the polypeptide comprising the CH2-CH3 domains of an immunoglobulin G (IgG) molecule, and in some cases, inclusive of all or part of the hinge, as well as variants thereof. In EU numbering for human IgG1, the CH2-CH3 domain comprises amino acids 231 to 447, and the hinge is 216 to 230. Thus, the definition of “Fc domain” includes both amino acids 231-447 (CH2-CH3) or 216-447 (hinge-CH2-CH3), or fragments thereof. A “Fc fragment” in this context may contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.). Herein, unless specifically outlined, a “Fc domain” generally refers to the CH2-CH3 domains (and optionally all or part of the hinge) of human IgG1. Described below are various human IgG1 Fc domains, at least some of which comprise one or more modifications. Unless the context clearly indicates otherwise, amino acid position numbering with respect to a Fc domain (e.g., a first Fc domain, a second Fc domain, etc.) is according to the EU index. The “EU index” or “EU index as in Kabat” or “EU numbering” scheme, which can be used interchangeably, refers to the numbering of the EU antibody (Edelman et al., 1969, Proc Natl Acad Sci USA 63:78-85, hereby entirely incorporated by reference). For clarity, while the modifications are discussed herein primarily in the context of human IgG1 Fc domains for the sake of brevity, it is expressly contemplated that mutations to other immunoglobulins, such as, for example, IgG2, IgG3, IgG4, IgA, IgM, and IgE, can be made at residue positions corresponding to the mutations in human IgG1 that are described herein. Those skilled in the relevant art will readily be able to determine residue locations in other immunoglobulins that correspond with the modifications to human IgG1 described herein.

As used herein, the term “vector” refers to a nucleic acid molecule or sequence capable of transferring or transporting another nucleic acid molecule. The transferred nucleic acid molecule is generally linked to (e.g., inserted into) the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments thereof in vitro and/or in vivo. A vector may include sequences that direct autonomous replication in a cell or may include sequences sufficient to allow integration into host cell DNA. Useful vectors include, but are not limited to, plasmids (e.g., DNA plasmids or RNA plasmids), transposons, cosmids, bacterial artificial chromosomes, and viral vectors. Useful viral vectors include, but are not limited to, replication defective retroviruses and lentiviruses. In some embodiments, a vector is a gene delivery vector. In some embodiments, a vector is used as a gene delivery vehicle to transfer a gene into a cell.

As used herein, the term “recombinant,” with respect to a nucleic acid molecule, refers to a polynucleotide of genomic, cDNA, viral, semisynthetic, and/or synthetic origin which, by virtue of its own origin or manipulation, is not associated with all or a portion of the polynucleotide with which it is associated in nature. The term “recombinant,” as used with respect to a protein or polypeptide, refers to a polypeptide produced by expression of a recombinant polynucleotide. The term “recombinant,” as used with respect to a host cell, refers to a host cell into which a recombinant polynucleotide or vector comprising a recombinant polynucleotide has been introduced.

As used herein, the term “operably linked” refers to a physical or functional linkage between two or more elements (e.g., polypeptide sequences or polynucleotide sequences) that permits them to operate in their intended fashion. For example, an operable linkage between a polynucleotide of interest and a regulatory sequence (such as, for example, a promoter) is a functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. Thus, a promoter is in an operable linkage with a nucleic acid sequence if it can mediate transcription of the nucleic acid sequence. It should be understood that elements that are operably linked may be contiguous or non-contiguous. In the context of a polypeptide, “operably linked” refers to a physical linkage (e.g., directly, or indirectly linked) between amino acid sequences (e.g., different segments, modules, or domains) to provide for a described activity of the polypeptide.

As used herein, the term “binding affinity” refers to the “strength” of binding of a given molecule (such as, for example, a variant IL-12 p35 subunit domain, or an antigen binding domain) to its ligand (such as, for example, IL-12Rβ2, PD1, LAG3, OX40, ICOS, 41BB, CCR7, CCR8, etc.) and/or the rate at which the molecule associates with and/or dissociates from its ligand. Binding affinity is often expressed in terms of the dissociation constant (K_{D}). The binding activity of the IL-12 Fc fusion proteins of the disclosure can be assayed by any suitable method known in the art, such as, for example, a surface plasmon resonance (SPR) assay, an enzyme-linked immunosorbent assay (ELISA), an ELISpot assay, Biacore assays, KinExA assays, and the like. As the IL-12 Fc fusion proteins bind to more than one target (e.g., IL-12Rβ2 and at least one target antigen (PD1, LAG3, OX40, ICOS, 41BB, CCR7, CCR8)), such IL-12 Fc fusion proteins will exhibit separate binding affinities for IL-12Rβ2 and the at least one target antigen. As such, the term “binding affinity” is intended to refer to the “strength” of binding to IL-12Rβ2 when referring to the “IL-12 binding arm” of a subject IL-12 Fc fusion protein, or to the “strength” of binding to the target antigen when referring to the ABD of a subject IL-12 Fc fusion protein. Further, some configurations of IL-12 Fc fusion proteins described herein comprise more than one ABD and are therefore capable of binding more than one target antigen. As such, in such embodiments, the term “binding affinity” is intended to refer to the “strength” of binding to a first target antigen by a first ABD (e.g., the strength of binding to PD1 by an ABD that “specifically binds” to PD1) or the “strength” of binding to a second target antigen by a second ABD (e.g., the strength of binding to LAG3 by an ABD that “specifically binds” to LAG3).

As used herein, the terms “specific binding,” “specifically binds to,” or is “specific for” a particular antigen or an “epitope,” which can be used interchangeably, means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

As used herein, the term “epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

As used herein, the term “potency” refers to the ability of a given protein, cytokine, fusion protein, antibody, and the like (such as, for example, an IL-12 Fc fusion protein) to elicit a response at a certain dose or concentration in a given biological system or experimental setting. The potency of the IL-12 Fc fusion proteins of the disclosure can be assayed by any suitable method known in the art, such as, for example, an IL-12 HEK reporter assay (e.g., InvivoGen's IL-12 HEK reporter assay (Catalog No. hkb-il12)), a ligand-binding assay (e.g., ELISA or flow cytometry), and/or a functional assay (e.g., AlphaLISA). Generally, changes in potency may be demonstrated graphically as a leftward or rightward shift in a response curve compared to a control. A rightward shift of the response curve is generally indicative of a reduction in potency, whereas as a leftward shift of the response curve is generally indicative of an increase in potency.

As used herein, the term “activity” refers to the specific response of a given protein, cytokine, fusion protein, antibody, and the like (such as, for example, an IL-12 Fc fusion protein) elicited in a particular biological system or experimental setting at a given dose or concentration. The activity of the IL-12 Fc fusion proteins of the disclosure can be assayed by any suitable method known in the art, such as, for example, a ligand-binding assay and/or a functional assay. Generally, changes in activity may be demonstrated graphically as an upward or downward shift in a response curve compared to a control. An upward shift of the response curve is generally indicative of an increase in activity, whereas a downward shift of the response curve is generally indicative of a decrease in activity. As described in further detail below, a change in activity may or may not be related to and/or caused by a change in potency.

As used herein, the term “manufacturability” refers to any property that may impact the process of producing and/or storing a given protein, cytokine, fusion protein, antibody, and the like at a scale and quantity sufficient for administration to an individual. Examples of properties that impact manufacturability include, but are not limited to, the stability, purity, aggregation levels, and/or yield of expression of a given protein, cytokine, fusion protein, antibody, and the like (such as, for example, an IL-12 Fc fusion protein).

As used herein, the term “stability” refers to the ability of a given protein, cytokine, fusion protein, antibody, and the like (such as, for example, an IL-12 Fc fusion protein) to retain the same properties and characteristics that it possessed at the time of its manufacture within specified limits and/or storage and/or use parameters. The stability of the IL-12 Fc fusion proteins of the disclosure can be assayed by any suitable method known in the art, such as, for example ELISA, Western blot, Biacore assay, SDS-PAGE, size exclusion chromatography, dynamic light scattering, differential scanning calorimetry, and differential scanning fluorimetry.

As used herein, the term “yield of expression” refers to the amount or quantity of a given protein, cytokine, fusion protein, antibody, and the like (such as, for example, an IL-12 Fc fusion protein) produced using a prokaryotic or eukaryotic host system. Recombinant expression of proteins, cytokines, fusion proteins, antibodies, and the like are well-known in relevant arts. Any suitable method for quantitating or determining a yield of expression may be employed, such as, for example, UV absorption measurements, colorimetric assays (e.g., Bradford assay, BCA assay, and Lowry assay), and fluorometric assays.

As used herein, the terms “subject” or an “individual” for purposes of treatment can generally refer to any animal classified as a mammal (including but not limited to humans, primates, and/or non-human primates), domestic animals, farm animals, zoo animals, research animals, sports animals, and/or pet animals, such as dogs, horses, cats, cows, etc. However, unless the context clearly dictates otherwise, references to a “subject” are intended to refer to a human subject.

As used herein, the singular forms “a,” “an,” and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone),” and “B (alone).”

As used herein, the use of ordinal terms, such as, for example, “first,” “second,” “third,” “fourth”, “fifth,” etc., to modify an element of the disclosure does not by itself connote any priority, precedence, or order of one element over another or the temporal order in which acts of a method are performed, but are used merely as labels to distinguish one element having a certain name (e.g., an assay, a domain linker, etc.) from another element having a same name but for the use of the ordinal term to distinguish the elements (e.g., “a first assay,” “a second assay,” “a third assay,” “a fourth assay,” “a fifth assay,” etc.). For example, a “first assay” may be performed to detect changes in potency of an IL-12 Fc fusion protein and a “second assay” may be performed to detect changes in binding affinity for IL-12Rβ2. In such an example, the terms “first assay” and “second assay” are not used to describe any priority, precedence, or order in which the assays are performed, but rather to denote that the elements are distinct despite having the same name except for the use of the ordinal terms “first” and “second.” As a separate example, a “third assay” may be performed to detect changes in capability to stimulate IFNγ without a “first assay” or a “second assay” having been performed. Alternatively, it is also contemplated that a “first assay” could be performed before or after the “third assay,” and that the “second assay” of the example may not be performed, may be performed before or after the “first assay,” or before or after the “third assay.” While the examples set forth above utilize specific ordinal terms, it is contemplated that the concepts described above apply mutatis mutandis to other examples using any number of ordinal terms in various configurations.

As used herein, the term “about” a value (or parameter) refers to +10% of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Overview

The present application generally relates to compositions and methods for modulating signal transduction mediated by IL-12. In particular, the disclosure provides low potency IL-12 Fc fusion proteins comprising: (1) a variant IL-12 p35 subunit domain that reduces binding affinity to interleukin 12 receptor beta 2 (IL-12Rβ2), (2) an IL-12 p40 subunit domain, and (3) an antigen binding domain (ABD) to facilitate trafficking of the fusion protein to a target cell of interest, as well as methods of making and using the same. In some preferred embodiments, the ABD binds to OX40. In some further preferred embodiments, the ABD binds to the extracellular domain (ECD) of OX40. In still some further preferred embodiments, the ABD binds to the ECD of human OX40.

As noted above, IL-12 is a potent, pro-inflammatory cytokine with a high affinity for its receptors (p35 subunit for IL-12Rβ2; p40 subunit for IL-12Rβ1). Due to this high affinity, IL-12 therapies configured to target various cell populations utilizing wild-type IL-12 in combination with an ABD have generally not been successful, as the affinity and/or potency of the “IL-12 binding arm” of the therapy is generally greater than the binding affinity/potency of the “ABD arm” of the therapy. Fusion of reduced potency IL-12 variants to targeting ABD has the potential to overcome this limitation. However, the properties of the resultant fusion protein are heavily influenced by the identity of the ABD target. For example, the type and frequency of immune cells that express the ABD target can impact the efficacy, pharmacokinetics, and tolerability of the IL-12 ABD fusion protein. As demonstrated herein, targeting of low potency IL-12 using ABD specific for Lag-3, CCR7, or CCR8 did not enhance IL-12 anti-tumor activity. While some other targeting arms (for example, PD-1 targeting, ICOS-targeting, or 41BB-targeting) enhanced IL-12 anti-tumor activity, the resultant IL-12 ABD fusion proteins exhibited a less preferred pharmacokinetic profile (e.g., faster clearance) and/or a worse safety profile, potentially due to the relatively broad expression of these targets. The present application describes, inter alia, OX40-targeted reduced potency IL-12 fusion proteins that exhibit enhanced properties relative to other targeted IL-12 therapeutics described herein. OX40-targeted reduced potency IL-12 fusion proteins demonstrated potent anti-tumor activity and importantly, only minimally impacted pharmacokinetic profile and tolerability. Accordingly, OX40 targeting resulted in the largest improvement of therapeutic index of any of the molecules tested. Relatedly, coadministration of an OX40-targeted reduced potency IL-12 fusion protein with an anti-PD-1 antibody further enhanced the anti-tumor activity without significantly affecting the pharmacokinetic profile and tolerability, whereas the PD-1-targeted reduced potency IL-12 fusion protein targeted additional cell types resulting in an increase in toxicity (as is described in further detail herein).

In some embodiments, the compositions and methods described herein facilitate the targeting of a target cell using an IL-12 Fc fusion protein, wherein the target cell expresses: (1) IL-12Rβ2, and (2) a cell surface antigen selected from a group that includes (a) human OX40, (b) human LAG3, (c) human PD1, (d) human ICOS, (e) human 41BB, (f) human CCR7, or (g) human CCR8.

In some embodiments, the compositions and methods described herein facilitate the trafficking of an IL-12 Fc fusion protein to a target cell expressing: (1) IL-12Rβ2, and (2) a cell surface antigen selected from a group that includes: (a) human OX40, (b) human LAG3, (c) human PD1, (d) human ICOS, (e) human 41BB, (f) human CCR7, (g) human CCR8, or any combination thereof. In still some further embodiments, the cell surface antigen is PD1, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is LAG3, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is OX40, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is ICOS, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is 41BB, and the target cell comprises: (i) activated and/or exhausted CD4⁺ T cell(s), (ii) activated and/or exhausted NK cell(s), (iii) activated and/or exhausted CD8⁺ T cell(s), or (iv) activated and/or exhausted regulatory T cell(s). In other further embodiments, the cell surface antigen is CCR7, and the target cell comprises: (i) naïve CD4⁺ T cell(s), (ii) naïve CD8⁺ T cell(s), or (iii) naïve regulatory T cell(s). In other further embodiments, the cell surface antigen is CCR8, and the target cell comprises: (i) activated and/or exhausted CD4⁺ T cell(s), or (ii) activated and/or exhausted regulatory T cell(s).

### III. Compositions

As will be appreciated by one skilled in the art, any of the aspects and embodiments of the compositions described herein can be used in any of the aspects and/or embodiments of the methods of making described below or in the methods of use also described below.

### A. Low Potency Interleukin 12 (IL-12) Fc Fusion Proteins

The present disclosure provides several aspects and embodiments comprising low potency IL-12 Fc fusion proteins (sometimes referred to herein as an “IL-12 Fc fusion protein” for the sake of brevity). The IL-12 Fc fusion proteins described herein comprise: (1) a first IL-12 subunit domain and a second IL-12 subunit domain, wherein (i) the first IL-12 subunit domain comprises a variant IL-12 p35 subunit domain and the second IL-12 subunit domain comprises a (variant) IL-12 p40 subunit domain, or the first IL-12 subunit domain comprises a (variant) IL-12 p40 subunit domain and the second IL-12 subunit domain comprises a variant IL-12 p35 subunit domain, and (ii) the variant IL-12 p35 subunit domain comprises a set of amino acid substitutions that reduces binding affinity of the resultant IL-12 Fc fusion protein for IL-12Rβ2 as compared to a reference IL-12, wherein the set of amino acid substitutions is selected from a group including: (a) Y40S/K170A, (b) Y40S/K170T, (c) Y40P/K168A, (d) Y40P/K170A, and (e) Y40E/K170A; (2) at least one antigen binding domain (ABD), wherein the ABD binds a target antigen selected from a group including: (a) OX40, (b) LAG3, (c) PD1, (d) ICOS, (e) 41BB, (f) CCR7, and (g) CCR8; (3) one or more domain linkers; and (4) a first Fc domain and a second Fc domain. In some embodiments, the target antigen comprises a human target antigen (e.g., human PD1, human LAG3, human OX40, human ICOS, human 41BB, human CCR7, or human CCR8). In some embodiments, the target antigen comprises a mouse target antigen. In some embodiments, the ABD comprises a humanized ABD.

As will be appreciated by those skilled in the art, a subject IL-12 Fc fusion protein can take on one of several different configurations. In some instances, the IL-12 Fc fusion protein is configured such that the ABD is in a Fab format (i.e., the IL-12 Fc fusion protein comprises a variable heavy (VH) domain, a variable light (VL) domain, a constant heavy (CH1) domain, and a constant light (CL) domain). In some other instances, the IL-12 Fc fusion protein is configured such that the ABD is in an scFv format (i.e., the IL-12 Fc fusion protein comprises, from N-terminus to C-terminus, a VH domain, a domain linker, and a VL domain; or the reverse where the IL-12 Fc fusion protein comprises, from N-terminus to C-terminus, a VL domain, a domain linker, and a VH domain). In still other instances, the IL-12 Fc fusion protein is configured such that the ABD is in a nanobody format (i.e., the IL-12 Fc fusion protein comprises a VHH domain, wherein the VHH domain comprises a complementarity determining region (CDR) 1, a CDR2, and a CDR3, wherein the CDR1, CDR2, and CDR3 bind to a target antigen).

Due to the modular nature of the IL-12 Fc fusion protein's configuration, a discussion of exemplary components comprising a subject IL-12 Fc fusion protein is first provided below (such as, for example, variant IL-12 p35 subunit domains, (variant) IL-12 p40 subunit domains, ABDs, VH domains, VL domains, Fc domains, domain linkers, and the like), followed by a discussion of exemplary IL-12 Fc fusion protein configurations finding utility in some of the embodiments described herein.

### 1. Interleukin 12:

The present disclosure provides IL-12 Fc fusion proteins comprising: (1) a first IL-12 subunit domain and a second IL-12 subunit domain, wherein (i) the first IL-12 subunit domain comprises a variant IL-12 p35 subunit domain and the second IL-12 subunit domain comprises a (variant) IL-12 p40 subunit domain, or the first IL-12 subunit domain comprises a (variant) IL-12 p40 subunit domain and the second IL-12 subunit domain comprises a variant IL-12 p35 subunit domain, and (ii) the variant IL-12 p35 subunit domain comprises a set of amino acid substitutions that reduces binding affinity of the resultant IL-12 Fc fusion protein for IL-12Rβ2 as compared to a reference IL-12, wherein the set of amino acid substitutions is selected from a group including: (a) Y40S/K170A, (b) Y40S/K170T, (c) Y40P/K168A, (d) Y40P/K170A, and (e) Y40E/K170A; (2) at least one antigen binding domain (ABD), wherein the ABD binds a target antigen selected from a group including: (a) OX40, (b) LAG3, (c) PD1, (d) ICOS, (e) 41BB, (f) CCR7, and (g) CCR8; (3) one or more domain linkers; and (4) a first Fc domain and a second Fc domain.

In some embodiments, the first IL-12 subunit domain and the second IL-12 subunit domain are configured such that the first IL-12 subunit domain and the second IL-12 subunit domain are on separate monomers of the IL-12 Fc fusion protein (e.g., a first monomer of the IL-12 Fc fusion protein comprises the first IL-12 subunit domain and the second monomer comprises the second IL-12 subunit domain, or the reverse where the first monomer comprises the second IL-12 subunit domain and the second monomer comprises the first IL-12 subunit domain). In other embodiments, the first IL-12 subunit domain and the second IL-12 subunit domain are configured such that they are on the same monomer of the IL-12 Fc fusion protein, often in a single chain format linked by a domain linker (e.g., the first monomer comprises the first and second IL-12 subunit domains and the second monomer does not comprise an IL-12 subunit domain; the second monomer comprises the first and second IL-12 subunit domains and the first monomer does not comprise an IL-12 subunit domain).

In some instances where the first and second IL-12 subunit domains are on separate monomers of the IL-12 Fc fusion protein, the first and second IL-12 subunit domains may be configured such that they are on the N-terminus of their respective monomers, or they may be configured such that they are on the C-terminus of their respective monomers. In instances where the first and second IL-12 subunit domain are on one monomer of the IL-12 Fc fusion protein, a single chain IL-12 comprising a first IL-12 subunit domain, a domain linker, and a second IL-12 subunit domain may be configured such that the single chain IL-12 is on the N-terminus of a monomer or configured such that the single chain IL-12 is on the C-terminus of the monomer. In either configuration, the variant p35 subunit domain may be on the N-terminus of the single chain IL-12 and the (variant) p40 subunit domain is on the C-terminus of the single chain IL-12, or the (variant) p40 subunit domain may be on the N-terminus of the single chain IL-12 and the variant p35 subunit domain is on the C-terminus of the single chain IL-12.

While exemplary IL-12 Fc fusion proteins are generally set forth such that either: (1) the first IL-12 subunit domain and the second IL-12 subunit domain are on the same monomer of a subject IL-12 Fc fusion protein (covalently linked using a domain linker in several embodiments (e.g., from N-terminus to C-terminus, variant IL-12 p35 subunit domain-domain linker-(variant) IL-12 p40 subunit domain, or (variant) IL-12 p40 subunit domain-domain linker-variant IL-12 p35 subunit domain)), or (2) the first IL-12 subunit domain and the second IL-12 subunit domain are on separate monomers of a subject IL-12 Fc fusion protein (e.g., a first monomer and a second monomer, respectively, or the reverse), it is also expressly contemplated that a subject IL-12 Fc fusion protein can be configured such that the first IL-12 subunit domain is covalently attached to a monomer of the subject IL-12 Fc fusion protein and the second IL-12 subunit domain is expressed as a soluble protein, wherein the soluble IL-12 subunit interacts with/binds the IL-12 subunit covalently attached to a monomer of the subject IL-12 Fc fusion protein (or the reverse, where the first IL-12 subunit domain is expressed as a soluble protein and the second IL-12 subunit domain is covalently attached to a monomer of a subject IL-12 Fc fusion protein). Exemplary configurations wherein one IL-12 subunit is covalently attached to a subject IL-12 Fc fusion protein and one IL-12 subunit is expressed as a soluble fragment is described in, for example, PCT Publication No. WO 2023/242769 A1, which is incorporated by reference in its entirety.

Binding affinities to IL-12Rβ2 can be assessed using any number of assays known to those skilled in the relevant art. Non-limiting examples of suitable assays for evaluating binding affinity include: a surface plasmon resonance (SPR) assay, an enzyme-linked immunosorbent assay (ELISA), an ELISpot assay, Biacore assays, KinExA assays, flow cytometry, and the like.

### a. p35 Subunit Domain:

In some embodiments, the variant IL-12 p35 subunit domain comprises one or more amino acid substitutions selected from a group including: (a) Y40S/K170A, (b) Y40S/K170T, (c) Y40P/K168A, (d) Y40P/K170A, and (e) Y40E/K170A. In some embodiments, the one or more amino acid substitutions comprise Y40P/K168A. In other embodiments, the one or more amino acid substitutions comprise Y40S/K170T. In other embodiments, the one or more amino acid substitutions comprise Y40S/K170A. In other embodiments, the one or more amino acid substitutions comprise Y40P/K170A. In other embodiments, the one or more amino acid substitutions comprise Y40E/K170A.

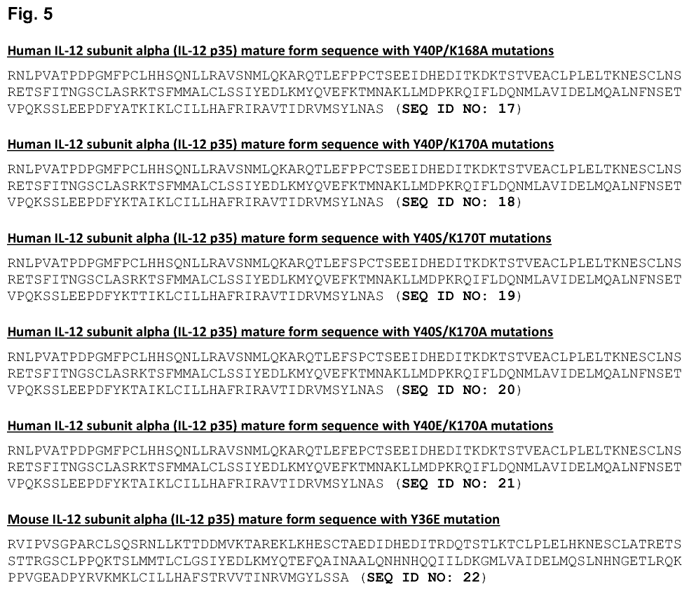

In some embodiments, the variant IL-12 p35 subunit domain comprises an amino acid sequence selected from a group including any one of: (i) SEQ ID NO: 17 (Y40P/K168A), (ii) SEQ ID NO: 18 (Y40P/K170A), (iii) SEQ ID NO: 19 (Y40S/K170T), (iv) SEQ ID NO: 20 (Y40S/K170A), and (v) SEQ ID NO: 21 (Y40E/K170A), as shown in FIG. 5.

In some embodiments, the variant IL-12 p35 subunit domain comprises an amino acid sequence with at least 90%, 91%, 92%, 93%, 94%, 95%, 96%, 97%, 98%, 99% or higher sequence identity to an amino acid sequence selected from a group including any one of SEQ ID NOs: 17-21 (as shown in FIG. 5).

In some embodiments, the variant IL-12 p35 subunit domain comprises a first substitution mutation, comprising: Y40P, and further comprises a second substitution mutation selected from a group including: K168A, and K170A. In some embodiments, the variant IL-12 p35 subunit domain comprises any one of SEQ ID NOs: 17 and 18, as shown in FIG. 5.

In some embodiments, the variant IL-12 p35 subunit domain comprises a first substitution mutation, comprising: Y40S, and further comprises a second substitution mutation selected from a group including: K170T, and K170A. In some embodiments, the variant IL-12 p35 subunit domain comprises any one of SEQ ID NOs: 19 and 20, as shown in FIG. 5.

In some embodiments, the variant IL-12 p35 subunit domain comprises a first substitution mutation, comprising: Y40E, and further comprises a second substitution mutation selected from a group including: K170A. In some embodiments, the variant IL-12 p35 subunit domain comprises SEQ ID NO: 21, as shown in FIG. 5.

In some embodiments, the variant IL-12 p35 subunit domain further comprises a C74S substitution mutation as compared to SEQ ID NO: 2 (finding particular utility in embodiments where the subject IL-12 Fc fusion protein comprises a variant IL-12 p40 subunit domain that comprises C177S or C177S/C252S substitution mutation(s)).

In addition to the IL-12 Fc fusion proteins described above, additional modifications to various residues are known in relevant arts. Non-limiting examples of residues that may be modified include: Q20, N21, Q35, L37, E38, F39, P41, T43, 544, E45, E46, 147, H49, E50, 152, K54, D55, T59, V60, E61, C63, L64, P65, E67, L68, T69, N71, S73, C74, L75, N76, E79, N85, L89, F96, M97, M98, A99, L123, L124, M125, D126, K128, Q130, 1131, Q135, N136, E143, Q146, N151, E153, K158, E162, E163, P164, D165, F166, Y167, T169, 1171, K172, L173, 1175, R181, 1182, R183, V185, T186, D188, R189, V190, M191, S192, Y193, N195, A196, and S197. In some embodiments, the (variant) IL-12 p35 subunit domain described herein or in the figures (see, e.g., FIGS. 1, 3, and 5) comprises one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more additional amino acid substitutions. In some embodiments, the (variant) IL-12 p35 subunit domain described herein or in the figures (see, e.g., FIGS. 1, 3, and 5) comprises about 1 to about 5, about 6 to about 10, about 11 to about 15, about 16 to about 20, about 21 to about 25, about 26 to about 30, about 31 to about 35, about 36 to about 40, about 1 to about 10, about 11 to about 20, about 21 to about 30, about 31 to about 40, about 1 to about 20, or about 21 to about 40 additional amino acid substitutions.

Mutations in the variant IL-12 p35 subunit domain may result in changes to one or more of the following parameters: (i) binding affinity, (ii) potency, (iii) activity, (iv) manufacturability, or (v) stability; however, changes to one or more of the preceding parameters (such as, for example, binding affinity) may not necessarily be correlated with changes in one or more of the other preceding parameters (such as, for example, potency or activity). In some embodiments, the one or more amino acid substitutions in the variant IL-12 p35 subunit domain result in an altered binding affinity to IL-12Rβ2, as compared to the binding affinity of a reference IL-12. In some embodiments, the one or more amino acid substitutions decreases the binding affinity of the variant IL-12 p35 subunit domain to IL-12Rβ2 compared to a reference IL-12. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof. In other embodiments, the one or more amino acid substitutions in the variant IL-12 p35 subunit do not affect binding affinity but may result in changes to one or more of the following parameters: (i) potency, (ii) activity, (iii) manufacturability, (iv) stability, or (v) any combination thereof.

A variety of assay formats may be used to select IL-12 Fc fusion proteins that bind to a ligand of interest (e.g., IL-12Rβ2 and/or IL-12Rβ1). Non-limiting examples include: solid-phase ELISA immunoassay, immunoprecipitation, a Biacore assay, a KinExA assay, fluorescence-activated cell sorting (FACS), an Octet assay, Western blot analysis, and the like. The binding activity of the IL-12 Fc fusion proteins of the disclosure can be assayed by any suitable method known in the art, such as, for example, a surface plasmon resonance (SPR) assay, an enzyme-linked immunosorbent assay (ELISA), an ELISpot assay, Biacore assays, KinExA assays, and the like.

One of ordinary skill in the art will appreciate that binding affinity can also be used as a measure of the “strength” of a non-covalent interaction between two binding partners (e.g., a variant IL-12 p35 subunit domain and IL-12Rβ2). Binding affinity between two molecules may be quantified by determination of the dissociation constant (K_{D}). In turn, K_{D} can be determined by measurement of the kinetics of complex formation and dissociation using suitable assays known in the art, such as, for example, an SPR assay. The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation: K_{D}=k_{d}/kₐ. The value of the dissociation constant can be determined directly by well-known methods.

As used herein, the term “potency” refers to the ability of a given protein, cytokine, fusion protein, antibody, and the like (such as, for example, an IL-12 Fc fusion protein) to elicit a response at a certain dose or concentration in a given biological system or experimental setting. The potency of the IL-12 Fc fusion proteins of the disclosure can be assayed by any suitable method known in the art, such as, for example, an IL-12 HEK reporter assay (e.g., InvivoGen's IL-12 HEK reporter assay (Catalog No. hkb-il12)), a ligand-binding assay (e.g., ELISA or flow cytometry), and/or a functional assay (e.g., AlphaLISA). Generally, changes in potency may be demonstrated graphically as a leftward or rightward shift in a response curve compared to a control. A rightward shift of the response curve is generally indicative of a reduction in potency, whereas as a leftward shift of the response curve is generally indicative of an increase in potency. As used herein, the term “activity” refers to the specific response of a given protein, cytokine, fusion protein, antibody, and the like (such as, for example, an IL-12 Fc fusion protein) elicited in a particular biological system or experimental setting at a given dose or concentration. The activity of the IL-12 Fc fusion proteins of the disclosure can be assayed by any suitable method known in the art, such as, for example, a ligand-binding assay and/or a functional assay. Generally, changes in activity may be demonstrated graphically as an upward or downward shift in a response curve compared to a control. An upward shift of the response curve is generally indicative of an increase in activity, whereas a downward shift of the response curve is generally indicative of a decrease in activity.

As used herein, the term “manufacturability” refers to any property that may impact the process of producing and/or storing a given protein, cytokine, fusion protein, antibody, and the like at a scale and quantity sufficient for administration to an individual. Examples of properties that impact manufacturability include, but are not limited to, the stability, purity, aggregation levels, and/or yield of expression of a given protein, cytokine, fusion protein, antibody, and the like (such as, for example, an IL-12 Fc fusion protein). As used herein, the term “stability” refers to the ability of a given protein, cytokine, fusion protein, antibody, and the like (such as, for example, an IL-12 Fc fusion protein) to retain the same properties and characteristics that it possessed at the time of its manufacture within specified limits and/or storage and/or use parameters. The stability of the IL-12 Fc fusion proteins of the disclosure can be assayed by any suitable method known in the art, such as, for example ELISA, Western blot, Biacore assay, SDS-PAGE, size exclusion chromatography, dynamic light scattering, differential scanning calorimetry, and differential scanning fluorimetry. As used herein, the term “yield of expression” refers to the amount or quantity of a given protein, cytokine, fusion protein, antibody, and the like (such as, for example, an IL-12 Fc fusion protein) produced using a prokaryotic or eukaryotic host system. Recombinant expression of proteins, cytokines, fusion proteins, antibodies, and the like are well-known in relevant arts. Any suitable method for quantitating or determining a yield of expression may be employed, such as, for example, UV absorption measurements, colorimetric assays (e.g., Bradford assay, BCA assay, and Lowry assay), and fluorometric assays.

In some embodiments, the IL-12 Fc fusion protein and/or the variant IL-12 p35 subunit domain has binding affinity for IL-12Rβ2 reduced by at least about 50%, about 60%, about 70%, about 80%, about 90%, about 91%, about 92%, about 93%, about 94%, about 95%, about 96%, about 97%, about 98%, about 99%, or about 100% or more compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has binding affinity for IL-12Rβ2 reduced by about 50% to about 60%, about 60% to about 70%, about 70% to about 80%, about 80% to about 90%, or about 90% to about 100% or higher compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has binding affinity for IL-12Rβ2 reduced by at least about 1.5-fold, about 1.6-fold, about 1.7-fold, about 1.8-fold, about 1.9-fold, about 2.0-fold, about 2.1-fold, about 2.2-fold, about 2.3-fold, about 2.4-fold, about 2.5-fold, about 2.6-fold, about 2.7-fold, about 2.8-fold, about 2.9-fold, about 3.0-fold, about 3.1-fold, about 3.2-fold, about 3.3-fold, about 3.4-fold, about 3.5-fold, about 3.6-fold, about 3.7-fold, about 3.8-fold, about 3.9-fold, about 4.0-fold, about 4.1-fold, about 4.2-fold, about 4.3-fold, about 4.4-fold, about 4.5-fold, about 4.6-fold, about 4.7-fold, about 4.8-fold, about 4.9-fold, about 5.0-fold, about 5.1-fold, about 5.2-fold, about 5.3-fold, about 5.4-fold, about 5.5-fold, about 6.0-fold, about 7.0-fold, about 8.0-fold, about 9.0-fold, about 10.0-fold, about 20.0-fold, about 30.0-fold, about 40.0-fold, about 50.0-fold, about 100.0-fold, about 150.0-fold, about 200.0-fold, about 250.0-fold, about 300.0-fold, about 350.0-fold, about 400.0-fold, about 450.0-fold, about 500.0-fold, about 550.0-fold, about 600.0-fold, about 650.0-fold, about 700.0-fold, about 750.0-fold, about 800.0-fold, about 850.0-fold, about 900.0-fold, about 950.0-fold, about 1,000.0-fold, about 2,000.0-fold, about 3,000.0-fold, about 4,000.0-fold, about 5,000.0-fold, about 6,000.0-fold, about 7,000.0-fold, about 8,000.0-fold, about 9,000.0-fold, about 10,000.0-fold, about 20,000.0-fold, about 30,000.0-fold, about 40,000.0-fold, about 50,000.0-fold, about 100,000.0-fold, about 150,000.0-fold, about 200,000.0-fold, about 250,000.0-fold, about 300,000.0-fold, about 350,000.0-fold, about 400,000.0-fold, about 450,000.0-fold, about 500,000.0-fold or more compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has binding affinity for IL-12Rβ2 reduced by about 1.5-fold to about 2.0-fold, about 2.0-fold to about 3.0-fold, about 3.0-fold to about 4.0-fold, about 4.0-fold to about 5.0-fold, about 5.0-fold to about 10.0-fold, about 10.0-fold to about 20.0-fold, about 20.0-fold to about 30.0-fold, about 30.0-fold to about 40.0-fold, about 40.0-fold to about 50.0-fold, about 50.0-fold to about 100.0-fold, about 100.0-fold to about 150.0-fold, about 150.0-fold to about 200.0-fold, about 200.0-fold to about 250.0-fold, about 250.0-fold to about 300.0-fold, about 300.0-fold to about 350.0-fold, about 350.0-fold to about 400.0-fold, about 400.0-fold to about 450.0-fold, about 450.0-fold to about 500.0-fold, about 500.0-fold to about 550.0-fold, about 550.0-fold to about 600.0-fold, about 600.0-fold to about 650.0-fold, about 650.0-fold to about 700.0-fold, about 700.0-fold to about 750.0-fold, about 750.0-fold to about 800.0-fold, about 800.0-fold to about 850.0-fold, about 850.0-fold to about 900.0-fold, about 900.0-fold to about 950.0-fold, about 950.0-fold to about 1,000.0-fold, about 1,000.0-fold to about 2,000.0-fold, about 2,000.0-fold to about 3,000.0-fold, about 3,000.0-fold to about 4,000.0-fold, about 4,000.0-fold to about 5,000.0-fold, about 5,000.0-fold to about 6,000.0-fold, about 6,000.0-fold to about 7,000.0-fold, about 7,000.0-fold to about 8,000.0-fold, about 8,000.0-fold to about 9,000.0-fold, about 9,000.0-fold to about 10,000.0-fold, about 10,000.0-fold to about 20,000.0-fold, about 20,000.0-fold to about 30,000.0-fold, about 30,000.0-fold to about 40,000.0-fold, about 40,000.0-fold to about 50,000.0-fold, about 50,000.0-fold to about 100,000.0-fold, about 100,000.0-fold to about 150,000.0-fold, about 150,000.0-fold to about 200,000.0-fold, about 200,000.0-fold to about 250,000.0-fold, about 250,000.0-fold to about 300,000.0-fold, about 300,000.0-fold to about 350,000.0-fold, about 350,000.0-fold to about 400,000.0-fold, about 400,000.0-fold to about 450,000.0-fold, about 450,000.0-fold to about 500,000.0-fold or higher compared to binding affinity of a reference IL-12, as determined by an assay. In some embodiments, the assay comprises an SPR assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has binding affinity for IL-12Rβ2 below the lower level of detectability of an assay, and binding affinity of a reference IL-12 for IL-12Rβ2 is between or equivalent to the lower or upper level of detectability of an assay (i.e., the binding affinity of the reference IL-12 may be equal to the lower level of detectability, the upper level of detectability, or a value between the lower and upper levels of detectability; in other words, the binding affinity is “detectable” for the reference IL-12 and the binding affinity is “undetectable” for the IL-12 Fc fusion protein). In some embodiments, the assay comprises an SPR assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has potency reduced by at least about 50.0-fold, about 100.0-fold, about 150.0-fold, about 200.0-fold, about 250.0-fold, about 300.0-fold, about 350.0-fold, about 400.0-fold, about 450.0-fold, about 500.0-fold, about 550.0-fold, about 600.0-fold, about 650.0-fold, about 700.0-fold, about 750.0-fold, about 800.0-fold, about 850.0-fold, about 900.0-fold, about 950.0-fold, about 1,000.0-fold, about 2,000.0-fold, about 3,000.0-fold, about 4,000.0-fold, about 5,000.0-fold, about 6,000.0-fold, about 7,000.0-fold, about 8,000.0-fold, about 9,000.0-fold, about 10,000.0-fold, about 20,000.0-fold, about 30,000.0-fold, about 40,000.0-fold, about 50,000.0-fold, about 100,000.0-fold, about 200,000-fold, or about 300,000-fold or more compared to potency of a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has potency reduced by at least 50.0-fold to about 100.0-fold, about 100.0-fold to about 150.0-fold, about 150.0-fold to about 200.0-fold, about 200.0-fold to about 250.0-fold, about 250.0-fold to about 300.0-fold, about 300.0-fold to about 350.0-fold, about 350.0-fold to about 400.0-fold, about 400.0-fold to about 450.0-fold, about 450.0-fold to about 500.0-fold, about 500.0-fold to about 550.0-fold, about 550.0-fold to about 600.0-fold, about 600.0-fold to about 650.0-fold, about 650.0-fold to about 700.0-fold, about 700.0-fold to about 750.0-fold, about 750.0-fold to about 800.0-fold, about 800.0-fold to about 850.0-fold, about 850.0-fold to about 900.0-fold, about 900.0-fold to about 950.0-fold, about 950.0-fold to about 1,000.0-fold, about 1,000.0-fold to about 2,000.0-fold, about 2,000.0-fold to about 3,000.0-fold, about 3,000.0-fold to about 4,000.0-fold, about 4,000.0-fold to about 5,000.0-fold, about 5,000.0-fold to about 6,000.0-fold, about 6,000.0-fold to about 7,000.0-fold, about 7,000.0-fold to about 8,000.0-fold, about 8,000.0-fold to about 9,000.0-fold, about 9,000.0-fold to about 10,000.0-fold, about 10,000.0-fold to about 20,000.0-fold, about 20,000.0-fold to about 30,000.0-fold, about 30,000.0-fold to about 40,000.0-fold, about 40,000.0-fold to about 50,000.0-fold, about 50,000.0-fold to about 60,000.0-fold, about 60,000.0-fold to about 70,000.0-fold, about 70,000.0-fold to about 80,000.0-fold, about 80,000.0-fold to about 90,000.0-fold, about 90,000.0-fold to about 100,000.0-fold, about 100,000.0-fold to about 200,000.0-fold, or about 200,000.0-fold to about 300,000.0-fold or higher compared to potency of a reference IL-12 as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain exhibits a potency that is too weak to accurately be determined, and potency of a reference IL-12 is between or equivalent to the lower or upper level of detectability of an assay (i.e., the potency of the reference IL-12 may be equal to the lower level of detectability, the upper level of detectability, or a value between the lower and upper levels of detectability; in other words, the potency is “detectable” for the reference IL-12 and the potency is “undetectable” for the IL-12 Fc fusion protein). In some embodiments, the assay comprises an IL-12 HEK reporter assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has a reduced capability to stimulate STAT4 signaling compared to a reference IL-12, as determined by an assay. A reduction in the capability to stimulate STAT4 signaling may refer to a reduction in the maximal response observed and/or shifting in the EC₅₀ value. In some embodiments, the capability of the IL-12 Fc fusion protein to stimulate STAT4 signaling is reduced by at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, about 100% or more compared to a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the capability of the IL-12 Fc fusion protein and/or the variant IL-12 p35 subunit domain to stimulate STAT4 signaling is reduced by about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to a reference IL-12, as determined by an assay. In some embodiments, the assay comprises an IL-12 HEK reporter assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain has a reduced capability to stimulate IFNγ production compared to a reference IL-12, as determined by an assay. A reduction in the capability to stimulate IFNγ production may refer to a reduction in the maximal response observed and/or shifting in the EC₅₀ value. In some embodiments, the capability of the IL-12 Fc fusion protein and/or the variant IL-12 p35 subunit domain to stimulate IFNγ production is reduced by about 50.0-fold, about 100.0-fold, about 150.0-fold, about 200.0-fold, about 250.0-fold, about 300.0-fold, about 350.0-fold, about 400.0-fold, about 450.0-fold, about 500.0-fold, about 550.0-fold, about 600.0-fold, about 650.0-fold, about 700.0-fold, about 750.0-fold, about 800.0-fold, about 850.0-fold, about 900.0-fold, about 950.0-fold, about 1,000.0-fold, about 2,000.0-fold, about 3,000.0-fold, about 4,000.0-fold, about 5,000.0-fold, about 6,000.0-fold, about 7,000.0-fold, about 8,000.0-fold, about 9,000.0-fold, about 10,000.0-fold, about 20,000.0-fold, about 30,000.0-fold, about 40,000.0-fold, about 50,000.0-fold, about 100,000.0-fold, about 200,000-fold, or about 300,000-fold or more compared to a reference IL-12, as determined by an assay. In some embodiments, the assay comprises an AlphaLISA assay, an intracellular cytokine stain assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the capability of the IL-12 Fc fusion protein and/or the variant IL-12 p35 subunit domain to stimulate IFNγ production is reduced by at least 50.0-fold to about 100.0-fold, about 100.0-fold to about 150.0-fold, about 150.0-fold to about 200.0-fold, about 200.0-fold to about 250.0-fold, about 250.0-fold to about 300.0-fold, about 300.0-fold to about 350.0-fold, about 350.0-fold to about 400.0-fold, about 400.0-fold to about 450.0-fold, about 450.0-fold to about 500.0-fold, about 500.0-fold to about 550.0-fold, about 550.0-fold to about 600.0-fold, about 600.0-fold to about 650.0-fold, about 650.0-fold to about 700.0-fold, about 700.0-fold to about 750.0-fold, about 750.0-fold to about 800.0-fold, about 800.0-fold to about 850.0-fold, about 850.0-fold to about 900.0-fold, about 900.0-fold to about 950.0-fold, about 950.0-fold to about 1,000.0-fold, about 1,000.0-fold to about 2,000.0-fold, about 2,000.0-fold to about 3,000.0-fold, about 3,000.0-fold to about 4,000.0-fold, about 4,000.0-fold to about 5,000.0-fold, about 5,000.0-fold to about 6,000.0-fold, about 6,000.0-fold to about 7,000.0-fold, about 7,000.0-fold to about 8,000.0-fold, about 8,000.0-fold to about 9,000.0-fold, about 9,000.0-fold to about 10,000.0-fold, about 10,000.0-fold to about 20,000.0-fold, about 20,000.0-fold to about 30,000.0-fold, about 30,000.0-fold to about 40,000.0-fold, about 40,000.0-fold to about 50,000.0-fold, about 50,000.0-fold to about 60,000.0-fold, about 60,000.0-fold to about 70,000.0-fold, about 70,000.0-fold to about 80,000.0-fold, about 80,000.0-fold to about 90,000.0-fold, about 90,000.0-fold to about 100,000.0-fold, about 100,000.0-fold to about 200,000.0-fold, or about 200,000.0-fold to about 300,000.0-fold or higher compared to a reference IL-12, as determined by an assay. In some embodiments, the assay comprises an AlphaLISA assay, an intracellular cytokine stain assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain exhibits a capability to stimulate IFNγ production that is too weak to accurately be determined, and capability to stimulate IFNγ production of a reference IL-12 is between or equivalent to the lower or upper level of detectability of an assay (i.e., the potency of the reference IL-12 may be equal to the lower level of detectability, the upper level of detectability, or a value between the lower and upper levels of detectability; in other words, the capability to stimulate IFNγ production is “detectable” for the reference IL-12 and the capability to stimulate IFNγ production is “undetectable” for the IL-12 Fc fusion protein). In some embodiments, the assay comprises an AlphaLISA assay, an intracellular cytokine stain assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, IL-12 Fc fusion protein and/or variant IL-12 p35 subunit domains are provided, wherein one or more amino acid substitutions of the variant IL-12 p35 subunit domain improves half-life, as compared to half-life of a reference IL-12. In some embodiments, the fusion of the variant IL-12 p35 subunit domain and/or the (variant) IL-12 p40 subunit domain improves half-life, as compared to half-life of a reference. In some embodiments, the half-life of the IL-12 Fc fusion protein and/or variant IL-12 p35 subunit domain is decreased or increased about 1.5-fold, about 2.0-fold, about 3.0-fold, about 4.0-fold, about 5.0-fold, about 6.0-fold, about 7.0-fold, about 8.0-fold, about 9.0-fold, about 10.0-fold, about 15.0-fold, about 20.0-fold, about 30.0-fold, about 40.0-fold, about 50.0-fold, or about 100.0-fold or more, as compared to the half-life of a reference IL-12. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, IL-12 Fc fusion protein and/or variant IL-12 p35 subunit domains are provided, wherein one or more amino acid substitutions of the variant IL-12 p35 subunit domain improves half-life, as compared to half-life of a reference IL-12. In some embodiments, the fusion of the variant IL-12 p35 subunit domain and/or the (variant) IL-12 p40 subunit domain improves half-life, as compared to half-life of a reference. In some embodiments, the half-life of the IL-12 Fc fusion protein and/or variant IL-12 p35 subunit domain is decreased or increased about 1.5-fold to about 2.0-fold, about 2.0-fold to about 3.0-fold, about 3.0-fold to about 4.0-fold, about 4.0-fold to about 5.0-fold, about 5.0-fold to about 6.0-fold, about 6.0-fold to about 7.0-fold, about 7.0-fold to about 8.0-fold, about 8.0-fold to about 9.0-fold, about 9.0-fold to about 10.0-fold, about 10.0-fold to about 15.0-fold, about 15.0-fold to about 20.0-fold, about 20.0-fold to about 30.0-fold, about 30.0-fold to about 40.0-fold, about 40.0-fold to about 50.0-fold, or about 50.0-fold to about 100.0-fold or more, as compared to the half-life of a reference IL-12. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

Various methods for determining the half-life of an agent, such as, for example, IL-12, are known in the art. Those skilled in the relevant art will readily be able to determine and employ any number of such methods for determining changes in half-life. In some embodiments, at least one sample selected from a group comprising: (i) one or more blood samples, (ii) one or more plasma samples, (iii) one or more serum samples, (iv) one or more tissue samples, and (v) any combination thereof, are utilized to measure half-life. To measure the half-life, one, two, three, four, five, six, seven, eight, nine, 10 or more samples (as described above) may be utilized to measure half-life.

In some embodiments, the IL-12 Fc fusion protein and/or the variant IL-12 p35 subunit domain exhibits a resistance to tachyphylaxis of at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, about 100% or more compared to a reference IL-12, as determined by an assay. In some embodiments, the assay comprises: a measurement of plasma IFNγ using an AlphaLISA assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

In some embodiments, the IL-12 Fc fusion protein and/or the variant IL-12 p35 subunit domain exhibits a resistance to tachyphylaxis of about 10% to about 100%, about 10% to about 50%, about 20% to about 70%, about 30% to about 80%, about 40% to about 90%, about 50% to about 100%, about 20% to about 50%, about 40% to about 70%, about 30% to about 60%, about 40% to about 100%, about 20% to about 80%, or about 10% to about 90% compared to a reference IL-12, as determined by an assay. In some embodiments, the assay comprises: a measurement of plasma IFNγ using an AlphaLISA assay, a Luminex bead-based cytokine release assay, an ELISA, or an ELISpot assay. In some embodiments, the reference IL-12 comprises one or more of the following: a wild-type IL-12, a human wild-type IL-12, a commercially available IL-12 molecule, an IL-12 Fc fusion protein that does not comprise one or more amino acid substitutions selected from a group including: (a) Y40S/K170A, (b) Y40S/K170T, (c) Y40P/K168A, (d) Y40P/K170A, and (e) Y40E/K170A, or any combination thereof.

### b. p40 Subunit Domain:

In some embodiments, the IL-12 p40 subunit domain comprises a wild-type IL-12 p40 subunit domain.

In some embodiments, the IL-12 p40 subunit domain comprises a variant IL-12 p40 subunit domain.

In some embodiments, the IL-12 p40 subunit domain comprises a variant IL-12 p40 subunit domain, and in further embodiments the variant IL-12 p40 subunit domain comprises one or more amino acid substitutions selected from a group that includes: C177S, C252S, and C177S/C252S. In still some further embodiments, in instances where the variant IL-12 p40 subunit domain comprises a C177S or C177S/C252S substitution mutation(s), the variant IL-12 p35 subunit domain may further comprise a C74S substitution mutation.

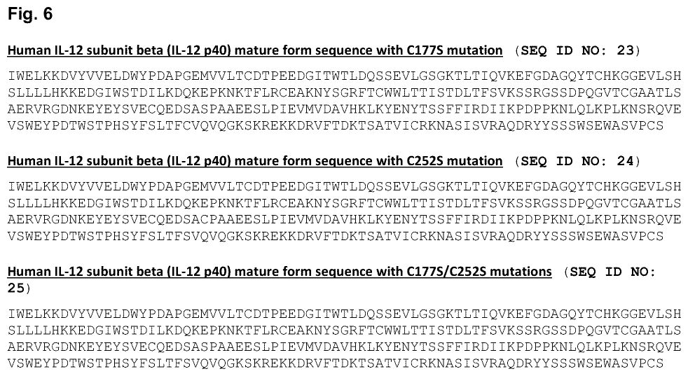

In some embodiments, the (variant) IL-12 p40 subunit domain comprises an amino acid sequence selected from a group that includes any one of: (i) SEQ ID NO: 4 (WT), (ii) SEQ ID NO: 23 (C177S), (iii) SEQ ID NO: 24 (C252S), and (iv) SEQ ID NO: 25 (C177S/C252S), as shown in FIGS. 1 and 6.

In some embodiments, the (variant) IL-12 p40 subunit domain comprises an amino acid sequence with at least 90%, 91%, 92%, 93%, 94%, 95%, 96%, 97%, 98%, 99% or higher sequence identity to an amino acid sequence selected from a group that includes any one of SEQ ID NOs: 4 and 23-25 (as shown in FIGS. 1 and 6).

In addition to the IL-12 Fc fusion protein described herein, various modifications to the IL-12 p40 subunit domain are known in the art and may further be included in IL-12 Fc fusion proteins described herein. Non-limiting examples of residues that may be modified include: E3, D7, E12, D14, W15, P17, D18, A19, P20, G21, E22, M23, D29, E32, E33, D34, L40, D41, Q42, S43, E45, L47, S49, T54, Q56, 155, Q56, K58, E59, F60, G61, D62, Y66, E73, H77, K84, K85, E86, D87, G88, 189, W90, D93, K99, E100, K102, N103, K104, T105, F106, R108, E110, N113, Y114, D129, D142, Q144, E156, R159, D161, N162, K163, E164, Y165, E166, S168, D170, Q172, D174, A176, C177, P178, A179, A180, E181, S183, P185, E187, M189, H194, K195, L196, K197, N200, S204, F206, R208, D209, D214, N218, Q220, N226, Q229, E231, E235, T242, P243, S245, Y246, F247, S248, C252, Q256, K258, S259, K260, R261, E262, K263, K264, D265, R266, V267, D270, N281, S283, S285, R287, Q289, D290, R291, Y292, Y293, and E299. In some embodiments, the (variant) IL-12 p40 subunit domain described herein or in the figures (see, e.g., FIGS. 1, 3, and 6) comprises one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more additional amino acid substitutions. In some embodiments, the (variant) IL-12 p40 subunit domain described herein or in the figures (see, e.g., FIGS. 1, 3, and 6) comprises about 1 to about 5, about 6 to about 10, about 11 to about 15, about 16 to about 20, about 21 to about 25, about 26 to about 30, about 31 to about 35, about 36 to about 40, about 41 to about 45, about 46 to about 50, about 51 to about 55, about 56 to about 60, about 1 to about 10, about 11 to about 20, about 21 to about 30, about 31 to about 40, about 41 to about 50, about 51 to about 60, about 1 to about 20, or about 21 to about 40, or about 41 to about 60 additional amino acid substitutions.

### 2. Antigen Binding Domains:

The present disclosure provides IL-12 Fc fusion proteins comprising: (1) a first IL-12 subunit domain and a second IL-12 subunit domain, wherein (i) the first IL-12 subunit domain comprises a variant IL-12 p35 subunit domain and the second IL-12 subunit domain comprises a (variant) IL-12 p40 subunit domain, or the first IL-12 subunit domain comprises a (variant) IL-12 p40 subunit domain and the second IL-12 subunit domain comprises a variant IL-12 p35 subunit domain, and (ii) the variant IL-12 p35 subunit domain comprises a set of amino acid substitutions that reduces binding affinity of the resultant IL-12 Fc fusion protein for IL-12Rβ2 as compared to a reference IL-12, wherein the set of amino acid substitutions is selected from a group including: (a) Y40S/K170A, (b) Y40S/K170T, (c) Y40P/K168A, (d) Y40P/K170A, and (e) Y40E/K170A; (2) at least one antigen binding domain (ABD), wherein the ABD binds a target antigen selected from a group including: (a) OX40, (b) LAG3, (c) PD1, (d) ICOS, (e) 41BB, (f) CCR7, and (g) CCR8; (3) one or more domain linkers; and (4) a first Fc domain and a second Fc domain.

In some instances, the IL-12 Fc fusion protein is configured such that the ABD is in a Fab format (i.e., the IL-12 Fc fusion protein comprises a variable heavy (VH) domain, a variable light (VL) domain, a constant heavy (CH1) domain, and a constant light (CL) domain). In some other instances, the IL-12 Fc fusion protein is configured such that the ABD is in an scFv format (i.e., the IL-12 Fc fusion protein comprises, from N-terminus to C-terminus, a VH domain, a linker, and a VL domain; or the reverse where the IL-12 Fc fusion protein comprises, from N-terminus to C-terminus, a VL domain, a linker, and a VH domain). In still other instances, the IL-12 Fc fusion protein is configured such that the ABD is in a nanobody format (i.e., the IL-12 Fc fusion protein comprises a VHH domain, wherein the VHH domain comprises a complementarity determining region (CDR) 1, a CDR2, and a CDR3, wherein the CDR1, CDR2, and CDR3 bind to a target antigen).

As will be appreciated by those skilled in the art, a subject IL-12 Fc fusion protein can be configured such that the IL-12 Fc fusion protein comprises one, two, three, or more ABDs. In one configuration, a subject IL-12 Fc fusion protein may consist of one ABD (i.e., the subject IL-12 Fc fusion protein is configured in a monovalent, monospecific format with respect to a target antigen). In other configurations, a subject IL-12 Fc fusion protein may comprise a first ABD and a second ABD. In some further embodiments, the first ABD and the second ABD bind the same target antigen (i.e., the subject IL-12 Fc fusion protein is configured in a bivalent, monospecific format with respect to a target antigen). In other further configurations, the first ABD binds a first target antigen, and the second ABD binds a second target antigen (i.e., the subject IL-12 Fc fusion protein is configured in a monovalent, bispecific format with respect to a first target antigen and a second target antigen). For the sake of brevity, exemplary ABDs are discussed in isolation without consideration of the number of ABDs that a subject IL-12 Fc fusion protein comprises. However, it is expressly contemplated that any of the ABDs described herein can be incorporated into a subject IL-12 Fc fusion protein, wherein the IL-12 Fc fusion protein is configured in: (1) a monovalent, monospecific format, (2) a bivalent, monospecific format, or (3) a monovalent, bispecific format, as if each configuration had been expressly set forth herein.

Binding affinity of a given ABD to its target antigen can be assessed using any number of assays known to those skilled in the relevant art. Non-limiting examples of suitable assays for evaluating binding affinity include: a surface plasmon resonance (SPR) assay, an enzyme-linked immunosorbent assay (ELISA), an ELISpot assay, Biacore assays, KinExA assays, flow cytometry, and the like.

Further included are ABDs that comprise at least about 90%, about 91%, about 92%, about 93%, about 94%, about 95%, about 96%, about 97%, about 98%, about 99% or more identity to any of the ABDs described herein so long as they retain their specific binding for their target antigen (e.g., when the target antigen comprises OX40, an ABD may comprise at least about 90%, about 91%, about 92%, about 93%, about 94%, about 95%, about 96%, about 97%, about 98%, about 99% or more identity to a given VH/VL pair described further herein, so long as the ABD comprising the variant VH/VL pair retains specific binding for OX40).

a. PD1:

In some embodiments, the target antigen comprises PD1 and the ABD binds PD1.

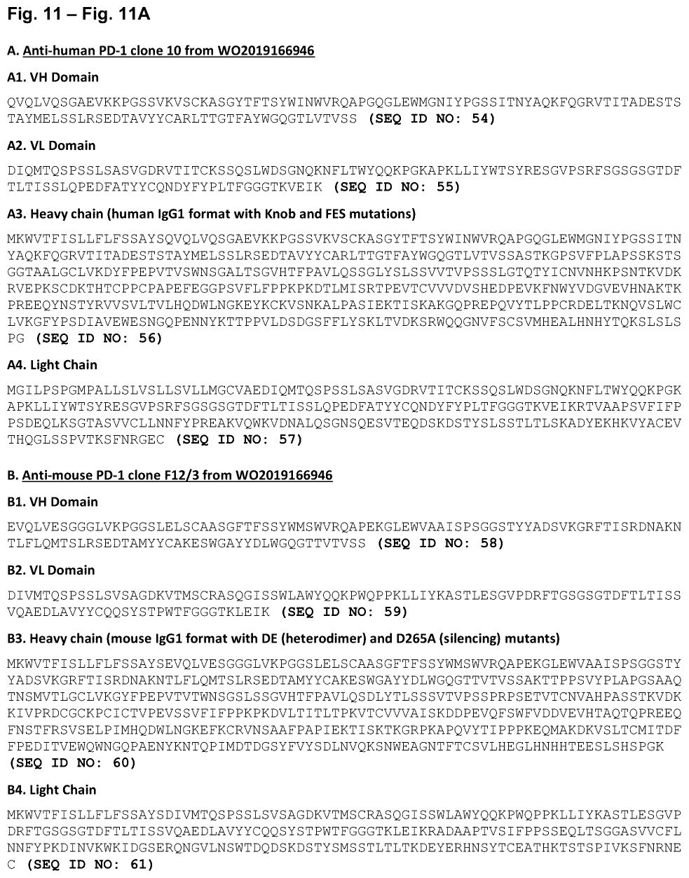

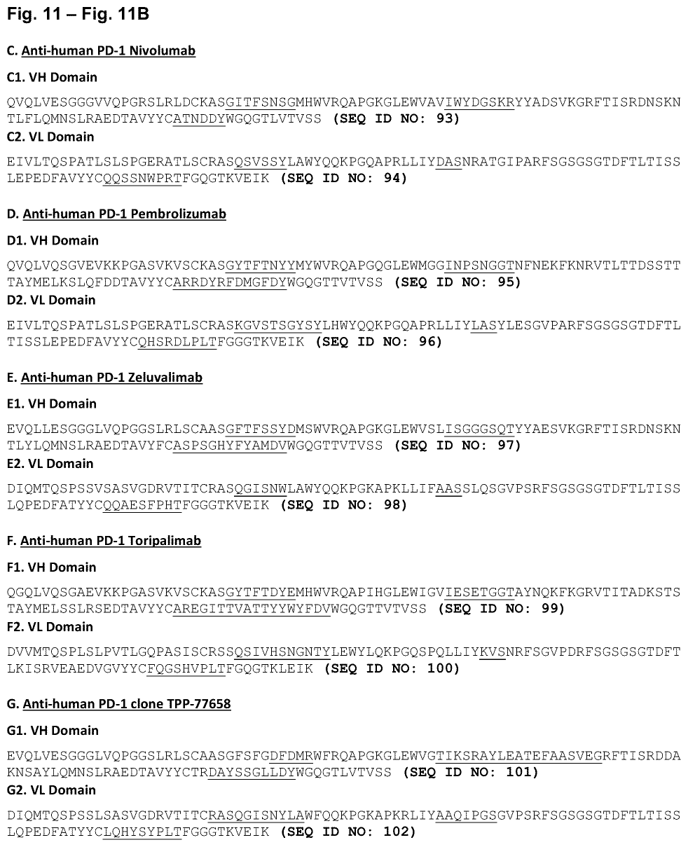

In some further embodiments, the ABD binds human PD1. In still some further embodiments, the ABD binds the extracellular domain (ECD) of human PD1. Suitable variable heavy (VH) and variable light (VL) domains that bind to the ECD of human PD1 include, but are not limited to, VH/VL pairs selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively, as shown in FIG. 11. It should be noted that these ABDs can be included in any of the IL-12 Fc fusion protein configurations described herein.

In some other further embodiments, the ABD binds mouse PD1. In still some further embodiments, the ABD binds the ECD of mouse PD1. Suitable VH and VL domains that bind to the ECD of mouse PD1 include, but are not limited to, VH/VL pairs selected from a group including: (i) SEQ ID NOs: 58 and 59, respectively, as shown in FIG. 11. It should be noted that these ABDs can be included in any of the IL-12 Fc fusion protein configurations described herein.

In still some other further embodiments, the ABD comprises a VHH domain, wherein the VHH domain comprises: (a) a complementarity determining region (CDR) 1; (b) a CDR2; and (C) a CDR3, wherein CDR1, CDR2, and CDR3 bind a target antigen, wherein the target antigen comprises PD1. It should be noted that these ABDs can be included in any of the IL-12 Fc fusion protein configurations described herein.

Further included herein are human anti-PD1 ABDs comprising a pair of variable heavy (VH) and variable light (VL) domains, wherein the VH/VL pair is selected from a group including: (i) SEQ ID NOs: 496 and 497, respectively, from publication WO2021067863A2, (ii) SEQ ID NOs: 528 and 529, respectively, from publication WO2021067863A2, (iii) SEQ ID NOs: 530 and 531, respectively, from publication WO2021067863A2, (iv) SEQ ID NOs: 532 and 533, respectively, from publication WO2021067863A2, (v) SEQ ID NOs: 534 and 535, respectively, from publication WO2021067863A2, (vi) SEQ ID NOs: 536 and 537, respectively, from publication WO2021067863A2, (vii) SEQ ID NOs: 542 and 543, respectively, from publication WO2021067863A2, (viii) SEQ ID NOs: 544 and 545, respectively, from publication WO2021067863A2, (ix) SEQ ID NOs: 546 and 547, respectively, from publication WO2021067863A2, (x) SEQ ID NOs: 548 and 549, respectively, from publication WO2021067863A2, (xi) SEQ ID NOs: 550 and 551, respectively, from publication WO2021067863A2, (xii) SEQ ID NOs: 552 and 553, respectively, from publication WO2021067863A2, (xiii) SEQ ID NOs: 554 and 555, respectively, from publication WO2021067863A2, (xiv) SEQ ID NOs: 556 and 557, respectively, from publication WO2021067863A2, (xv) SEQ ID NOs: 558 and 559, respectively, from publication WO2021067863A2, (xvi) SEQ ID NOs: 2 and 10, respectively, from publication WO2015112800A1, (xvii) SEQ ID NOs: 18 and 26, respectively, from publication WO2015112800A1, (xviii) SEQ ID NOs: 34 and 42, respectively, from publication WO2015112800A1, (xix) SEQ ID NOs: 50 and 58, respectively, from publication WO2015112800A1, (xx) SEQ ID NOs: 66 and 74, respectively, from publication WO2015112800A1, (xxi) SEQ ID NOs: 82 and 90, respectively, from publication WO2015112800A1, (xxii) SEQ ID NOs: 98 and 106, respectively, from publication WO2015112800A1, (xxiii) SEQ ID NOs: 114 and 122, respectively, from publication WO2015112800A1, (xxiv) SEQ ID NOs: 130 and 138, respectively, from publication WO2015112800A1, (xxv) SEQ ID NOs: 146 and 154, respectively, from publication WO2015112800A1, (xxvi) SEQ ID NOs: 162 and 170, respectively, from publication WO2015112800A1, (xxvii) SEQ ID NOs: 178 and 186, respectively, from publication WO2015112800A1, (xxviii) SEQ ID NOs: 194 and 202, respectively, from publication WO2015112800A1, (xxix) SEQ ID NOs: 210 and 202, respectively, from publication WO2015112800A1, (xxx) SEQ ID NOs: 218 and 202, respectively, from publication WO2015112800A1, (xxxi) SEQ ID NOs: 226 and 202, respectively, from publication WO2015112800A1, (xxxii) SEQ ID NOs: 234 and 202, respectively, from publication WO2015112800A1, (xxxiii) SEQ ID NOs: 242 and 202, respectively, from publication WO2015112800A1, (xxxiv) SEQ ID NOs: 250 and 202, respectively, from publication WO2015112800A1, (xxxv) SEQ ID NOs: 258 and 202, respectively, from publication WO2015112800A1, (xxxvi) SEQ ID NOs: 266 and 202, respectively, from publication WO2015112800A1, (xxxvii) SEQ ID NOs: 274 and 202, respectively, from publication WO2015112800A1, (xxxviii) SEQ ID NOs: 282 and 202, respectively, from publication WO2015112800A1, (xxxix) SEQ ID NOs: 290 and 202, respectively, from publication WO2015112800A1, (xl) SEQ ID NOs: 298 and 186, respectively, from publication WO2015112800A1, (xli) SEQ ID NOs: 306 and 186, respectively, from publication WO2015112800A1, (xlii) SEQ ID NOs: 314 and 186, respectively, from publication WO2015112800A1, (xliii) SEQ ID NOs: 1 and 8, respectively, from publication U.S. Pat. No. 8,008,449B2, (xliv) SEQ ID NOs: 2 and 9, respectively, from publication U.S. Pat. No. 8,008,449B2, (xlv) SEQ ID NOs: 3 and 10, respectively, from publication U.S. Pat. No. 8,008,449B2, (xlvi) SEQ ID NOs: 4 and 11, respectively, from publication U.S. Pat. No. 8,008,449B2, (xlvii) SEQ ID NOs: 5 and 12, respectively, from publication U.S. Pat. No. 8,008,449B2, (xlviii) SEQ ID NOs: 6 and 13, respectively, from publication U.S. Pat. No. 8,008,449B2, (xlix) SEQ ID NOs: 7 and 14, respectively, from publication U.S. Pat. No. 8,008,449B2, (1) SEQ ID NOs: 25 and 30, respectively, from publication US20110271358A1, (li) SEQ ID NOs: 26 and 31, respectively, from publication US20110271358A1, (lii) SEQ ID NOs: 27 and 32, respectively, from publication US20110271358A1, (liii) SEQ ID NOs: 28 and 33, respectively, from publication US20110271358A1, (liv) SEQ ID NOs: 27 and 33, respectively, from publication US20110271358A1, and (lv) SEQ ID NOs: 28 and 32, respectively, from publication US20110271358A1. All of the aforementioned publications are incorporated by reference in their entirety, with particularity for relevant disclosure pertaining to PD1 ABDs and the accompanying sequences described therein. Any such sequence described herein can be individually included or excluded from a subject IL-12 Fc fusion protein.

Further included herein are mouse anti-PD1 ABDs comprising a pair of variable heavy (VH) and variable light (VL) domains, wherein the VH/VL pair is selected from a group including: (i) SEQ ID NOs: 502 and 503, respectively, from publication WO2021067863A2, (ii) SEQ ID NOs: 504 and 505, respectively, from publication WO2021067863A2, (iii) SEQ ID NOs: 540 and 541, respectively, from publication WO2021067863A2, (iv) SEQ ID NOs: 560 and 561, respectively, from publication WO2021067863A2, (v) SEQ ID NOs: 55 and 56, respectively, from publication U.S. Pat. No. 8,168,757B2, (vi) SEQ ID NOs: 67 and 68, respectively, from publication U.S. Pat. No. 8,168,757B2, and (vii) SEQ ID NO: 63 and a variable light domain, respectively, from publication U.S. Pat. No. 8,168,757B2. All of the aforementioned publications are incorporated by reference in their entirety, with particularity for relevant disclosure pertaining to PD1 ABDs and the accompanying sequences described therein. Any such sequence described herein can be individually included or excluded from a subject IL-12 Fc fusion protein.

Further included herein are humanized anti-PD1 ABDs comprising a pair of variable heavy (VH) and variable light (VL) domains, wherein the VH/VL pair is selected from a group including: (i) SEQ ID NOs: 498 and 499, respectively, from publication WO2021067863A2, (ii) SEQ ID NOs: 508 and 509, respectively, from publication WO2021067863A2, (iii) SEQ ID NOs: 510 and 511, respectively, from publication WO2021067863A2, (iv) SEQ ID NOs: 512 and 513, respectively, from publication WO2021067863A2, (v) SEQ ID NOs: 514 and 515, respectively, from publication WO2021067863A2, (vi) SEQ ID NOs: 516 and 517, respectively, from publication WO2021067863A2, (vii) SEQ ID NOs: 518 and 519, respectively, from publication WO2021067863A2, (viii) SEQ ID NOs: 538 and 539, respectively, from publication WO2021067863A2, (ix) SEQ ID NOs: 562 and 563, respectively, from publication WO2021067863A2, (x) SEQ ID NOs: 2 and 3, respectively, from publication U.S. Ser. No. 11/034,765B2, (xi) SEQ ID NOs: 4 and 5, respectively, from publication U.S. Ser. No. 11/034,765B2, (xii) SEQ ID NOs: 4 and 66, respectively, from publication U.S. Ser. No. 11/034,765B2, (xiii) SEQ ID NOs: 6 and 7, respectively, from publication U.S. Ser. No. 11/034,765B2, (xiv) SEQ ID NOs: 8 and 9, respectively, from publication U.S. Ser. No. 11/034,765B2, (xv) SEQ ID NOs: 10 and 11, respectively, from publication U.S. Ser. No. 11/034,765B2, (xvi) SEQ ID NOs: 12 and 13, respectively, from publication U.S. Ser. No. 11/034,765B2, (xvii) SEQ ID NOs: 14 and 15, respectively, from publication U.S. Ser. No. 11/034,765B2, (xviii) SEQ ID NOs: 16 and 17, respectively, from publication U.S. Ser. No. 11/034,765B2, (xix) SEQ ID NOs: 164 and 178, respectively, from publication U.S. Ser. No. 10/294,299B2, (xx) SEQ ID NOs: 165 and 179, respectively, from publication U.S. Ser. No. 10/294,299B2, (xxi) SEQ ID NOs: 166 and 180, respectively, from publication U.S. Ser. No. 10/294,299B2, (xxii) SEQ ID NOs: 167 and 181, respectively, from publication U.S. Ser. No. 10/294,299B2, (xxiii) SEQ ID NOs: 168 and 182, respectively, from publication U.S. Ser. No. 10/294,299B2, (xxiv) SEQ ID NOs: 169 and 183, respectively, from publication U.S. Ser. No. 10/294,299B2, (xxv) SEQ ID NOs: 170 and 184, respectively, from publication U.S. Ser. No. 10/294,299B2, (xxvi) SEQ ID NOs: 171 and 185, respectively, from publication U.S. Ser. No. 10/294,299B2, (xxvii) SEQ ID NOs: 172 and 186, respectively, from publication U.S. Ser. No. 10/294,299B2, (xxviii) SEQ ID NOs: 173 and 187, respectively, from publication U.S. Ser. No. 10/294,299B2, (xxix) SEQ ID NOs: 174 and 188, respectively, from publication U.S. Ser. No. 10/294,299B2, (xxx) SEQ ID NOs: 175 and 189, respectively, from publication U.S. Ser. No. 10/294,299B2, (xxxi) SEQ ID NOs: 176 and 190, respectively, from publication U.S. Ser. No. 10/294,299B2, (xxxii) SEQ ID NOs: 177 and 190, respectively, from publication U.S. Ser. No. 10/294,299B2, (xxxiii) SEQ ID NOs: 23 and 31, respectively, from publication WO2008156712A1, and (xxxiv) SEQ ID NOs: 494 and 495, respectively, from publication WO2021067863A2. All of the aforementioned publications are incorporated by reference in their entirety, with particularity for relevant disclosure pertaining to PD1 ABDs and the accompanying sequences described therein. Any such sequence described herein can be individually included or excluded from a subject IL-12 Fc fusion protein.

Further included herein are anti-PD1 ABDs comprising a pair of variable heavy (VH) and variable light (VL) domains, wherein the VH/VL pair is selected from a group including: (i) SEQ ID NOs: 476 and 477, respectively, from publication WO2021067863A2, (ii) SEQ ID NOs: 478 and 479, respectively, from publication WO2021067863A2, (iii) SEQ ID NOs: 480 and 481, respectively, from publication WO2021067863A2, (iv) SEQ ID NOs: 482 and 483, respectively, from publication WO2021067863A2, (v) SEQ ID NOs: 484 and 485, respectively, from publication WO2021067863A2, (vi) SEQ ID NOs: 486 and 487, respectively, from publication WO2021067863A2, (vii) SEQ ID NOs: 488 and 489, respectively, from publication WO2021067863A2, (viii) SEQ ID NOs: 490 and 491, respectively, from publication WO2021067863A2, (ix) SEQ ID NOs: 492 and 493, respectively, from publication WO2021067863A2, (x) SEQ ID NOs: 520 and 521, respectively, from publication WO2021067863A2, (xi) SEQ ID NOs: 522 and 523, respectively, from publication WO2021067863A2, (xii) SEQ ID NOs: 524 and 525, respectively, from publication WO2021067863A2, (xiii) SEQ ID NOs: 526 and 527, respectively, from publication WO2021067863A2, (xiv) SEQ ID NOs: 564 and 565, respectively, from publication WO2021067863A2, (xv) SEQ ID NOs: 566 and 567, respectively, from publication WO2021067863A2, (xvi) SEQ ID NOs: 576 and 577, respectively, from publication WO2021067863A2, (xvii) SEQ ID NOs: 7 and 8, respectively, from publication WO2023242769A1, (xviii) SEQ ID NOs: 17 and 18, respectively, from publication WO2023242769A1, (xix) SEQ ID NOs: 33 and 34, respectively, from publication WO2023242769A1, (xx) SEQ ID NOs: 330 and 331, respectively, from publication US20150203579A1, and (xxi) any one of SEQ ID NOs: 568-576 and any one of SEQ ID NOs: 577-586, respectively, from publication WO2021067863A2. All of the aforementioned publications are incorporated by reference in their entirety, with particularity for relevant disclosure pertaining to PD1 ABDs and the accompanying sequences described therein. Any such sequence described herein can be individually included or excluded from a subject IL-12 Fc fusion protein.

b. LAG3:

In some embodiments, the target antigen comprises LAG3 and the ABD binds LAG3.

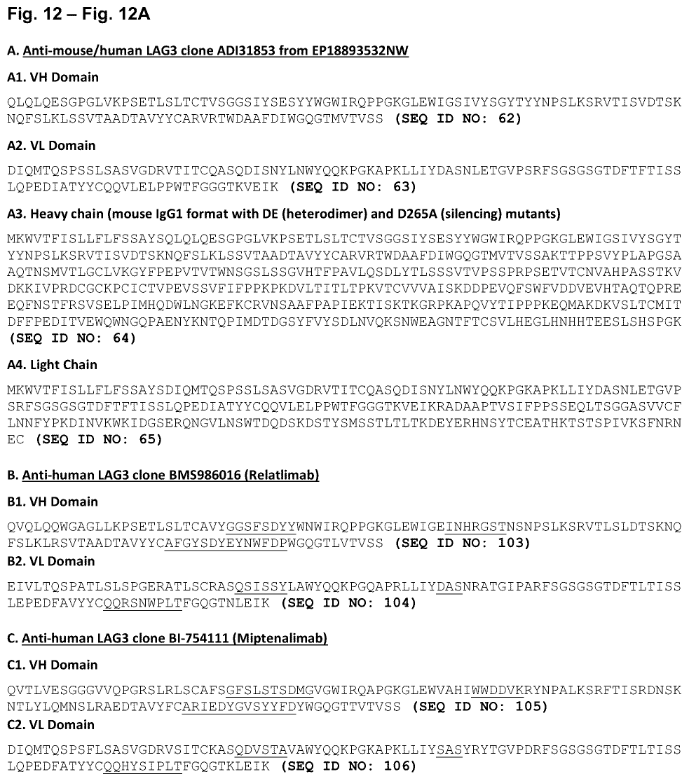

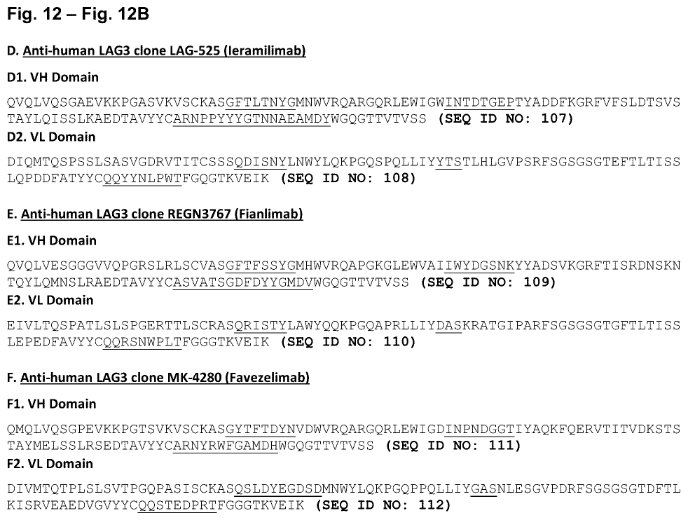

In some further embodiments, the ABD binds human LAG3. In still some further embodiments, the ABD binds the ECD of human LAG3. Suitable VH and VL domains that bind to the ECD of human LAG3 include, but are not limited to, VH/VL pairs selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively, as shown in FIG. 12. It should be noted that these ABDs can be included in any of the IL-12 Fc fusion protein configurations described herein.

In some other further embodiments, the ABD binds mouse LAG3. In still some further embodiments, the ABD binds the ECD of mouse LAG3. Suitable VH and VL domains that bind to the ECD of mouse LAG3 include, but are not limited to, VH/VL pairs selected from a group including: (i) SEQ ID NOs: 62 and 63, respectively, as shown in FIG. 12. It should be noted that these ABDs can be included in any of the IL-12 Fc fusion protein configurations described herein.

In still some other further embodiments, the ABD comprises a VHH domain, wherein the VHH domain comprises: (a) a CDR1; (b) a CDR2; and (C) a CDR3, wherein CDR1, CDR2, and CDR3 bind a target antigen, wherein the target antigen comprises LAG3. It should be noted that these ABDs can be included in any of the IL-12 Fc fusion protein configurations described herein.

Further included herein are human anti-LAG3 ABDs comprising a pair of variable heavy (VH) and variable light (VL) domains, wherein the VH/VL pair is selected from a group including: (i) SEQ ID NOs: 12 and 14, respectively, from publication U.S. Pat. No. 9,505,839B2, (ii) SEQ ID NOs: 3 and 4, respectively, from publication U.S. Ser. No. 11/390,676B2, (iii) SEQ ID NOs: 7 and 8, respectively, from publication U.S. Ser. No. 11/390,676B2, (iv) SEQ ID NOs: 11 and 12, respectively, from publication U.S. Ser. No. 11/390,676B2, (v) SEQ ID NOs: 15 and 16, respectively, from publication U.S. Ser. No. 11/390,676B2, (vi) SEQ ID NOs: 19 and 20, respectively, from publication U.S. Ser. No. 11/390,676B2, (vii) SEQ ID NOs: 23 and 24, respectively, from publication U.S. Ser. No. 11/390,676B2, (viii) SEQ ID NOs: 3 and 4, respectively, from publication WO2018201096A1, (ix) SEQ ID NOs: 254 and 303, respectively, from publication WO2020038397A1, (x) SEQ ID NOs: 255 and 304, respectively, from publication WO2020038397A1, (xi) SEQ ID NOs: 256 and 305, respectively, from publication WO2020038397A1, (xii) SEQ ID NOs: 257 and 306, respectively, from publication WO2020038397A1, (xiii) SEQ ID NOs: 258 and 307, respectively, from publication WO2020038397A1, (xiv) SEQ ID NOs: 259 and 308, respectively, from publication WO2020038397A1, (xv) SEQ ID NOs: 260 and 309, respectively, from publication WO2020038397A1, (xvi) SEQ ID NOs: 261 and 310, respectively, from publication WO2020038397A1, (xvii) SEQ ID NOs: 262 and 311, respectively, from publication WO2020038397A1, (xviii) SEQ ID NOs: 263 and 312, respectively, from publication WO2020038397A1, (xix) SEQ ID NOs: 264 and 313, respectively, from publication WO2020038397A1, (xx) SEQ ID NOs: 265 and 314, respectively, from publication WO2020038397A1, (xxi) SEQ ID NOs: 266 and 315, respectively, from publication WO2020038397A1, (xxii) SEQ ID NOs: 267 and 316, respectively, from publication WO2020038397A1, (xxiii) SEQ ID NOs: 268 and 317, respectively, from publication WO2020038397A1, (xxiv) SEQ ID NOs: 269 and 318, respectively, from publication WO2020038397A1, (xxv) SEQ ID NOs: 270 and 319, respectively, from publication WO2020038397A1, (xxvi) SEQ ID NOs: 271 and 320, respectively, from publication WO2020038397A1, (xxvii) SEQ ID NOs: 272 and 321, respectively, from publication WO2020038397A1, (xxviii) SEQ ID NOs: 273 and 322, respectively, from publication WO2020038397A1, (xxix) SEQ ID NOs: 274 and 323, respectively, from publication WO2020038397A1, (xxx) SEQ ID NOs: 275 and 324, respectively, from publication WO2020038397A1, (xxxi) SEQ ID NOs: 276 and 325, respectively, from publication WO2020038397A1, (xxxii) SEQ ID NOs: 277 and 326, respectively, from publication WO2020038397A1, (xxxiii) SEQ ID NOs: 278 and 327, respectively, from publication WO2020038397A1, (xxxiv) SEQ ID NOs: 279 and 328, respectively, from publication WO2020038397A1, (xxxv) SEQ ID NOs: 280 and 329, respectively, from publication WO2020038397A1, (xxxvi) SEQ ID NOs: 281 and 330, respectively, from publication WO2020038397A1, (xxxvii) SEQ ID NOs: 282 and 331, respectively, from publication WO2020038397A1, (xxxviii) SEQ ID NOs: 283 and 332, respectively, from publication WO2020038397A1, (xxxix) SEQ ID NOs: 284 and 333, respectively, from publication WO2020038397A1, (xl) SEQ ID NOs: 285 and 334, respectively, from publication WO2020038397A1, (xli) SEQ ID NOs: 286 and 335, respectively, from publication WO2020038397A1, (xlii) SEQ ID NOs: 287 and 336, respectively, from publication WO2020038397A1, (xliii) SEQ ID NOs: 288 and 337, respectively, from publication WO2020038397A1, (xliv) SEQ ID NOs: 289 and 338, respectively, from publication WO2020038397A1, (xlv) SEQ ID NOs: 290 and 339, respectively, from publication WO2020038397A1, (xlvi) SEQ ID NOs: 291 and 340, respectively, from publication WO2020038397A1, (xlvii) SEQ ID NOs: 292 and 341, respectively, from publication WO2020038397A1, (xlviii) SEQ ID NOs: 293 and 342, respectively, from publication WO2020038397A1, (xlix) SEQ ID NOs: 294 and 343, respectively, from publication WO2020038397A1, (1) SEQ ID NOs: 295 and 344, respectively, from publication WO2020038397A1, (li) SEQ ID NOs: 296 and 345, respectively, from publication WO2020038397A1, (lii) SEQ ID NOs: 297 and 346, respectively, from publication WO2020038397A1, (liii) SEQ ID NOs: 298 and 347, respectively, from publication WO2020038397A1, (liv) SEQ ID NOs: 299 and 348, respectively, from publication WO2020038397A1, (lv) SEQ ID NOs: 300 and 349, respectively, from publication WO2020038397A1, (lvi) SEQ ID NOs: 301 and 350, respectively, from publication WO2020038397A1, (lvii) SEQ ID NOs: 302 and 351, respectively, from publication WO2020038397A1, (lviii) SEQ ID NOs: 65 and 73, respectively, from publication U.S. Ser. No. 10/882,908B2, (lix) SEQ ID NOs: 220 and 221, respectively, from publication U.S. Ser. No. 10/882,908B2, (lx) SEQ ID NOs: 418 and 426, respectively, from publication U.S. Ser. No. 10/358,495B2, (lxi) SEQ ID NOs: 1 and 2, respectively, from publication U.S. Pat. No. 9,902,772, (lxii) SEQ ID NOs: 1 and 3, respectively, from publication U.S. Pat. No. 9,902,772, (lxiii) SEQ ID NOs: 1 and 4, respectively, from publication U.S. Pat. No. 9,902,772, (lxiv) SEQ ID NOs: 1 and 5, respectively, from publication U.S. Pat. No. 9,902,772, (lxv) SEQ ID NOs: 1 and 6, respectively, from publication U.S. Pat. No. 9,902,772, (lxvi) SEQ ID NOs: 1 and 7, respectively, from publication U.S. Pat. No. 9,902,772, (lxvii) SEQ ID NOs: 8 and 9, respectively, from publication U.S. Pat. No. 9,902,772, (lxviii) SEQ ID NOs: 10 and 11, respectively, from publication U.S. Pat. No. 9,902,772, (lxix) SEQ ID NOs: 12 and 13, respectively, from publication U.S. Pat. No. 9,902,772, and (lxx) SEQ ID NOs: 8 and 14, respectively, from publication U.S. Pat. No. 9,902,772. All of the aforementioned publications are incorporated by reference in their entirety, with particularity for relevant disclosure pertaining to LAG3 ABDs and the accompanying sequences described therein. Any such sequence described herein can be individually included or excluded from a subject IL-12 Fc fusion protein.

Further included herein are humanized anti-LAG3 ABDs comprising a pair of variable heavy (VH) and variable light (VL) domains, wherein the VH/VL pair is selected from a group including: (i) SEQ ID NOs: 27 and 28, respectively, from publication U.S. Ser. No. 11/390,676B2, (ii) SEQ ID NOs: 6 and 8, respectively, from publication WO2021147837A1, (iii) SEQ ID NOs: 10 and 12, respectively, from publication WO2021147837A1, (iv) SEQ ID NOs: 182 and 88, respectively, from publication US20210095026A1, (v) SEQ ID NOs: 20 and 21, respectively, from publication U.S. Ser. No. 11/285,207B2, (vi) SEQ ID NOs: 28 and 29, respectively, from publication U.S. Ser. No. 11/285,207B2, (vii) SEQ ID NOs: 36 and 37, respectively, from publication U.S. Ser. No. 11/285,207B2, (viii) SEQ ID NOs: 44 and 45, respectively, from publication U.S. Ser. No. 11/285,207B2, (ix) SEQ ID NOs: 52 and 53, respectively, from publication U.S. Ser. No. 11/285,207B2, and (x) SEQ ID NOs: 9 and 4, respectively, from publication WO2014140180A1. All of the aforementioned publications are incorporated by reference in their entirety, with particularity for relevant disclosure pertaining to LAG3 ABDs and the accompanying sequences described therein. Any such sequence described herein can be individually included or excluded from a subject IL-12 Fc fusion protein.

c. OX40:

In some embodiments, the target antigen comprises OX40 and the ABD binds OX40.

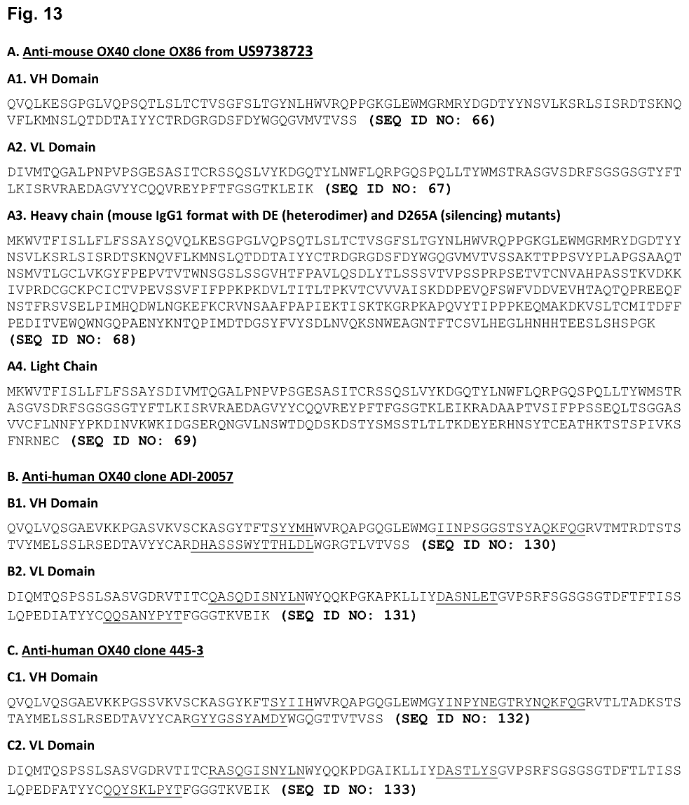

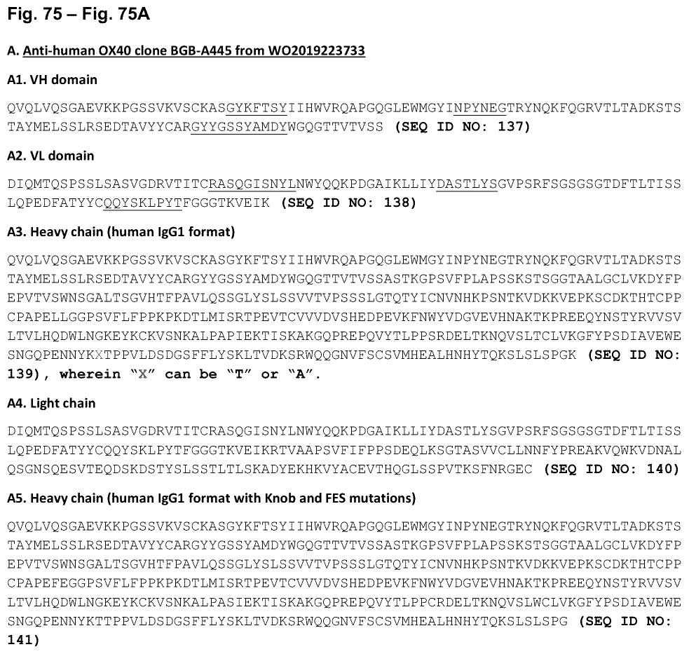

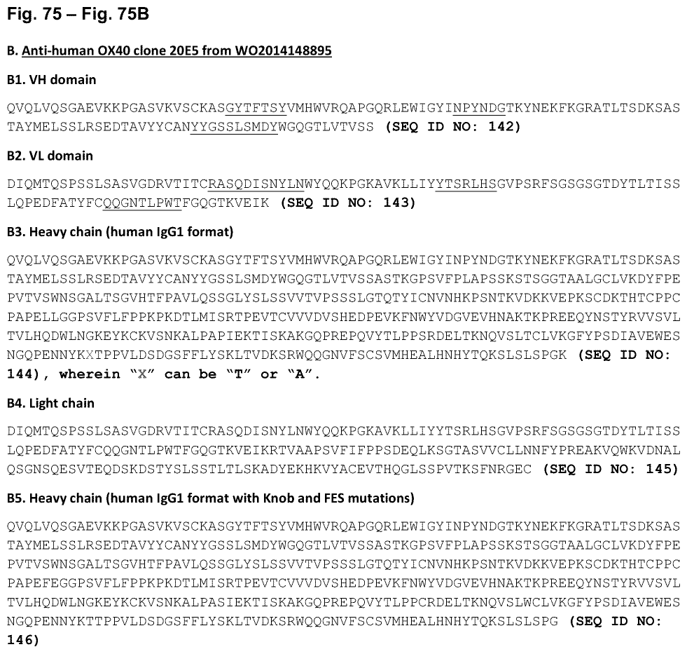

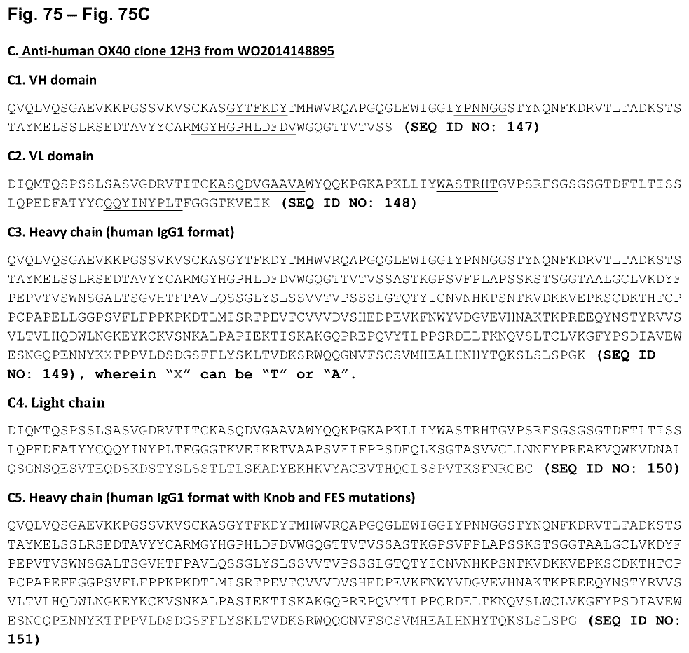

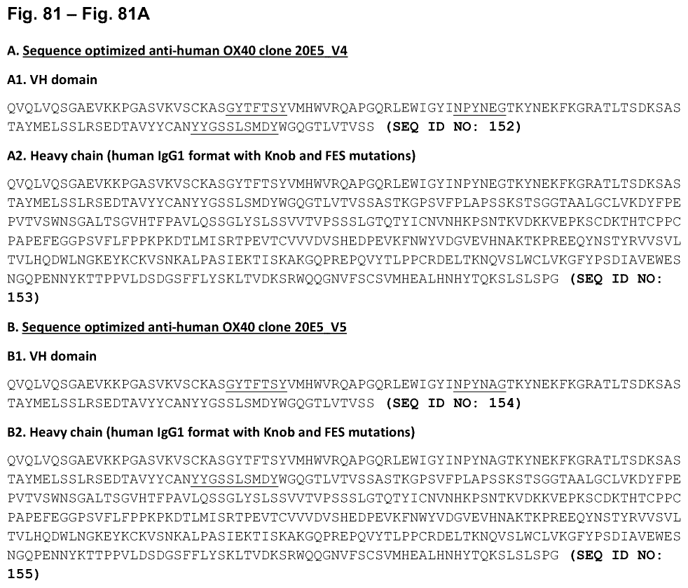

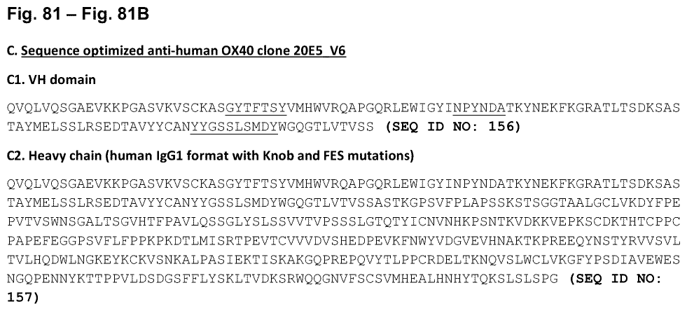

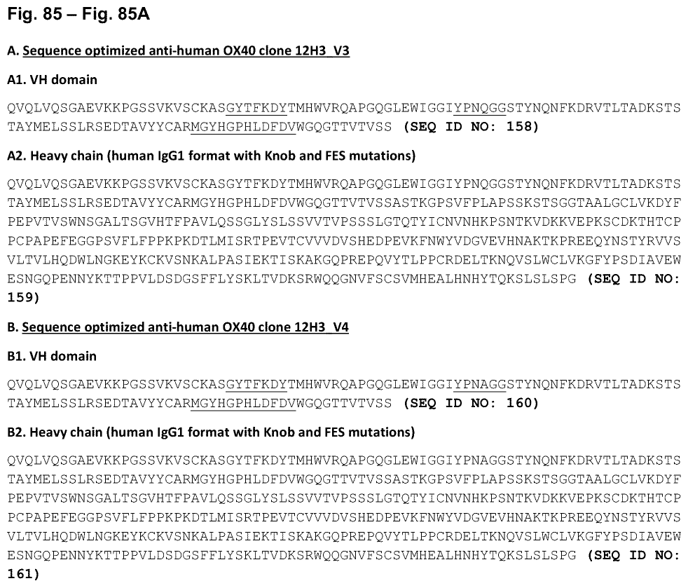

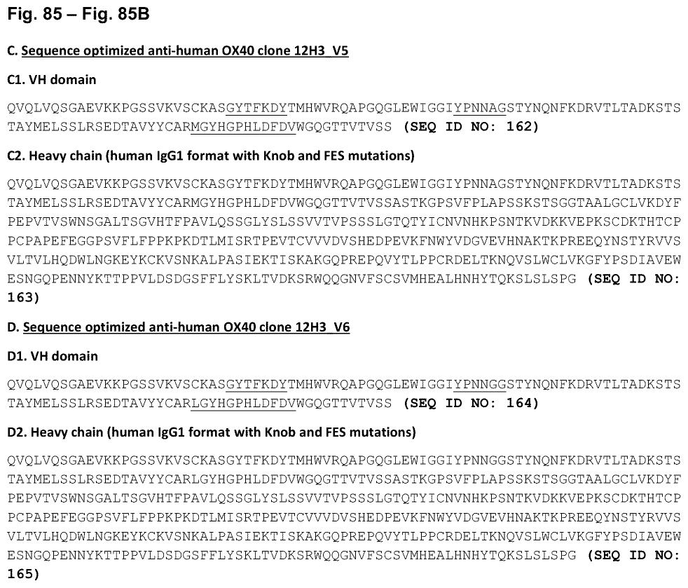

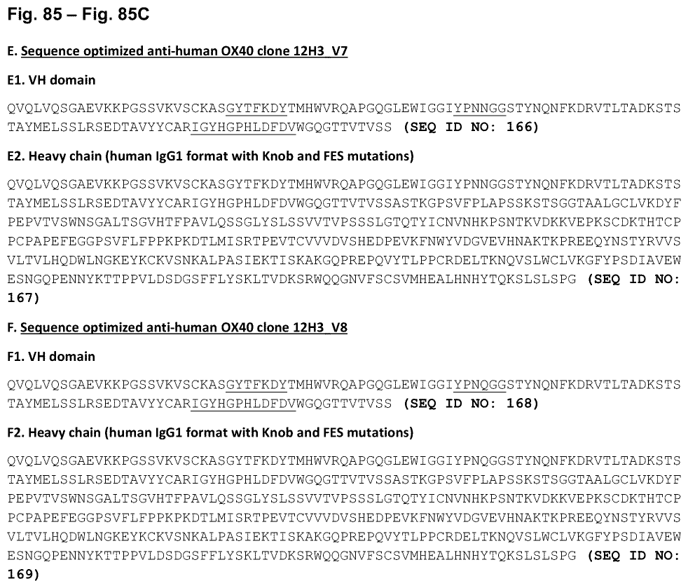

In some further embodiments, the ABD binds human OX40. In still some further embodiments, the ABD binds the ECD of human OX40. Suitable VH and VL domains that bind to the ECD of human OX40 include, but are not limited to, VH/VL pairs selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. It should be noted that these ABDs can be included in any of the IL-12 Fc fusion protein configurations described herein.

In some other further embodiments, the ABD binds mouse OX40. In still some further embodiments, the ABD binds the ECD of mouse OX40. Suitable VH and VL domains that bind to the ECD of mouse OX40 include, but are not limited to, VH/VL pairs selected from a group including: (i) SEQ ID NOs: 66 and 67, respectively, as shown in FIG. 13. It should be noted that these ABDs can be included in any of the IL-12 Fc fusion protein configurations described herein.

In still some other further embodiments, the ABD comprises a VHH domain, wherein the VHH domain comprises: (a) a CDR 1; (b) a CDR2; and (C) a CDR3, wherein CDR1, CDR2, and CDR3 bind a target antigen, wherein the target antigen comprises OX40. It should be noted that these ABDs can be included in any of the IL-12 Fc fusion protein configurations described herein.

In some embodiments, the ABD specifically binds to an epitope of (human) OX40, wherein the epitope of OX40 also has specific binding for (human) OX40 ligand (“(h)OX40L”). In other embodiments, the ABD specifically binds to an epitope of (human) OX40, wherein the epitope of OX40 does not have specific binding for (human) OX40L. Phrased differently, in some instances, the (human) anti-OX40 ABD comprises a blocking anti-OX40 ABD (i.e., when the anti-OX40 ABD competitively binds to an epitope of OX40 that is also specifically bound by OX40L), whereas in other instances the (human) anti-OX40 ABD comprises a non-blocking anti-OX40 ABD (i.e., when the anti-OX40 ABD binds to an epitope of OX40 that is not specifically bound by OX40L).

Further included herein are human anti-OX40 ABDs comprising a pair of variable heavy (VH) and variable light (VL) domains, wherein the VH/VL pair is selected from a group including: (i) SEQ ID NOs: 16 and 21, respectively, from publication CN108623685B. All of the aforementioned publications are incorporated by reference in their entirety, with particularity for relevant disclosure pertaining to OX40 ABDs and the accompanying sequences described therein. Any such sequence described herein can be individually included or excluded from a subject IL-12 Fc fusion protein.

Further included herein are anti-OX40 ABDs comprising a pair of variable heavy (VH) and variable light (VL) domains, wherein the VH/VL pair is selected from a group including: (i) SEQ ID NOs: 59 and 29, respectively, from publication U.S. Pat. No. 9,738,723, (ii) SEQ ID NOs: 14 and 16, respectively, from publication WO2019223733, (iii) SEQ ID NOs: 20 and 22, respectively, from publication WO2019223733, and (iv) SEQ ID NOs: 26 and 28, respectively, from publication WO2019223733. All of the aforementioned publications are incorporated by reference in their entirety, with particularity for relevant disclosure pertaining to OX40 ABDs and the accompanying sequences described therein. Any such sequence described herein can be individually included or excluded from a subject IL-12 Fc fusion protein.

There are several additional anti-OX40 antibodies, anti-OX40 ABDs comprising a pair of variable heavy (VH) domain and variable light (VL) domain pairs (VH/VL pairs), and anti-OX40 ABDs comprising a set of vhCDR1-3 and a set of vlCDR1-3 (sets of vhCDR1-3/vlCDR1-3 sequences) known in the art that can be individually included or excluded from a subject IL-12 Fc fusion protein as described herein. Examples include, but are not limited to: (i) any one of antibodies SCO2008, SCO2009, SCO2010, SCO2011, SCO2012, SCO2021, SCO2022, and SCO2023 as set forth in WO2003106498, (ii) any one of antibodies SAP29-50, SAP25-29, and SAP 29-23 as set forth in WO2017077085, (iii) any one of antibodies 14A4, 6E1, OX40.5, and OX40.8 as set forth in WO2016196228, (iv) MEDI6383 as set forth in WO2016057667, (v) DF004 as set forth in WO2017063162, (vi) INCAGN1949 as set forth in WO2016179517, (vii) IMG-007 as set forth in WO2023061424, (viii) any one of antibodies OX1A11, OX1B1, OX1B2, OX1B3, OX1C4, OX1C5, OX1D9, OX1E10, OX1E7, OX1F2, OX1G9, OX2B12, OX2B3, OX2B4, OX2B6, OX2F2, OX2G2, OX3C10, OX4A11, OX4A12, OX4B6, OX4D4, OX4D7, OX4D9, OX4G9, OX4H4, OX5B9, OX5C1, OX5D7, OX4B5, OX2E5, OX2B5, and OX5C11 as set forth in WO2018031490, (ix) any one of anti-OX40 ABDs comprising a VH/VL pair, wherein the VH/VL pair comprises amino acid sequences selected from a group including SEQ ID NOs: 106 and 130, SEQ ID NOs: 106 and 131, SEQ ID NOs: 107 and 130, SEQ ID NOs: 108 and 130, SEQ ID NOs: 109 and 130, SEQ ID NOs: 110 and 130, and SEQ ID NOs: 111 and 130 as set forth in U.S. Ser. No. 11/498,972B2, (x) any one of antibodies ADI-20112, ADI-25650, ADI-25651, ADI-25652, ADI-25653, ADI-25654, ADI-20078, ADI-23515, ADI-23518, ADI-23519, ADI-20048, ADI-20096, ADI-20051, ADI-20065, ADI-20066, ADI-20118, and ADI-20113 as set forth in U.S. Ser. No. 11/498,972B2, (xi) any one of anti-OX40 ABDs comprising a VH/VL pair, wherein the VH/VL pair comprises amino acid sequences selected from a group including SEQ ID NOs: 61 and 67, SEQ ID NOs: 61 and 68, SEQ ID NOs: 61 and 69, SEQ ID NOs: 91 and 97, SEQ ID NOs: 91 and 98, and SEQ ID NOs: 91 and 99 as set forth in U.S. Ser. No. 11/242,398B2, (xii) any one of anti-OX40 ABDs comprising a VH/VL pair, wherein the VH/VL pair comprises amino acid sequences selected from a group including SEQ ID NOs: 11 and 12, SEQ ID NOs: 13 and 14, SEQ ID NOs: 15 and 16, SEQ ID NOs: 17 and 18, and SEQ ID NOs: 19 and 20 as set forth in CN108822213A, (xiii) any one of antibodies 71H8, 68C8, 6A12, 68C8-VH0VL0, 68C8-VH2VL2, 68C8-VH2VL3, 68C8-VH3VL2, 68C8-VH3VL3, 71H8-VH0VL0, 71H8-VH2VL2, 71H8-VH2VL3, 71H8-VH3VL2, and 71H8-VH3VL3 as set forth in U.S. Ser. No. 11/091,558B2, (xiv) SP197 as set forth in U.S. Ser. No. 10/975,157B2, (xv) any one of antibodies 07-9H3, 07-9A4, 11-5C1, and 17-5D10 as set forth in U.S. Ser. No. 10/934,365B2, (xvi) any one of antibodies 106-108, 106-317, 106-107, 106-148, 119-204A, 119-220C, 119-33A, 119-58, 119-181A, 119-157A, 120-140A, 119-8B, 119-173B, 106-132, 106-222, 119-43, 119-122, 119-69A, 120-56, and 120-270 as set forth in U.S. Ser. No. 10/851,173B2, (xvii) any one of antibodies Hu3738, Hu3726, Hu3739, Hu3741 as set forth in U.S. Ser. No. 10/040,864B2, (xviii) any one of antibodies PR002055, PR002056, PR002057, PR002058, PR002059, PR002060, PR002061, PR002062, PR002063, PR002064, PR002065, PR002066, PR002067, PR002068, PR002069, PR002070, PR002071, PR002072, PR002073, PR002074, PR002075, PR002076, and PR002077 as set forth in US20230295324A1, (xix) HFB301001 as set forth in US20230061604A1, (xx) any one of antibodies AH02906, AH02913, AH02915, AH02916, AH02917, AH02919, AH02921, AH02922, AH02923, and AH02925 as set forth in US20220119540A1, (xxi) any one of antibodies 9C12, 1D9, 8A7, and 9D7 as set forth in US20220017632A1, (xxii) any one of antibodies 2G3-hu1, 2G3-hu2, 2G3-hu3, 2G3-hu4, hu4B5-V1, hu4B5-V2, hu4B5-V3, hu4B5-V4, hu4B5-V5, hu4B5-V6, hu4B5-V8, hu4B5-V9, hu4B5-V10, hu4B5-V11, hu4B5-V15, hu4B5-V16, hu4B5-V17, hu4B5-V7, hu4B5-V12, hu4B5-V18, hu4B5-V19, hu4B5-V20, hu4B5-V21, hu4B5-V13, hu4B5-V22, hu4B5-V23, hu4B5-V24, hu4B5-V25, and hu4B5-V14 as set forth in US20210355229A1, (xxiii) any one of antibodies MT01-C1, MT01-C1(G2), and MT01-L1 as set forth in US20210198375A1, (xxiv) any one of antibodies 1A7, 1A7.gr.1, 1A7.gr.2, 1A7.gr.3, 1A7.gr.4, 1A7.gr.5, 1A7.gr.6, 1A7.gr.7, 3C8, 3C8.gr.1, 3C8.gr.2, 3C8.gr.3, 3C8.gr.4, 3C8.gr.5, 3C8.gr.6, 3C8.gr.7, 3C8.gr.8, 3C8.gr.9, 3C8.gr.10, 3C8.gr.11, 3C8.gr.12, 3C8.gr.13, 3C8.gr.14, 3C8.gr.15, 1D2, 1D2.gr.1, 1D2.gr.2, and 1D2.gr.3 as set forth in U.S. Pat. No. 9,975,957B2, (xxv) any one of antibodies 1A06, 2B07, 2C09, 1D10, 2E4, 2H06, 3E11, 3G9, and G3 as set forth in U.S. Ser. No. 11/117,972B2, wherein the antibody comprises a single domain antibody comprising a VHH domain, (xxvi) any one of antibodies PN001, PN002, PN003, PN004, PN005, PN006, PN008, PN009, PN011, PN012, PN013, PN014, PN017, PN019, PN021, PN024, PN027, PN028, PN029, PN035, PN037, PN038, PN043, PN045, PN046, PN049, PN051, PN053, PN059, PN061, PN062, PN063, PN064, PN065, PN066, PN067, PN068, PN069, PN070, PN071, PN101, PN102, PN103, PN104, PN105, PN106, PN107, PN108, PN109, PN110, PN111, PN112, PN113, PN114, PN115, PN116, PN117, PN118, PN119, PN120, PN121, PN122, PN123, PN124, PN125, PN126, PN127, PN128, PN129, PN130, and PN131 as set forth in WO2022042659A1, (xxvii), (xxviii) any one of antibodies 5E10, 2B7, 1B3, 1E6, 2D3B, 1F4B, 2D9, 4A6B, 4F5, 4D10, 4E8, 4H2, 1B7, 1C8, 3B8, and 1G9B as set forth in WO2023109901A1, wherein the antibody comprises a single domain antibody comprising a VHH domain, (xxix) any one of antibodies Antibody M and Antibody M-KF as set forth in WO2023001118A1, (xxx) any one of antibodies HuEM1007-044-1, HuEM1007-044-2, HuEM1007-044-3, HuEM1007-044-4, HuEM1007-044-5, HuEM1007-044-6, HuEM1007-044-7, HuEM1007-044-8, EM1007-mAb044c-9, EM1007-mAb044c-10, EM1007-mAb044c-11, EM1007-mAb044c-12, EM1007-mAb044c-13, HuEM1007-044-14, EM1007-mAb044c-15, HuEM1007-044-16, and EM1007-mAb044c-17 as set forth in WO2022258015A1, (xxxi) any one of antibodies O410, O212, O15, O32, O34, O35, and O31C as set forth in US20240182593A1, (xxxii) any one of antibodies 25A7, 27G12, 11F7, hz25A7H1L1, hz25A7 ml, hz25A7m2, hz25A7m3, hz25A7m4, hz25A7m5, hz25A7m6, hz25A7m7, hz25A7m8, h27G12HL, h27G12H1L1, and h27G12H1L2 as set forth in WO2021143914A1, (xxxiii) any one of anti-OX40 ABDs comprising a VH/VL pair, wherein the VH/VL pair comprises amino acid sequences selected from a group including SEQ ID NOs: 4 and 12, SEQ ID NOs: 20 and 28, SEQ ID NOs: 36 and 44, SEQ ID NOs: 5 and 13, and SEQ ID NOs: 21 and 13 as set forth in WO2021129874A1, (xxxiii) any one of anti-OX40 ABDs comprising a VH/VL pair, wherein the VH/VL pair comprises amino acid sequences selected from a group including SEQ ID NOs: 3 and 4, and SEQ ID NOs: 5 and 6 as set forth in CN114591988B, (xxxiv) any one of antibodies A2, A3, B2, B11, C10, A2-1, A2-53, A2-54, B11-3, B11-11, B11-51, A2-10, A3-10, B2-10, B11-10, B11-1, C10-10, B2-1, and C10-1 as set forth in CN112794907B, (xxxv) any one of antibodies 3D9, 3C9, 1G4, 17C1, 3F5, 9G7, 15A7, and 1A2 as set forth in CN112574312B, (xxxvi) any one of anti-OX40 ABDs comprising a VH/VL pair, wherein the VH/VL pair comprises amino acid sequences selected from a group including SEQ ID NOs: 11 and 12, SEQ ID NOs: 13 and 14, SEQ ID NOs: 15 and 16, SEQ ID NOs: 17 and 18, and SEQ ID NOs: 19 and 20 as set forth in CN112442121A, (xxxvii) an anti-OX40 ABD comprising a VH/VL pair, wherein the VH/VL pair comprises amino acid sequences SEQ ID NOs: 4 and 8 as set forth in CN111704671B, (xxxviii) an anti-OX40 single chain antibody comprising the amino acid sequence SEQ ID NO: 1 as set forth in CN109957022A, (xxxix) any one of anti-OX40 ABDs comprising a VH/VL pair, wherein the VH/VL pair comprises amino acid sequences selected from a group including SEQ ID NOs: 1 and 3, SEQ ID NOs: 5 and 7, SEQ ID NOs: 9 and 11, SEQ ID NOs: 13 and 15, SEQ ID NOs: 17 and 19, SEQ ID NOs: 21 and 23, SEQ ID NOs: 25 and 27, SEQ ID NOs: 29 and 31, SEQ ID NOs: 33 and 35, SEQ ID NOs: 37 and 39, SEQ ID NOs: 41 and 43, SEQ ID NOs: 2 and 4, SEQ ID NOs: 6 and 8, SEQ ID NOs: 10 and 12, SEQ ID NOs: 14 and 16, SEQ ID NOs: 18 and 20, SEQ ID NOs: 22 and 24, SEQ ID NOs: 26 and 28, SEQ ID NOs: 30 and 32, SEQ ID NOs: 34 and 36, SEQ ID NOs: 38 and 40, and SEQ ID NOs: 42 and 44 as set forth in CN109232738B, (xl) any one of anti-OX40 ABDs comprising a VH/VL pair, wherein the VH/VL pair comprises amino acid sequences selected from a group including any one of SEQ ID NOs: 58, 59, 79, or 80 (VH) and any one of SEQ ID NOs: 60, 86, 87, or 89 (VL), or any one of SEQ ID NOs: 32-38 (VH) and any one of SEQ ID NOs: 39-49 (VL) as set forth in U.S. Ser. No. 11/407,839B2, (xli) any one of anti-OX40 ABDs comprising a VH/VL pair, wherein the VH/VL pair comprises amino acid sequences selected from a group including SEQ ID NOs: 7 and 8, SEQ ID NOs: 9 and 10, SEQ ID NOs: 44 and 45, SEQ ID NOs: 46 and 47, and SEQ ID NOs: 48 and 49 as set forth in U.S. Pat. No. 8,283,450B2, the contents of which are all incorporated by reference in their entirety, with particularity for their disclosure pertaining to anti-OX40 antibodies, anti-OX40 ABDs, VH/VL pairs corresponding to anti-OX40 ABDs, and sets of vhCDR1-3/vlCDR1-3 sequences corresponding to anti-OX40 ABDs, as well as corresponding information presented in the figures, specification, and sequence listings thereof. Further included herein are variant anti-OX40 antibodies, variant anti-OX40 ABDs comprising a pair of variable heavy (VH) domain and variable light (VL) domain pairs (VH/VL pairs), and variant anti-OX40 ABDs comprising a set of vhCDR1-3 and a set of vlCDR1-3 (sets of vhCDR1-3/vlCDR1-3 sequences) that have at least about 90%, about 91%, about 92%, about 93%, about 94%, about 95%, about 96%, about 97%, about 98%, about 99% or more identity to any of the foregoing antibodies or VH/VL pairs or sets of vhCDR1-3/vlCDR1-3 so long as they retain specific binding for OX40.

### d. 41BB:

In some embodiments, the target antigen comprises 41BB and the ABD binds 41BB.

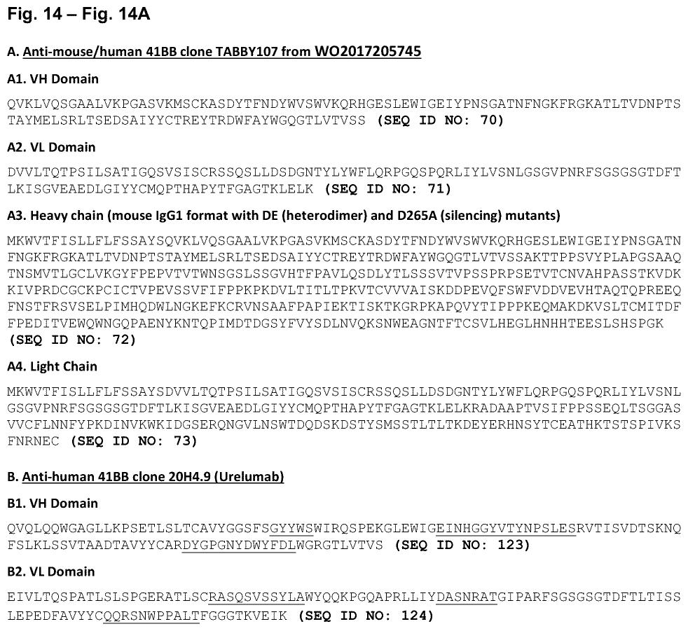

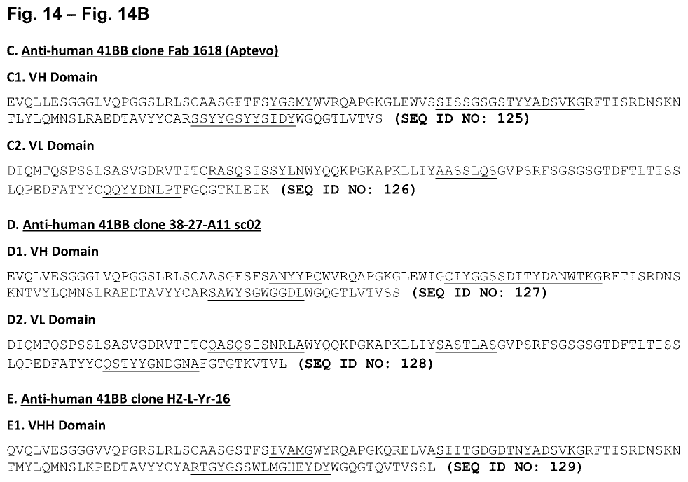

In some further embodiments, the ABD binds human 41BB. In still some further embodiments, the ABD binds the ECD of human 41BB. Suitable VH and VL domains that bind to the ECD of human 41BB include, but are not limited to, VH/VL pairs selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively, as shown in FIG. 14. It should be noted that these ABDs can be included in any of the IL-12 Fc fusion protein configurations described herein.

In some other further embodiments, the ABD binds mouse 41BB. In still some further embodiments, the ABD binds the ECD of mouse 41BB. Suitable VH and VL domains that bind to the ECD of mouse 41BB include, but are not limited to, VH/VL pairs selected from a group including: (i) SEQ ID NOs: 70 and 71, respectively, as shown in FIG. 14. It should be noted that these ABDs can be included in any of the IL-12 Fc fusion protein configurations described herein.

In still some other further embodiments, the ABD comprises a VHH domain, wherein the VHH domain comprises: (a) a CDR1; (b) a CDR2; and (C) a CDR3, wherein CDR1, CDR2, and CDR3 bind a target antigen, wherein the target antigen comprises 41BB. In some other embodiments, the ABD comprises a VHH domain, wherein the VHH domain comprises the amino acid sequence of SEQ ID NO: 129, as shown in FIG. 14. It should be noted that these ABDs can be included in any of the IL-12 Fc fusion protein configurations described herein.

Further included herein are human anti-41BB ABDs comprising a pair of variable heavy (VH) and variable light (VL) domains, wherein the VH/VL pair is selected from a group including: (i) SEQ ID NOs: 43 and 45, respectively, from publication US20120237498A1, (ii) SEQ ID NOs: 3 and 4, respectively, from publication WO2016029073A2, (iii) SEQ ID NOs: 28 and 16, respectively, from publication WO2019016402A1, and (iv) SEQ ID NOs: 13 and 25, respectively, from publication WO2020074584A1. All of the aforementioned publications are incorporated by reference in their entirety, with particularity for relevant disclosure pertaining to PD1 ABDs and the accompanying sequences described therein. Any such sequence described herein can be individually included or excluded from a subject IL-12 Fc fusion protein.

Further included herein are anti-41BB VHH domains, comprising: (i) SEQ ID NO: 2 from publication EP4215549A1. All of the aforementioned publications are incorporated by reference in their entirety, with particularity for relevant disclosure pertaining to 41BB VHH domains and the accompanying sequences described therein. Any such sequence described herein can be individually included or excluded from a subject IL-12 Fc fusion protein.

e. ICOS:

In some embodiments, the target antigen comprises ICOS and the ABD binds ICOS.

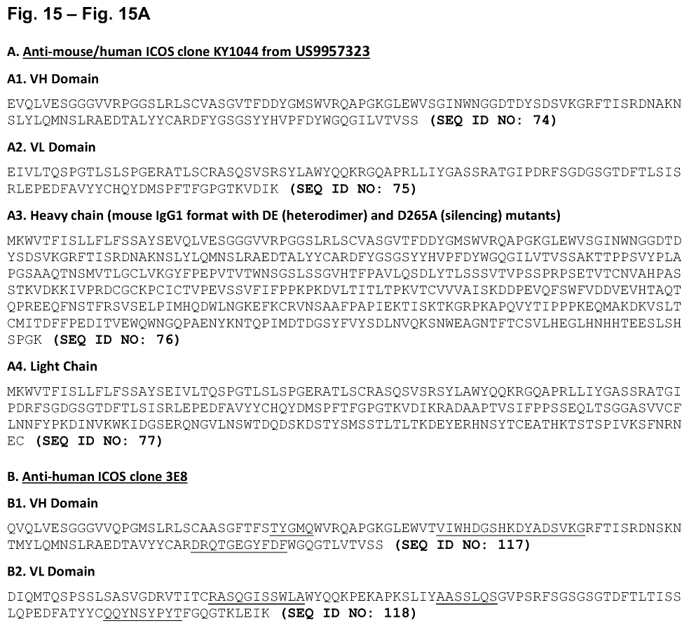

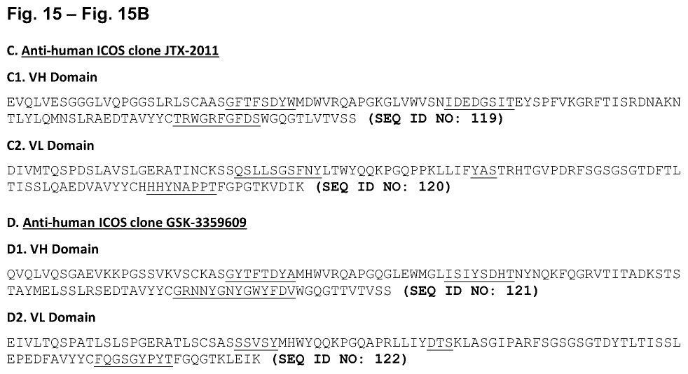

In some further embodiments, the ABD binds human ICOS. In still some further embodiments, the ABD binds the ECD of human ICOS. Suitable VH and VL domains that bind to the ECD of human ICOS include, but are not limited to, VH/VL pairs selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively, as shown in FIG. 15. It should be noted that these ABDs can be included in any of the IL-12 Fc fusion protein configurations described herein.

In some other further embodiments, the ABD binds mouse ICOS. In still some further embodiments, the ABD binds the ECD of mouse ICOS. Suitable VH and VL domains that bind to the ECD of mouse ICOS include, but are not limited to, VH/VL pairs selected from a group including: (i) SEQ ID NOs: 74 and 75, respectively, as shown in FIG. 15. It should be noted that these ABDs can be included in any of the IL-12 Fc fusion protein configurations described herein.

In still some other further embodiments, the ABD comprises a VHH domain, wherein the VHH domain comprises: (a) a CDR1; (b) a CDR2; and (C) a CDR3, wherein CDR1, CDR2, and CDR3 bind a target antigen, wherein the target antigen comprises ICOS. It should be noted that these ABDs can be included in any of the IL-12 Fc fusion protein configurations described herein.

Further included herein are anti-ICOS ABDs comprising a pair of variable heavy (VH) and variable light (VL) domains, wherein the VH/VL pair is selected from a group including: (i) SEQ ID NOs: 16 and 17, respectively, from publication WO2018187613A2, (ii) SEQ ID NOs: 24 and 25, respectively, from publication WO2018187613A2, (iii) SEQ ID NOs: 32 and 33, respectively, from publication WO2018187613A2, (iv) SEQ ID NOs: 40 and 41, respectively, from publication WO2018187613A2, (v) SEQ ID NOs: 40 and 48, respectively, from publication WO2018187613A2, (vi) SEQ ID NOs: 186 and 189, respectively, from publication WO2018187613A2, (vii) SEQ ID NOs: 10 and 11, respectively, from publication WO2016154177A2, (viii) SEQ ID NOs: 20 and 21, respectively, from publication WO2016154177A2, (ix) SEQ ID NOs: 30 and 31, respectively, from publication WO2016154177A2, (x) SEQ ID NOs: 40 and 41, respectively, from publication WO2016154177A2, (xi) SEQ ID NOs: 50 and 51, respectively, from publication WO2016154177A2, (xii) SEQ ID NOs: 60 and 61, respectively, from publication WO2016154177A2, (xiii) SEQ ID NOs: 70 and 71, respectively, from publication WO2016154177A2, (xiv) SEQ ID NOs: 80 and 81, respectively, from publication WO2016154177A2, (xv) SEQ ID NOs: 90 and 91, respectively, from publication WO2016154177A2, (xvi) SEQ ID NOs: 100 and 101, respectively, from publication WO2016154177A2, (xvii) SEQ ID NOs: 110 and 111, respectively, from publication WO2016154177A2, (xviii) SEQ ID NOs: 120 and 121, respectively, from publication WO2016154177A2, (xix) SEQ ID NOs: 130 and 131, respectively, from publication WO2016154177A2, (xx) SEQ ID NOs: 140 and 141, respectively, from publication WO2016154177A2, (xxi) SEQ ID NOs: 150 and 151, respectively, from publication WO2016154177A2, (xxii) SEQ ID NOs: 160 and 161, respectively, from publication WO2016154177A2, (xxiii) SEQ ID NOs: 170 and 171, respectively, from publication WO2016154177A2, (xxiv) SEQ ID NOs: 180 and 181, respectively, from publication WO2016154177A2, (xxv) SEQ ID NOs: 13 and 14, respectively, from publication WO2016120789A1, (xxvi) SEQ ID NOs: 19 and 20, respectively, from publication WO2016120789A1, and (xxvii) SEQ ID NOs: 7 and 8, respectively, from publication WO2016120789A1. All of the aforementioned publications are incorporated by reference in their entirety, with particularity for relevant disclosure pertaining to ICOS ABDs and the accompanying sequences described therein. Any such sequence described herein can be individually included or excluded from a subject IL-12 Fc fusion protein.

f. CCR7:

In some embodiments, the target antigen comprises CCR7 and the ABD binds CCR7.

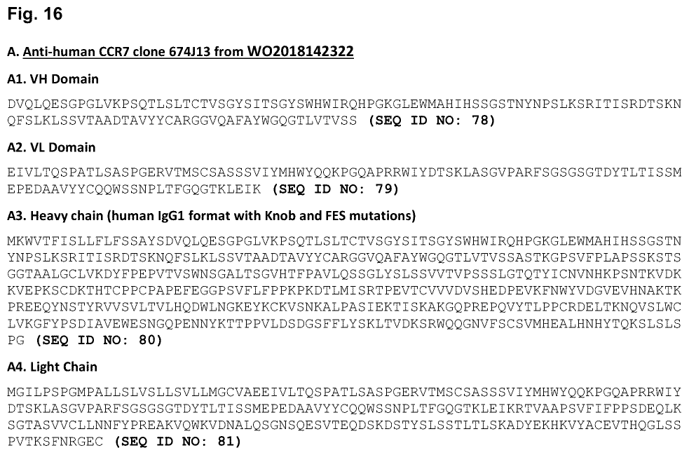

In some further embodiments, the ABD binds human CCR7. In still some further embodiments, the ABD binds the ECD of human CCR7. Suitable VH and VL domains that bind to the ECD of human CCR7 include, but are not limited to, VH/VL pairs selected from a group including: (i) SEQ ID NOs: 78 and 79, respectively, as shown in FIG. 16. It should be noted that these ABDs can be included in any of the IL-12 Fc fusion protein configurations described herein.

In some other further embodiments, the ABD binds mouse CCR7. In still some further embodiments, the ABD binds the ECD of mouse CCR7.

In still some other further embodiments, the ABD comprises a VHH domain, wherein the VHH domain comprises: (a) a CDR 1; (b) a CDR2; and (C) a CDR3, wherein CDR1, CDR2, and CDR3 bind a target antigen, wherein the target antigen comprises CCR7. It should be noted that these ABDs can be included in any of the IL-12 Fc fusion protein configurations described herein.

g. CCR8:

In some embodiments, the target antigen comprises CCR8 and the ABD binds CCR8.

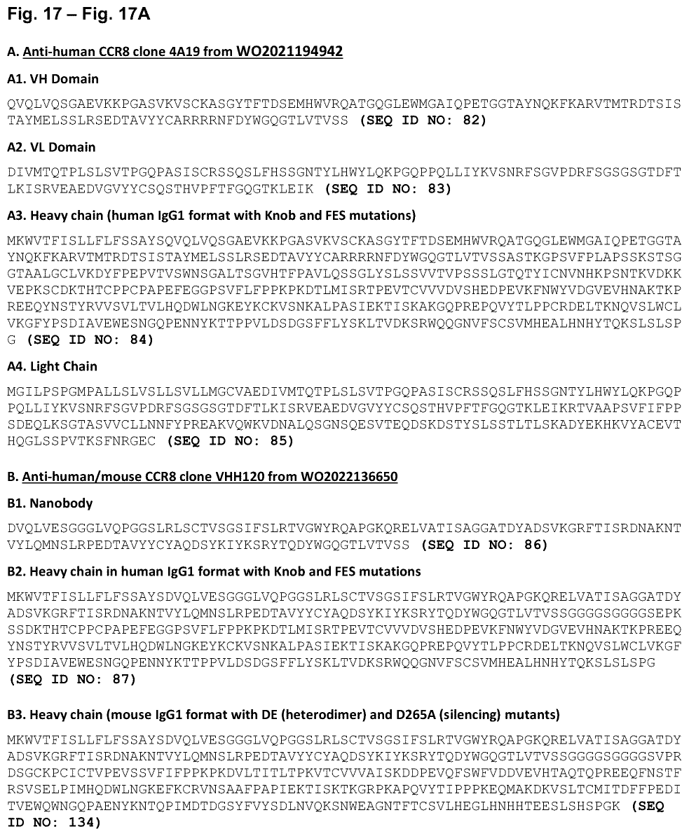

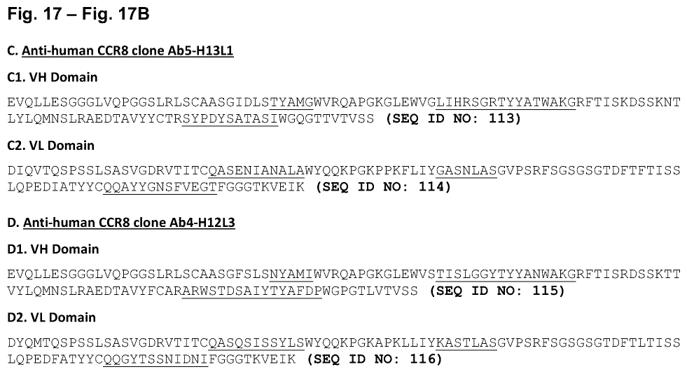

In some further embodiments, the ABD binds human CCR8. In still some further embodiments, the ABD binds the ECD of human CCR8. Suitable VH and VL domains that bind to the ECD of human CCR8 include, but are not limited to, VH/VL pairs selected from a group including: (i) SEQ ID NOs: 82 and 83, respectively, (ii) SEQ ID NOs: 113 and 114, respectively, and (iii) SEQ ID NOs: 115 and 116, respectively, as shown in FIG. 17. It should be noted that these ABDs can be included in any of the IL-12 Fc fusion protein configurations described herein.

In some other further embodiments, the ABD binds mouse CCR8. In still some further embodiments, the ABD binds the ECD of mouse CCR8.

In still some other further embodiments, the ABD comprises a VHH domain, wherein the VHH domain comprises: (a) a CDR1; (b) a CDR2; and (C) a CDR3, wherein CDR1, CDR2, and CDR3 bind a target antigen, wherein the target antigen comprises CCR8. In some other embodiments, the ABD comprises a VHH domain, wherein the VHH domain comprises the amino acid sequence of SEQ ID NO: 86, as shown in FIG. 17. It should be noted that these ABDs can be included in any of the IL-12 Fc fusion protein configurations described herein.

Further included herein are anti-CCR8 ABDs comprising a pair of variable heavy (VH) and variable light (VL) domains, wherein the VH/VL pair is selected from a group including: (i) SEQ ID NOs: 47 and 48, respectively, from publication WO2023288241, (ii) SEQ ID NOs: 21 and 24, respectively, from publication WO2023288241, (iii) SEQ ID NOs: 344 and 300, respectively, from publication EP4186926A1, (iv) SEQ ID NOs: 344 and 301, respectively, from publication EP4186926A1, (v) SEQ ID NOs: 344 and 302, respectively, from publication EP4186926A1, (vi) SEQ ID NOs: 344 and 303, respectively, from publication EP4186926A1, (vii) SEQ ID NOs: 344 and 304, respectively, from publication EP4186926A1, (viii) SEQ ID NOs: 344 and 305, respectively, from publication EP4186926A1, (ix) SEQ ID NOs: 344 and 306, respectively, from publication EP4186926A1, (x) SEQ ID NOs: 344 and 307, respectively, from publication EP4186926A1, (xi) SEQ ID NOs: 344 and 308, respectively, from publication EP4186926A1, (xii) SEQ ID NOs: 344 and 309, respectively, from publication EP4186926A1, (xiii) SEQ ID NOs: 344 and 310, respectively, from publication EP4186926A1, (xiv) SEQ ID NOs: 344 and 311, respectively, from publication EP4186926A1, (xv) SEQ ID NOs: 344 and 312, respectively, from publication EP4186926A1, (xvi) SEQ ID NOs: 344 and 299, respectively, from publication EP4186926A1, (xvii) SEQ ID NOs: 344 and 313, respectively, from publication EP4186926A1, (xviii) SEQ ID NOs: 344 and 317, respectively, from publication EP4186926A1, (xix) SEQ ID NOs: 344 and 314, respectively, from publication EP4186926A1, (xx) SEQ ID NOs: 344 and 318, respectively, from publication EP4186926A1, (xxi) SEQ ID NOs: 344 and 315, respectively, from publication EP4186926A1, (xxii) SEQ ID NOs: 344 and 316, respectively, from publication EP4186926A1, (xxiii) SEQ ID NOs: 29 and 22, respectively, from publication WO2022078277A1, (xxiv) SEQ ID NOs: 29 and 23, respectively, from publication WO2022078277A1, (xxv) SEQ ID NOs: 30 and 22, respectively, from publication WO2022078277A1, (xxvi) SEQ ID NOs: 30 and 23, respectively, from publication WO2022078277A1, (xxvii) SEQ ID NOs: 31 and 22, respectively, from publication WO2022078277A1, (xxviii) SEQ ID NOs: 31 and 23, respectively, from publication WO2022078277A1, (xxix) SEQ ID NOs: 39 and 42, respectively, from publication WO2022078277A1, (xxx) SEQ ID NOs: 39 and 43, respectively, from publication WO2022078277A1, (xxxi) SEQ ID NOs: 40 and 42, respectively, from publication WO2022078277A1, and (xxxii) SEQ ID NOs: 40 and 43, respectively, from publication WO2022078277A1. All of the aforementioned publications are incorporated by reference in their entirety, with particularity for relevant disclosure pertaining to CCR8 ABDs and the accompanying sequences described therein. Any such sequence described herein can be individually included or excluded from a subject IL-12 Fc fusion protein.

Further included herein are anti-CCR8 VHH domains, comprising: (i) SEQ ID NO: 21, from publication WO2022136647A1, (ii) SEQ ID NO: 22, from publication WO2022136647A1, (iii) SEQ ID NO: 23, from publication WO2022136647A1, (iv) SEQ ID NO: 24, from publication WO2022136647A1, or (v) SEQ ID NO: 25, from publication WO2022136647A1.

All of the aforementioned publications are incorporated by reference in their entirety, with particularity for relevant disclosure pertaining to CCR8 VHH domains and the accompanying sequences described therein. Any such sequence described herein can be individually included or excluded from a subject IL-12 Fc fusion protein.

### 3. Fc Domains:

The present disclosure provides IL-12 Fc fusion proteins comprising: (1) a first IL-12 subunit domain and a second IL-12 subunit domain, wherein (i) the first IL-12 subunit domain comprises a variant IL-12 p35 subunit domain and the second IL-12 subunit domain comprises a (variant) IL-12 p40 subunit domain, or the first IL-12 subunit domain comprises a (variant) IL-12 p40 subunit domain and the second IL-12 subunit domain comprises a variant IL-12 p35 subunit domain, and (ii) the variant IL-12 p35 subunit domain comprises a set of amino acid substitutions that reduces binding affinity of the resultant IL-12 Fc fusion protein for IL-12Rβ2 as compared to a reference IL-12, wherein the set of amino acid substitutions is selected from a group including: (a) Y40S/K170A, (b) Y40S/K170T, (c) Y40P/K168A, (d) Y40P/K170A, and (e) Y40E/K170A; (2) at least one antigen binding domain (ABD), wherein the ABD binds a target antigen selected from a group including: (a) OX40, (b) LAG3, (c) PD1, (d) ICOS, (e) 41BB, (f) CCR7, and (g) CCR8; (3) one or more domain linkers; and (4) a first Fc domain and a second Fc domain.

In some embodiments, the first Fc domain and/or the second Fc domain comprises the CH2-CH3 domains of human immunoglobulin G1 (IgG1), and optionally all or part of the hinge of human IgG1, as well as fragments thereof. In EU numbering, the CH2-CH3 domain for human IgG1 comprises amino acids 231 to 447, and the hinge comprises amino acids 216 to 230. A “Fc fragment” may contain one, two, three, four, five, six, seven, eight, nine, 10, 11, 12, 13, 14, 15, 16, 17, 18, 19, 20 or more amino acids removed from either or both of the N- and C-termini, but still retains the ability to form a dimer with another Fc domain or Fc fragment as can be detected using standard methods (such as, for example, non-denaturing chromatography, size exclusion chromatograph, and the like).

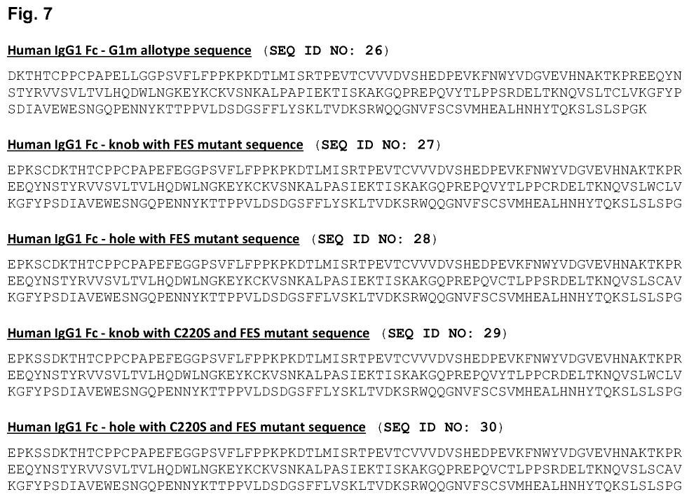

In some embodiments, the first Fc domain and/or the second Fc domain comprises an amino acid sequence selected from a group that includes any one of: (i) SEQ ID NO: 26 (human IgG1 Fc; G1m allotype), (ii) SEQ ID NO: 27 (human IgG1 Fc; knob with FES mutant sequence), (iii) SEQ ID NO: 28 (human IgG1; hole with FES mutant sequence), (iv) SEQ ID NO: 29 (human IgG1 Fc; knob with C220S and FES mutant sequence), and (v) SEQ ID NO: 30 (human IgG1 Fc; hole with C220S and FES mutant sequence), as shown in FIG. 7. In some embodiments, the first Fc domain and the second Fc domain comprises: (i) SEQ ID NOs: 26 and 26, respectively; (ii) SEQ ID NOs: 26 and 27, respectively; (iii) SEQ ID NOs: 27 and 26, respectively; (iv) SEQ ID NOs: 26 and 28, respectively; (v) SEQ ID NOs: 28 and 26, respectively; (vi) SEQ ID NOs: 26 and 29, respectively; (vii) SEQ ID NOs: 29 and 26, respectively; (viii) SEQ ID NOs: 26 and 30, respectively; (ix) SEQ ID NOs: 30 and 26, respectively; (x) SEQ ID NOs: 27 and 28, respectively; (xi) SEQ ID NOs: 28 and 27, respectively; (xii) SEQ ID NOs: 27 and 30, respectively; (xiii) SEQ ID NOs: 30 and 27, respectively; (xiv) SEQ ID NOs: 28 and 29, respectively; (xv) SEQ ID NOs: 29 and 28, respectively; (xvi) SEQ ID NOs: 29 and 30, respectively; or (xvii) SEQ ID NOs: 30 and 29, respectively. In some further embodiments, the first Fc domain further comprises a set of substitution mutations comprising T366W/S354C, and the second Fc domain further comprises a set of substitution mutations comprising T366S/L368A/Y407V/Y349C. In some other further embodiments, the first Fc domain further comprises a set of substitution mutations comprising T366S/L368A/Y407V/Y349C, and the second Fc domain further comprises a set of substitution mutations comprising T366W/S354C.

In some embodiments, the first Fc domain and/or the second Fc domain comprises an amino acid sequence with at least 90%, 91%, 92%, 93%, 94%, 95%, 96%, 97%, 98%, 99% or higher identity to an amino acid sequence selected from a group that includes any one of SEQ ID NOs: 26-30 (as shown in FIG. 7). In some embodiments, the first Fc domain and the second Fc domain individually comprise an amino acid sequence with at least 90%, 91%, 92%, 93%, 94%, 95%, 96%, 97%, 98%, 99% or higher to: (i) SEQ ID NOs: 26 and 26, respectively; (ii) SEQ ID NOs: 26 and 27, respectively; (iii) SEQ ID NOs: 27 and 26, respectively; (iv) SEQ ID NOs: 26 and 28, respectively; (v) SEQ ID NOs: 28 and 26, respectively; (vi) SEQ ID NOs: 26 and 29, respectively; (vii) SEQ ID NOs: 29 and 26, respectively; (viii) SEQ ID NOs: 26 and 30, respectively; (ix) SEQ ID NOs: 30 and 26, respectively; (x) SEQ ID NOs: 27 and 28, respectively; (xi) SEQ ID NOs: 28 and 27, respectively; (xii) SEQ ID NOs: 27 and 30, respectively; (xiii) SEQ ID NOs: 30 and 27, respectively; (xiv) SEQ ID NOs: 28 and 29, respectively; (xv) SEQ ID NOs: 29 and 28, respectively; (xvi) SEQ ID NOs: 29 and 30, respectively; or (xvii) SEQ ID NOs: 30 and 29, respectively. In some further embodiments, the first Fc domain further comprises a set of substitution mutations comprising T366W/S354C, and the second Fc domain further comprises a set of substitution mutations comprising T366S/L368A/Y407V/Y349C. In some other further embodiments, the first Fc domain further comprises a set of substitution mutations comprising T366S/L368A/Y407V/Y349C, and the second Fc domain further comprises a set of substitution mutations comprising T366W/S354C.

In some embodiments, the first Fc domain and/or the second Fc domain comprises one, two, three, four, five, six, seven, eight, nine, 10, 11, 12, 13, 14, 15, 16, 17, 18, 19, 20, 25, 30, 40, 50, 60, 70, 80, 90, 100 or more amino acid substitutions.

In some embodiments, the first Fc domain and the second Fc domain comprise modifications that promote heterodimerization of the first and second Fe domains. In further embodiments, the first Fc domain comprises an amino acid sequence selected from a group that includes: SEQ ID NO: 27 and SEQ ID NO: 29, and the second Fc domain comprises an amino acid sequence selected from a group that includes: SEQ ID NO: 28 and SEQ ID NO: 30. In some other further embodiments, the first Fc domain comprises an amino acid sequence selected from a group that includes: SEQ ID NO: 28 and SEQ ID NO: 30, and the second Fc domain comprises an amino acid sequence selected from a group that includes: SEQ ID NO: 27 and SEQ ID NO: 29. In some further embodiments, the first Fc domain further comprises a set of substitution mutations comprising T366W/S354C, and the second Fc domain further comprises a set of substitution mutations comprising T366S/L368A/Y407V/Y349C. In some other further embodiments, the first Fc domain further comprises a set of substitution mutations comprising T366S/L368A/Y407V/Y349C, and the second Fc domain further comprises a set of substitution mutations comprising T366W/S354C. In other embodiments, the first Fc domain and the second Fc domain do not comprise modifications that promote heterodimerization of the first and second Fc domains.

In some embodiments, the first Fc domain and/or the second Fc domain comprises one or more modifications that alter Fc binding. In some embodiments, the first Fc domain and/or the second Fc domain comprises one or more modifications that alter Fc half-life. In some embodiments, the first Fc domain and/or the second Fc domain comprises one or more modifications that alter the binding of the first Fc domain and/or the second Fc domain to the neonatal Fc receptor (FcRn).

Described above are various human IgG1 Fc domains, at least some of which comprise one or more modifications. For clarity, while the modifications are discussed in the context of human IgG1 Fc domains for the sake of brevity, it is expressly contemplated that mutations to other immunoglobulins, such as, for example, IgG2, IgG3, IgG4, IgA, IgM, and IgE, can be made at residue positions corresponding to the mutations in human IgG1 that are described herein. Those skilled in the relevant art will readily be able to determine residue locations in other immunoglobulins that correspond with the modifications to human IgG1 described herein.

### 4. Domain Linkers:

The present disclosure provides IL-12 Fc fusion proteins comprising: (1) a first IL-12 subunit domain and a second IL-12 subunit domain, wherein (i) the first IL-12 subunit domain comprises a variant IL-12 p35 subunit domain and the second IL-12 subunit domain comprises a (variant) IL-12 p40 subunit domain, or the first IL-12 subunit domain comprises a (variant) IL-12 p40 subunit domain and the second IL-12 subunit domain comprises a variant IL-12 p35 subunit domain, and (ii) the variant IL-12 p35 subunit domain comprises a set of amino acid substitutions that reduces binding affinity of the resultant IL-12 Fc fusion protein for IL-12Rβ2 as compared to a reference IL-12, wherein the set of amino acid substitutions is selected from a group including: (a) Y40S/K170A, (b) Y40S/K170T, (c) Y40P/K168A, (d) Y40P/K170A, and (e) Y40E/K170A; (2) at least one antigen binding domain (ABD), wherein the ABD binds a target antigen selected from a group including: (a) OX40, (b) LAG3, (c) PD1, (d) ICOS, (e) 41BB, (f) CCR7, and (g) CCR8; (3) one or more domain linkers; and (4) a first Fc domain and a second Fc domain.

As used herein, the terms “linker” and “domain linker,” which can be used interchangeably, generally refer to a structure (such as, for example, a polymer (e.g., any one of the amino acid sequences comprising SEQ ID NOs: 31-40)) or a bond (such as, for example, a covalent bond) that “links” a first domain to a second domain (such as, for example, a CH1 domain and a Fc domain, a VH domain and a VL domain, a VL domain and a CL domain, a subunit of a cytokine (e.g., the p35 subunit domain of IL-12, the p40 subunit domain of IL-12, etc.) and a subunit of an antibody (e.g., a VH domain, a VL domain, a CH1 domain, a Fc domain, etc.), a first subunit of a cytokine and a second subunit of the cytokine, and the like). Such a linkage may be “direct” (i.e., in instances where the domain linker comprises a covalent bond) or “indirect” (i.e., in instances where the domain linker comprises a structure, such as, for example, any one of the amino acid sequences of SEQ ID NOs: 31-40). In many embodiments, a plurality of domain linkers is incorporated into a subject “low potency IL-12 Fc fusion proteins,” as is described in further detail herein. In some instances, a subject low potency IL-12 Fc fusion protein may comprise one or more domain linkers. In some further instances, the one or more domain linkers comprise one or more of the amino acid sequences of SEQ ID NOs: 31-40, and may optionally comprise one or more covalent bonds. In some other further instances, the one or more domain linkers comprise one or more covalent bonds, and may optionally comprise one or more of the amino acid sequences of SEQ ID NOs: 31-40. In some instances, at least one domain linker comprises a hinge region of a Fc domain.

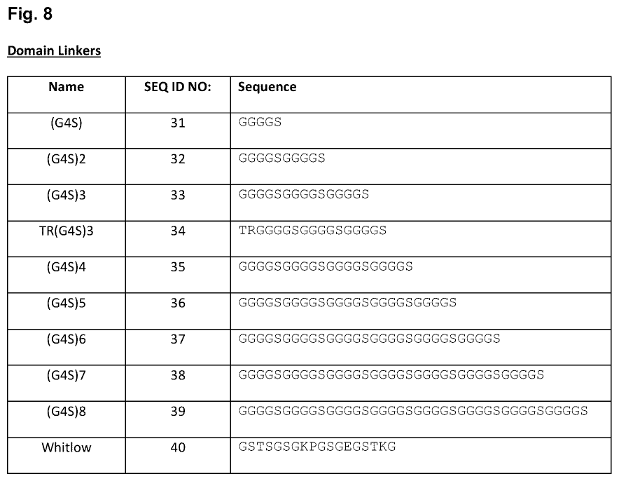

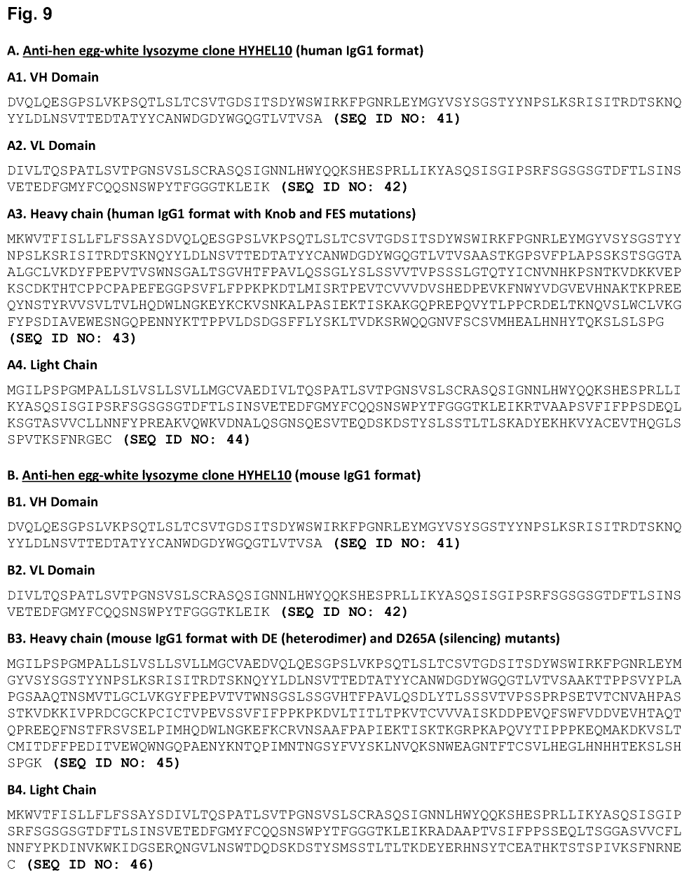

In some embodiments, the C-terminus of the variant IL-12 p35 subunit domain is covalently linked to the N-terminus of: (a) the (variant) IL-12 p40 IL-12 subunit domain, (b) the first Fc domain, or (c) the second Fc domain, using a domain linker. In some further embodiments, the domain linker is selected from a group that includes any one of: (i) SEQ ID NO: 31 ((G4S)), (ii) SEQ ID NO: 32 ((G4S)2), (iii) SEQ ID NO: 33 ((G4S)3), (iv) SEQ ID NO: 34 (TR(G4S)3), (v) SEQ ID NO: 35 ((G4S)4), (vi) SEQ ID NO: 36 ((G4S)5), (vii) SEQ ID NO: 37 ((G4S)6), (viii) SEQ ID NO: 38 ((G4S)7), (ix) SEQ ID NO: 39 ((G4S)8), and (x) SEQ ID NO: 40 (Whitlow), as shown in FIG. 8. In some other further embodiments, the domain linker comprises a covalent bond or a hinge region of a Fc domain. In some other embodiments, the N-terminus of the variant IL-12 p35 subunit domain is covalently linked to the C-terminus of: (a) the (variant) IL-12 p40 IL-12 subunit domain, (b) the first Fc domain, or (c) the second Fc domain, using a domain linker. In some further embodiments, the domain linker is selected from a group that includes any one of SEQ ID NOs: 31-40 (as shown in FIG. 8). In some other further embodiments, the domain linker comprises a covalent bond or a hinge region of a Fc domain. In still other embodiments, the variant IL-12 p35 subunit domain is not attached to: (a) the (variant) IL-12 p40 IL-12 subunit domain, (b) the first Fc domain, or (c) the second Fc domain, via a domain linker. Phrased differently, in some embodiments, the variant IL-12 p35 subunit domain is covalently linked to: (a) the (variant) IL-12 p40 IL-12 subunit domain, (b) the first Fc domain, or (c) the second Fc domain, without the use of a domain linker.

In some embodiments, the C-terminus of the (variant) IL-12 p40 IL-12 subunit domain is covalently linked to the N-terminus of: (a) the variant IL-12 p35 subunit domain, (b) the first Fc domain, or (c) the second Fc domain, using a domain linker. In some further embodiments, the domain linker is selected from a group that includes any one of SEQ ID NOs: 31-40 (as shown in FIG. 8). In some other further embodiments, the domain linker comprises a covalent bond or a hinge region of a Fc domain. In some embodiments, the N-terminus of the (variant) IL-12 p40 IL-12 subunit domain is covalently linked to the C-terminus of: (a) the variant IL-12 p35 subunit domain, (b) the first Fc domain, or (c) the second Fc domain, using a domain linker. In some further embodiments, the domain linker is selected from a group that includes any one of SEQ ID NOs: 31-40 (as shown in FIG. 8). In some other further embodiments, the domain linker comprises a covalent bond or a hinge region of a Fc domain. In still other embodiments, the (variant) IL-12 p40 IL-12 subunit domain is not attached to: (a) the variant IL-12 p35 subunit domain, (b) the first Fc domain, or (c) the second Fc domain, via a domain linker. Phrased differently, in some embodiments, the (variant) IL-12 p40 IL-12 subunit domain is covalently linked to: (a) the variant IL-12 p35 subunit domain, (b) the first Fc domain, or (c) the second Fc domain, without the use of a domain linker.

In some embodiments, the ABD comprises a VH domain and a VL domain. In some further embodiments, the C-terminus of the VH domain is covalently linked to the N-terminus of: (a) the VL domain, (b) the first Fc domain, or (c) the second Fc domain, using a domain linker. In still some further embodiments, the domain linker is selected from a group that includes any one of SEQ ID NOs: 31-40 (as shown in FIG. 8). In some other further embodiments, the domain linker comprises a covalent bond or a hinge region of a Fc domain. In some other embodiments, the N-terminus of the VH domain is covalently linked to the C-terminus of: (a) the VL domain, (b) the first Fc domain, or (c) the second Fc domain, using a domain linker. In still some further embodiments, the domain linker is selected from a group that includes any one of SEQ ID NOs: 31-40 (as shown in FIG. 8). In some other further embodiments, the domain linker comprises a covalent bond or a hinge region of a Fc domain. In still other embodiments, the VH domain is not attached to: (a) the VL domain, (b) the first Fc domain, or (c) the second Fc domain, via a domain linker. Phrased differently, in some embodiments, the VH domain is covalently linked to: (a) the VL domain, (b) the first Fc domain, or (c) the second Fc domain, without the use of a domain linker.

In some embodiments, the ABD comprises a VH domain and a VL domain. In some further embodiments, the C-terminus of the VL domain is covalently linked to the N-terminus of: (a) the VH domain, (b) the first Fc domain, or (c) the second Fc domain, using a domain linker. In still some further embodiments, the domain linker is selected from a group that includes any one of SEQ ID NOs: 31-40 (as shown in FIG. 8). In some other further embodiments, the domain linker comprises a covalent bond or a hinge region of a Fc domain. In some other further embodiments, the N-terminus of the VL domain is covalently linked to the C-terminus of: (a) the VH domain, (b) the first Fc domain, or (c) the second Fc domain, using a domain linker. In still some further embodiments, the domain linker is selected from a group that includes any one of SEQ ID NOs: 31-40 (as shown in FIG. 8). In some other further embodiments, the domain linker comprises a covalent bond or a hinge region of a Fc domain. In still other embodiments, the VL domain is not attached to: (a) the VH domain, (b) the first Fc domain, or (c) the second Fc domain, via a domain linker. Phrased differently, in some embodiments, the VL domain is covalently linked to: (a) the VH domain, (b) the first Fc domain, or (c) the second Fc domain, without the use of a domain linker.

In some embodiments, the ABD comprises a VHH domain. In some further embodiments, the C-terminus of the VHH domain is covalently linked to the N-terminus of: (a) the first Fc domain, or (b) the second Fc domain, using a domain linker. In still some further embodiments, the domain linker is selected from a group that includes any one of SEQ ID NOs: 31-40 (as shown in FIG. 8). In some other further embodiments, the domain linker comprises a covalent bond or a hinge region of a Fc domain. In some other embodiments, the N-terminus of the VHH domain is covalently linked to the C-terminus of: (a) the first Fc domain, or (b) the second Fc domain, using a domain linker. In still some further embodiments, the domain linker is selected from a group that includes any one of SEQ ID NOs: 31-40 (as shown in FIG. 8). In some other further embodiments, the domain linker comprises a covalent bond or a hinge region of a Fc domain. In still other embodiments, the VHH domain is not attached to: (a) the first Fc domain, or (b) the second Fc domain, via a domain linker. Phrased differently, in some embodiments, the VHH domain is covalently linked to: (a) the first Fc domain, or (b) the second Fc domain, without the use of a domain linker.

In some embodiments, the C-terminus of the first Fc domain is covalently linked to the N-terminus of: (a) the variant IL-12 p35 subunit domain, (b) the (variant) IL-12 p40 subunit domain, (c) a VH domain, (d) a VL domain, or (e) a VHH domain, using a domain linker. In still some further embodiments, the domain linker is selected from a group that includes any one of SEQ ID NOs: 31-40 (as shown in FIG. 8). In some other further embodiments, the domain linker comprises a covalent bond. In some other embodiments, the N-terminus of the first Fc domain is covalently linked to the C-terminus of: (a) the variant IL-12 p35 subunit domain, (b) the (variant) IL-12 p40 subunit domain, (c) a VH domain, (d) a VL domain, or (e) a VHH domain, using a domain linker. In still some further embodiments, the domain linker is selected from a group that includes any one of SEQ ID NOs: 31-40 (as shown in FIG. 8). In some other further embodiments, the domain linker comprises a covalent bond. In still other embodiments, the first Fc domain is not attached to: (a) the variant IL-12 p35 subunit domain, (b) the (variant) IL-12 p40 subunit domain, (c) a VH domain, (d) a VL domain, or (e) a VHH domain, via a domain linker. Phrased differently, in some embodiments, the first Fc domain is covalently linked to: (a) the variant IL-12 p35 subunit domain, (b) the (variant) IL-12 p40 subunit domain, (c) a VH domain, (d) a VL domain, or (e) a VHH domain, without the use of a domain linker.

In some embodiments, the C-terminus of the second Fc domain is covalently linked to the N-terminus of: (a) the variant IL-12 p35 subunit domain, (b) the (variant) IL-12 p40 subunit domain, (c) a VH domain, (d) a VL domain, or (e) a VHH domain, using a domain linker. In still some further embodiments, the domain linker is selected from a group that includes any one of SEQ ID NOs: 31-40 (as shown in FIG. 8). In some other further embodiments, the domain linker comprises a covalent bond. In some other embodiments, the N-terminus of the second Fc domain is covalently linked to the C-terminus of: (a) the variant IL-12 p35 subunit domain, (b) the (variant) IL-12 p40 subunit domain, (c) a VH domain, (d) a VL domain, or (e) a VHH domain, using a domain linker. In still some further embodiments, the domain linker is selected from a group that includes any one of SEQ ID NOs: 31-40 (as shown in FIG. 8). In some other further embodiments, the domain linker comprises a covalent bond. In still other embodiments, the second Fc domain is not attached to: (a) the variant IL-12 p35 subunit domain, (b) the (variant) IL-12 p40 subunit domain, (c) a VH domain, (d) a VL domain, or (e) a VHH domain, via a domain linker. Phrased differently, in some embodiments, the second Fc domain is covalently linked to: (a) the variant IL-12 p35 subunit domain, (b) the (variant) IL-12 p40 subunit domain, (c) a VH domain, (d) a VL domain, or (e) a VHH domain, without the use of a domain linker.

### 5. Nucleic Acids and Vectors:

In accordance with any of the aspects and the embodiments described herein, the present disclosure provides nucleic acid compositions encoding IL-12 Fc fusion proteins, as well as domains thereof (such as, for example, variant IL-12 p35 subunit domains, (variant) IL-12 p40 subunit domains, antigen binding domains, variable heavy domains, variable light domains, Fc domains, domain linkers, and the like).

In some embodiments, one or more of the nucleic acids encoding the components of the present invention are incorporated into an expression cassette or an expression vector. It will be understood by one skilled in the art that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure includes a coding sequence for the polypeptide as disclosed herein, which is operably linked to expression control elements, such as a promoter, and optionally, any or a combination of other nucleic acid sequences that affect the transcription or translation of the coding sequence (such as, for example, an origin of replication, selectable markers, ribosomal binding sites, inducers, and the like).

In some embodiments, the nucleotide sequence is incorporated into an expression vector.

It will be understood by one skilled in the art that the term “vector” generally refers to a recombinant polynucleotide construct designed for transfer between host cells, and that may be used for the purpose of transformation (e.g., the introduction of heterologous DNA into a host cell). As such, in some embodiments, the vector can be a replicon, such as, for example, a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitates transfer of the nucleic acid molecule or integration into the genome of a cell or to a virus particle that mediates nucleic acid transfer. Viral particles typically include various viral components, and sometimes also host cell components, in addition to the nucleic acid of interest. The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is a baculoviral vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Accordingly, also provided herein are vectors, plasmids, or viruses containing one or more of the nucleic acids encoding any of the IL-12 Fc fusion protein (or domains thereof), disclosed herein. The nucleic acids can be contained within a vector that is capable of directing their expression in, for example, a cell line that has been transformed/transduced with the vector. Suitable vectors for use in prokaryotic and eukaryotic cells are known in the art and are commercially available, or readily prepared by a skilled artisan.

As will be appreciated by those skilled in the art, the nucleic acid compositions will depend on the configuration of the subject IL-12 Fc fusion protein. Thus, for example, when the configuration requires three amino acid sequences, three nucleic acid sequences can be incorporated into one or more expression vectors for expression. Similarly for other configurations, when only two nucleic acids are needed, they can be incorporated into one or two expression vectors.

DNA vectors can be introduced into host cells, such as, for example, eukaryotic cells or prokaryotic cells, via conventional transformation or transfection techniques, including, but not limited to, one or more of the following: transfection, calcium phosphate transfection, DEAE-dextran-mediated transfection, microinjection, cationic lipid-mediated transfection, electroporation, transduction, scrape loading, ballistic introduction, nucleoporation, hydrodynamic shock, infection, and the like.

Viral vectors that can be used in the disclosure include, but are not limited to, baculoviral vectors, retrovirus vectors, adenovirus vectors, adeno-associated virus vectors, lentivirus vectors, herpes virus, simian virus 40 (SV40), bovine papilloma virus vectors, and the like.

### 6. Recombinant Cells and Cell Cultures:

In another aspect, provided herein are cell cultures including at least one recombinant cell (also referred to herein as a “host cell”) as disclosed herein, and a culture medium. Generally, the culture medium can be any suitable culture medium for culturing the cells described herein. Techniques for transforming a wide variety of the above-mentioned cells and species are known in the art. Accordingly, cell cultures including at least one recombinant cell as disclosed herein are also within the scope of this application. Methods and systems suitable for generating and maintaining cell cultures are known in the art.

A host cell can be used for preparative purposes to propagate one or more nucleic acids encoding IL-12 Fc fusion protein, as well as domains thereof (such as, for example, variant IL-12 p35 subunit domains, (variant) IL-12 p40 subunit domains, antigen binding domains, variable heavy domains, variable light domains, Fe domains, domain linkers, and the like), as well as combinations and/or components thereof. A host cell can include a prokaryotic cell or eukaryotic cell in which production of the IL-12 Fc fusion proteins and/or components thereof is specifically intended. Non-limiting examples of host cells include bacterial cells (such as, for example, cells of Gram-positive bacteria (e.g., species of the genus Bacillus, Streptomyces, and Staphylococcus) or cells of Gram-negative bacteria (e.g., cells of the genus Escherichia and Pseudomonas)), fungal cells or yeast cells (e.g., Saccharomyces, Pichia pastoris, and Hansenula polymorpha), insect cells (e.g., cells of Drosophila and Sf9 cells), plant cells (e.g., cells from crop plants, medicinal or ornamental plants or bulbs), mammalian cells (e.g., epithelial cell lines, osteosarcoma cell lines, neuroblastoma cell lines, epithelial carcinomas, glial cells, liver cell lines, Chinese hamster ovary (CHO) cells, COS cells, BHK cells, HeLa cells, D3 cells of the line of murine embryonic stem cells (mESCs), human embryonic stem cells (e.g., HS293 cells and BG01V cells), NIH 3T3 cells, human embryonic kidney (HEK) 293T cells, human mesenchymal stem cells (hMSCSs), and the like), and the like.

### B. Useful Embodiments of the Invention

The present disclosure provides IL-12 Fc fusion proteins comprising: (1) a first IL-12 subunit domain and a second IL-12 subunit domain, wherein (i) the first IL-12 subunit domain comprises a variant IL-12 p35 subunit domain and the second IL-12 subunit domain comprises a (variant) IL-12 p40 subunit domain, or the first IL-12 subunit domain comprises a (variant) IL-12 p40 subunit domain and the second IL-12 subunit domain comprises a variant IL-12 p35 subunit domain, and (ii) the variant IL-12 p35 subunit domain comprises a set of amino acid substitutions that reduces binding affinity of the resultant IL-12 Fc fusion protein for IL-12Rβ2 as compared to a reference IL-12, wherein the set of amino acid substitutions is selected from a group including: (a) Y40S/K170A, (b) Y40S/K170T, (c) Y40P/K168A, (d) Y40P/K170A, and (e) Y40E/K170A; (2) at least one antigen binding domain (ABD), wherein the ABD binds a target antigen selected from a group including: (a) OX40, (b) LAG3, (c) PD1, (d) ICOS, (e) 41BB, (f) CCR7, and (g) CCR8; (3) one or more domain linkers; and (4) a first Fc domain and a second Fc domain.

As will be appreciated by those skilled in the art, a subject IL-12 Fc fusion protein can take on one of several different configurations (including, but not limited to, those described herein and shown in the figures (see, e.g., FIGS. 18A-18H)). A subject IL-12 Fc fusion protein can be configured such that the ABD is in a Fab format, a scFv format, a nanobody format, as well as other formats known in the relevant art (including, but not limited to: a diabody format, a triabody format, a minibody format, a single-chain Fab (scFab) format, a F(ab)₂ format, a scFv-Fc format, a BiTE format, an intrabody format, a tandem scFv format, a DART format, and the like). In some instances, the IL-12 Fc fusion protein is configured such that the ABD is in a Fab format (i.e., the IL-12 Fc fusion protein comprises a variable heavy (VH) domain, a variable light (VL) domain, a constant heavy (CH1) domain, and a constant light (CL) domain). In some other instances, the IL-12 Fc fusion protein is configured such that the ABD is in an scFv format (i.e., the IL-12 Fc fusion protein comprises, from N-terminus to C-terminus, a VH domain, a linker, and a VL domain; or the reverse where the IL-12 Fc fusion protein comprises, from N-terminus to C-terminus, a VL domain, a linker, and a VH domain). In still other instances, the IL-12 Fc fusion protein is configured such that the ABD is in a nanobody format (i.e., the IL-12 Fc fusion protein comprises a VHH domain, wherein the VHH domain comprises a complementarity determining region (CDR) 1, a CDR2, and a CDR3, wherein the CDR1, CDR2, and CDR3 bind to a target antigen).

Further, some configurations of IL-12 Fc fusion proteins described herein comprise more than one ABD and are therefore capable of binding more than one target antigen. For the sake of brevity, where an antibody configuration is amenable to more than one ABD, the individual ABDs are described in the same format (for example, in the case of an scFv-formatted fusion protein, the first and second ABD are characterized as a first target antigen scFv and a second target antigen scFv). However, it is expressly contemplated that a subject IL-12 Fc fusion protein can be configured such that a first ABD is present in one format (e.g., a nanobody format) and the second ABD is present in another format (e.g., an scFv format, or a Fab format).

Further, while exemplary IL-12 Fc fusion proteins are generally set forth such that either: (1) the first IL-12 subunit domain and the second IL-12 subunit domain are on the same monomer of a subject IL-12 Fc fusion protein (covalently linked using a domain linker in several embodiments (e.g., from N-terminus to C-terminus, variant IL-12 p35 subunit domain-domain linker-(variant) IL-12 p40 subunit domain, or (variant) IL-12 p40 subunit domain-domain linker-variant IL-12 p35 subunit domain)), or (2) the first IL-12 subunit domain and the second IL-12 subunit domain are on separate monomers of a subject IL-12 Fc fusion protein (e.g., a first monomer and a second monomer, respectively, or the reverse), it is also expressly contemplated that a subject IL-12 Fc fusion protein can be configured such that the first IL-12 subunit domain is covalently attached to a monomer of the subject IL-12 Fc fusion protein and the second IL-12 subunit domain is expressed as a soluble protein, wherein the soluble IL-12 subunit interacts with/binds the IL-12 subunit covalently attached to a monomer of the subject IL-12 Fc fusion protein. Exemplary configurations wherein one IL-12 subunit is covalently attached to a subject IL-12 Fc fusion protein and one IL-12 subunit is expressed as a soluble fragment is described in, for example, PCT Publication No. WO 2023/242769 A1, which is incorporated by reference in its entirety.

### 1. Fab Formats:

In some embodiments, the IL-12 Fc fusion protein is configured such that the ABD comprises a VH domain and a VL domain, and the IL-12 Fc fusion protein further comprises a CH1 domain and a CL domain. Any of the VH/VL domain pairs described herein may be incorporated into a subject Fab-formatted fusion protein.

### a. Embodiment (A)(1)

In one exemplary embodiment, the IL-12 Fc fusion protein comprises: (a) a first monomer comprising, from N-terminus to C-terminus: (i) a first IL-12 subunit domain, (ii) a first domain linker, (iii) a second IL-12 subunit domain, (iv) a second domain linker, and (v) a first Fc domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; (b) a second monomer comprising, from N-terminus to C-terminus: (i) a variable heavy (VH) domain, (ii) a constant heavy (CH1) domain, and (iii) a second Fc domain; and (c) a third monomer comprising, from N-terminus to C-terminus: (i) a variable light (VL) domain, and (ii) a constant light (CL) domain, wherein the VH domain and VL domain form an antigen binding domain (ABD) that binds a target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the ABD binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the ABD binds an extracellular domain (ECD) of the target antigen.

In some further embodiments, the target antigen comprises human PD1, and the ABD comprises a VH/VL domain pair, or a set of vhCDR1-3 and a set of vlCDR1-3, as described herein (see, e.g., Section (III)(A)(2)(a)). In other embodiments, the target antigen comprises human LAG3, and the ABD comprises a VH/VL domain pair, or a set of vhCDR1-3 and a set of vlCDR1-3, as described herein (see, e.g., Section (III)(A)(2)(b)). In other embodiments, the target antigen comprises human OX40, and the ABD comprises a VH/VL domain pair, or a set of vhCDR1-3 and a set of vlCDR1-3, as described herein (see, e.g., Section (III)(A)(2)(c)). In other embodiments, the target antigen comprises human 41BB, and the ABD comprises a VH/VL domain pair, or a set of vhCDR1-3 and a set of vlCDR1-3, as described herein (see, e.g., Section (III)(A)(2)(d)). In other embodiments, the target antigen comprises human ICOS, and the ABD comprises a VH/VL domain pair, or a set of vhCDR1-3 and a set of vlCDR1-3, as described herein (see, e.g., Section (III)(A)(2)(e)). In other embodiments, the target antigen comprises human CCR7, and the ABD comprises a VH/VL domain pair, or a set of vhCDR1-3 and a set of vlCDR1-3, as described herein (see, e.g., Section (III)(A)(2)(f)). In other embodiments, the target antigen comprises human CCR8, and the ABD comprises a VH/VL domain pair, or a set of vhCDR1-3 and a set of vlCDR1-3, as described herein (see, e.g., Section (III)(A)(2)(g)). In some further embodiments, the ABD binds an extracellular domain (ECD) of the target antigen.

In some further embodiments, the first domain linker and/or the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

### b. Embodiment (A)(2)

In one exemplary embodiment, the IL-12 Fc fusion protein comprises: (a) a first monomer comprising, from N-terminus to C-terminus: (i) a VH domain, (ii) a CH1 domain, and (iii) a first Fc domain; (b) a second monomer comprising, from N-terminus to C-terminus: (i) a VL domain, and (ii) a CL domain, wherein the VH domain and VL domain form an ABD that binds a first target antigen; (c) a third monomer comprising, from N-terminus to C-terminus: (i) a second Fc domain, (ii) a first domain linker, (iii) a first IL-12 subunit domain, (iv) a second domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the ABD binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the ABD binds an extracellular domain (ECD) of the target antigen.

In some further embodiments, the target antigen comprises human PD1, and the ABD comprises a VH/VL domain pair, or a set of vhCDR1-3 and a set of vlCDR1-3, as described herein (see, e.g., Section (III)(A)(2)(a)). In other embodiments, the target antigen comprises human LAG3, and the ABD comprises a VH/VL domain pair, or a set of vhCDR1-3 and a set of vlCDR1-3, as described herein (see, e.g., Section (III)(A)(2)(b)). In other embodiments, the target antigen comprises human OX40, and the ABD comprises a VH/VL domain pair, or a set of vhCDR1-3 and a set of vlCDR1-3, as described herein (see, e.g., Section (III)(A)(2)(c)). In other embodiments, the target antigen comprises human 41BB, and the ABD comprises a VH/VL domain pair, or a set of vhCDR1-3 and a set of vlCDR1-3, as described herein (see, e.g., Section (III)(A)(2)(d)). In other embodiments, the target antigen comprises human ICOS, and the ABD comprises a VH/VL domain pair, or a set of vhCDR1-3 and a set of vlCDR1-3, as described herein (see, e.g., Section (III)(A)(2)(e)). In other embodiments, the target antigen comprises human CCR7, and the ABD comprises a VH/VL domain pair, or a set of vhCDR1-3 and a set of vlCDR1-3, as described herein (see, e.g., Section (III)(A)(2)(f)). In other embodiments, the target antigen comprises human CCR8, and the ABD comprises a VH/VL domain pair, or a set of vhCDR1-3 and a set of vlCDR1-3, as described herein (see, e.g., Section (III)(A)(2)(g)). In some further embodiments, the ABD binds an extracellular domain (ECD) of the target antigen.

In some further embodiments, the first domain linker and/or the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

### c. Embodiment (A)(3)

In one exemplary embodiment, the IL-12 Fc fusion protein comprises: (a) a first monomer comprising, from N-terminus to C-terminus: (i) a first IL-12 subunit domain, (ii) a first domain linker, and (iii) a first Fc domain; (b) a second monomer comprising, from N-terminus to C-terminus: (i) a second IL-12 subunit domain, (ii) a second domain linker, (iii) a second Fc domain, (iv) a third domain linker, (v) a VH domain, and (vi) a CH1 domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (c) a third monomer comprising, from N-terminus to C-terminus: (i) a VL domain, and (ii) a CL domain, wherein the VH domain and VL domain form an ABD that binds a target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the ABD binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the ABD binds an extracellular domain (ECD) of the target antigen.

In some further embodiments, the target antigen comprises human PD1, and the ABD comprises a VH/VL domain pair, or a set of vhCDR1-3 and a set of vlCDR1-3, as described herein (see, e.g., Section (III)(A)(2)(a)). In other embodiments, the target antigen comprises human LAG3, and the ABD comprises a VH/VL domain pair, or a set of vhCDR1-3 and a set of vlCDR1-3, as described herein (see, e.g., Section (III)(A)(2)(b)). In other embodiments, the target antigen comprises human OX40, and the ABD comprises a VH/VL domain pair, or a set of vhCDR1-3 and a set of vlCDR1-3, as described herein (see, e.g., Section (III)(A)(2)(c)). In other embodiments, the target antigen comprises human 41BB, and the ABD comprises a VH/VL domain pair, or a set of vhCDR1-3 and a set of vlCDR1-3, as described herein (see, e.g., Section (III)(A)(2)(d)). In other embodiments, the target antigen comprises human ICOS, and the ABD comprises a VH/VL domain pair, or a set of vhCDR1-3 and a set of vlCDR1-3, as described herein (see, e.g., Section (III)(A)(2)(e)). In other embodiments, the target antigen comprises human CCR7, and the ABD comprises a VH/VL domain pair, or a set of vhCDR1-3 and a set of vlCDR1-3, as described herein (see, e.g., Section (III)(A)(2)(f)). In other embodiments, the target antigen comprises human CCR8, and the ABD comprises a VH/VL domain pair, or a set of vhCDR1-3 and a set of vlCDR1-3, as described herein (see, e.g., Section (III)(A)(2)(g)). In some further embodiments, the ABD binds an extracellular domain (ECD) of the target antigen.

In some further embodiments, the first domain linker, the second domain linker, and/or the third domain linker comprises any one of SEQ ID NOs: 31-40. In some other further embodiments, the first domain linker, the second domain linker, and/or the third domain linker comprises a covalent bond. In some other further embodiments, at least one domain linker comprises any one of SEQ ID NOs: 31-40, and at least one domain linker comprises a covalent bond. In some embodiments, at least one of the domain linkers comprises a hinge region of a Fc domain.

### d. Embodiment (A)(4)

In one exemplary embodiment, the IL-12 Fc fusion protein comprises: (a) a first monomer comprising, from N-terminus to C-terminus: (i) a VH domain, (ii) a CH1 domain, (iii) a first Fc domain, (iv) a first domain linker, and (v) a first IL-12 subunit domain; and (b) a second monomer comprising, from N-terminus to C-terminus: (i) a VL domain, (ii) a CL domain, (iii) a second Fc domain, (iv) a second domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the VH domain and VL domain form an ABD that binds a target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the ABD binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the ABD binds an extracellular domain (ECD) of the target antigen.

In some further embodiments, the target antigen comprises human PD1, and the ABD comprises a VH/VL domain pair, or a set of vhCDR1-3 and a set of vlCDR1-3, as described herein (see, e.g., Section (III)(A)(2)(a)). In other embodiments, the target antigen comprises human LAG3, and the ABD comprises a VH/VL domain pair, or a set of vhCDR1-3 and a set of vlCDR1-3, as described herein (see, e.g., Section (III)(A)(2)(b)). In other embodiments, the target antigen comprises human OX40, and the ABD comprises a VH/VL domain pair, or a set of vhCDR1-3 and a set of vlCDR1-3, as described herein (see, e.g., Section (III)(A)(2)(c)). In other embodiments, the target antigen comprises human 41BB, and the ABD comprises a VH/VL domain pair, or a set of vhCDR1-3 and a set of vlCDR1-3, as described herein (see, e.g., Section (III)(A)(2)(d)). In other embodiments, the target antigen comprises human ICOS, and the ABD comprises a VH/VL domain pair, or a set of vhCDR1-3 and a set of vlCDR1-3, as described herein (see, e.g., Section (III)(A)(2)(e)). In other embodiments, the target antigen comprises human CCR7, and the ABD comprises a VH/VL domain pair, or a set of vhCDR1-3 and a set of vlCDR1-3, as described herein (see, e.g., Section (III)(A)(2)(f)). In other embodiments, the target antigen comprises human CCR8, and the ABD comprises a VH/VL domain pair, or a set of vhCDR1-3 and a set of vlCDR1-3, as described herein (see, e.g., Section (III)(A)(2)(g)). In some further embodiments, the ABD binds an extracellular domain (ECD) of the target antigen.

In some further embodiments, the first domain linker and/or the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

### e. Embodiment (A)(5)

In one exemplary embodiment, the IL-12 Fc fusion protein comprises: (a) a first monomer comprising, from N-terminus to C-terminus: (i) a VH domain, (ii) a CH1 domain, and (iii) a first Fc domain; and (b) a second monomer comprising, from N-terminus to C-terminus: (i) a VL domain, (ii) a CL domain, (iii) a second Fc domain, (iv) a first domain linker, (v) a first IL-12 subunit domain, (vi) a second domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the VH domain and VL domain form an ABD that binds a target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the ABD binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the ABD binds an extracellular domain (ECD) of the target antigen.

In some further embodiments, the target antigen comprises human PD1, and the ABD comprises a VH/VL domain pair, or a set of vhCDR1-3 and a set of vlCDR1-3, as described herein (see, e.g., Section (III)(A)(2)(a)). In other embodiments, the target antigen comprises human LAG3, and the ABD comprises a VH/VL domain pair, or a set of vhCDR1-3 and a set of vlCDR1-3, as described herein (see, e.g., Section (III)(A)(2)(b)). In other embodiments, the target antigen comprises human OX40, and the ABD comprises a VH/VL domain pair, or a set of vhCDR1-3 and a set of vlCDR1-3, as described herein (see, e.g., Section (III)(A)(2)(c)). In other embodiments, the target antigen comprises human 41BB, and the ABD comprises a VH/VL domain pair, or a set of vhCDR1-3 and a set of vlCDR1-3, as described herein (see, e.g., Section (III)(A)(2)(d)). In other embodiments, the target antigen comprises human ICOS, and the ABD comprises a VH/VL domain pair, or a set of vhCDR1-3 and a set of vlCDR1-3, as described herein (see, e.g., Section (III)(A)(2)(e)). In other embodiments, the target antigen comprises human CCR7, and the ABD comprises a VH/VL domain pair, or a set of vhCDR1-3 and a set of vlCDR1-3, as described herein (see, e.g., Section (III)(A)(2)(f)). In other embodiments, the target antigen comprises human CCR8, and the ABD comprises a VH/VL domain pair, or a set of vhCDR1-3 and a set of vlCDR1-3, as described herein (see, e.g., Section (III)(A)(2)(g)). In some further embodiments, the ABD binds an extracellular domain (ECD) of the target antigen.

In some further embodiments, the first domain linker and/or the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

### f. Embodiment (A)(6)

In one exemplary embodiment, the IL-12 Fc fusion protein comprises: (a) a first monomer comprising a first Fc domain; (b) a second monomer comprising, from N-terminus to C-terminus: (i) a VL domain, and (ii) a CL domain; and (c) a third monomer comprising, from N-terminus to C-terminus: (i) a first IL-12 subunit domain, (ii) a first domain linker, (iii) a second IL-12 subunit domain, (iv) a second domain linker, (v) a second Fc domain, (vi) a VH domain, and (vii) a CH1 domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the VH domain and VL domain form an ABD that binds a target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the ABD binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In still other embodiments, the target antigen comprises human OX40, and the ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In still other embodiments, the target antigen comprises human ICOS, and the ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In still other embodiments, the target antigen comprises human 41BB, and the ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In still other embodiments, the target antigen comprises human CCR7, and the ABD comprises a VH/VL domain pair selected from a group including: (i) SEQ ID NOs: 78 and 79, respectively. In still other embodiments, the target antigen comprises human CCR8, and the ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the ABD binds an extracellular domain (ECD) of the target antigen.

In some further embodiments, the target antigen comprises human PD1, and the ABD comprises a VH/VL domain pair, or a set of vhCDR1-3 and a set of vlCDR1-3, as described herein (see, e.g., Section (III)(A)(2)(a)). In other embodiments, the target antigen comprises human LAG3, and the ABD comprises a VH/VL domain pair, or a set of vhCDR1-3 and a set of vlCDR1-3, as described herein (see, e.g., Section (III)(A)(2)(b)). In other embodiments, the target antigen comprises human OX40, and the ABD comprises a VH/VL domain pair, or a set of vhCDR1-3 and a set of vlCDR1-3, as described herein (see, e.g., Section (III)(A)(2)(c)). In other embodiments, the target antigen comprises human 41BB, and the ABD comprises a VH/VL domain pair, or a set of vhCDR1-3 and a set of vlCDR1-3, as described herein (see, e.g., Section (III)(A)(2)(d)). In other embodiments, the target antigen comprises human ICOS, and the ABD comprises a VH/VL domain pair, or a set of vhCDR1-3 and a set of vlCDR1-3, as described herein (see, e.g., Section (III)(A)(2)(e)). In other embodiments, the target antigen comprises human CCR7, and the ABD comprises a VH/VL domain pair, or a set of vhCDR1-3 and a set of vlCDR1-3, as described herein (see, e.g., Section (III)(A)(2)(f)). In other embodiments, the target antigen comprises human CCR8, and the ABD comprises a VH/VL domain pair, or a set of vhCDR1-3 and a set of vlCDR1-3, as described herein (see, e.g., Section (III)(A)(2)(g)). In some further embodiments, the ABD binds an extracellular domain (ECD) of the target antigen.

In some further embodiments, the first domain linker and/or the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

### g. Embodiment (A)(7)

In one exemplary embodiment, the IL-12 Fc fusion protein comprises: (a) a first monomer comprising, from N-terminus to C-terminus: (i) a first VH (VH1) domain, (ii) a first CH1 domain, and (iii) a first Fc domain; (b) a second monomer comprising, from N-terminus to C-terminus: (i) a first VL (VL1) domain, and (ii) a first CL domain, wherein the VH1 domain and VL1 domain form a first ABD that binds a first target antigen; (c) a third monomer comprising, from N-terminus to C-terminus: (i) a second VH (V12) domain, (ii) a second CH1 domain, (iii) a second Fc domain, (iv) a first domain linker, (v) a first IL-12 subunit domain, (vi) a second domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (d) a fourth monomer comprising, from N-terminus to C-terminus: (i) a second VL (VL2) domain, and (ii) a second CL domain, wherein the VH2 domain and VL2 domain form a second ABD that binds a second target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first ABD binds a first target antigen selected from a group including: (i) human OX40, (ii) human LAG3, (iii) human PD1, (iv) human ICOS, (v) human 41BB, (vi) human CCR7, and (vii) human CCR8.

In some further embodiments, the second ABD binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first ABD and the second ABD both bind a target antigen selected from a group including: (i) human OX40, (ii) human LAG3, (iii) human PD1, (iv) human ICOS, (v) human 41BB, (vi) human CCR7, and (vii) human CCR8.

In some further embodiments, the first ABD binds a first target antigen selected from a group including: (i) human OX40, (ii) human LAG3, (iii) human PD1, (iv) human ICOS, (v) human 41BB, (vi) human CCR7, and (vii) human CCR8; and the second ABD binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first target antigen and/or the second target antigen comprises human PD1, and the first ABD and/or the second ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first ABD and/or the second ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first ABD and/or the second ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first ABD and/or the second ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first ABD and/or the second ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first ABD and/or the second ABD comprises a VH/VL domain pair selected from a group including: (i) SEQ ID NOs: 78 and 79, respectively. In other embodiments, the first target antigen and/or the second target antigen comprises human CCR8, and the first ABD and/or the second ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the first ABD binds an ECD of the first target antigen. In some further embodiments, the second ABD binds an ECD of the second target antigen. In some further embodiments, the first ABD binds an ECD of the first target antigen, and the second ABD binds an ECD of the second target antigen.

In some further embodiments, the first target antigen and/or the second target antigen comprises human PD1, and the first ABD and/or the second ABD comprises a VH/VL domain pair, or a set of vhCDR1-3 and a set of vlCDR1-3, as described herein (see, e.g., Section (III)(A)(2)(a)). In other embodiments, the first target antigen and/or the second target antigen comprises human LAG3, and the first ABD and/or the second ABD comprises a VH/VL domain pair, or a set of vhCDR1-3 and a set of vlCDR1-3, as described herein (see, e.g., Section (III)(A)(2)(b)). In other embodiments, the first target antigen and/or the second target antigen comprises human OX40, and the first ABD and/or the second ABD comprises a VH/VL domain pair, or a set of vhCDR1-3 and a set of vlCDR1-3, as described herein (see, e.g., Section (III)(A)(2)(c)). In other embodiments, the first target antigen and/or the second target antigen comprises human 41BB, and the first ABD and/or the second ABD comprises a VH/VL domain pair, or a set of vhCDR1-3 and a set of vlCDR1-3, as described herein (see, e.g., Section (III)(A)(2)(d)). In other embodiments, the first target antigen and/or the second target antigen comprises human ICOS, and the first ABD and/or the second ABD comprises a VH/VL domain pair, or a set of vhCDR1-3 and a set of vlCDR1-3, as described herein (see, e.g., Section (III)(A)(2)(e)). In other embodiments, the first target antigen and/or the second target antigen comprises human CCR7, and the first ABD and/or the second ABD comprises a VH/VL domain pair, or a set of vhCDR1-3 and a set of vlCDR1-3, as described herein (see, e.g., Section (III)(A)(2)(f)). In other embodiments, the first target antigen and/or the second target antigen comprises human CCR8, and the first ABD and/or the second ABD comprises a VH/VL domain pair, or a set of vhCDR1-3 and a set of vlCDR1-3, as described herein (see, e.g., Section (III)(A)(2)(g)). In some further embodiments, the first ABD binds an ECD of the first target antigen. In some further embodiments, the second ABD binds an ECD of the second target antigen. In some further embodiments, the first ABD binds an ECD of the first target antigen, and the second ABD binds an ECD of the second target antigen.

In some further embodiments, the first domain linker and/or the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

### h. Embodiment (A)(8)

In one exemplary embodiment, the IL-12 Fc fusion protein comprises: (a) a first monomer comprising, from N-terminus to C-terminus: (i) a first VH (VH1) domain, (ii) a first CH1 domain, (iii) a first Fc domain, (iv) a first domain linker, and (v) a first IL-12 subunit domain; (b) a second monomer comprising, from N-terminus to C-terminus: (i) a first VL (VL1) domain, and (ii) a first CL domain, wherein the VH1 domain and VL1 domain form a first ABD that binds a first target antigen; (c) a third monomer comprising, from N-terminus to C-terminus: (i) a second VH (V12) domain, (ii) a second CH1 domain, (iii) a second Fc domain, (iv) a second domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (d) a fourth monomer comprising, from N-terminus to C-terminus: (i) a second VL (VL2) domain, and (ii) a second CL domain, wherein the VH2 domain and VL2 domain form a second ABD that binds a second target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 26-30, and the second Fc domain comprises any one of SEQ ID NOs: 26-30. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first ABD binds a first target antigen selected from a group including: (i) human OX40, (ii) human LAG3, (iii) human PD1, (iv) human ICOS, (v) human 41BB, (vi) human CCR7, and (vii) human CCR8.

In some further embodiments, the second ABD binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first ABD and the second ABD both bind a target antigen selected from a group including: (i) human OX40, (ii) human LAG3, (iii) human PD1, (iv) human ICOS, (v) human 41BB, (vi) human CCR7, and (vii) human CCR8.

In some further embodiments, the first ABD binds a first target antigen selected from a group including: (i) human OX40, (ii) human LAG3, (iii) human PD1, (iv) human ICOS, (v) human 41BB, (vi) human CCR7, and (vii) human CCR8; and the second ABD binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first target antigen and/or the second target antigen comprises human PD1, and the first ABD and/or the second ABD comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first ABD and/or the second ABD comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first ABD and/or the second ABD comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first ABD and/or the second ABD comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first ABD and/or the second ABD comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first ABD and/or the second ABD comprises a VH/VL domain pair selected from a group including: (i) SEQ ID NOs: 78 and 79, respectively. In other embodiments, the first target antigen and/or the second target antigen comprises human CCR8, and the first ABD and/or the second ABD comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the first ABD binds an ECD of the first target antigen. In some further embodiments, the second ABD binds an ECD of the second target antigen. In some further embodiments, the first ABD binds an ECD of the first target antigen, and the second ABD binds an ECD of the second target antigen.

In some further embodiments, the first target antigen and/or the second target antigen comprises human PD1, and the first ABD and/or the second ABD comprises a VH/VL domain pair, or a set of vhCDR1-3 and a set of vlCDR1-3, as described herein (see, e.g., Section (III)(A)(2)(a)). In other embodiments, the first target antigen and/or the second target antigen comprises human LAG3, and the first ABD and/or the second ABD comprises a VH/VL domain pair, or a set of vhCDR1-3 and a set of vlCDR1-3, as described herein (see, e.g., Section (III)(A)(2)(b)). In other embodiments, the first target antigen and/or the second target antigen comprises human OX40, and the first ABD and/or the second ABD comprises a VH/VL domain pair, or a set of vhCDR1-3 and a set of vlCDR1-3, as described herein (see, e.g., Section (III)(A)(2)(c)). In other embodiments, the first target antigen and/or the second target antigen comprises human 41BB, and the first ABD and/or the second ABD comprises a VH/VL domain pair, or a set of vhCDR1-3 and a set of vlCDR1-3, as described herein (see, e.g., Section (III)(A)(2)(d)). In other embodiments, the first target antigen and/or the second target antigen comprises human ICOS, and the first ABD and/or the second ABD comprises a VH/VL domain pair, or a set of vhCDR1-3 and a set of vlCDR1-3, as described herein (see, e.g., Section (III)(A)(2)(e)). In other embodiments, the first target antigen and/or the second target antigen comprises human CCR7, and the first ABD and/or the second ABD comprises a VH/VL domain pair, or a set of vhCDR1-3 and a set of vlCDR1-3, as described herein (see, e.g., Section (III)(A)(2)(f)). In other embodiments, the first target antigen and/or the second target antigen comprises human CCR8, and the first ABD and/or the second ABD comprises a VH/VL domain pair, or a set of vhCDR1-3 and a set of vlCDR1-3, as described herein (see, e.g., Section (III)(A)(2)(g)). In some further embodiments, the first ABD binds an ECD of the first target antigen. In some further embodiments, the second ABD binds an ECD of the second target antigen. In some further embodiments, the first ABD binds an ECD of the first target antigen, and the second ABD binds an ECD of the second target antigen.

In some further embodiments, the first domain linker and/or the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

2. scFv Formats:

In some embodiments, the IL-12 Fc fusion protein is configured such that the ABD comprises a target antigen scFv, wherein the scFv comprises, from N-terminus to C-terminus: (1) a VH domain, a domain linker, and a VL domain, or (2) a VL domain, a domain linker, and a VH domain. Any of the VH/VL domain pairs described herein may be incorporated into a subject scFv-formatted fusion protein.

### a. Embodiment (B)(1)

In one exemplary embodiment, the IL-12 Fc fusion protein comprises: (a) a first monomer comprising, from N-terminus to C-terminus: (i) a first IL-12 subunit domain, (ii) a first domain linker, (iii) a second IL-12 subunit domain, and (iv) a first Fc domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising: (i) an anti-target antigen scFv, wherein the anti-target antigen scFv comprises a VH domain, a second domain linker, and a VL domain, (ii) a third domain linker, and (iii) a second Fc domain, wherein the anti-target antigen scFv is covalently attached to the third domain linker at the N-terminus of the second Fc domain.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the anti-target antigen scFv binds an extracellular domain (ECD) of the target antigen.

In some further embodiments, the target antigen comprises human PD1, and the anti-target antigen scFv comprises a VH/VL domain pair, or a set of vhCDR1-3 and a set of vlCDR1-3, as described herein (see, e.g., Section (III)(A)(2)(a)). In other embodiments, the target antigen comprises human LAG3, and the anti-target antigen scFv comprises a VH/VL domain pair, or a set of vhCDR1-3 and a set of vlCDR1-3, as described herein (see, e.g., Section (III)(A)(2)(b)). In other embodiments, the target antigen comprises human OX40, and the anti-target antigen scFv comprises a VH/VL domain pair, or a set of vhCDR1-3 and a set of vlCDR1-3, as described herein (see, e.g., Section (III)(A)(2)(c)). In other embodiments, the target antigen comprises human 41BB, and the anti-target antigen scFv comprises a VH/VL domain pair, or a set of vhCDR1-3 and a set of vlCDR1-3, as described herein (see, e.g., Section (III)(A)(2)(d)). In other embodiments, the target antigen comprises human ICOS, and the anti-target antigen scFv comprises a VH/VL domain pair, or a set of vhCDR1-3 and a set of vlCDR1-3, as described herein (see, e.g., Section (III)(A)(2)(e)). In other embodiments, the target antigen comprises human CCR7, and the anti-target antigen scFv comprises a VH/VL domain pair, or a set of vhCDR1-3 and a set of vlCDR1-3, as described herein (see, e.g., Section (III)(A)(2)(f)). In other embodiments, the target antigen comprises human CCR8, and the anti-target antigen scFv comprises a VH/VL domain pair, or a set of vhCDR1-3 and a set of vlCDR1-3, as described herein (see, e.g., Section (III)(A)(2)(g)). In some further embodiments, the anti-target antigen scFv binds an extracellular domain (ECD) of the target antigen.

In some further embodiments, the VH domain of the anti-target antigen scFv is covalently attached to the third domain linker at the N-terminus of the second Fc domain. In other embodiments, the VL domain of the anti-target antigen scFv is covalently attached to the third domain linker at the N-terminus of the second Fc domain.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, and the third domain linker comprises any one of SEQ ID NOs: 31-40. In some other further embodiments, the first domain linker, the second domain linker, and/or the third domain linker comprises a covalent bond. In some other further embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

### b. Embodiment (B)(2)

In one exemplary embodiment, the IL-12 Fc fusion protein comprises: (a) a first monomer comprising: (i) an anti-target antigen scFv, wherein the anti-target antigen scFv comprises a VH domain, a first domain linker, and a VL domain, (ii) a second domain linker, and (iii) a first Fc domain, wherein the anti-target antigen scFv is covalently attached to the second domain linker at the N-terminus of the first Fc domain; and (b) a second monomer comprising, from N-terminus to C-terminus: (i) a second Fc domain, (ii) a third domain linker, (iii) a first IL-12 subunit domain, (iv) a fourth domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the anti-target antigen scFv binds an extracellular domain (ECD) of the target antigen.

In some further embodiments, the target antigen comprises human PD1, and the anti-target antigen scFv comprises a VH/VL domain pair, or a set of vhCDR1-3 and a set of vlCDR1-3, as described herein (see, e.g., Section (III)(A)(2)(a)). In other embodiments, the target antigen comprises human LAG3, and the anti-target antigen scFv comprises a VH/VL domain pair, or a set of vhCDR1-3 and a set of vlCDR1-3, as described herein (see, e.g., Section (III)(A)(2)(b)). In other embodiments, the target antigen comprises human OX40, and the anti-target antigen scFv comprises a VH/VL domain pair, or a set of vhCDR1-3 and a set of vlCDR1-3, as described herein (see, e.g., Section (III)(A)(2)(c)). In other embodiments, the target antigen comprises human 41BB, and the anti-target antigen scFv comprises a VH/VL domain pair, or a set of vhCDR1-3 and a set of vlCDR1-3, as described herein (see, e.g., Section (III)(A)(2)(d)). In other embodiments, the target antigen comprises human ICOS, and the anti-target antigen scFv comprises a VH/VL domain pair, or a set of vhCDR1-3 and a set of vlCDR1-3, as described herein (see, e.g., Section (III)(A)(2)(e)). In other embodiments, the target antigen comprises human CCR7, and the anti-target antigen scFv comprises a VH/VL domain pair, or a set of vhCDR1-3 and a set of vlCDR1-3, as described herein (see, e.g., Section (III)(A)(2)(f)). In other embodiments, the target antigen comprises human CCR8, and the anti-target antigen scFv comprises a VH/VL domain pair, or a set of vhCDR1-3 and a set of vlCDR1-3, as described herein (see, e.g., Section (III)(A)(2)(g)). In some further embodiments, the anti-target antigen scFv binds an extracellular domain (ECD) of the target antigen.

In some further embodiments, the VH domain of the anti-target antigen scFv is covalently attached to the second domain linker at the N-terminus of the first Fc domain. In other embodiments, the VL domain of the anti-target antigen scFv is covalently attached to the second domain linker at the N-terminus of the first Fc domain.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, the third domain linker comprises any one of SEQ ID NOs: 31-40, and the fourth domain linker comprises any one of SEQ ID NOs: 31-40.

In some further embodiments, the first domain linker comprises any one of SEQ ID NOs: 31-40. In still some further embodiments, the second domain linker, the third domain linker, and/or the fourth domain linker comprises any one of SEQ ID NOs: 31-40. In still some other further embodiments, the second domain linker, the third domain linker, and/or the fourth domain linker comprises a covalent bond. In still some other further embodiments, at least one domain linker comprises any one of SEQ ID NOs: 31-40, and at least one domain linker comprises a covalent bond. In some embodiments, at least one of the domain linkers comprises a hinge region of a Fc domain.

### c. Embodiment (B)(3)

In one exemplary embodiment, the IL-12 Fc fusion protein comprises: (a) a first monomer comprising, from N-terminus to C-terminus: (i) a first IL-12 subunit domain, (ii) a first domain linker, and (iii) a first Fc domain; and (b) a second monomer comprising: (i) a second IL-12 subunit domain, (ii) a second domain linker, (iii) a second Fc domain, wherein the second IL-12 subunit domain is covalently attached to the second domain linker at the N-terminus of the second Fc domain, (iv) a third domain linker, and (v) an anti-target antigen scFv, wherein: (1) the second Fc domain is covalently attached to the third domain linker at the N-terminus of the anti-target antigen scFv, (2) the anti-target antigen scFv comprises a VH domain, a fourth domain linker, and a VL domain, (3) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (4) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the anti-target antigen scFv binds an extracellular domain (ECD) of the target antigen.

In some further embodiments, the target antigen comprises human PD1, and the anti-target antigen scFv comprises a VH/VL domain pair, or a set of vhCDR1-3 and a set of vlCDR1-3, as described herein (see, e.g., Section (III)(A)(2)(a)). In other embodiments, the target antigen comprises human LAG3, and the anti-target antigen scFv comprises a VH/VL domain pair, or a set of vhCDR1-3 and a set of vlCDR1-3, as described herein (see, e.g., Section (III)(A)(2)(b)). In other embodiments, the target antigen comprises human OX40, and the anti-target antigen scFv comprises a VH/VL domain pair, or a set of vhCDR1-3 and a set of vlCDR1-3, as described herein (see, e.g., Section (III)(A)(2)(c)). In other embodiments, the target antigen comprises human 41BB, and the anti-target antigen scFv comprises a VH/VL domain pair, or a set of vhCDR1-3 and a set of vlCDR1-3, as described herein (see, e.g., Section (III)(A)(2)(d)). In other embodiments, the target antigen comprises human ICOS, and the anti-target antigen scFv comprises a VH/VL domain pair, or a set of vhCDR1-3 and a set of vlCDR1-3, as described herein (see, e.g., Section (III)(A)(2)(e)). In other embodiments, the target antigen comprises human CCR7, and the anti-target antigen scFv comprises a VH/VL domain pair, or a set of vhCDR1-3 and a set of vlCDR1-3, as described herein (see, e.g., Section (III)(A)(2)(f)). In other embodiments, the target antigen comprises human CCR8, and the anti-target antigen scFv comprises a VH/VL domain pair, or a set of vhCDR1-3 and a set of vlCDR1-3, as described herein (see, e.g., Section (III)(A)(2)(g)). In some further embodiments, the anti-target antigen scFv binds an extracellular domain (ECD) of the target antigen.

In some further embodiments, the VH domain of the anti-target antigen scFv is covalently attached to the third domain linker at the C-terminus of the second Fc domain. In other embodiments, the VL domain of the anti-target antigen scFv is covalently attached to the third domain linker at the C-terminus of the second Fc domain.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, the third domain linker comprises any one of SEQ ID NOs: 31-40, and the fourth domain linker comprises any one of SEQ ID NOs: 31-40.

In some further embodiments, the fourth domain linker comprises any one of SEQ ID NOs: 31-40. In still some further embodiments, the first domain linker, the second domain linker, and/or the third domain linker comprises any one of SEQ ID NOs: 31-40. In still some other further embodiments, the first domain linker, the second domain linker, and/or the third domain linker comprises a covalent bond. In still some other further embodiments, at least one domain linker comprises any one of SEQ ID NOs: 31-40, and at least one domain linker comprises a covalent bond. In some embodiments, at least one of the domain linkers comprises a hinge region of a Fc domain.

### d. Embodiment (B)(4)

In one exemplary embodiment, the IL-12 Fc fusion protein comprises: (a) a first monomer comprising: (i) a first IL-12 subunit domain, (ii) a first domain linker, (iii) a second IL-12 subunit domain, wherein the first IL-12 subunit domain is covalently attached to the first domain linker at the N-terminus of the second IL-12 subunit domain, (iv) a second domain linker, (v) a first Fc domain, wherein the second IL-12 subunit domain is covalently attached to the second domain linker at the N-terminus of the first Fc domain, (vi) a third domain linker, and (vii) an anti-target antigen scFv, wherein: (1) the first Fc domain is covalently attached to the third domain linker at the N-terminus of the anti-target antigen scFv, (2) the anti-target antigen scFv comprises a VH domain, a fourth domain linker, and a VL domain, (3) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (4) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising a second Fc domain.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the anti-target antigen scFv binds a target antigen selected from a group including: (1) human OX40, (2) human LAG3, (3) human PD1, (4) human ICOS, (5) human 41BB, (6) human CCR7, and (7) human CCR8.

In some further embodiments, the target antigen comprises human PD1, and the anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the target antigen comprises human LAG3, and the anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the target antigen comprises human OX40, and the anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the target antigen comprises human ICOS, and the anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the target antigen comprises human 41BB, and the anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the target antigen comprises human CCR7, and the anti-target antigen scFv comprises a VH/VL domain pair selected from a group including: (i) SEQ ID NOs: 78 and 79, respectively. In other embodiments, the target antigen comprises human CCR8, and the anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the anti-target antigen scFv binds an extracellular domain (ECD) of the target antigen.

In some further embodiments, the target antigen comprises human PD1, and the anti-target antigen scFv comprises a VH/VL domain pair, or a set of vhCDR1-3 and a set of vlCDR1-3, as described herein (see, e.g., Section (III)(A)(2)(a)). In other embodiments, the target antigen comprises human LAG3, and the anti-target antigen scFv comprises a VH/VL domain pair, or a set of vhCDR1-3 and a set of vlCDR1-3, as described herein (see, e.g., Section (III)(A)(2)(b)). In other embodiments, the target antigen comprises human OX40, and the anti-target antigen scFv comprises a VH/VL domain pair, or a set of vhCDR1-3 and a set of vlCDR1-3, as described herein (see, e.g., Section (III)(A)(2)(c)). In other embodiments, the target antigen comprises human 41BB, and the anti-target antigen scFv comprises a VH/VL domain pair, or a set of vhCDR1-3 and a set of vlCDR1-3, as described herein (see, e.g., Section (III)(A)(2)(d)). In other embodiments, the target antigen comprises human ICOS, and the anti-target antigen scFv comprises a VH/VL domain pair, or a set of vhCDR1-3 and a set of vlCDR1-3, as described herein (see, e.g., Section (III)(A)(2)(e)). In other embodiments, the target antigen comprises human CCR7, and the anti-target antigen scFv comprises a VH/VL domain pair, or a set of vhCDR1-3 and a set of vlCDR1-3, as described herein (see, e.g., Section (III)(A)(2)(f)). In other embodiments, the target antigen comprises human CCR8, and the anti-target antigen scFv comprises a VH/VL domain pair, or a set of vhCDR1-3 and a set of vlCDR1-3, as described herein (see, e.g., Section (III)(A)(2)(g)). In some further embodiments, the anti-target antigen scFv binds an extracellular domain (ECD) of the target antigen.

In some further embodiments, the VH domain of the anti-target antigen scFv is covalently attached to the third domain linker at the C-terminus of the first Fc domain. In other embodiments, the VL domain of the anti-target antigen scFv is covalently attached to the third domain linker at the C-terminus of the first Fc domain.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, the third domain linker comprises any one of SEQ ID NOs: 31-40, and the fourth domain linker comprises any one of SEQ ID NOs: 31-40.

In some further embodiments, the fourth domain linker comprises any one of SEQ ID NOs: 31-40. In still some further embodiments, the first domain linker, the second domain linker, and/or the third domain linker comprises any one of SEQ ID NOs: 31-40. In still some other further embodiments, the first domain linker, the second domain linker, and/or the third domain linker comprises a covalent bond. In still some other further embodiments, at least one domain linker comprises any one of SEQ ID NOs: 31-40, and at least one domain linker comprises a covalent bond. In some embodiments, at least one of the domain linkers comprises a hinge region of a Fc domain.

### e. Embodiment (B)(5)

In one exemplary embodiment, the IL-12 Fc fusion protein comprises: (a) a first monomer comprising: (i) a first anti-target antigen scFv, wherein the first anti-target antigen scFv comprises a first VH (VH1) domain, a first domain linker, and a first VL (VL1) domain, (ii) a second domain linker, and (iii) a first Fc domain, wherein the first anti-target antigen scFv is covalently attached to the second domain linker at the N-terminus of the first Fc domain; and (b) a second monomer comprising: (i) a second anti-target antigen scFv, wherein the second anti-target antigen scFv comprises a second VH (VH2) domain, a third domain linker, and a second VL (VL2) domain, (ii) a fourth domain linker, (iii) a second Fc domain, wherein the second anti-target antigen scFv is covalently attached to the fourth domain linker at the N-terminus of the second Fc domain, (iv) a fifth domain linker, (v) a first IL-12 subunit domain, (vi) a sixth domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, (3) the second Fc domain is covalently attached to the fifth domain linker at the N-terminus of the first IL-12 subunit domain, and (4) the first IL-12 subunit domain is covalently attached to the sixth domain linker at the N-terminus of the second IL-12 subunit domain.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8.

In some further embodiments, the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first anti-target antigen scFv and the second anti-target antigen scFv both bind a target antigen selected from a group including: (i) human OX40, (ii) human LAG3, (iii) human PD1, (iv) human ICOS, (v) human 41BB, (vi) human CCR7, and (vii) human CCR8.

In some further embodiments, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8, and the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first target antigen and/or the second target antigen comprises human PD1, and the first anti-target antigen scFv and/or the second anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first anti-target antigen scFv and/or the second anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first anti-target antigen scFv and/or the second anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first anti-target antigen scFv and/or the second anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first anti-target antigen scFv and/or the second anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first anti-target antigen scFv and/or the second anti-target antigen scFv comprises a VH/VL domain pair selected from a group including: (i) SEQ ID NOs: 78 and 79, respectively. In other embodiments, the first target antigen and/or the second target antigen comprises human CCR8, and the first anti-target antigen scFv and/or the second anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the first anti-target antigen scFv binds an ECD of the first target antigen. In some further embodiments, the second anti-target antigen scFv binds an ECD of the second target antigen. In some further embodiments, the first anti-target antigen scFv binds an ECD of the first target antigen, and the second anti-target antigen scFv binds an ECD of the second target antigen.

In some further embodiments, the first target antigen and/or the second target antigen comprises human PD1, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(a)). In other embodiments, the first target antigen and/or the second target antigen comprises human LAG3, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(b)). In other embodiments, the first target antigen and/or the second target antigen comprises human OX40, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(c)). In other embodiments, the first target antigen and/or the second target antigen comprises human 41BB, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(d)). In other embodiments, the first target antigen and/or the second target antigen comprises human ICOS, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(e)). In other embodiments, the first target antigen and/or the second target antigen comprises human CCR7, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(f)). In other embodiments, the first target antigen and/or the second target antigen comprises human CCR8, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(g). In some further embodiments, the first anti-target antigen scFv binds an ECD of the first target antigen. In some further embodiments, the second anti-target antigen scFv binds an ECD of the second target antigen. In some further embodiments, the first anti-target antigen scFv binds an ECD of the first target antigen, and the second anti-target antigen scFv binds an ECD of the second target antigen.

In some further embodiments, the VH1 domain of the first anti-target antigen scFv is covalently attached to the second domain linker at the N-terminus of the first Fc domain. In other embodiments, the VL1 domain of the anti-target antigen scFv is covalently attached to the second domain linker at the N-terminus of the first Fc domain.

In some further embodiments, the VH2 domain of the second anti-target antigen scFv is covalently attached to the fourth domain linker at the N-terminus of the second Fc domain. In other embodiments, the VL2 domain of the second anti-target antigen scFv is covalently attached to the fourth domain linker at the N-terminus of the second Fc domain.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, the third domain linker comprises any one of SEQ ID NOs: 31-40, the fourth domain linker comprises any one of SEQ ID NOs: 31-40, the fifth domain linker comprises any one of SEQ ID NOs: 31-40, and the sixth domain linker comprises any one of SEQ ID NOs: 31-40.

In some further embodiments, the first domain linker comprises any one of SEQ ID NOs: 31-40, and the third domain linker comprises any one of SEQ ID NOs: 31-40. In still some further embodiments, the second domain linker, the fourth domain linker, the fifth domain linker, and/or the sixth domain linker comprises any one of SEQ ID NOs: 31-40. In some other further embodiments, the second domain linker, the fourth domain linker, the fifth domain linker, and/or the sixth domain linker comprises a covalent bond. In some other further embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

### f. Embodiment (B)(6)

In one exemplary embodiment, the IL-12 Fc fusion protein comprises: (a) a first monomer comprising: (i) a first anti-target antigen scFv, wherein the first anti-target antigen scFv comprises a first VH (VH1) domain, a first domain linker, and a first VL (VL1) domain, (ii) a second domain linker, (iii) a first Fc domain, wherein the first anti-target antigen scFv is covalently attached to the second domain linker at the N-terminus of the first Fc domain, (iv) a third domain linker, and (v) a first IL-12 subunit domain, wherein the first Fc domain is covalently attached to the third domain linker at the N-terminus of the first IL-12 subunit domain; and (b) a second monomer comprising: (i) a second anti-target antigen scFv, wherein the second anti-target antigen scFv comprises a second VH (VH2) domain, a fourth domain linker, and a second VL (VL2) domain, (ii) a fifth domain linker, (iii) a second Fc domain, wherein the second anti-target antigen scFv is covalently attached to the fifth domain linker at the N-terminus of the second Fc domain, (iv) a sixth domain linker, and (v) a second IL-12 subunit domain, wherein: (1) the second Fc domain is covalently attached to the sixth domain linker at the N-terminus of the second IL-12 subunit domain, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8.

In some further embodiments, the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first anti-target antigen scFv and the second anti-target antigen scFv both bind a target antigen selected from a group including: (i) human OX40, (ii) human LAG3, (iii) human PD1, (iv) human ICOS, (v) human 41BB, (vi) human CCR7, and (vii) human CCR8.

In some further embodiments, the first anti-target antigen scFv binds a first target antigen selected from a group including: (i) human OX40, (ii) human LAG3, (iii) human PD1, (iv) human ICOS, (v) human 41BB, (vi) human CCR7, and (vii) human CCR8, and the second anti-target antigen scFv binds a second target antigen selected from a group including: (i) human OX40, (ii) human LAG3, (iii) human PD1, (iv) human ICOS, (v) human 41BB, (vi) human CCR7, and (vii) human CCR8.

In some further embodiments, the first target antigen and/or the second target antigen comprises human PD1, and the first anti-target antigen scFv and/or the second anti-target antigen scFv comprises a VH/VL domain pair selected from a group including: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively. In other embodiments, the first target antigen and/or the second target antigen comprises human LAG3, and the first anti-target antigen scFv and/or the second anti-target antigen scFv comprises a VH/VL domain pair selected from a group including: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively. In other embodiments, the first target antigen and/or the second target antigen comprises human OX40, and the first anti-target antigen scFv and/or the second anti-target antigen scFv comprises a VH/VL domain pair selected from a group including: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112. In other embodiments, the first target antigen and/or the second target antigen comprises human ICOS, and the first anti-target antigen scFv and/or the second anti-target antigen scFv comprises a VH/VL domain pair selected from a group including: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively. In other embodiments, the first target antigen and/or the second target antigen comprises human 41BB, and the first anti-target antigen scFv and/or the second anti-target antigen scFv comprises a VH/VL domain pair selected from a group including: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively. In other embodiments, the first target antigen and/or the second target antigen comprises human CCR7, and the first anti-target antigen scFv and/or the second anti-target antigen scFv comprises a VH/VL domain pair selected from a group including: (i) SEQ ID NOs: 78 and 79, respectively. In other embodiments, the first target antigen and/or the second target antigen comprises human CCR8, and the first anti-target antigen scFv and/or the second anti-target antigen scFv comprises a VH/VL domain pair selected from a group including: (i) SEQ ID NOs: 82 and 83, respectively, (ii) SEQ ID NOs: 113 and 114, respectively, and (iii) SEQ ID NOs: 115 and 116, respectively. In some further embodiments, the first anti-target antigen scFv binds an ECD of the first target antigen. In some further embodiments, the second anti-target antigen scFv binds an ECD of the second target antigen. In some further embodiments, the first anti-target antigen scFv binds an ECD of the first target antigen, and the second anti-target antigen scFv binds an ECD of the second target antigen.

In some further embodiments, the first target antigen and/or the second target antigen comprises human PD1, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(a)). In other embodiments, the first target antigen and/or the second target antigen comprises human LAG3, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(b)). In other embodiments, the first target antigen and/or the second target antigen comprises human OX40, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(c)). In other embodiments, the first target antigen and/or the second target antigen comprises human 41BB, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(d)). In other embodiments, the first target antigen and/or the second target antigen comprises human ICOS, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(e)). In other embodiments, the first target antigen and/or the second target antigen comprises human CCR7, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(f)). In other embodiments, the first target antigen and/or the second target antigen comprises human CCR8, and the first anti-target antigen scFv and/or the second anti-target antigen scFv comprises a VH/VL domain pair, or a set of vhCDR1-3 and a set of vlCDR1-3, as described herein (see, e.g., Section (III)(A)(2)(g). In some further embodiments, the first anti-target antigen scFv binds an ECD of the first target antigen. In some further embodiments, the second anti-target antigen scFv binds an ECD of the second target antigen. In some further embodiments, the first anti-target antigen scFv binds an ECD of the first target antigen, and the second anti-target antigen scFv binds an ECD of the second target antigen.

In some further embodiments, the VH1 domain of the first anti-target antigen scFv is covalently attached to the second domain linker at the N-terminus of the first Fc domain. In other embodiments, the VL1 domain of the anti-target antigen scFv is covalently attached to the second domain linker at the N-terminus of the first Fc domain.

In some further embodiments, the VH2 domain of the second anti-target antigen scFv is covalently attached to the fifth domain linker at the N-terminus of the second Fc domain. In other embodiments, the VL2 domain of the second anti-target antigen scFv is covalently attached to the fifth domain linker at the N-terminus of the second Fc domain.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, the third domain linker comprises any one of SEQ ID NOs: 31-40, the fourth domain linker comprises any one of SEQ ID NOs: 31-40, the fifth domain linker comprises any one of SEQ ID NOs: 31-40, and the sixth domain linker comprises any one of SEQ ID NOs: 31-40.

In some further embodiments, the first domain linker comprises any one of SEQ ID NOs: 31-40, and the fourth domain linker comprises any one of SEQ ID NOs: 31-40. In still some further embodiments, the second domain linker, the third domain linker, the fifth domain linker, and/or the sixth domain linker comprises any one of SEQ ID NOs: 31-40. In still some other further embodiments, the second domain linker, the third domain linker, the fifth domain linker, and/or the sixth domain linker comprises a covalent bond. In still some other further embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

### 3. Nanobody Formats:

In some embodiments, the IL-12 Fc fusion protein is configured such that the ABD comprises a VHH domain, wherein the VHH domain comprises a complementarity determining region (CDR) 1, a CDR2, and a CDR3, wherein the CDR1, CDR2, and CDR3 bind to a target antigen.

### a. Embodiment (C)(1)

In one exemplary embodiment, the IL-12 Fc fusion protein comprises: (a) a first monomer comprising, from N-terminus to C-terminus: (i) a first IL-12 subunit domain, (ii) a first domain linker, (iii) a second IL-12 subunit domain, and (iv) a first Fc domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising, from N-terminus to C-terminus: (i) a VHH domain, (ii) a second domain linker, and (iii) a second Fc domain, wherein the VHH domain binds a target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, and the second domain linker comprises any one of SEQ ID NOs: 31-40. In some other further embodiments, the first domain linker and/or the second domain linker comprises a covalent bond. In some other further embodiments, the first domain linker comprises any one of SEQ ID NOs: 31-40, and the second domain linker comprises a covalent bond. In some other further embodiments, the first domain linker comprises a covalent bond, and the second domain linker comprises any one of SEQ ID NOs: 31-40. In some embodiments, at least one of the domain linkers comprises a hinge region of a Fc domain.

In some further embodiments, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In some further embodiments, the target antigen comprises 41BB, and the VHH domain comprises: SEQ ID NO: 129. In other embodiments, the target antigen comprises CCR8, and the VHH domain comprises: SEQ ID NO: 86. In some further embodiments, the VHH domain is humanized.

In some further embodiments, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)). In some further embodiments, the VHH domain is humanized.

### b. Embodiment (C)(2)

In one exemplary embodiment, the IL-12 Fc fusion protein comprises: (a) a first monomer comprising, from N-terminus to C-terminus: (i) a VHH domain, (ii) a first domain linker, and (iii) a first Fc domain; and (b) a second monomer comprising, from N-terminus to C-terminus: (i) a second Fc domain, (ii) a second domain linker, (iii) a first IL-12 subunit domain, (iv) a third domain linker, and (v) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, and the third domain linker comprises any one of SEQ ID NOs: 31-40. In some other further embodiments, the first domain linker, the second domain linker, and/or the third domain linker comprises a covalent bond. In some other further embodiments, at least one domain linker comprises any one of SEQ ID NOs: 31-40, and at least one domain linker comprises a covalent bond. In some embodiments, at least one of the domain linkers comprises a hinge region of a Fc domain.

In some further embodiments, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In some further embodiments, the target antigen comprises 41BB, and the VHH domain comprises: SEQ ID NO: 129. In other embodiments, the target antigen comprises CCR8, and the VHH domain comprises: SEQ ID NO: 86. In some further embodiments, the VHH domain is humanized.

In some further embodiments, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)). In some further embodiments, the VHH domain is humanized.

### c. Embodiment (C)(3)

In one exemplary embodiment, the IL-12 Fc fusion protein comprises: (a) a first monomer comprising, from N-terminus to C-terminus: (i) a first IL-12 subunit domain, (ii) a first domain linker, and (iii) a first Fc domain; and (b) a second monomer comprising, from N-terminus to C-terminus: (i) a second IL-12 subunit domain, (ii) a second domain linker, (iii) a second Fc domain, (iv) a third domain linker, and (v) a VHH domain, wherein: (1) the VHH domain binds a target antigen, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, and the third domain linker comprises any one of SEQ ID NOs: 31-40. In some other further embodiments, the first domain linker, the second domain linker, and/or the third domain linker comprises a covalent bond. In some other further embodiments, at least one domain linker comprises any one of SEQ ID NOs: 31-40, and at least one domain linker comprises a covalent bond. In some embodiments, at least one of the domain linkers comprises a hinge region of a Fc domain.

In some further embodiments, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In some further embodiments, the target antigen comprises 41BB, and the VHH domain comprises: SEQ ID NO: 129. In other embodiments, the target antigen comprises CCR8, and the VHH domain comprises: SEQ ID NO: 86. In some further embodiments, the VHH domain is humanized.

In some further embodiments, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)). In some further embodiments, the VHH domain is humanized.

### d. Embodiment (C)(4)

In one exemplary embodiment, the IL-12 Fc fusion protein comprises: (a) a first monomer comprising, from N-terminus to C-terminus: (i) a first IL-12 subunit domain, (ii) a first domain linker, (iii) a second IL-12 subunit domain, (iv) a second domain linker, (v) a first Fc domain, (vi) a third domain linker, and (vii) a VHH domain, wherein: (1) the VHH domain binds a target antigen, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A; and (b) a second monomer comprising a second Fc domain.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, and the third domain linker comprises any one of SEQ ID NOs: 31-40. In some other further embodiments, the first domain linker, the second domain linker, and/or the third domain linker comprises a covalent bond. In some other further embodiments, at least one domain linker comprises any one of SEQ ID NOs: 31-40, and at least one domain linker comprises a covalent bond. In some embodiments, at least one of the domain linkers comprises a hinge region of a Fc domain.

In some further embodiments, the VHH domain binds a target antigen selected from a group including: (1) OX40, (2) LAG3, (3) PD1, (4) ICOS, (5) 41BB, (6) CCR7, and (7) CCR8. In some further embodiments, the VHH domain is humanized.

In some further embodiments, the target antigen comprises 41BB, and the VHH domain comprises: SEQ ID NO: 129. In other embodiments, the target antigen comprises CCR8, and the VHH domain comprises: SEQ ID NO: 86. In some further embodiments, the VHH domain is humanized.

In some further embodiments, the target antigen comprises PD1, and the VHH domain comprises a VHH domain described herein (see, e.g., Section (III)(A)(2)(a)). In other embodiments, the target antigen comprises LAG3, and the VHH domain comprises a VHH domain described herein (see, e.g., Section (III)(A)(2)(b)). In other embodiments, the target antigen comprises OX40, and the VHH domain comprises a VHH domain described herein (see, e.g., Section (III)(A)(2)(c)). In other embodiments, the target antigen comprises 41BB, and the VHH domain comprises a VHH domain described herein (see, e.g., Section (III)(A)(2)(d)). In other embodiments, the target antigen comprises ICOS, and the VHH domain comprises a VHH domain described herein (see, e.g., Section (III)(A)(2)(e)). In other embodiments, the target antigen comprises CCR7, and the VHH domain comprises a VHH domain described herein (see, e.g., Section (III)(A)(2)(f)). In other embodiments, the target antigen comprises CCR8, and the VHH domain comprises a VHH domain described herein (see, e.g., Section (III)(A)(2)(g)). In some further embodiments, the VHH domain is humanized.

### e. Embodiment (C)(5)

In one exemplary embodiment, the IL-12 Fc fusion protein comprises: (a) a first monomer comprising, from N-terminus to C-terminus: (i) a first VHH domain, (ii) a first domain linker, (iii) a first Fc domain, (iv) a second domain linker, (v) a first IL-12 subunit domain, (vi) a third domain linker, and (vii) a second IL-12 subunit domain, wherein: (1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, (2) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A, and (3) the first VHH domain binds a first target antigen; and (b) a second monomer comprising, from N-terminus to C-terminus: (i) a second VHH domain, (ii) a fourth domain linker, and (iii) a second Fc domain, wherein the second VHH domain binds a second target antigen.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, the third domain linker comprises any one of SEQ ID NOs: 31-40, and the fourth domain linker comprises any one of SEQ ID NOs: 31-40. In some other further embodiments, the first domain linker, the second domain linker, the third domain linker, and/or the fourth domain linker comprises any one of SEQ ID NOs: 31-40. In some other further embodiments, the first domain linker, the second domain linker, the third domain linker, and/or the fourth domain linker comprises a covalent bond. In some other further embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

In some further embodiments, the first VHH domain binds a first target antigen selected from a group including: (i) OX40, (ii) LAG3, (iii) PD1, (iv) ICOS, (v) 41BB, (vi) CCR7, and (vii) CCR8.

In some further embodiments, the second VHH domain binds a second target antigen selected from a group including: (i) OX40, (ii) LAG3, (iii) PD1, (iv) ICOS, (v) 41BB, (vi) CCR7, and (vii) CCR8.

In some further embodiments, the first VHH domain and the second VHH domain both bind a target antigen selected from a group including: (i) OX40, (ii) LAG3, (iii) PD1, (iv) ICOS, (v) 41BB, (vi) CCR7, and (vii) CCR8.

In some further embodiments, the first VHH domain binds a first target antigen selected from a group including: (i) OX40, (ii) LAG3, (iii) PD1, (iv) ICOS, (v) 41BB, (vi) CCR7, and (vii) CCR8, and the second VHH domain binds a second target antigen selected from a group including: (i) OX40, (ii) LAG3, (iii) PD1, (iv) ICOS, (v) 41BB, (vi) CCR7, and (vii) CCR8.

In some further embodiments, the first target antigen and/or the second target antigen comprises 41BB, and the first VHH domain and/or the second VHH domain comprises: SEQ ID NO: 129. In other embodiments, the first target antigen and/or the second target antigen comprises CCR8, and the first VHH domain and/or the second VHH domain comprises: SEQ ID NO: 86. In some further embodiments, the first VHH domain is humanized. In some further embodiments, the second VHH domain is humanized. In some further embodiments, the first VHH domain is humanized, and the second VHH domain is humanized.

In some further embodiments, the first target antigen and/or the second target antigen comprises PD1, and the first VHH domain and/or the second VHH domain comprises a VHH domain described herein (see, e.g., Section (III)(A)(2)(a)). In other embodiments, the first target antigen and/or the second target antigen comprises LAG3, and the first VHH domain and/or the second VHH domain comprises a VHH domain described herein (see, e.g., Section (III)(A)(2)(b)). In other embodiments, the first target antigen and/or the second target antigen comprises OX40, and the first VHH domain and/or the second VHH domain comprises a VHH domain described herein (see, e.g., Section (III)(A)(2)(c)). In other embodiments, the first target antigen and/or the second target antigen comprises 41BB, and the first VHH domain and/or the second VHH domain comprises a VHH domain described herein (see, e.g., Section (III)(A)(2)(d)). In other embodiments, the first target antigen and/or the second target antigen comprises ICOS, and the first VHH domain and/or the second VHH domain comprises a VHH domain described herein (see, e.g., Section (III)(A)(2)(e)). In other embodiments, the first target antigen and/or the second target antigen comprises CCR7, and the first VHH domain and/or the second VHH domain comprises a VHH domain described herein (see, e.g., Section (III)(A)(2)(f)). In other embodiments, the first target antigen and/or the second target antigen comprises CCR8, and the first VHH domain and/or the second VHH domain comprises a VHH domain described herein (see, e.g., Section (III)(A)(2)(g)). In some further embodiments, the first VHH domain is humanized. In some further embodiments, the second VHH domain is humanized. In some further embodiments, the first VHH domain is humanized, and the second VHH domain is humanized.

### f. Embodiment (C)(6)

In one exemplary embodiment, the IL-12 Fc fusion protein comprises: (a) a first monomer comprising, from N-terminus to C-terminus: (i) a first VHH domain, (ii) a first domain linker, (iii) a first Fc domain, (iv) a second domain linker, and (v) a first IL-12 subunit domain, wherein the first VHH domain binds a first target antigen; and (b) a second monomer comprising, from N-terminus to C-terminus: (i) a second VHH domain, (ii) a third domain linker, (iii) a second Fc domain, (iv) a fourth domain linker, and (v) a second IL-12 subunit domain, wherein: (1) the second VHH domain binds a second target antigen, (2) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and (3) the variant IL-12 p35 subunit domain comprises a set of amino acid substitutions selected from a group including: (a) Y40S/K170A, (b) Y40S/K170T, (c) Y40P/K168A, (d) Y40P/K170A, and (e) Y40E/K170A.

In some further embodiments, the variant IL-12 p35 subunit domain comprises any one of SEQ ID NOs: 17-21.

In some further embodiments, the IL-12 p40 subunit domain comprises SEQ ID NO: 4. In other embodiments, the IL-12 p40 subunit domain comprises a variant IL-12 p40 subunit domain. In some further embodiments wherein the IL-12 p40 subunit domain comprises a variant IL-12 p40 subunit domain, the variant IL-12 p40 subunit domain comprises any one of SEQ ID NOs: 23-25. In still some further embodiments, the variant IL-12 p40 subunit domain comprises SEQ ID NO: 23 or SEQ ID NO: 25, and the variant IL-12 p35 subunit domain further comprises a C74S substitution mutation.

In some further embodiments, the first Fc domain comprises any one of SEQ ID NOs: 29 or 30, and the second Fc domain comprises any one of SEQ ID NOs: 30 or 29. In some further embodiments, the first Fc domain further comprises a set of substitution mutations comprising T366W/S354C, and the second Fc domain further comprises a set of substitution mutations comprising T366S/L368A/Y407V/Y349C, whereas in other embodiments the first Fc domain further comprises a set of substitution mutations comprising T366S/L368A/Y407V/Y349C, and the second Fc domain further comprises a set of substitution mutations comprising T366W/S354C.

In some further embodiments, the first domain linker comprises any one of SEQ ID NOs: 31-40, the second domain linker comprises any one of SEQ ID NOs: 31-40, the third domain linker comprises any one of SEQ ID NOs: 31-40, and the fourth domain linker comprises any one of SEQ ID NOs: 31-40. In some other further embodiments, the first domain linker, the second domain linker, the third domain linker, and/or the fourth domain linker comprises any one of SEQ ID NOs: 31-40. In some other further embodiments, the first domain linker, the second domain linker, the third domain linker, and/or the fourth domain linker comprises a covalent bond. In some other further embodiments, at least one of the domain linkers comprises any one of SEQ ID NOs: 31-40, and at least one of the domain linkers comprises a covalent bond. In some embodiments, at least one of the domain linkers comprises a hinge region of a Fc domain.

In some further embodiments, the first VHH domain binds a first target antigen selected from a group including: (i) OX40, (ii) LAG3, (iii) PD1, (iv) ICOS, (v) 41BB, (vi) CCR7, and (vii) CCR8.

In some further embodiments, the second VHH domain binds a second target antigen selected from a group including: (i) OX40, (ii) LAG3, (iii) PD1, (iv) ICOS, (v) 41BB, (vi) CCR7, and (vii) CCR8.

In some further embodiments, the first VHH domain and the second VHH domain both bind a target antigen selected from a group including: (i) OX40, (ii) LAG3, (iii) PD1, (iv) ICOS, (v) 41BB, (vi) CCR7, and (vii) CCR8.

In some further embodiments, the VHH domain binds a first target antigen selected from a group including: (i) OX40, (ii) LAG3, (iii) PD1, (iv) ICOS, (v) 41BB, (vi) CCR7, and (vii) CCR8, and the second VHH domain binds a second target antigen selected from a group including: (i) OX40, (ii) LAG3, (iii) PD1, (iv) ICOS, (v) 41BB, (vi) CCR7, and (vii) CCR8.

In some further embodiments, the first target antigen and/or the second target antigen comprises 41BB, and the first VHH domain and/or the second VHH domain comprises: SEQ ID NO: 129. In other embodiments, the first target antigen and/or the second target antigen comprises CCR8, and the first VHH domain and/or the second VHH domain comprises: SEQ ID NO: 86. In some further embodiments, the first VHH domain is humanized. In some further embodiments, the second VHH domain is humanized. In some further embodiments, the first VHH domain is humanized, and the second VHH domain is humanized.

In some further embodiments, the first target antigen and/or the second target antigen comprises PD1, and the first VHH domain and/or the second VHH domain comprises a VHH domain described herein (see, e.g., Section (III)(A)(2)(a)). In other embodiments, the first target antigen and/or the second target antigen comprises LAG3, and the first VHH domain and/or the second VHH domain comprises a VHH domain described herein (see, e.g., Section (III)(A)(2)(b)). In other embodiments, the first target antigen and/or the second target antigen comprises OX40, and the first VHH domain and/or the second VHH domain comprises a VHH domain described herein (see, e.g., Section (III)(A)(2)(c)). In other embodiments, the first target antigen and/or the second target antigen comprises 41BB, and the first VHH domain and/or the second VHH domain comprises a VHH domain described herein (see, e.g., Section (III)(A)(2)(d)). In other embodiments, the first target antigen and/or the second target antigen comprises ICOS, and the first VHH domain and/or the second VHH domain comprises a VHH domain described herein (see, e.g., Section (III)(A)(2)(e)). In other embodiments, the first target antigen and/or the second target antigen comprises CCR7, and the first VHH domain and/or the second VHH domain comprises a VHH domain described herein (see, e.g., Section (III)(A)(2)(f)). In other embodiments, the first target antigen and/or the second target antigen comprises CCR8, and the first VHH domain and/or the second VHH domain comprises a VHH domain described herein (see, e.g., Section (III)(A)(2)(g)). In some further embodiments, the first VHH domain is humanized. In some further embodiments, the second VHH domain is humanized. In some further embodiments, the first VHH domain is humanized, and the second VHH domain is humanized.

### 4. Exemplary IL-12 Fc Fusion Proteins:

In some embodiments, the IL-12 Fc fusion protein comprises: (a) a first monomer; (b) a second monomer; and (c) a third monomer. In one exemplary embodiment, the first monomer, the second monomer, and the third monomer comprise: (i) SEQ ID NOs: 49, 153, and 145, respectively, (ii) SEQ ID NOs: 48, 146, and 145, respectively, (iii) SEQ ID NOs: 48, 151, and 150, respectively, (iv) SEQ ID NOs: 48, 153, and 145, respectively, (v) SEQ ID NOs: 48, 155, and 145, respectively, (vi) SEQ ID NOs: 48, 157, and 145, respectively, (vii) SEQ ID NOs: 48, 159, and 150, respectively, (viii) SEQ ID NOs: 48, 161, and 150, respectively, (ix) SEQ ID NOs: 48, 163, and 150, respectively, (x) SEQ ID NOs: 48, 165, and 150, respectively, (xi) SEQ ID NOs: 48, 167, and 150, respectively, (xii) SEQ ID NOs: 48, 169, and 150, respectively, (xiii) SEQ ID NOs: 49, 141, and 140, respectively, (xiv) SEQ ID NOs: 49, 146, and 145, respectively, (xv) SEQ ID NOs: 49, 151, and 150, respectively, (xvi) SEQ ID NOs: 48, 141, and 140, respectively, (xvii) SEQ ID NOs: 49, 155, and 145, respectively, (xviii) SEQ ID NOs: 49, 157, and 145, respectively, (xix) SEQ ID NOs: 49, 159, and 150, respectively, (xx) SEQ ID NOs: 49, 161, and 150, respectively, (xxi) SEQ ID NOs: 49, 163, and 150, respectively, (xxii) SEQ ID NOs: 49, 165, and 150, respectively, (xxiii) SEQ ID NOs: 49, 167, and 150, respectively, (xxiv) SEQ ID NOs: 49, 169, and 150, respectively, (xxv) SEQ ID NOs: 50, 141, and 140, respectively, (xxvi) SEQ ID NOs: 50, 146, and 145, respectively, (xxvii) SEQ ID NOs: 50, 151, and 150, respectively, (xxviii) SEQ ID NOs: 50, 153, and 145, respectively, (xxix) SEQ ID NOs: 50, 155, and 145, respectively, (xxx) SEQ ID NOs: 50, 157, and 145, respectively, (xxxi) SEQ ID NOs: 50, 159, and 150, respectively, (xxxii) SEQ ID NOs: 50, 161, and 150, respectively, (xxxiii) SEQ ID NOs: 50, 163, and 150, respectively, (xxxiv) SEQ ID NOs: 50, 165, and 150, respectively, (xxxv) SEQ ID NOs: 50, 167, and 150, respectively, (xxxvi) SEQ ID NOs: 50, 169, and 150, respectively, (xxxvii) SEQ ID NOs: 51, 141, and 140, respectively, (xxxviii) SEQ ID NOs: 51, 146, and 145, respectively, (xxxix) SEQ ID NOs: 51, 151, and 150, respectively, (xl) SEQ ID NOs: 51, 153, and 145, respectively, (xli) SEQ ID NOs: 51, 155, and 145, respectively, (xlii) SEQ ID NOs: 51, 157, and 145, respectively, (xliii) SEQ ID NOs: 51, 159, and 150, respectively, (xliv) SEQ ID NOs: 51, 161, and 150, respectively, (xlv) SEQ ID NOs: 51, 163, and 150, respectively, (xlvi) SEQ ID NOs: 51, 165, and 150, respectively, (xlvii) SEQ ID NOs: 51, 167, and 150, respectively, (xlviii) SEQ ID NOs: 51, 169, and 150, respectively, (xlix) SEQ ID NOs: 52, 141, and 140, respectively, (1) SEQ ID NOs: 52, 146, and 145, respectively, (li) SEQ ID NOs: 52, 151, and 150, respectively, (lii) SEQ ID NOs: 52, 153, and 145, respectively, (liii) SEQ ID NOs: 52, 155, and 145, respectively, (liv) SEQ ID NOs: 52, 157, and 145, respectively, (lv) SEQ ID NOs: 52, 159, and 150, respectively, (lvi) SEQ ID NOs: 52, 161, and 150, respectively, (lvii) SEQ ID NOs: 52, 163, and 150, respectively, (lviii) SEQ ID NOs: 52, 165, and 150, respectively, (lix) SEQ ID NOs: 52, 167, and 150, respectively, (lx) SEQ ID NOs: 52, 169, and 150, respectively, (lxi) SEQ ID NOs: 48, 174, and 173, respectively, (lxii) SEQ ID NOs: 49, 174, and 173, respectively, (lxiii) SEQ ID NOs: 50, 174, and 173, respectively, (lxiv) SEQ ID NOs: 51, 174, and 173, respectively, or (lxv) SEQ ID NOs: 52, 174, and 173, respectively.

In another exemplary embodiment, the first monomer comprises the amino acid sequence of SEQ ID NO: 47, the second monomer comprises the amino acid sequence of SEQ ID NO: 56, and the third monomer comprises the amino acid sequence of SEQ ID NO: 57.

In another exemplary embodiment, the first monomer comprises the amino acid sequence of SEQ ID NO: 48, the second monomer comprises the amino acid sequence of SEQ ID NO: 56, and the third monomer comprises the amino acid sequence of SEQ ID NO: 57.

In another exemplary embodiment, the first monomer comprises the amino acid sequence of SEQ ID NO: 49, the second monomer comprises the amino acid sequence of SEQ ID NO: 56, and the third monomer comprises the amino acid sequence of SEQ ID NO: 57.

In another exemplary embodiment, the first monomer comprises the amino acid sequence of SEQ ID NO: 50, the second monomer comprises the amino acid sequence of SEQ ID NO: 56, and the third monomer comprises the amino acid sequence of SEQ ID NO: 57.

In another exemplary embodiment, the first monomer comprises the amino acid sequence of SEQ ID NO: 51, the second monomer comprises the amino acid sequence of SEQ ID NO: 56, and the third monomer comprises the amino acid sequence of SEQ ID NO: 57.

In another exemplary embodiment, the first monomer comprises the amino acid sequence of SEQ ID NO: 52, the second monomer comprises the amino acid sequence of SEQ ID NO: 56, and the third monomer comprises the amino acid sequence of SEQ ID NO: 57.

In another exemplary embodiment, the first monomer comprises the amino acid sequence of SEQ ID NO: 48, the second monomer comprises the amino acid sequence of SEQ ID NO: 80, and the third monomer comprises the amino acid sequence of SEQ ID NO: 81.

In another exemplary embodiment, the first monomer comprises the amino acid sequence of SEQ ID NO: 48, the second monomer comprises the amino acid sequence of SEQ ID NO: 84, and the third monomer comprises the amino acid sequence of SEQ ID NO: 85.

In another exemplary embodiment, the first monomer comprises the amino acid sequence of any one of SEQ ID NOs: 47-52, the second monomer comprises the amino acid sequence of SEQ ID NO: 43, and the third monomer comprises the amino acid sequence of SEQ ID NO: 44.

In another exemplary embodiment, the first monomer comprises the amino acid sequence of SEQ ID NO: 53, the second monomer comprises the amino acid sequence of SEQ ID NO: 60, and the third monomer comprises the amino acid sequence of SEQ ID NO: 61.

In another exemplary embodiment, the first monomer comprises the amino acid sequence of SEQ ID NO: 53, the second monomer comprises the amino acid sequence of SEQ ID NO: 64, and the third monomer comprises the amino acid sequence of SEQ ID NO: 65.

In another exemplary embodiment, the first monomer comprises the amino acid sequence of SEQ ID NO: 53, the second monomer comprises the amino acid sequence of SEQ ID NO: 68, and the third monomer comprises the amino acid sequence of SEQ ID NO: 69.

In another exemplary embodiment, the first monomer comprises the amino acid sequence of SEQ ID NO: 53, the second monomer comprises the amino acid sequence of SEQ ID NO: 72, and the third monomer comprises the amino acid sequence of SEQ ID NO: 73.

In another exemplary embodiment, the first monomer comprises the amino acid sequence of SEQ ID NO: 53, the second monomer comprises the amino acid sequence of SEQ ID NO: 76, and the third monomer comprises the amino acid sequence of SEQ ID NO: 77.

In another exemplary embodiment, the first monomer comprises the amino acid sequence of SEQ ID NO: 53, the second monomer comprises the amino acid sequence of SEQ ID NO: 45, and the third monomer comprises the amino acid sequence of SEQ ID NO: 46.

In another exemplary embodiment, the first monomer comprises the amino acid sequence of SEQ ID NO: 49, the second monomer comprises the amino acid sequence of SEQ ID NO: 141, and the third monomer comprises the amino acid sequence of SEQ ID NO: 140.

In another exemplary embodiment, the first monomer comprises the amino acid sequence of SEQ ID NO: 49, the second monomer comprises the amino acid sequence of SEQ ID NO: 146, and the third monomer comprises the amino acid sequence of SEQ ID NO: 145.

In another exemplary embodiment, the first monomer comprises the amino acid sequence of SEQ ID NO: 49, the second monomer comprises the amino acid sequence of SEQ ID NO: 151, and the third monomer comprises the amino acid sequence of SEQ ID NO: 150.

In another exemplary embodiment, the first monomer comprises the amino acid sequence of SEQ ID NO: 52, the second monomer comprises the amino acid sequence of SEQ ID NO: 141, and the third monomer comprises the amino acid sequence of SEQ ID NO: 140.

In another exemplary embodiment, the first monomer comprises the amino acid sequence of SEQ ID NO: 49, the second monomer comprises the amino acid sequence of SEQ ID NO: 153, and the third monomer comprises the amino acid sequence of SEQ ID NO: 145.

In another exemplary embodiment, the first monomer comprises the amino acid sequence of SEQ ID NO: 49, the second monomer comprises the amino acid sequence of SEQ ID NO: 155, and the third monomer comprises the amino acid sequence of SEQ ID NO: 145.

In another exemplary embodiment, the first monomer comprises the amino acid sequence of SEQ ID NO: 49, the second monomer comprises the amino acid sequence of SEQ ID NO: 157, and the third monomer comprises the amino acid sequence of SEQ ID NO: 145.

In another exemplary embodiment, the first monomer comprises the amino acid sequence of SEQ ID NO: 49, the second monomer comprises the amino acid sequence of SEQ ID NO: 159, and the third monomer comprises the amino acid sequence of SEQ ID NO: 150.

In another exemplary embodiment, the first monomer comprises the amino acid sequence of SEQ ID NO: 49, the second monomer comprises the amino acid sequence of SEQ ID NO: 161, and the third monomer comprises the amino acid sequence of SEQ ID NO: 150.

In another exemplary embodiment, the first monomer comprises the amino acid sequence of SEQ ID NO: 49, the second monomer comprises the amino acid sequence of SEQ ID NO: 163, and the third monomer comprises the amino acid sequence of SEQ ID NO: 150.

In another exemplary embodiment, the first monomer comprises the amino acid sequence of SEQ ID NO: 49, the second monomer comprises the amino acid sequence of SEQ ID NO: 165, and the third monomer comprises the amino acid sequence of SEQ ID NO: 150.

In another exemplary embodiment, the first monomer comprises the amino acid sequence of SEQ ID NO: 49, the second monomer comprises the amino acid sequence of SEQ ID NO: 167, and the third monomer comprises the amino acid sequence of SEQ ID NO: 150.

In another exemplary embodiment, the first monomer comprises the amino acid sequence of SEQ ID NO: 49, the second monomer comprises the amino acid sequence of SEQ ID NO: 169, and the third monomer comprises the amino acid sequence of SEQ ID NO: 150.

In another exemplary embodiment, the first monomer comprises the amino acid sequence of SEQ ID NO: 49, the second monomer comprises the amino acid sequence of SEQ ID NO: 153, and the third monomer comprises the amino acid sequence of SEQ ID NO: 145. In a further exemplary embodiment, the first monomer comprises an amino acid sequence with at least about 90%, about 91%, about 92%, about 93%, about 94%, about 95%, about 96% about 97%, about 98%, about 99% or more identity to SEQ ID NO: 49, the second monomer comprises an amino acid sequence with at least about 90%, about 91%, about 92%, about 93%, about 94%, about 95%, about 96% about 97%, about 98%, about 99% or more identity to SEQ ID NO: 153, and the third monomer comprises an amino acid sequence with at least about 90%, about 91%, about 92%, about 93%, about 94%, about 95%, about 96% about 97%, about 98%, about 99% or more identity to SEQ ID NO: 145.

In another exemplary embodiment, the first monomer comprises the amino acid sequence of SEQ ID NO: 48, the second monomer comprises the amino acid sequence of SEQ ID NO: 174, and the third monomer comprises the amino acid sequence of SEQ ID NO: 173. In a further exemplary embodiment, the first monomer comprises an amino acid sequence with at least about 90%, about 91%, about 92%, about 93%, about 94%, about 95%, about 96% about 97%, about 98%, about 99% or more identity to SEQ ID NO: 48, the second monomer comprises an amino acid sequence with at least about 90%, about 91%, about 92%, about 93%, about 94%, about 95%, about 96% about 97%, about 98%, about 99% or more identity to SEQ ID NO: 174, and the third monomer comprises an amino acid sequence with at least about 90%, about 91%, about 92%, about 93%, about 94%, about 95%, about 96% about 97%, about 98%, about 99% or more identity to SEQ ID NO: 173.

In another exemplary embodiment, the first monomer comprises the amino acid sequence of SEQ ID NO: 49, the second monomer comprises the amino acid sequence of SEQ ID NO: 174, and the third monomer comprises the amino acid sequence of SEQ ID NO: 173. In a further exemplary embodiment, the first monomer comprises an amino acid sequence with at least about 90%, about 91%, about 92%, about 93%, about 94%, about 95%, about 96% about 97%, about 98%, about 99% or more identity to SEQ ID NO: 49, the second monomer comprises an amino acid sequence with at least about 90%, about 91%, about 92%, about 93%, about 94%, about 95%, about 96% about 97%, about 98%, about 99% or more identity to SEQ ID NO: 174, and the third monomer comprises an amino acid sequence with at least about 90%, about 91%, about 92%, about 93%, about 94%, about 95%, about 96% about 97%, about 98%, about 99% or more identity to SEQ ID NO: 173.

In another exemplary embodiment, the first monomer comprises the amino acid sequence of SEQ ID NO: 50, the second monomer comprises the amino acid sequence of SEQ ID NO: 174, and the third monomer comprises the amino acid sequence of SEQ ID NO: 173. In a further exemplary embodiment, the first monomer comprises an amino acid sequence with at least about 90%, about 91%, about 92%, about 93%, about 94%, about 95%, about 96% about 97%, about 98%, about 99% or more identity to SEQ ID NO: 50, the second monomer comprises an amino acid sequence with at least about 90%, about 91%, about 92%, about 93%, about 94%, about 95%, about 96% about 97%, about 98%, about 99% or more identity to SEQ ID NO: 174, and the third monomer comprises an amino acid sequence with at least about 90%, about 91%, about 92%, about 93%, about 94%, about 95%, about 96% about 97%, about 98%, about 99% or more identity to SEQ ID NO: 173.

In another exemplary embodiment, the first monomer comprises the amino acid sequence of SEQ ID NO: 51, the second monomer comprises the amino acid sequence of SEQ ID NO: 174, and the third monomer comprises the amino acid sequence of SEQ ID NO: 173. In a further exemplary embodiment, the first monomer comprises an amino acid sequence with at least about 90%, about 91%, about 92%, about 93%, about 94%, about 95%, about 96% about 97%, about 98%, about 99% or more identity to SEQ ID NO: 51, the second monomer comprises an amino acid sequence with at least about 90%, about 91%, about 92%, about 93%, about 94%, about 95%, about 96% about 97%, about 98%, about 99% or more identity to SEQ ID NO: 174, and the third monomer comprises an amino acid sequence with at least about 90%, about 91%, about 92%, about 93%, about 94%, about 95%, about 96% about 97%, about 98%, about 99% or more identity to SEQ ID NO: 173.

In another exemplary embodiment, the first monomer comprises the amino acid sequence of SEQ ID NO: 52, the second monomer comprises the amino acid sequence of SEQ ID NO: 174, and the third monomer comprises the amino acid sequence of SEQ ID NO: 173. In a further exemplary embodiment, the first monomer comprises an amino acid sequence with at least about 90%, about 91%, about 92%, about 93%, about 94%, about 95%, about 96% about 97%, about 98%, about 99% or more identity to SEQ ID NO: 52, the second monomer comprises an amino acid sequence with at least about 90%, about 91%, about 92%, about 93%, about 94%, about 95%, about 96% about 97%, about 98%, about 99% or more identity to SEQ ID NO: 174, and the third monomer comprises an amino acid sequence with at least about 90%, about 91%, about 92%, about 93%, about 94%, about 95%, about 96% about 97%, about 98%, about 99% or more identity to SEQ ID NO: 173.

In some embodiments, the IL-12 Fc fusion protein comprises: (a) a first monomer; and (b) a second monomer. In one exemplary embodiment, the first monomer comprises the amino acid sequence of SEQ ID NO: 48, and the second monomer comprises the amino acid sequence of SEQ ID NO: 87.

### IV. Methods of Making

As will be appreciated by one skilled in the art, any aspects and embodiments of the methods of making low potency IL-12 fusion proteins described herein can utilize any of the aspects and/or embodiments of the compositions described herein and can further be used in any of the methods of use also described herein.

### A. Low Potency IL-12 Fc Fusion Proteins

In another aspect, the present disclosure provides a method of producing low potency IL-12 fusion proteins of the disclosure, the method comprising culturing a host cell under conditions wherein the IL-12 Fc fusion protein is expressed.

Nucleic acids encoding a desired fusion protein, such as, for example, an IL-12 Fc fusion protein (and/or fragments, domains, or components thereof), can be introduced into a host cell by standard techniques for transforming or transfecting cells. Alternatively, or in addition to, nucleic acids encoding a desired protein, such as, for example, an IL-12 Fc fusion protein (and/or fragments, domains, or components thereof), can be incorporated into a vector (e.g., “an expression vector”) or “expression cassette,” as described in further detail herein. Vectors (or expression cassettes) can be introduced into a host cell via conventional transformation or transfection techniques. Suitable methods for transforming or transfecting host cells using a nucleic acid or vector include, but are not limited to, calcium phosphate transfection, DEAE-dextran-mediated transfection, transfection, microinjection, cationic lipid-mediated transfection, lipofection, electroporation, transduction, scrape loading, ballistic introduction, nucleoporation, hydrodynamic shock, infection, and the like.

In some embodiments, the method further comprises isolating and/or purifying the IL-12 Fc fusion protein. In the process, a host cell (as described above), into which has been introduced a nucleic acid encoding the fusion protein of interest operatively linked to a regulatory sequence, is grown at production scale in a culture medium to stimulate transcription of the nucleotide sequence(s) encoding the fusion protein of interest. Subsequently, the fusion protein is isolated from harvested host cells or from the culture medium. Standard protein purification techniques can be used to isolate the fusion protein of interest from the medium or from the harvested cells.

In particular, the purification techniques can be used to express and purify a desired fusion protein, such as, for example, an IL-12 Fc fusion protein, on a large-scale (i.e., in at least milligram (1.00 mg) quantities) or on a small-scale (i.e., in at least nanogram (1.00 ng) quantities up to milligram (<1.00 mg) quantities) from a variety of implementations including, but not limited to, roller bottles, spinner flasks, tissue culture plates, one or more bioreactors, one or more fermenters, and/or any combination thereof.

An expressed fusion protein, such as, for example, an IL-12 Fc fusion protein, can be isolated and purified by known methods. Generally, the culture medium is centrifuged and/or filtered, and then the supernatant is purified by affinity or immunoaffinity chromatography. The fusion proteins of the present invention can be separated and/or purified by appropriate combination(s) of known techniques. These methods include, but are not limited to, methods utilizing solubility (e.g., salt precipitation or solvent precipitation), methods utilizing a difference in molecular weight (e.g., dialysis, ultra-filtration, gel-filtration, SDS-polyacrylamide gel electrophoresis (SDS-PAGE)), methods utilizing a difference in electrical charge (e.g., ion-exchange column chromatography), methods utilizing specific affinity (e.g., affinity chromatography), methods utilizing a difference in hydrophobicity (e.g., reverse-phase high-performance liquid chromatography), methods utilizing a difference in isoelectric point (e.g., isoelectric focusing electrophoresis or metal affinity columns, such as, for example, Ni-NTA), and the like.

### V. Methods of Use

As will be appreciated by one skilled in the art, any aspects and embodiments of the methods of use described herein can utilize any of the aspects and/or embodiments of the compositions described above and can further utilize any of the methods of making the low potency IL-12 fusion proteins also described above.

### A. Low Potency IL-12 Fc Fusion Proteins

In another aspect, the present disclosure provides methods of using one or more of the low potency IL-12 fusion proteins of the disclosure. Once made, the compositions of the invention find use in a number of applications, including, but not limited to: (1) treating a cancer in a human subject; (2) activating a target cell (such as, for example, a hematopoietic cell); (3) stimulating IL-12 signaling in a target cell; (4) inducing the production and/or secretion of one or more cytokines (e.g., IFNγ) in a target cell; or (5) reprogramming and/or altering a differentiation state of a target cell.

Generally, all of the aforementioned methods of use rely upon a method comprising administering a therapeutically effective amount of an IL-12 Fc fusion protein described herein to a human subject in need thereof, wherein the IL-12 Fc fusion protein binds to a target cell subsequent to the administering, to effectuate the stated method of use (e.g., treating a cancer in a human subject; activating a target cell; stimulating IL-12 signaling in a target cell; inducing the production and/or secretion of one or more cytokines in a target cell; reprogramming and/or altering a differentiation state of a target cell).

In some further embodiments, the target cell expresses: (1) IL-12Rβ2, and (2) a cell surface antigen selected from a group including: (a) human OX40, (b) human LAG3, (c) human PD1, (d) human ICOS, (e) human 41BB, (f) human CCR7, and (g) human CCR8. In still some further embodiments, the cell surface antigen is human PD1, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is human LAG3, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is human OX40, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is human ICOS, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is human 41BB, and the target cell comprises: (i) activated and/or exhausted CD4⁺ T cell(s), (ii) activated and/or exhausted NK cell(s), (iii) activated and/or exhausted CD8⁺ T cell(s), or (iv) activated and/or exhausted regulatory T cell(s). In other further embodiments, the cell surface antigen is human CCR7, and the target cell comprises: (i) naïve CD4⁺ T cell(s), (ii) naïve CD8⁺ T cell(s), or (iii) naïve regulatory T cell(s). In other further embodiments, the cell surface antigen is human CCR8, and the target cell comprises: (i) activated and/or exhausted CD4⁺ T cell(s), or (ii) activated and/or exhausted regulatory T cell(s).

In some further embodiments, the target cell expresses: (1) IL-12Rβ2, and (2) a cell surface antigen selected from a group including: (a) mouse PD1, (b) mouse LAG3, (c) mouse OX40, (d) mouse ICOS, (e) mouse 41BB, (f) mouse CCR7, and (g) mouse CCR8. In still some further embodiments, the cell surface antigen is mouse PD1, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is mouse LAG3, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is mouse OX40, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is mouse ICOS, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is mouse 41BB, and the target cell comprises: (i) activated and/or exhausted CD4⁺ T cell(s), (ii) activated and/or exhausted NK cell(s), (iii) activated and/or exhausted CD8⁺ T cell(s), or (iv) activated and/or exhausted regulatory T cell(s). In other further embodiments, the cell surface antigen is mouse CCR7, and the target cell comprises: (i) naïve CD4⁺ T cell(s), (ii) naïve CD8⁺ T cell(s), or (iii) naïve regulatory T cell(s). In other further embodiments, the cell surface antigen is mouse CCR8, and the target cell comprises: (i) activated and/or exhausted CD4⁺ T cell(s), or (ii) activated and/or exhausted regulatory T cell(s).

In some further embodiments, the target cell expresses: (1) IL-12Rβ2, and (2) a cell surface antigen selected from a group including: (a) OX40, (b) LAG3, (c) PD1, (d) ICOS, (e) 41BB, (f) CCR7, and (g) CCR8. In still some further embodiments, the cell surface antigen is PD1, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is LAG3, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is OX40, and the target cell comprises: (i) activated and/or exhausted CD8⁺ T cell(s), (ii) activated and/or exhausted CD4⁺ T cell(s), (iii) activated and/or exhausted NK cell(s), or (iv) activated and/or exhausted regulatory T cell(s). In other further embodiments, the cell surface antigen is ICOS, and the target cell comprises: (i) activated and/or exhausted CD4⁺ T cell(s), (ii) activated and/or exhausted CD8⁺ T cell(s), (iii) activated and/or exhausted NK cell(s), or (iv) activated and/or exhausted regulatory T cell(s). In other further embodiments, the cell surface antigen is 41BB, and the target cell comprises: (i) activated and/or exhausted CD4⁺ T cell(s), (ii) activated and/or exhausted NK cell(s), (iii) activated and/or exhausted CD8⁺ T cell(s), or (iv) activated and/or exhausted regulatory T cell(s). In other further embodiments, the cell surface antigen is CCR7, and the target cell comprises: (i) naïve CD4⁺ T cell(s), (ii) naïve CD8⁺ T cell(s), or (iii) naïve regulatory T cell(s). In other further embodiments, the cell surface antigen is CCR8, and the target cell comprises: (i) activated and/or exhausted CD4⁺ T cell(s), or (ii) activated and/or exhausted regulatory T cell(s).

In some embodiments, a therapeutically effective amount of an IL-12 Fc fusion protein, as described above in the compositions and/or methods of making sections, is administered to a subject in need thereof. As used herein, the term “therapeutically effective amount” generally refers to an amount sufficient for a composition to accomplish a stated purpose relative to the absence of the composition (e.g., achieve the effect for which it is administered, treat a disease/disorder/condition, reduce a signaling pathway, or reduce one or more symptoms of a disease/disorder/condition). An example of a “therapeutically effective amount” is an amount sufficient to contribute to the treatment, prevention, or reduction of one or more symptoms of a disease/disorder/condition. A “reduction” of a symptom means decreasing the severity and/or frequency of the symptom, and/or an elimination of the symptom. The exact amount of a composition including a “therapeutically effective amount” will depend on the purpose of the treatment, and will be ascertainable by one skilled in the relevant art.

In some embodiments, a therapeutically effective amount of an IL-12 Fc fusion protein, as described above in the compositions and/or methods of making sections, is administered to a subject in need thereof. In further embodiments, the IL-12 Fc fusion protein is administered to the subject in need thereof to treat a disease, a disorder, or a condition. In yet further embodiments, the disease, disorder, or condition comprises cancer.

In some embodiments, a therapeutically effective amount of an IL-12 Fc fusion protein, as described above in the compositions and/or methods of making sections, is administered to a subject in need thereof. In further embodiments, the IL-12 Fc fusion protein is administered to the subject in need thereof to treat a disease, a disorder, or a condition. In yet further embodiments, the disease, disorder, or condition comprises cancer. In even further embodiments, the cancer comprises a benign tumor, a metastatic tumor, or a mixed-type tumor.

In some embodiments, a therapeutically effective amount of an IL-12 Fc fusion protein, as described above in the compositions and/or methods of making sections, is administered to a subject in need thereof. In further embodiments, the IL-12 Fc fusion protein is administered to the subject in need thereof to treat a disease, a disorder, or a condition. In yet further embodiments, the disease, disorder, or condition comprises cancer. In even further embodiments, the cancer comprises recurrent cancer and/or refractory cancer. In some embodiments, the refractory cancer is a primary refractory cancer (i.e., a cancer that is resistant or immune to a given therapy at baseline). In other embodiments, the refractory cancer is a secondary refractory cancer (i.e., a cancer that was previously susceptible or partially susceptible to a given therapy at a first time-point but has subsequently become resistant or immune to a given therapy at a second time point, such as, for example, at or after recurrence).

In some embodiments, the IL-12 Fc fusion protein is administered as a first-line, second-line, third-line, fourth-line, fifth-line, or higher therapy.

In some embodiments, the IL-12 Fc fusion protein is administered alone. In other embodiments, the IL-12 Fc fusion protein is administered as a part of a combination therapy.

Several of the IL-12 Fc fusion proteins described herein can be used to treat a cancer in a human subject in need thereof. As is described in further detail above, these methods generally comprise administering a therapeutically effective amount of an IL-12 Fc fusion protein to the human subject to effectuate treatment of the cancer (or symptoms thereof). In some embodiments, the cancer is refractory to one or more current front-line therapies (such as, for example, treatment with a(n) (human) anti-PD-1 antibody). In some further embodiments, the cancer is sensitive to treatment with a therapeutically effective amount of an IL-12 Fc fusion protein described herein. In still some further embodiments, treatment with the therapeutically effective amount of the IL-12 Fc fusion protein sensitizes the cancer to the one or more current front-line therapies to which it is refractory to treatment. As such, a method for treating a refractory cancer in a human subject in need thereof is also contemplated, the method comprising: administering a therapeutically effective amount of an IL-12 Fc fusion protein in combination with a therapeutically effective amount of a current front-line therapy to the human subject in need thereof. In some further embodiments, the combination therapy is administered simultaneously or sequentially. In some exemplary embodiments, the cancer is refractory to treatment with a(n) (human) anti-PD-1 antibody, and the method comprises administering a therapeutically effective amount of an IL-12 Fc fusion protein in combination with a therapeutically effective amount of an anti-PD-1 antibody. In still some further exemplary embodiments, the anti-PD-1 antibody comprises a human anti-PD-1 antibody.

### EXAMPLES

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### A. Example 1—Expression and Purification of PD-1-Targeted Reduced Potency IL-12-ABD Fusion Proteins

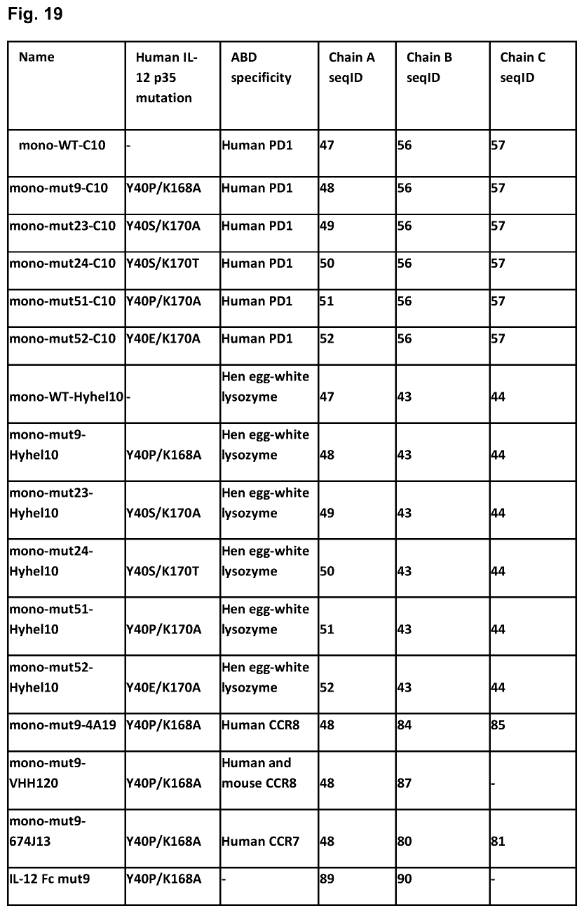

Targeting of wild type IL-12 to specific cell types or tissues via fusion to an ABD can be ineffective due to the extremely high affinity of IL-12 for the TL-12 receptor. To overcome this limitation, a series of IL-12 Fc fusion proteins (sometimes referred to herein as “IL-12-ABD fusion proteins”) were generated whereby the p35 subunit of IL-12 was either wild-type or contained substitution mutations that reduced the affinity of the resultant fusion protein for IL-12Rβ2. Instead of a tumor-targeting ABD, such as those commonly used within the field, the fusion protein was initially configured to target IL-12 to activated T cells via fusion to an ABD specific for human PD-1 (clone C10), or to an irrelevant target (hen egg-white lysozyme, clone Hyhel10) as a control. An exemplary TL-12-ABD fusion protein configuration is depicted in FIG. 18, wherein the fusion protein comprises a single chain TL-12 (p40-linker-p35) containing a C252S mutation within the p40 subunit, an anti-PD-1 Fab binding arm, and a heterodimeric human IgG1 Fc that contained mutations to abrogate Fc effector function. Details regarding which IL-12 mutants were combined with C10 and Hyhel10 ABDs, and associated sequences thereof, are depicted in FIG. 19.

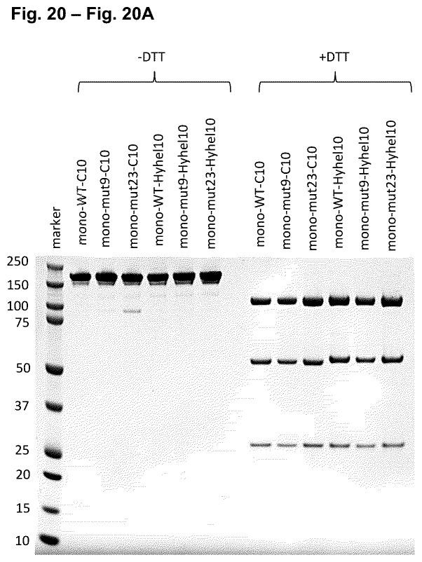

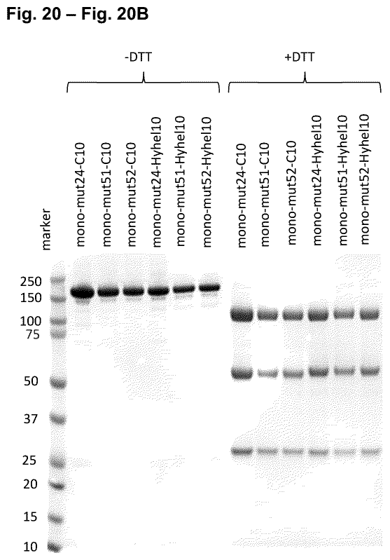

The resultant IL-12-ABD fusion proteins were independently produced by transient transfection of expi-CHO cells according to standard protocols and purified by a combination of protein A and cation exchange chromatography. SDS-PAGE analysis of purified IL-12-ABD fusion proteins is shown in FIG. 20.

### B. Example 2—Capacity of PD-1-Targeted IL-12-ABD Fusion Proteins to Trigger IFNγ Release in PD1⁺ and PD1—Primary Immune Cell Subsets

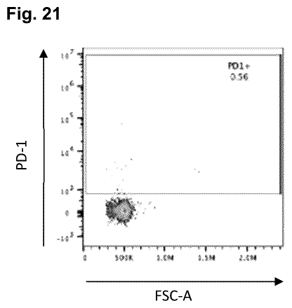

The toxicity of systemically administered IL-12 is caused at least in part due to excessive production of proinflammatory cytokines such as IFNγ by circulating NK cells. Accordingly, to avoid toxicity, it is advantageous for an IL-12 based therapeutic to limit IFNγ production by naïve NK cells. Since non-activated NK cells do not typically express PD-1 (see, e.g., FIG. 21 for a representative example), the capacity of five reduced potency IL-12-PD-1 fusion proteins to trigger IFNγ production by naïve NK cells was assessed in vitro. NK cells were isolated from healthy donor-derived peripheral blood mononuclear cells (PBMCs) using an NK cell isolation kit (Miltenyi) and 15,000 cells per well were resuspended in 384-well plates in SCGM medium (CellGenix) containing 10% human serum, 1× GlutaMAX, and varying concentrations of commercially available recombinant IL-12 (rIL-12, Miltenyi) or reduced potency IL-12-ABD fusion proteins for 18 hours, after which the levels of IFNγ within the supernatant were measured using an AlphaLISA High Performance Human IFNγ Detection Kit (Perkin Elmer).

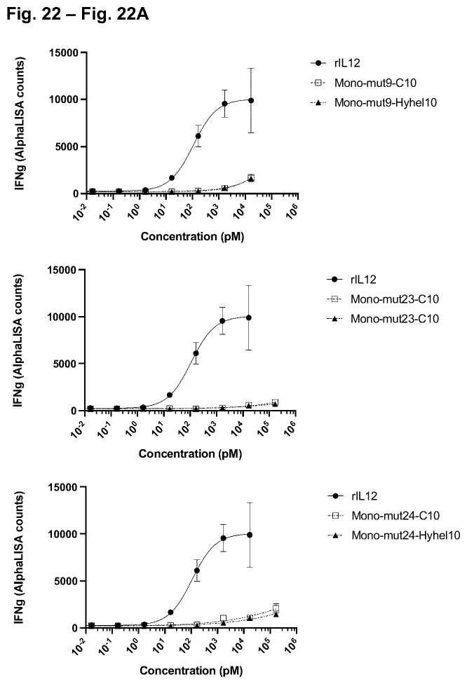

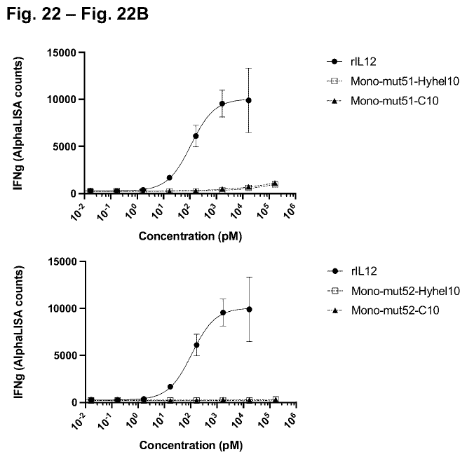

Results depicting IFNγ production by the various test articles are shown in FIG. 22A and FIG. 22B. Each of the reduced potency IL-12 mutants tested elicited considerably lower IFNγ production compared to rIL-12. The magnitude of this effect was similar irrespective of whether the IL-12 mutant was fused to a PD-1-targeting (C10) ABD, or the control hen egg-white lysozyme-targeting (Hyhel10) ABD. These data demonstrated that the test articles (reduced potency IL-12 fused to anti-PD-1 ABD) are likely to exhibit an improved safety profile relative to wild type IL-12 when administered systemically.

While high levels of IFNγ in the blood are undesirable, the production of IFNγ locally within the tumor microenvironment is beneficial as this cytokine is known to be cytostatic/cytotoxic, anti-angiogenic, and can upregulate MHC I and II expression on tumor cells to enable immune recognition. Indeed, the anti-tumor activity of IL-12 has at least in part been attributed to its capacity to induce IFNγ secretion within the tumor microenvironment. Accordingly, the capacity of the PD-1-targeted reduced potency IL-12-ABD fusion proteins to produce IFNγ in PBMC-derived activated primary T cells was investigated, since these represent a cell type that would be specifically located within an inflamed tumor microenvironment.

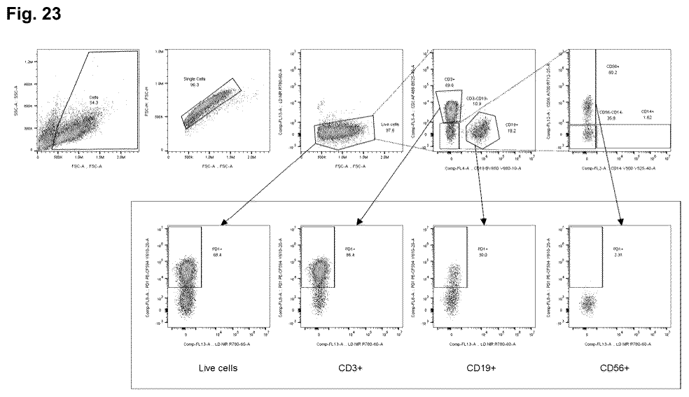

PBMCs from healthy donors were expanded/activated for 2 days in RPMI 1640 media containing 10% FCS, 1× GlutaMAX, 1% pen/strep, 0.1% β-mercaptoethanol, 10 mM HEPES, 1% non-essential amino acids, 1% sodium pyruvate, 600 U/mL IL-2, and 32 ng/mL OKT3. Following activation, cells were analyzed by flow cytometry to assess purity and PD-1 expression (see, e.g., FIG. 23). Different immune cell subsets were defined according to their expression of the following lineage markers: T cells, CD3⁺; B cells, CD19⁺; NK cells, CD3⁻/CD19⁻/CD56⁺. Among the population as a whole, the majority of cells (68.4%) expressed PD-1 due to its widespread expression on CD3⁺ T cells (86.4% positive), which represented the major cell population (69.6% of live cells).

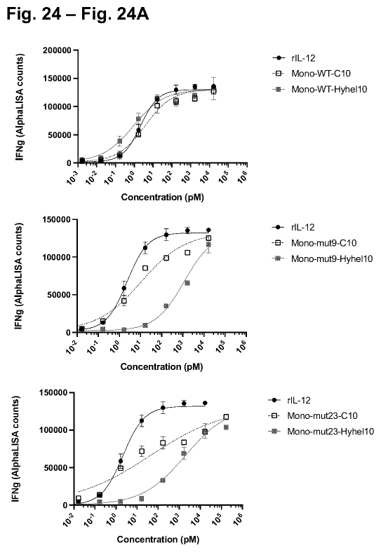

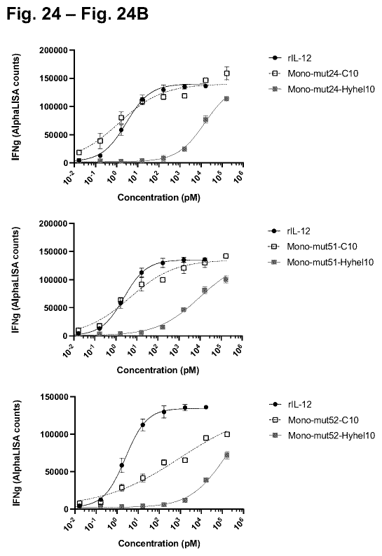

For the assay, 15,000 cells were dispensed into 384-well plates in RPMI 1640 media containing 10% FCS, 1× GlutaMAX, 1% pen/strep, 0.1% β-mercaptoethanol, 10 mM HEPES, 1% non-essential amino acids, 1% sodium pyruvate, 50 U/mL human IL-2 and incubated with varying concentrations of commercially available recombinant IL-12 or the various IL-12-ABD fusion proteins for 2 days, after which the levels IFNγ within the supernatant were measured using an AlphaLISA High Performance Human IFNγ Detection Kit (Perkin Elmer), the results of which are shown in FIG. 24A and FIG. 24B. Within this context, the potency of each of the Hyhel10-fused IL-12-ABD fusion proteins were considerably lower than that of the recombinant IL-12 control with respect to their capacity to stimulate IFNγ. However, all of the PD-1-fused IL-12-ABD fusion proteins demonstrated higher potency compared to the corresponding Hyhel10-fused variants. In contrast, fusion to the PD-1-specific ABD did not impact the level of IFNγ production induced by WT IL-12 (mono-WT-C10 sample). Together, these data demonstrate that fusion to an ABD can enhance the activity of reduced potency IL-12 mutants (but not WT IL-12) on cells that express the ABD target.

### C. Example 3—in Silico Analysis to Identify Additional ABD Targets

At this stage, it is unknown and unpredictable as to which ABD should be combined with reduced potency IL-12 to generate a therapeutic agent that will provide the most benefit for the treatment of human diseases such as cancer. Indeed, several characteristics of the ABD target could potentially impact the effectiveness of such a therapeutic agent, including but not limited to, target expression profile, cell surface density, clustering, and rate of internalization. IL-12 exhibits pleiotropic effects on cells within the tumor microenvironment, reflecting the IL-12Rβ1 and IL-12Rβ2 expression profile across multiple cell types. This includes activation and proliferation of T and NK cells, induction of Th1 differentiation, inhibition or reprogramming of suppressive cells such as tumor-associated macrophages (TAMs) and regulatory T cells (Treg), and induction of MHC-I/II expression on tumor cells. Accordingly, the expression profile of the ABD target will directly impact which cell types/states receive potent IL-12 activity. As such, it was hypothesized that the ideal ABD target might be specifically expressed on the various tumor infiltrating lymphocyte populations that contribute to the therapeutic effects of IL-12 and not expressed (or expressed at lower levels) on normal tissue and immune cell subsets that have been implicated in IL-12 mediated toxicity (e.g., circulating NK cells).

To identify potential ABD targets that have a desirable expression profile, we performed an in silico analysis of single cell RNA-seq data sets containing tumor samples, and where available, matched blood and/or normal healthy tissue samples, specifically: clear cell Renal cell carcinoma (ccRCC) data (PMID: 35668194); lung adenocarcinoma (LUAD) data (PMID: 32385277); non-small cell lung cancer (NSCLC) data (PMID: 37001526); and a pan-cancer data set with breast cancer (BRCA), esophageal carcinoma (ESCA), Uterine Corpus Endometrial Carcinoma (UCEC), thyroid cancer (THCA), and renal carcinoma (RC) (PMID: 34914499).

Briefly, pre-processed count matrices were obtained from the respective studies and cell types were re-classified, first annotating different immune cells using Seurat's label transfer with the 10× Genomics PBMC transcriptome and surface epitope (CITE-seq) reference data (PMID: 34062119), and then using scMatch to further classify cell subsets within resulting Seurat clusters. Activated CD8 T cells were defined by the presence of either PDCD1 or LAG3 transcripts and the absence of both TCF7 and SELL, while naïve CD8 T cells were defined by the presence of both TCF7 and SELL, and the absence of both LAG3 and PDCD1. Pseudo-bulking (PB) was performed within each donor, tissue source (if applicable), and cell subset (when comparing CD8 sub-populations), and differential expression analyses were performed using voom-limma to compare either tumor vs normal tissue or blood CD8 T cells (regardless of their naïve/active status), or to compare defined subpopulations of CD8 T cells (active, naïve, and other groups).

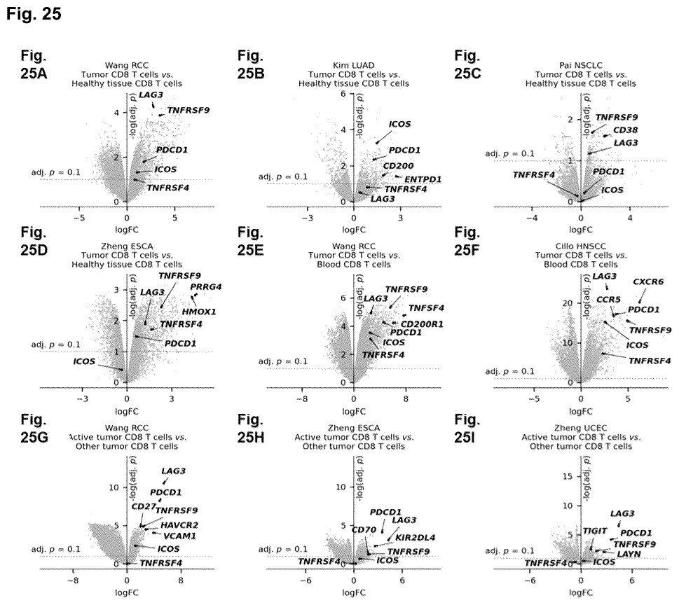

Volcano plots showing the log-transformed fold change in RNA transcript abundance and measure of statistical significance (negative log-transformed adjusted p-value) for selected studies and comparisons are provided in FIG. 25 (see, e.g., FIGS. 25A-25I). To identify cell surface components which may show consistent upregulation in anti-tumor CD8 T cells across a range of different cancer types, a meta-analysis across studies and conditions was performed, determining the rank of each gene by transcript upregulation (from the product of log FC*(1-adjusted p-value)) and calculating the product of ranks across all conditions. Genes for cell surface targets were then subsequently ranked by their product of ranks (see, e.g., FIG. 25J). Of note, PDCD1 and LAG3 performed particularly well in the product of ranks, presumably driven by their use in classifying ‘active’ CD8 T cells within the tumor and associated ranks within comparisons to other tumor infiltrating CD8 T cell subsets. Examples of additional genes that scored highly in the product of ranks were those classified as immune checkpoints (HAVCR2, encoding TIM3; TIGIT), co-stimulatory molecules (ICOS; TNFRSF9, encoding 41BB; TNFRSF4, encoding OX40), markers of T cell activation (ENTPD1, encoding CD39; LAYN, encoding Layilin; CD27) and chemokine receptors (CCR5; CXCR6).

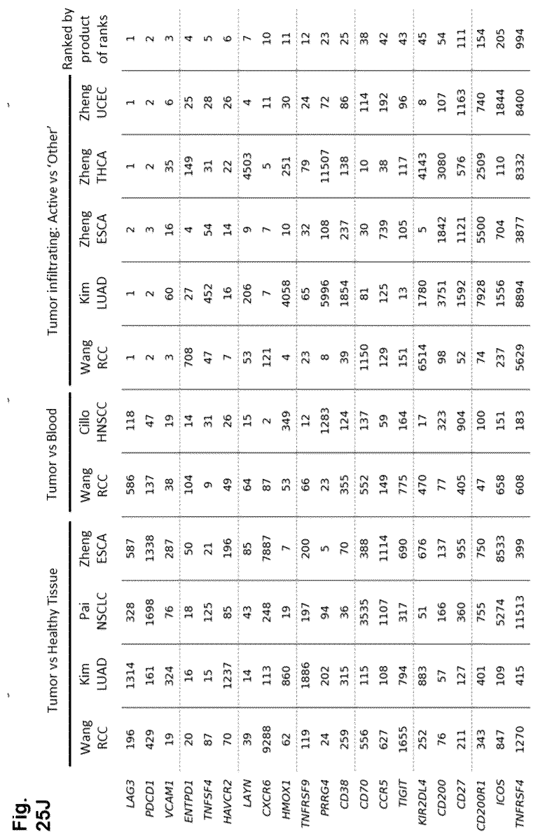

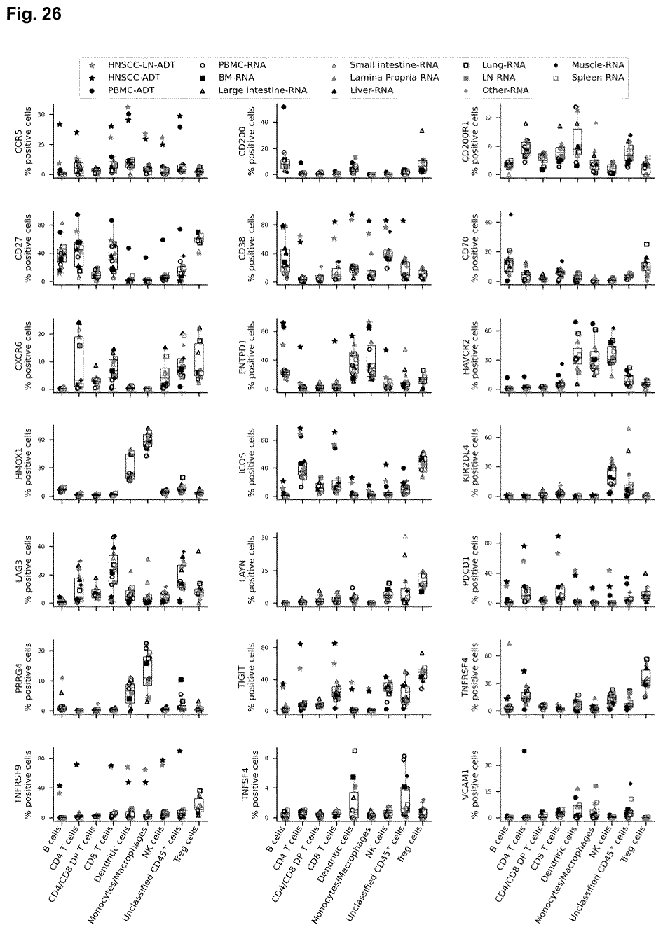

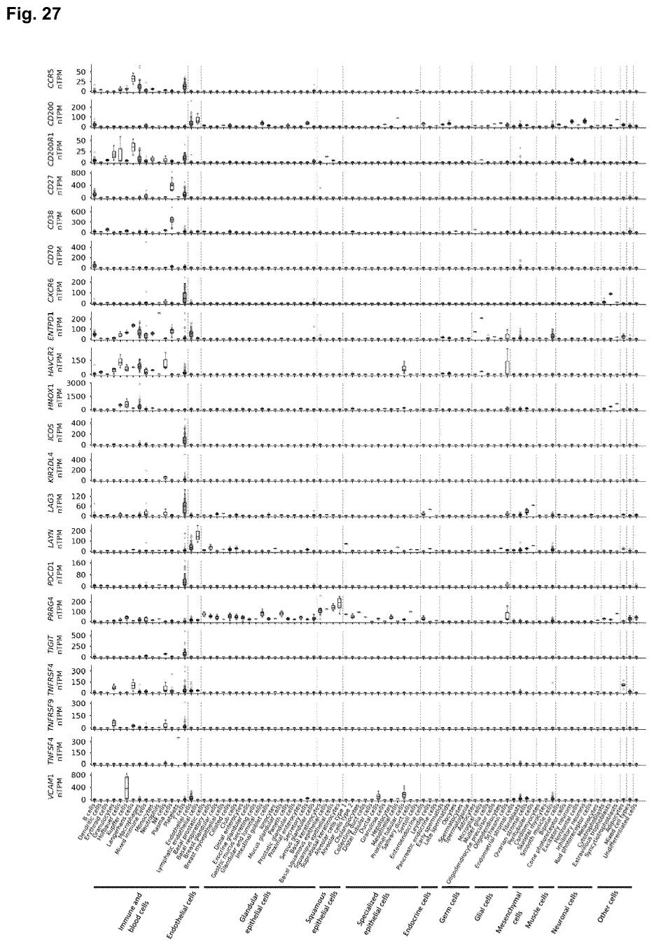

To exclude ABD targets that are expressed on cellular subsets/tissues that may contribute to IL-12 mediated toxicities, a single-cell RNA-seq data set across 12 patients, containing immune cells from the peripheral blood as well as several solid tissues, including liver, lung, skeletal muscle and bone marrow (PMID: 35549406), was examined. For genes of surface targets that performed well across the product of ranks (FIG. 25J), the proportion of cells from each subset that had at least 1 detectable RNA transcript for the corresponding gene in normal healthy tissue was examined (see, e.g., FIG. 26). In parallel, the Human Protein Atlas ‘ #11 RNA single cell type tissue cluster data’ was used (retrieved from the Downloads page (https://www.proteinatlas.org/about/download) December 2023), and mean transcript abundance (as measured by the consensus normalized transcripts per kilobase million; nTPM) of these targets within their respective sub-populations was examined (see, e.g., FIG. 27).

Together, these analyses revealed that, although several targets show relatively strong upregulation within tumor-resident and/or activated CD8 T cells, their absolute abundance within these cells is relatively low compared to other cell types found throughout the body. For example, CD200, ENTPD1, and LAYN have relatively high expression within endothelial cells, while PRRG4 has high expression within many different epithelial cell types (FIG. 27).

Accordingly, these targets were not selected for the ABD due to the potential for targeting to peripheral tissues. As noted above, the potential for IL-12 to drive immune-related toxicities was a major concern in the design considerations, and accordingly HMOX1 and VCAM1 were discarded due to their high expression on Kupffer cells and the associated potential in driving liver toxicity. Similarly, CD38, HAVCR2, TIGIT and KIR2DL4 transcripts are expressed within a relatively large proportion of natural killer cells (FIG. 26) and at a relatively high level compared to other cell types (FIG. 27), and hence these targets were discarded due to the potential for NK cell mediated IFNγ release and associated toxicities.

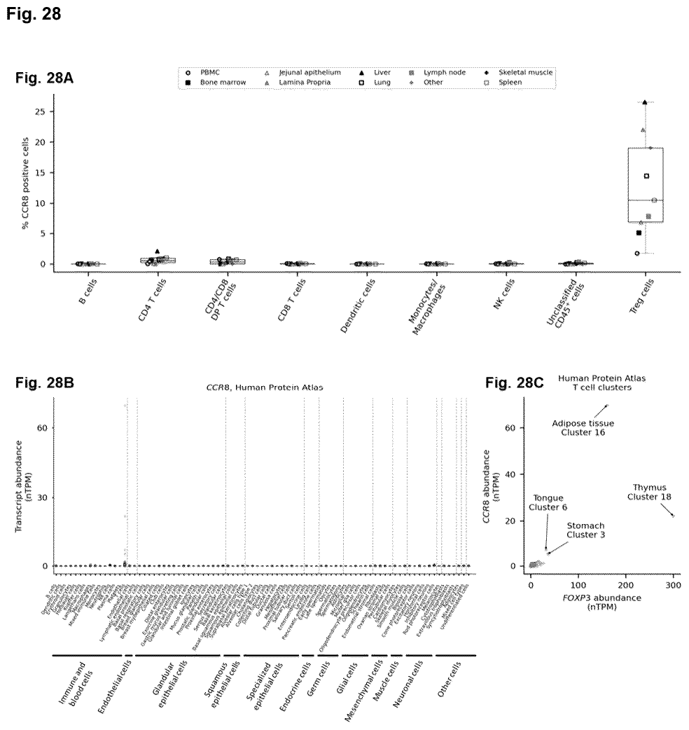

IL-12 has several putative anti-tumor effects mediated through Treg cells, in particular, reduced proliferation of Treg cells and polarization of these cells towards a TH1-like phenotype. Thus, it was hypothesized that it may be advantageous to specifically target reduced potency IL-12 variants to Treg cells. Chemokine receptor 8 (CCR8) has been proposed as a tumor Treg-specific cell surface marker. To examine this further, single cell RNA-seq data (as described above) were further analyzed and the RNA transcript abundance for the CCR8 gene was examined across different cell types and tissue sources. As shown in FIG. 28A, the proportion of cells positive for CCR8 transcript is very low across all immune populations, with the exception of Tregs, for all tissues examined. While the proportion of CCR8-positive Tregs within the liver is relatively high at approximately 25%, the proportion of Tregs within the liver is relatively low, constituting approximately 0.35% of cells within this dataset (compared to approximately 10% of cells within the spleen). The expression pattern for CCR8 is particularly restricted when looking across cellular populations from normal healthy tissue (see, e.g., FIG. 28B), with most clusters showing a very low (and in most cases, zero) nTPM abundance. Here, the T cell clusters with high CCR8 transcript abundance also have high FOXP3 transcript abundance (see, e.g., FIG. 28C), indicating that they correspond to tissue-resident Treg populations.

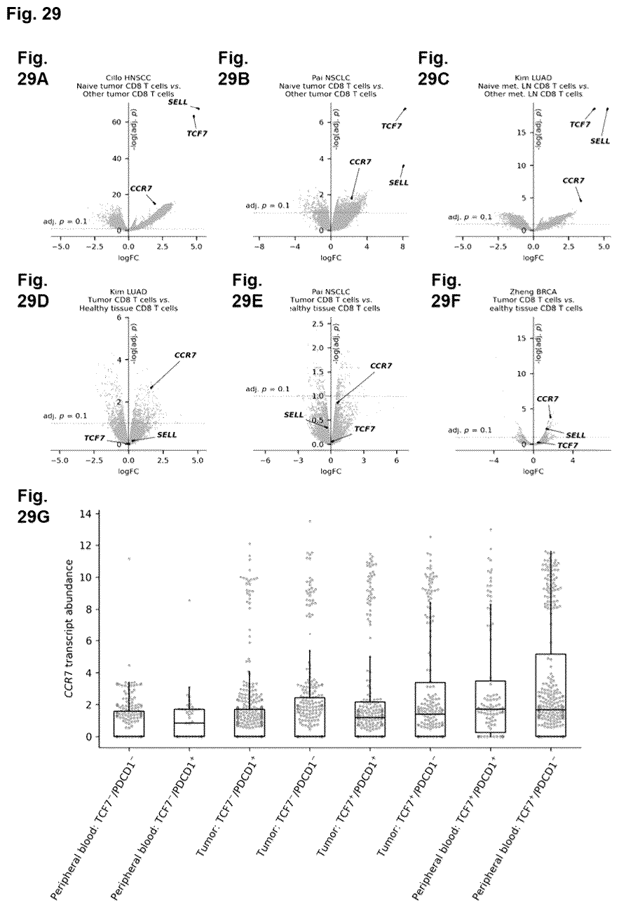

As an alternative approach, targeting reduced potency IL-12 variants to naïve and stem cell memory T cells (T_{scm}) was investigated. Naïve and T_{scm} T cells have extensive renewal capacity and are important for anti-tumor responses. As IL-12 promotes T cell priming and activation, it was hypothesized that targeting IL-12 to naïve and T_{scm} could potentiate T cell responses to antigen. Using single-cell RNA-seq data from head and neck squamous cell carcinoma (HNSCC; PMID 31924475), non-small cell lung cancer (NSCLC; PMID: 37001526, lung adenocarcinoma (LUAD; PMID 32385277), and breast cancer (BRCA; PMID 34914499), a variety of differential expression analyses using pseudo-bulked data were performed. As above, naïve CD8 T cells were defined by the presence of both TCF7 and SELL, and the absence of both LAG3 and PDCD1. Accordingly, both TCF7 and SELL were strongly upregulated when comparing naïve CD8 T cells to other T cell subsets within HNSCC, NSCLC, and LUAD lymph node metastases, while CCR7 was also strongly upregulated (see, e.g., FIGS. 29A-29C). When a pseudo-bulked differential expression analysis was performed between tumor samples and corresponding healthy tissue controls, it was also noted that CCR7 showed strong upregulation, even in instances where the naïve markers TCF7 and SELL were not upregulated to the same extent (see, e.g., FIGS. 29D-29F). To investigate this further at the single-cell level, a colorectal cancer data set (PMID: 32302573) generated using SmartSeq2 was identified, with the aim of minimizing ‘dropout’ effects that can occur for genes with low expression levels when using other single-cell technologies. CD8⁺ T cells from the periphery and tumor were extracted and classified as either positive or negative for PDCD1 and TCF7, using an expression threshold of zero, and the relative expression levels of CCR7 transcript were examined. As shown in FIG. 29G, CCR7 is highest within peripheral blood TCF7⁺/PDCD1⁻ cells, which may benefit from IL-12 priming, as well as peripheral blood TCF7⁺/PDCD1⁺ cells, corresponding to T_{scm} cells which may have been activated within the tumor before returning to the periphery. It was noted that all four tumor subsets exhibited higher expression levels than the TCF7⁻ (i.e., terminally differentiated) peripheral blood subsets, with the highest levels again observed for tumor resident (TCF7⁺/PDCD1⁻) T cells, and (TCF7⁺/PDCD1⁺) double-positive cells, corresponding to the T_{scm} subset.

### D. Example 4—Expression Profile of Potential ABD Targets on Ex Vivo Mouse Immune Cells

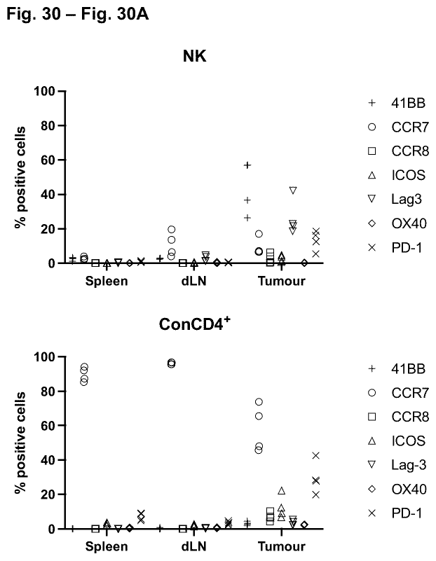

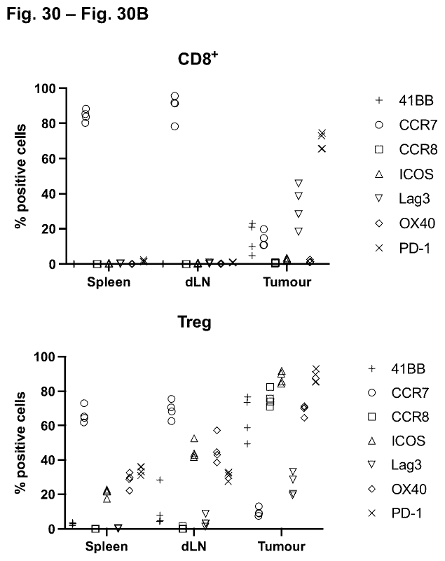

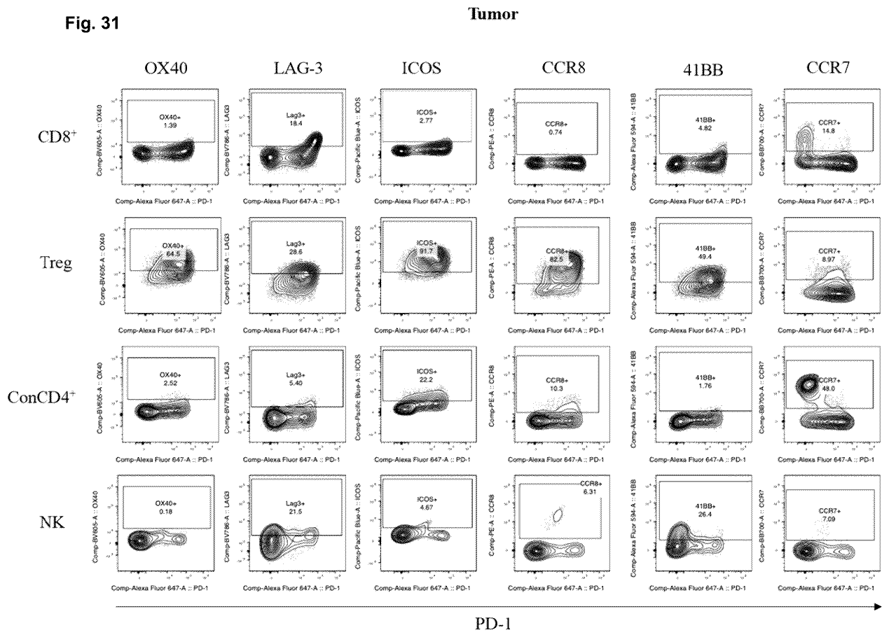

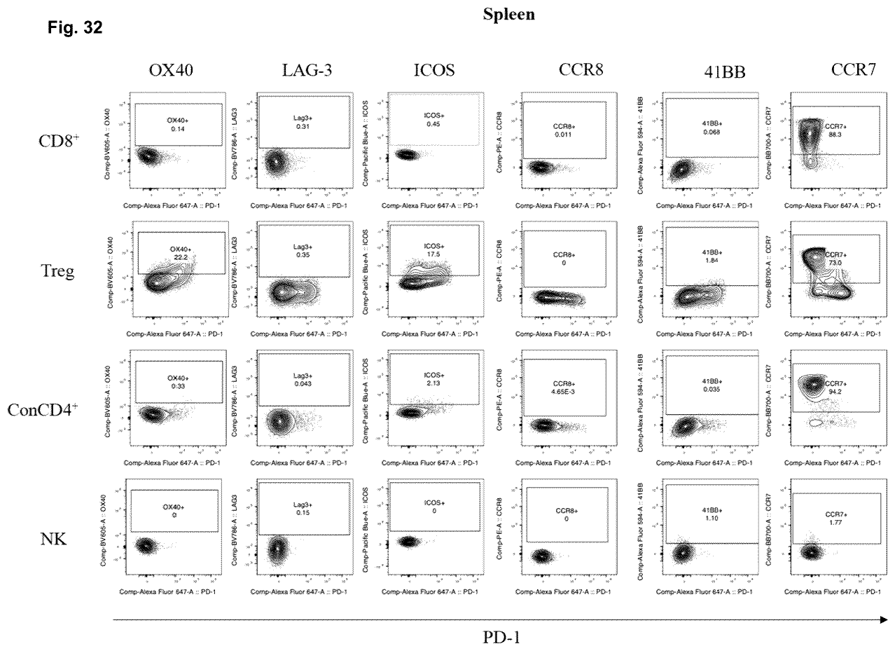

Based on the data outlined in Example 3, PD-1, LAG-3, 41BB, OX40, ICOS, CCR7 and CCR8 were selected as potential ABD targets. To further validate these, their expression profile on mouse immune cells was evaluated. Immune cells were isolated from MC38 tumors, blood, and tumor draining lymph node (dLN) derived from C57/BL6 mice that were unresponsive to IL-12 Fc therapy (14 days after treatment) and analyzed by flow cytometry using a Cytek Aurora. The % expression of each ABD target on CD8⁺, Conventional CD4⁺ (ConCD4, CD4⁺Foxp3⁻), Treg (CD4⁺Foxp3⁺) T cells, and NK (NKp46⁺ CD122⁺ CD8⁻ CD4⁻) cells isolated from the various organs is depicted in FIG. 30. Overall, the expression profile of the various ABD targets on mouse cells matched well with that determined based on in silico analysis of human-derived data (see, e.g., Example 3). Briefly, with the exception of CCR7, the expression of all of the potential ABD targets (41BB, CCR8, ICOS, LAG-3, OX40 and PD-1) was very low or absent on NK, ConCD4⁺, and CD8⁺ cells in the spleen and dLN. However, their expression was upregulated on distinct cell populations located within the tumor, including CD8⁺ T cells (PD-1, LAG-3, 41BB), ConCD4⁺ T cells (PD-1, ICOS), NK cells (PD-1, LAG-3, 41BB), and Tregs (PD-1, OX40, LAG-3, ICOS, CCR8 and 41BB). Conversely, CCR7 expression was highly expressed on CD8⁺ and ConCD4⁺ within the dLN and spleen, but was downregulated on cells within the tumor, where it was largely restricted to PD-1 negative cells, demonstrating that its expression is biased away from activated and exhausted T cells (see, e.g., FIG. 31). CCR8 was observed to be highly specific to tumor-infiltrating Treg cells (see, e.g., FIGS. 30-32).

### E. Example 5—PD-1, LAG-3, 41BB, OX40, and ICOS-Targeted Reduced Potency IL-12 Fusion Proteins

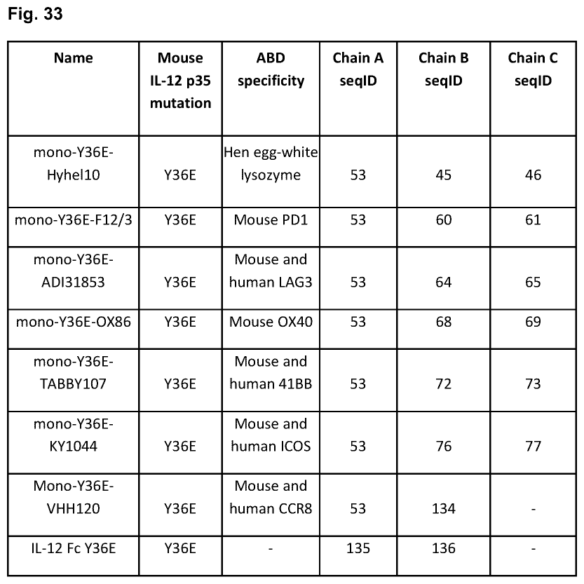

To investigate the utility of the ABD targets identified in Example 3 and validated in Example 4, a series of additional reduced potency IL-12-ABD fusion proteins were generated (see, e.g., FIG. 33). In this instance, an ABD that bound to mouse LAG-3 (clone ADI31853), OX40 (clone OX86), 41BB (clone TABBY107), PD-1 (clone F12/3), or ICOS (clone KY1044) was utilized, and fused to a reduced potency variant of mouse IL-12 that contained a Y36E mutation within the p35 subunit. The construct configuration was identical to that depicted in FIG. 18 except that the human IgG1 Fc was replaced with a mouse IgG1 Fc containing a D265A mutation to abrogate Fc effector function. As an additional control, the same Y36E reduced potency mouse IL-12 mutant was fused to an ABD specific for hen egg-white lysozyme (Hyhel10).

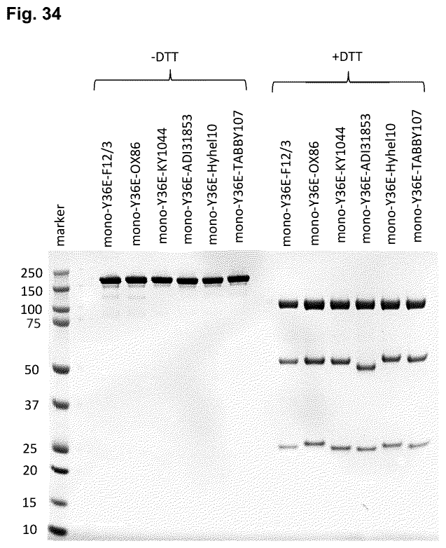

IL-12-ABD fusion proteins were produced by transient transfection of expi-CHO cells according to standard protocols and purified by a combination of either protein A and/or protein G followed by cation exchange chromatography. SDS-PAGE analysis of purified IL-12-ABD fusion proteins is shown in FIG. 34.

The activity of LAG-3, PD-1, 41BB, and ICOS-targeted IL-12 fusion proteins was initially assessed in activated mouse T cells. Briefly, splenocytes were isolated from C57BL/6 mice and activated overnight in RPMI 1640 media containing 10% FCS, 1× GlutaMAX, 1% pen/strep, 0.1% β-mercaptoethanol, 10 mM HEPES, 1% non-essential amino acids, 1% sodium pyruvate, 1 μg/mL anti-CD3 (clone 17A2, eBioscience) and 600 U/mL human IL-2 (Miltenyi). Mononuclear cells were isolated using Ficoll and T cells were purified by magnetic-activated cell sorting (MACS) using a mixture of biotinylated anti-CD19, anti-CD11 b, anti F4/80 and anti-TER-119 antibodies to remove non-T cells. Following purification, cells were analyzed by flow cytometry, the results of which are depicted in FIG. 35. Purified cells were predominantly comprised of T cells (32.3% CD8⁺, 40.8% CD4⁺) and expressed clearly detectable levels of ICOS (19.4% of cells), 41BB (23.8% of cells), and PD-1 (18.9% of cells). In contrast, LAG-3 expression was not detected on any cell population.

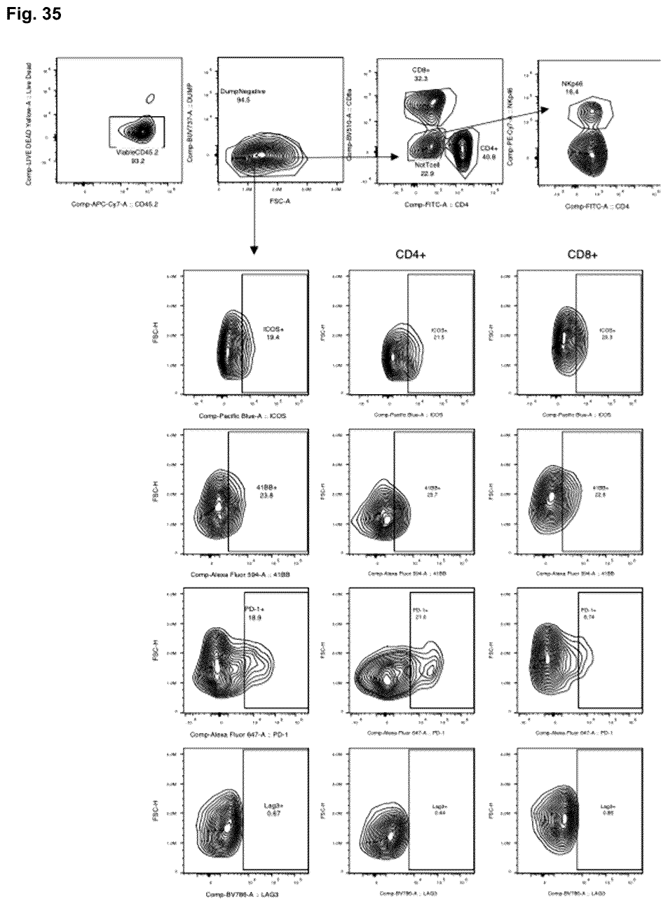

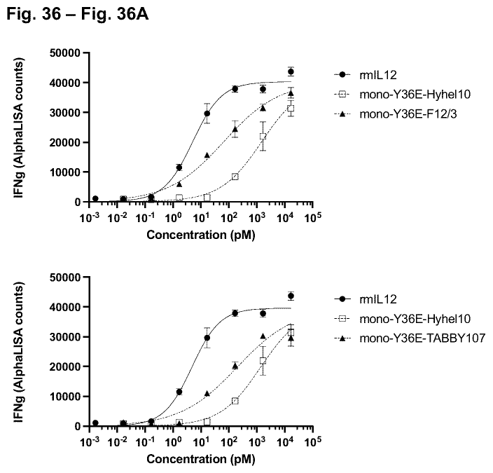

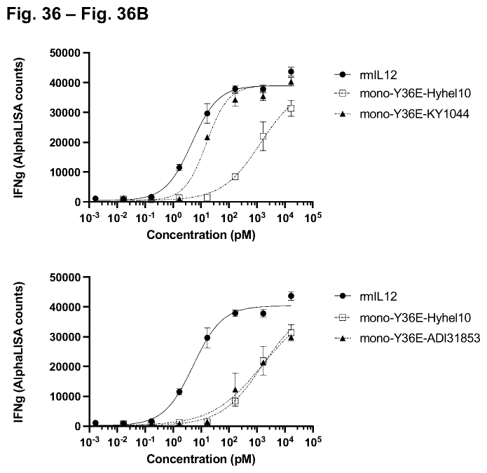

Purified cells were incubated for 2 days in the presence of 50 U/mL IL-2 and varying concentrations of commercially available recombinant mouse IL-12 (rmIL-12, BioLegend) or reduced potency IL-12-ABD fusion proteins, after which the levels IFNγ within the supernatant were measured using an AlphaLISA High Performance Human IFNγ Detection Kit (Perkin Elmer). The results of the AlphaLISA assay are shown in FIG. 36. Surprisingly, despite the moderate expression levels of PD-1, 41BB, and ICOS on the cells, each of the PD-1-targeted (mono-Y36E-F12/3), 41BB-targeted (mono-Y36E-TABBY107), and ICOS-targeted (mono-Y36E-KY1044) IL-12-ABD fusion proteins more potently stimulated IFNγ production relative to the corresponding irrelevant-targeting control (mono-Y36E-Hyhel10). Remarkably, in the case of the ICOS-targeted (mono-Y36E-KY1044) IL-12-ABD fusion protein, the potency of the IFNγ response was similar to that of the rmIL-12 control. Importantly, the LAG-3 targeted IL-12-ABD fusion protein (mono-Y36E-ADI31853) did not stimulate IFNγ production more potently than the irrelevant-targeting control (mono-Y36E-Hyhel10), which corresponds well with the absence of detectable LAG-3 expression on the cells used in the assay. These data demonstrate the utility of PD-1, ICOS, and 41BB ABD for targeting reduced potency IL-12 variants, and further reaffirm that expression of the ABD target is required to enhance the activity of reduced potency IL-12 variants on specific cell types.

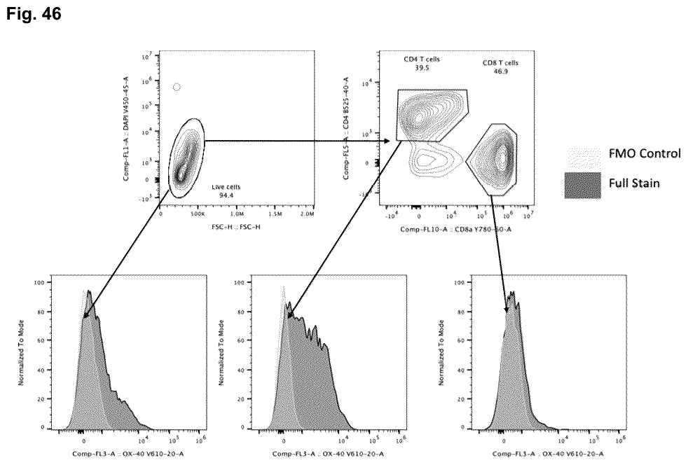

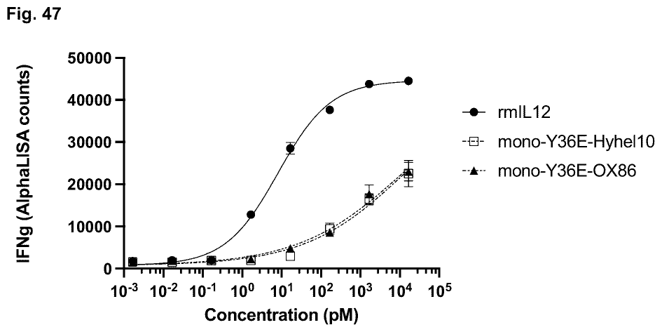

The activity of the OX40-targeted IL-12 fusion protein was assessed in a separate experiment using mouse T cells that were activated and purified using the same experimental workflow as described above for the LAG-3, PD-1, 41BB, and ICOS-targeted IL-12 fusion proteins, except that the activation step included 0.5 ug/mL anti-CD3 (clone 17A2, eBioscience), 0.5 ug/mL anti-CD28 (clone 37.51, eBioscience) and 600 U/mL human TL-2 (Miltenyi). Flow cytometry analysis of these cells and the results of the IFNγ assay are depicted in FIG. 46 and FIG. 47, respectively. Here, despite clearly detectable expression of OX40 on the activated cells (particularly the CD4⁺ T cells), the OX40-targeted IL-12-ABD fusion protein (“mono-Y36E-OX86) stimulated IFNγ production to the same extent as the hen egg-white lysozyme-targeted control (mono-Y36E-Hyhel10).

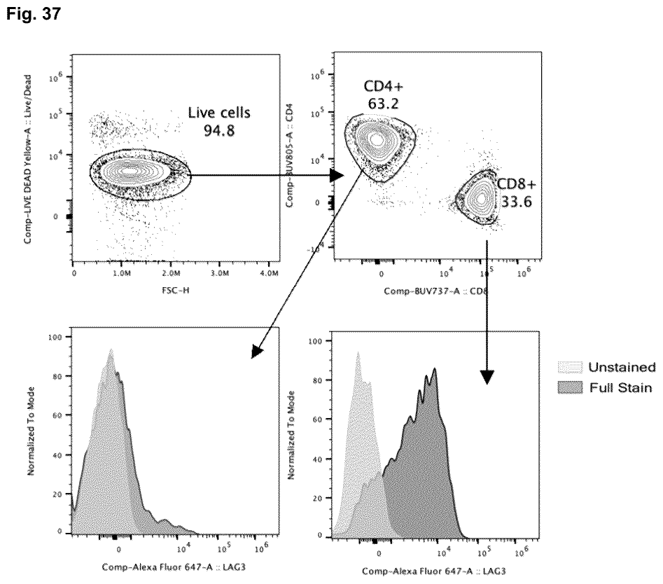

Since LAG-3 expression was not inducible on mouse cells in vitro, the activity of the LAG-3-targeted IL-12-ABD fusion protein was next assessed using activated and expanded human T cells. This was possible because mouse IL-12 cross-reacts with human IL-12 receptor, and the LAG-3-ABD (clone ADI31853) that was incorporated into the construct cross-reacts with human LAG-3. For the experiment, PBMCs from healthy donors were activated for 2 days in RPMI 1640 media containing 10% FCS, 1× GlutaMAX, 1% pen/strep, 0.1% β-mercaptoethanol, 10 mM HEPES, 1% non-essential amino acids, 1% sodium pyruvate, 600 U/mL IL-2, and 32 ng/mL OKT3 and then expanded for 5 days in the same media lacking OKT3. Following expansion, the cells were analyzed by flow cytometry (see, e.g., FIG. 37) and observed to be almost exclusively T cells (63.2% CD4⁺ and 33.6% CD8⁺), of which the CD8⁺ fraction expressed high levels of LAG-3.

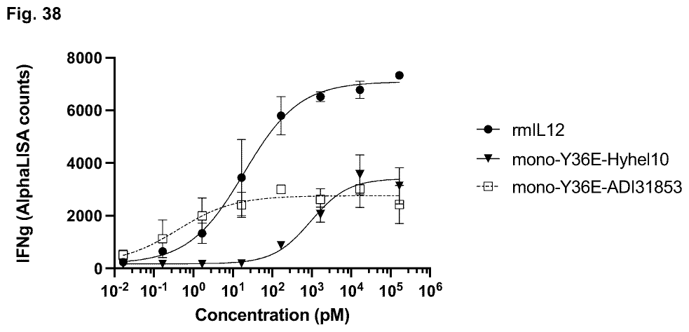

Expanded cells were incubated for 2 days in the presence of 50 U/mL TL-2 and varying concentrations of commercially available recombinant mouse IL-12 (rmIL-12, BioLegend) or reduced potency IL-12-ABD fusion proteins, after which the levels IFNγ within the supernatant were measured using an AlphaLISA High Performance Human IFNγ Detection Kit (Perkin Elmer). The results of the AlphaLISA assay are shown in FIG. 38. While the rmIL-12 sample potently induced a dose-dependent increase in IFNγ production (EC50, 19.3 pM), the hen egg-white lysozyme targeted mouse IL-12 Y36E mutant (mono-Y36E-Hyhel10) exhibited considerably reduced IFNγ production, thereby validating the use of this experimental system for the assessment of targeted IL-12-ABD fusion proteins. Indeed, the LAG-3 targeted mouse IL-12 Y36E mutant (mono-Y36E-ADI31853) more potently stimulated IFNγ production compared to the mono-Y36E-Hyhel10 sample, thereby demonstrating the utility of targeting reduced potency IL-12 variants using ABD specific for LAG-3.

### F. Example 6—CCR8-Targeted Reduced Potency IL-12-ABD Fusion Proteins

Next, the feasibility of targeting reduced potency IL-12 variants to cells that expressed CCR8 was evaluated. To demonstrate this principle, two CCR8-targeted reduced potency IL-12-ABD fusion proteins were generated, whereby human IL-12 containing a Y40P/K168A mutation within the p35 subunit was fused to either an anti-CCR8 Fab (clone 4A19) or nanobody (clone VHH120). Details regarding amino acid sequences of the CCR8-targeted IL-12 fusion proteins are shown in FIG. 19.

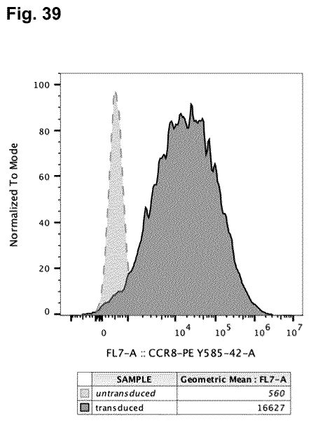

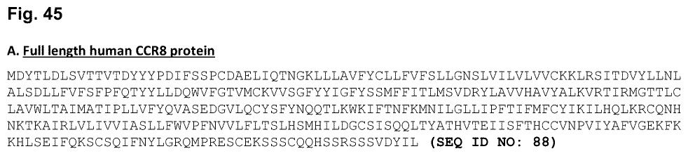

Due to the tightly restricted expression profile of CCR8, the activity of CCR8-targeted IL-12 fusion proteins were assessed using a modified version of a commercially available IL-12 reporter cell line (HEK-Blue IL-12 cells, InvivoGen), whereby IL-12-induced triggering of the STAT4 pathway leads to the production of secreted embryonic alkaline phosphatase (SEAP), which can readily be detected using QUANTI-Blue reagent. HEK-Blue IL-12 cells were modified by transduction with a lentiviral vector containing the human CCR8 gene (SEQ ID NO: 88, as shown in FIG. 45). Transduced cells were then sorted using a CCR8-specific antibody (clone L263G8, BioLegend) to obtain a homogeneous population of CCR8 expressing cells, termed CCR8-HEK-Blue IL-12 cells (see, e.g., FIG. 39).

Varying concentrations of recombinant IL-12, IL-12 Fc variant or IL-12-ABD fusion proteins were incubated with 12,500 CCR8-HEK-Blue IL-12 cells overnight at 37° C. in a 384-well plate resuspended with DMEM containing 4.5 g/L glucose, 1% GlutaMAX, 10% heat inactivated FBS, and 100 U/mL penicillin/streptomycin. The following day, SEAP was quantified using QUANTI-Blue detection reagent by measuring optical density (OD) at 650 nm using a EnVision 2105 Multimode plate reader and results were plotted in Prism software.

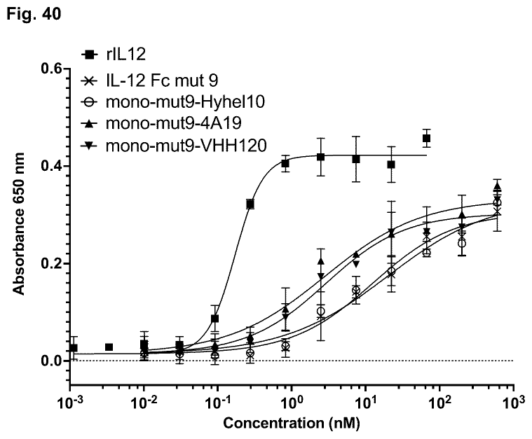

The results of the CCR8-HEK-Blue IL-12 assay are shown in FIG. 40. While recombinant IL-12 potently activated the reporter cells (EC50, 0.17 nM), the Hyhel10-targeted Y40P/K168A IL-12 variant (mono-mut9-Hyhel10) exhibited a marked reduction in potency (EC50, 19.57 nM) that was similar to that observed for an untargeted IL-12 Fc fusion protein (IL-12 Fc mut9) wherein the p35 subunit contained the same Y40P/K168A mutation (EC50, 12.52 nM). However, IL-12 fusion proteins containing either of the CCR8 ABDs (mono-mut9-4A19 or mono-mut9-VHH120) exhibited markedly more potent activity (approximately 7-fold greater) than the mono-mut9-Hyhel10 molecule. Interestingly, the EC50 values for mono-mut9-4A19 and mono-mut9-VHH120 samples (2.8 nM and 2.9 nM, respectively) were indistinguishable, indicating that geometric constraints associated with changing the format of the ABD from a Fab to a nanobody did not alter the functional attributes of the molecule.

### G. Example 7—CCR7-Targeted IL-12-ABD Fusion Proteins

Next, the feasibility of targeting reduced potency IL-12 variants to cells that expressed CCR7 was assessed. To demonstrate this principle, a CCR7-targeted reduced potency IL-12-ABD fusion protein was generated, whereby human IL-12 containing a Y40P/K168A mutation within the p35 subunit was fused to an anti-CCR7 Fab (clone 674J13). Details regarding the amino acid sequences of the CCR7-targeted IL-12 fusion protein are shown in FIG. 19.

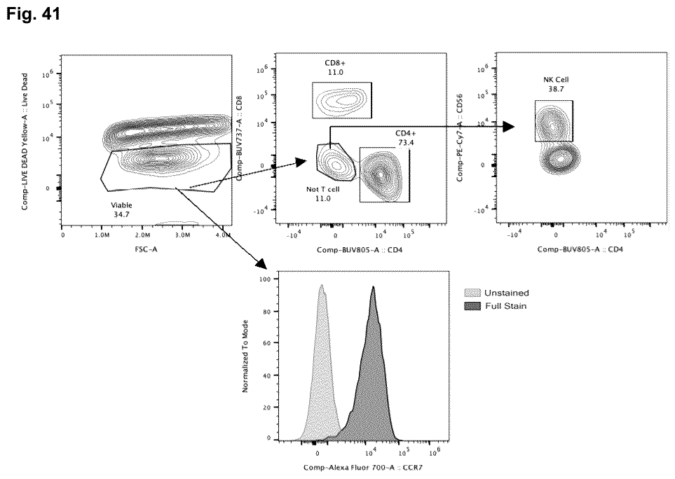

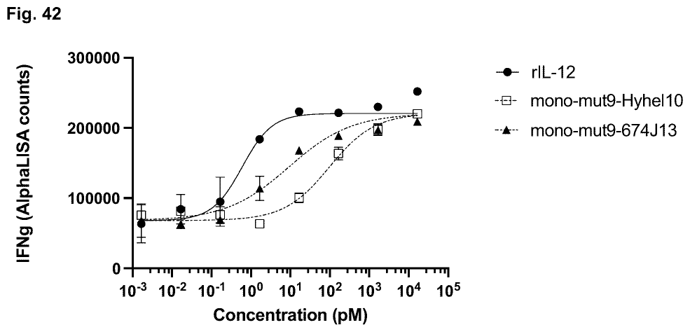

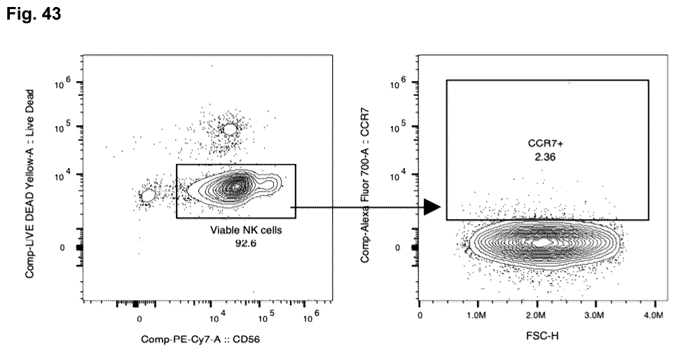

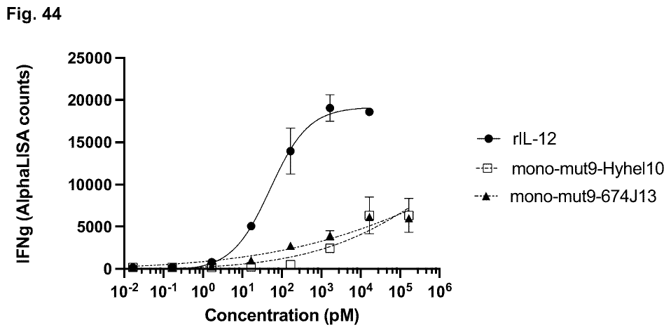

To assess the activity of the CCR7-targeted reduced potency IL-12-ABD fusion protein, PBMCs from healthy donors were activated for 2 days in RPMI 1640 media containing 10% FCS, 1× GlutaMAX, 1% pen/strep, 0.1% β-mercaptoethanol, 10 mM HEPES, 1% non-essential amino acids, 1% sodium pyruvate, 600 U/mL IL-2, and 32 ng/mL OKT3. Following activation, the cells were analyzed by flow cytometry (see, e.g., FIG. 41). Among the population as a whole, the majority of live cells expressed CCR7, indicating that this population could be used to assess the activity of a CCR7-targeted reduced potency IL-12-ABD fusion proteins. Indeed, cells treated for 2 days with the CCR7-targeted reduced potency IL-12-ABD fusion protein (mono-mut9-674J13) more potently stimulated IFNγ production than cells treated with the corresponding Hyhel10 targeted reduced potency IL-12-ABD (mono-mut9-Hyhel10) (FIG. 42). In addition, the activity of mono-mut9-674J13 in naïve human NK cells that did not express CCR7 was examined (FIG. 43) using the same experimental workflow as described in Example 2. Here, CCR7-targeting had no impact on the capacity of the reduced potency IL-12 variant to stimulate IFNγ production (FIG. 44). Thus, CCR7 targeting has the capacity to enhance the activity of reduced potency IL-12 variants specifically on CCR7-expressing cells.

### H. Example 8—In Vivo Assessment of PD-1, LAG-3, OX40, 41BB, ICOS, and CCR8-Targeted IL-12-ABD Fusion Proteins

Next, the anti-tumor activity of various IL-12-ABD fusion proteins was investigated in C57/BL6 mice. Six- to eight-week-old female mice were implanted subcutaneously with 0.5×10⁶ MC38 colorectal cancer cells and randomized into treatment groups (n=6/group) when tumors reached an average size of approximately 35 mm³ (referred to as day 0). Mice were treated via intraperitoneal (IP) injections on days 0, 7 and 14 with either PBS (untreated), 0.03 mg/kg of untargeted mouse IL-12 Fc containing a Y36E mutation within the IL-12 p35 subunit but lacking an ABD (IL-12 Fc Y36E) or an equimolar amount of a specific, targeted IL-12-ABD fusion protein (PD-1, LAG-3, OX40, 41BB, ICOS, or CCR8). As an additional control, one group of tumor-bearing mice was treated with a ten-fold higher dose (0.3 mg/kg) of IL-12 Fc Y36E.

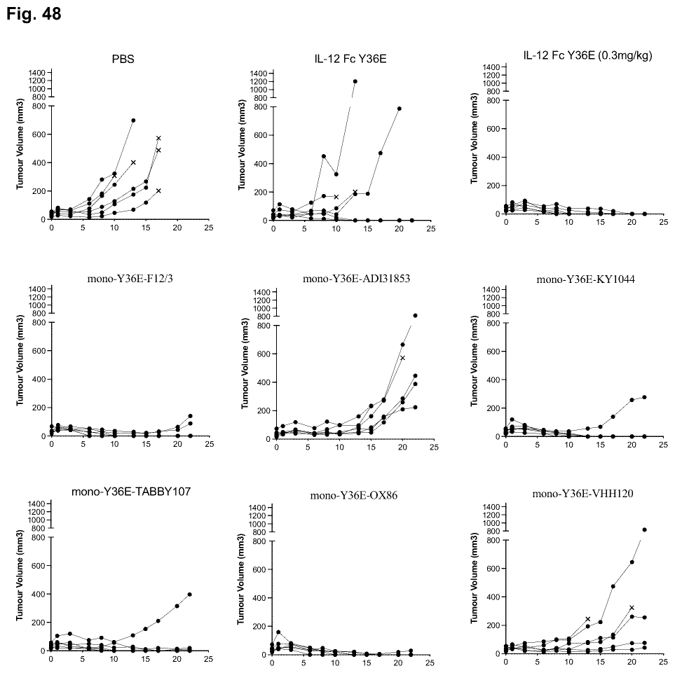

Tumor measurements were performed three times per week and tumor growth curves were generated (as depicted in FIG. 48). While tumors in 100% of PBS-treated mice grew in an uncontrolled manner, treatment with 0.03 mg/kg of untargeted IL-12 Fc Y36E resulted in a partial response, whereby 2 out of 6 mice exhibited tumor regression. Mice treated with an equivalent dose of LAG-3-targeted (mono-Y36E-ADI31853) or CCR8-targeted (mono-Y36E-VHH120) IL-12-ABD fusion proteins exhibited similar or marginally improved anti-tumor activity on average, relative to the 0.03 mg/kg IL-12 Fc Y36E treated group. However, treatment with PD-1-targeted (mono-Y36E-F12/3; 4 out of 6 mice exhibited complete response), 41BB-targeted (mono-Y36E-TABBY107; 5 out of 6 mice exhibited complete response), ICOS-targeted (mono-Y36E-KY1044; 5 out of 6 mice exhibited complete response), or OX40-targeted (mono-Y36E-OX86; 5 out of 6 mice exhibited complete response) IL-12-ABD resulted in potent anti-tumor activity that was equivalent to that observed with the ten-fold higher molar equivalent dose (0.3 mg/kg) of untargeted IL-12 Fc Y36E. The strong anti-tumor effect observed in mice treated with mono-Y36E-OX86 was particularly surprising, given the inability of OX40 targeting to restore IL-12 activity on OX40 expressing cells in vitro (see, e.g., Example 5).

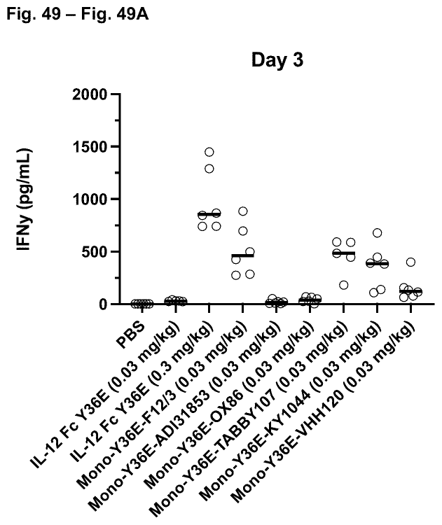

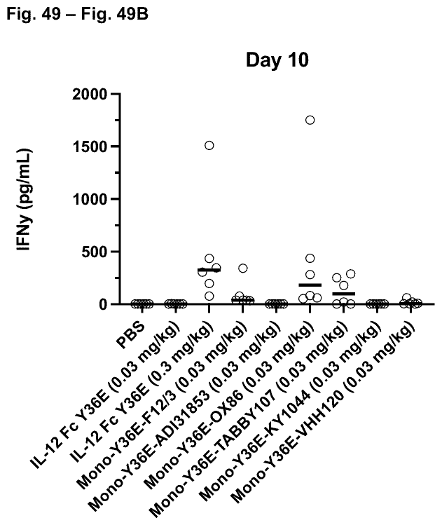

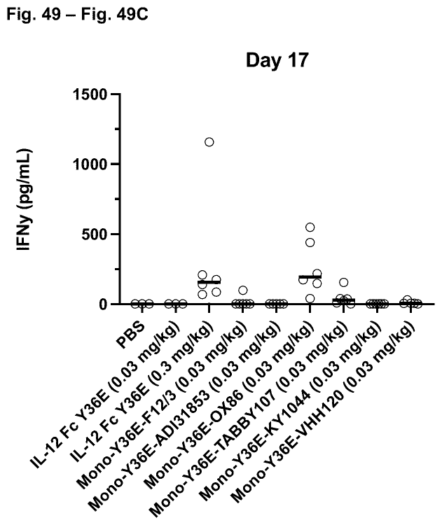

Elevated levels of IFNγ in the blood are correlated with IL-12-mediated toxicity and typically peak 2-4 days following the first dose of treatment. Accordingly, to provide an indication of the safety profile of the targeted IL-12-ABD fusion proteins, plasma IFNγ levels were quantified at various time points (FIGS. 49A-49C) using an AlphaLISA High Performance Mouse IFNγ Detection Kit (Perkin Elmer). On day 3, modest levels of IFNγ were observed in mice treated with PD-1-targeted (mono-Y36E-F12/3; mean IFNγ=509.9 pg/mL), 41BB-targeted (mono-Y36E-TABBY107; mean IFNγ=457.6 pg/mL), and ICOS-targeted (mono-Y36E-KY1044; mean IFNγ=356.4 pg/mL) IL-12 ABD, as shown in FIG. 49A. Notably, these were lower than those observed in mice treated with the 0.3 mg/kg dose of untargeted IL-12 Fc Y36E that resulted in equivalent anti-tumor activity (mean IFNγ=987.8 pg/mL). Surprisingly, mice treated with OX40-targeted (mono-Y36E-OX86) IL-12-ABD fusion protein exhibited very low levels of IFNγ (mean=39.4 pg/mL) in the plasma on day 3, which was comparable to that observed in mice treated with an equivalent molar dose (0.03 mg/kg) of untargeted IL-12 Fc Y36E (mean IFNγ of 29.2 pg/mL). The mono-Y36E-OX86 treatment group was also unique in that the pharmacodynamic response (as assessed by plasma IFNγ concentration) was increased after the second treatment dose (day 10; see, e.g., FIG. 49B), and largely maintained after the third treatment dose (day 17; see, e.g., FIG. 49C). This resistance to tachyphylaxis could represent an unexpected, but desirable property for an IL-12 based therapy.

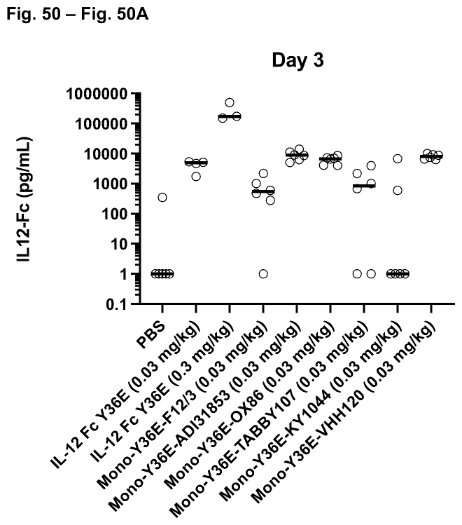

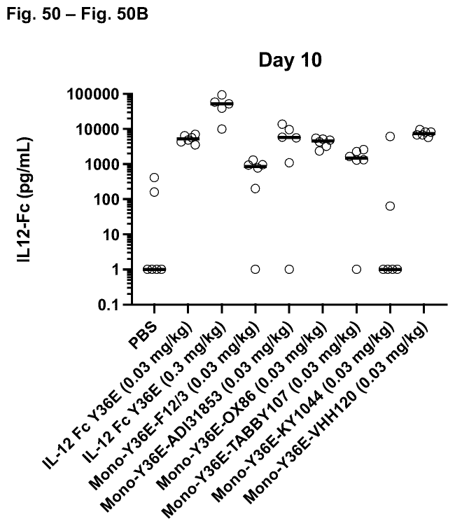

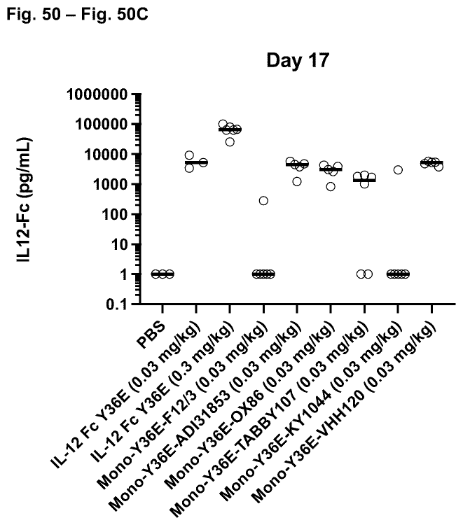

The concentration of IL-12 Fc and IL-12 ABD fusion proteins in the plasma of mice on days 3, 10, and 17 was determined using a sandwich ELISA approach using a monoclonal rat anti-mouse p40 antibody (Clone 30517, R&D systems) as a capture reagent and an HRP-conjugated goat anti-mouse IgG1 antibody (#1070-05, Southern Biotech) as a detection reagent. Quantification of IL-12 Fc and IL-12 ABD fusion proteins is depicted in FIG. 50 (FIG. 50A—day 3; FIG. 50B—day 10; FIG. 50C—day 17). Overall, the concentration of LAG-3-targeted (mono-Y36E-ADI31853), OX40-targeted (mono-Y36E-OX86), and CCR8-targeted (mono-Y36E-VHH120) IL-12 ABD fusion proteins were similar to that of the equimolar (0.03 mg/kg) dose of untargeted IL-12 Fc Y36E, indicating that fusion with LAG-3, OX40, or CCR8-specific ABD did not adversely affect the pharmacokinetic profile of the molecules. Lower plasma concentrations were observed for 41BB-targeted (mono-Y36E-TABBY107), PD-1-targeted (mono-Y36E-F12/3), and ICOS-targeted (mono-Y36E-KY1044) IL-12 ABD fusion proteins, indicating that fusion of reduced potency IL-12 to ABD specific for 41BB, PD-1 or ICOS may enhance targeted mediated clearance. The apparent short half-life of the ICOS-targeted (mono-Y36E-KY1044) IL-12 ABD fusion protein was particularly surprising given the durable anti-tumor activity observed in 5 out of 6 mice that received this treatment.

### L. Example 9—In Vivo Effects of Targeted IL-12-ABD Fusion Proteins on Tumor and Non-Tumor Tissues

Six- to eight-week-old female C57BL/6 mice were implanted subcutaneously with 0.5×10⁶ MC38 colorectal cancer cells. When tumors reached an average size of approximately 23 mm³ (referred to as day 0), mice were randomized into treatment groups (n=6/group) and treated via IP injections with either PBS, 0.03 mg/kg of WT IL-12 Fc, or an equimolar amount of a specific targeted IL-12-ABD fusion protein (mono-Y36E-Hyhel10, mono-Y36E-F12/3, mono-Y36E-OX86, mono-Y36E-Tabby107, mono-Y36E-KY1044, or mono-Y36E-VHH120). As an additional comparison, one group of mice were treated with a ten-fold higher dose (equivalent to 0.3 mg/kg of WT TL-12 Fc) of mono-Y36E-Hyhel10. Five days after treatment, mice were euthanized, and various tissues were processed for analysis. Briefly, blood samples were collected in EDTA tubes and centrifuged for 10 minutes at 4° C. to allow for retrieval of plasma. Spleens were either dissociated in PBS buffer for 2 hours at 37° C. prior to collection of the supernatant (for cytokine analysis), or passed through a 70 μm strainer, resuspended in red cell lysis buffer for 1 minute at room temperature, washed in FACS buffer, and subsequently resuspended in Fc block buffer containing unconjugated anti-mouse CD16/CD32, BioXCell (for flow cytometry). Tumors were either processed in the same manner as spleens (for cytokine analysis), or treated with 1 mg/mL collagenase IV and 0.1 mg/mL DNase for 20 minutes at 37° C. prior to being washed in ice-cold RPMI media containing 10% FCS, followed by passage through a 70 μm strainer and resuspension in Fc block buffer containing unconjugated anti-mouse CD16/CD32, BioXCell (for flow cytometry).

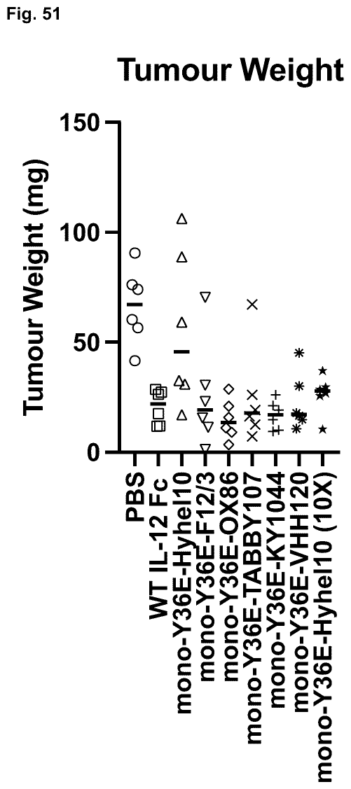

Data pertaining to tumor weight as measured on day 5 is depicted in FIG. 51. Even at this early time point, mice in all the treatment groups exhibited smaller tumors on average than mice that received PBS. Overall, tumor sizes were comparable between mice that received WT IL-12 Fe, each of the targeted IL-12 ABD fusion proteins, and the 10-fold higher dose of mono-Y36E-Hyhel10, with the tumor sizes of mice that received the equimolar dose of mono-Y36E-Hyhel10 exhibiting a greater spread in tumor size. Thus, any observed differences in tumor infiltrating lymphocytes were unlikely to be due to variability in tumor size amongst the different treatment groups.

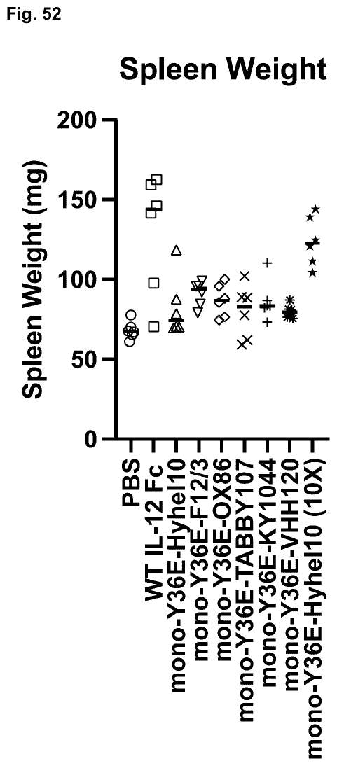

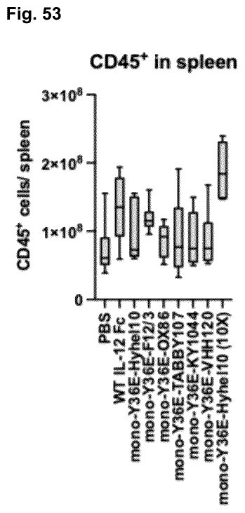

Recruitment and/or expansion of immune cells in peripheral organs is a hallmark of toxicity. Accordingly, spleen weights were measured on day 5, the data for which is presented in FIG. 52. Indeed, mice treated with WT IL-12 Fc (mean=129.6±37.2 mg) and the 10-fold higher dose of mono-Y36E-Hyhel10 (mean=124.1±15.4 mg) exhibited markedly enlarged spleen sizes relative to PBS treated mice (mean=68.2±5.6 mg). However, mice treated with equimolar doses of the non-targeted mono-Y36E-Hyhel10 or each of the targeted 11-12 ABD fusion proteins exhibited only marginal increases in spleen weight (group means ranging from 79.7-91.1 mg). In general, spleen sizes were correlated with the frequency of CD45⁺ cells within the spleens, as depicted in FIG. 53.

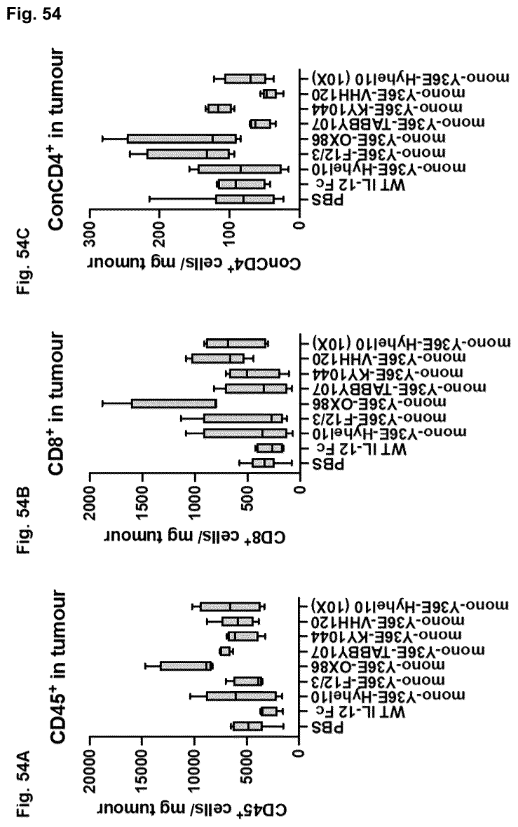

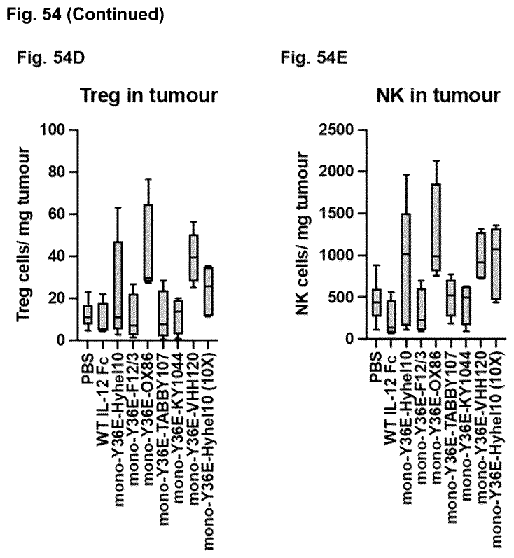

In clinical studies, high levels of immune infiltrate within ‘hot’ tumors are typically correlated with a favorable outcome relative to less infiltrated ‘cold’ tumors. To investigate if any of the test articles impacted the tumor immune infiltrate, the number and phenotype of CD45⁺ lymphocytes within the tumor was quantified using flow cytometry (FIG. 54). While most of the test articles resulted in similar amounts of CD45⁺ lymphocytes within tumors relative to the PBS control, mice treated with the OX40-targeted IL-12 ABD fusion protein (mono-Y36E-OX86) exhibited an approximately two-fold increase in CD45⁺ lymphocytes within their tumors. This increase was not attributed to any single cell type. Rather, treatment with mono-Y36E-OX86 broadly increased the representation of CD8⁺ T cells, conventional CD4⁺ T cells, NK cells, and Treg cells within tumors.

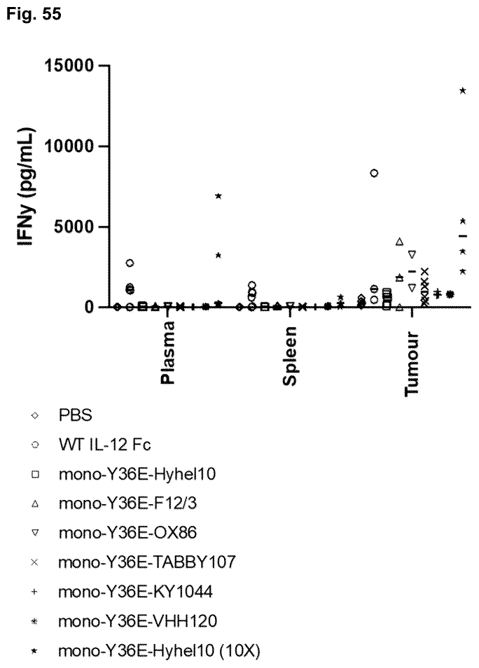

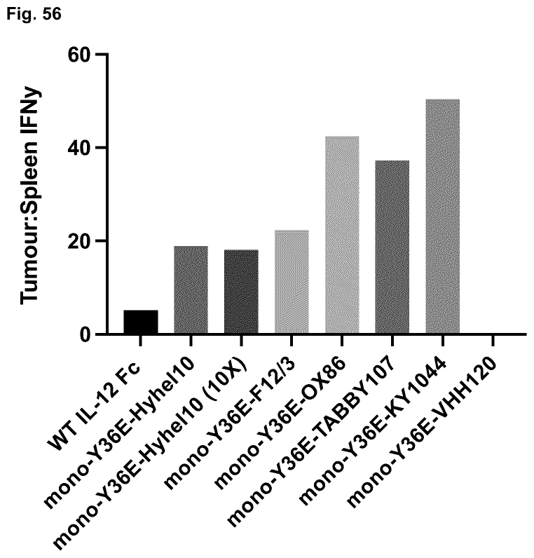

IFNγ levels in different tissues were quantified using an AlphaLISA High Performance Mouse IFNγ Detection Kit (Perkin Elmer), the results of which are depicted in FIG. 55. Overall, mice treated with mono-Y36E-Hyhel10 and each of the targeted IL-12 ABD fusion proteins exhibited considerably lower IFNγ levels in peripheral organs (e.g., plasma and spleen) than the equimolar dose of WT IL-12 Fc. However, within the tumor, mono-Y36E-F12/3 (mean IFNγ=1992.5 pg/mL), mono-Y36E-OX86 (mean IFNγ=2231.4 pg/mL) and mono-Y36E-TABBY107 (mean IFNγ=1066.9 pg/mL) exhibited elevated IFNγ levels compared to the equimolar dose of mono-Y36E-Hyhel10 (mean IFNγ=525.2 pg/mL). To quantify the extent to which each ABD was capable of biasing IL-12 activity towards the tumor relative to peripheral organs, the tumor:spleen ratio of IFNγ was plotted (FIG. 56). This analysis demonstrated that the non-targeted reduced potency IL-12 variant (mono-Y36E-Hyhel10) exhibited a clear increase in tumor:spleen IFNγ compared to WT IL-12 Fc. While the PD-1 targeted IL-12 ABD fusion protein exhibited only a marginal increase in tumor:spleen IFNγ relative to mono-Y36E-Hyhel10, each of mono-Y36E-OX86, mono-Y36E-TABBY107 and mono-Y36E-KY1044 exhibited the highest tumor:spleen IFNγ (approximately double that of mono-Y36E-Hyhel10). Therefore, of the molecules tested, targeting of reduced potency IL-12 variants via ABD specific for OX40, 41BB and/or ICOS targeting is likely to provide the largest increase in therapeutic index.

### J. Example 10—In Vivo Assessment of Targeted IL-12-ABD Fusion Proteins in the A20 Model

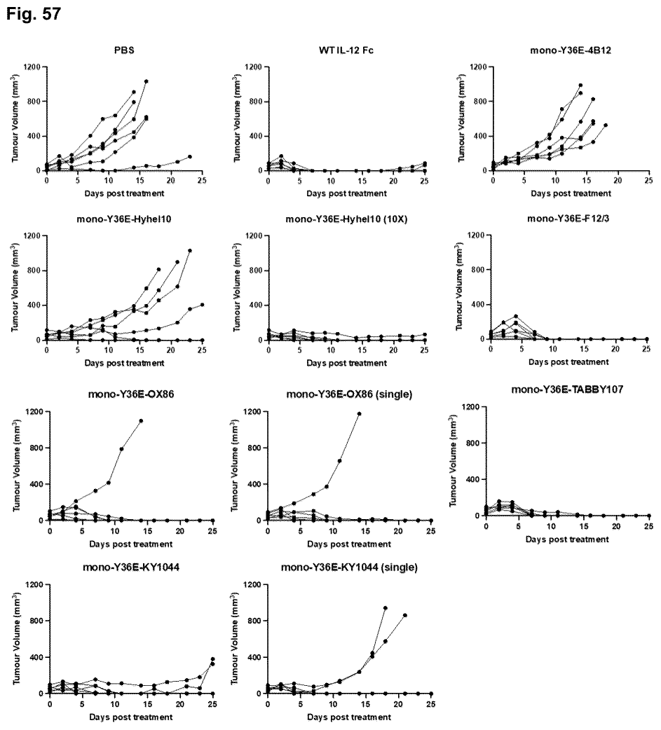

Next, selected targeted IL-12 ABD fusion proteins were assessed in BALB/C mice that were subcutaneously injected with 1×10⁶ A20 B cell lymphoma cells. When tumors reached a size of approximately 20 mm² (referred to as day 0), mice were randomized into treatment groups (n=6/group) and treated via IP injections on days 0, 7 and 14 with either PBS, 0.05 mg/kg of WT IL-12 Fc or IL-12 ABD fusion protein at a dose that was molar equivalent to 0.1 mg/kg of WT IL-12 Fc. Of the IL-12 ABD fusion proteins, a CCR7-targeted IL-12 fusion protein was also assessed, wherein the IL-12 p35 subunit harbored a Y36E mutation and was fused to the non-blocking anti-mouse CCR7 clone 4B12 (BioLegend). As an additional comparison, one group of mice was treated with a ten-fold higher dose (molar equivalent to 1 mg/kg of WT IL-12 Fc) of non-targeted IL-12 ABD fusion protein (mono-Y36E-Hyhel10). Tumor measurements were performed three times per week and tumor growth curves are depicted in FIG. 57.

Overall, tumors in PBS-treated mice grew in an uncontrolled manner, whereas treatment with 0.1 mg/kg of mono-Y36E-Hyhel10 resulted in a partial response, whereby only two out of six mice exhibited tumor regression. Mice treated with an equivalent dose of PD-1-targeted (mono-Y36E-F12/3, six out of six mice exhibiting complete response), 41BB-targeted (mono-Y36E-TABBY107, six out of six mice exhibiting complete response), ICOS-targeted (mono-Y36E-KY1044, four out of six mice exhibiting complete response), or OX40-targeted (mono-Y36E-OX86, five out of six mice exhibiting complete response) IL-12-ABD resulted in potent anti-tumor activity that was similar to that observed with the ten-fold higher molar equivalent dose of mono-Y36E-Hyhel10. In contrast, mice treated with CCR7-targeted IL-12 ABD fusion protein (mono-Y36E-4B12) exhibited uncontrolled tumor growth, similar to that observed in the PBS treated mice. These data demonstrate that the identity of ABD that enhance IL-12 activity is not obvious nor predictable.

Within the same experiment, certain groups of mice were treated with only a single dose (on day 0) with OX40-targeted (mono-Y36E-OX86 single) or ICOS-targeted (mono-Y36E-KY1044 single) IL-12 ABD fusion proteins. Surprisingly, the tumor growth in mice treated with a single dose of mono-Y36E-OX86 and mono-Y36E-KY1044 was very similar to that observed in mice treated with weekly doses of the same test articles. Moreover, mice that exhibited a complete response following a single dose of mono-Y36E-OX86 or mono-Y36E-KY1044 maintained the complete response over the time course of the experiment. This contrasted with mice treated weekly with WT IL-12 Fc, where two of the six mice initially exhibited a complete response, but eventually relapsed and exhibited tumor outgrowth.

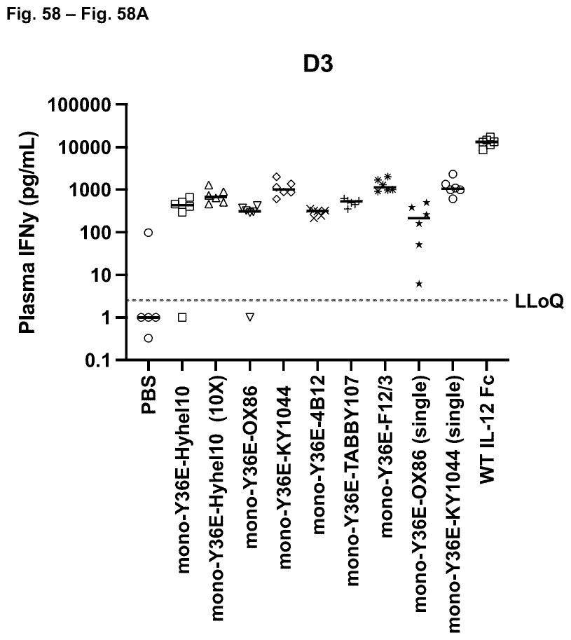

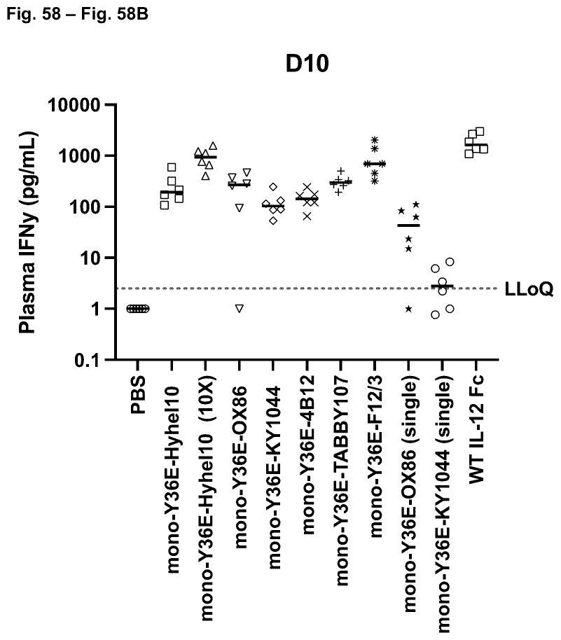

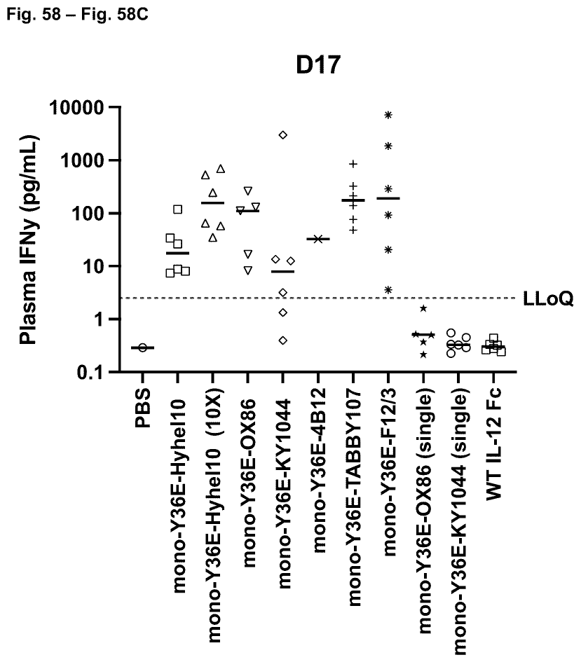

Plasma IFNγ levels were quantified three days after each treatment (on days 3, 10 and 17) using an AlphaLISA High Performance Mouse IFNγ Detection Kit (Perkin Elmer), the data for which is shown in FIG. 58. On day 3, mice treated WT Il-12 Fc exhibited high levels of plasma IFNγ (mean 13,131 pg/mL). Subsequently (on day 7), this group of mice developed significant toxicity that warranted cessation of treatment prior to the second dose. While IFNγ levels in mice treated with IL-12 ABD fusion proteins were considerably lower than those observed in WT IL-12 Fc treated mice, some notable differences were evident. Notably, ICOS-targeted and PD-1-targeted IL-12 ABD fusion proteins exhibited higher IFNγ levels (mean=1148 μg/mL and 1323 pg/mL, respectively) than the equivalent dose of the hyhel-10 targeted control (mean=391 pg/mL). In contrast, IFNγ levels in mice treated with the OX40-targeted IL-12 ABD fusion protein (mean=286 pg/mL) were similar to that observed for the equivalent dose of the hyhel-10 targeted control. Measurements at later time points (days 10 and 17) indicated that the pharmacodynamic response to mono-Y36E-OX86 was relatively sustained, but diminished rapidly for mono-Y36E-KY1044, both in the single and multi-dose treated animals.

### K. Example 11—Expanded Study of ICOS-, OX40- and 41BB-Targeted IL-12 ABD Fusion Proteins in MC38 Model

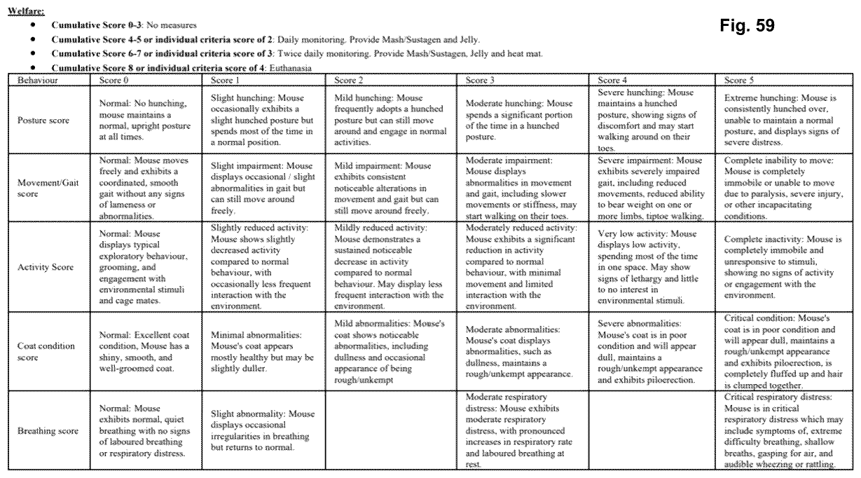

Next, different doses of selected IL-12 ABD fusion proteins were tested in the MC38 tumor model using a similar experimental workflow as described in Example 8. Although weekly dosing was potentially not required for full anti-tumor activity, a weekly dosing regimen was implemented to allow an assessment of the safety profile of each test article. Toxicity was assessed via measurement of plasma IFNγ levels on days 3, 10 and 17, as well as via a welfare monitoring system, as depicted in FIG. 59.

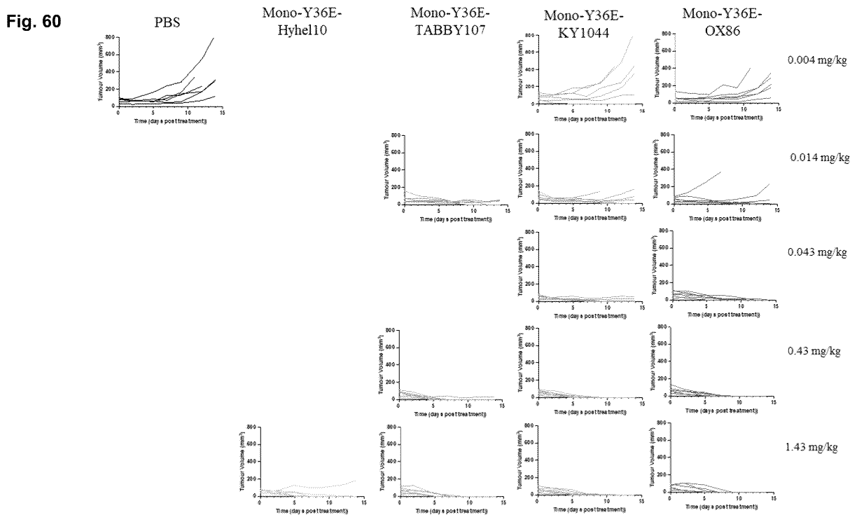

As evident in the tumor growth curves (shown in FIG. 60), each of the 41BB-, ICOS- and OX40-targeted IL-12 ABD fusion proteins exhibited potent anti-tumor activity, with reductions in tumor growth relative to the PBS control being observed at doses as low as 0.014 mg/kg. At the highest dose tested (1.43 mg/kg), 100% of mice treated with mono-Y36E-TABBY107, mono-Y36E-KY1044, and mono-Y36E-OX86 exhibited complete responses after 14 days of treatment. Complete responses were also observed in 100% of mice treated with 0.43 mg/kg and 0.043 mg/kg of mono-Y36E-OX86, and 0.43 mg/kg of mono-Y36E-KY1044.

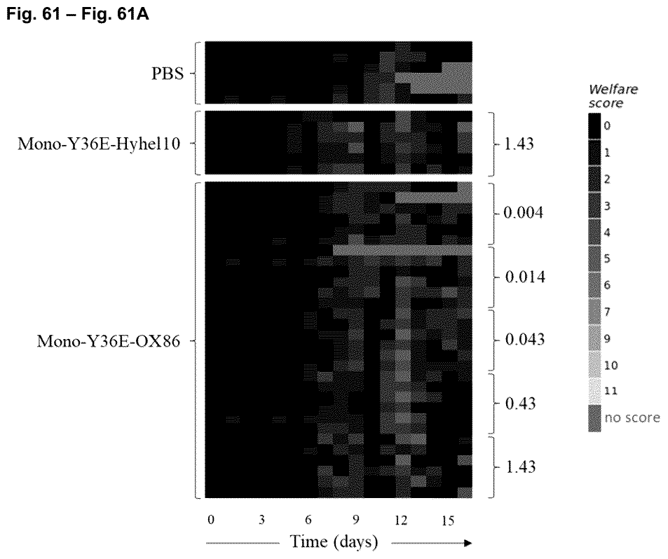

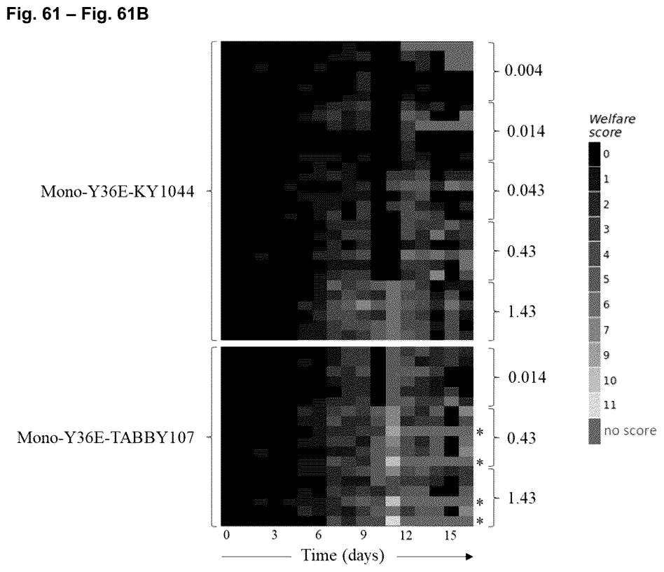

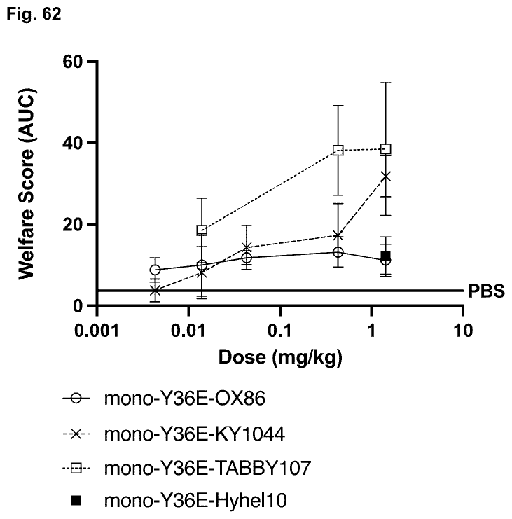

Welfare scores for individual mice are plotted in FIG. 61 and quantified for each group of mice in FIG. 62. Welfare score was quantified by calculation of area-under-the curve (AUC) using a standard linear interpolation between data points. Where mice were removed from the experiment (for example, due to welfare), an order 0 extrapolation was applied, such that the final observation was extrapolated to a constant value for the remaining duration of the experiment. Surprisingly, the targeted IL-12 ABD fusion proteins differed markedly in their capacity to induce toxicity. While mono-Y36E-OX86 was well tolerated across all dose levels (0.004-1.43 mg/kg) and did not appear to induce more pronounced toxicity than the mono-Y36E-Hyhel10 control, significant toxicity was evident in mice treated with higher doses of mono-Y36E-KY1044, and to a greater extent, mono-Y36E-TABBY107. Indeed, two of the six mice (33%) in groups treated with 0.43 mg/kg and 1.43 mg/kg of mono-Y36E-TABBY107 were euthanized due to treatment related toxicity.

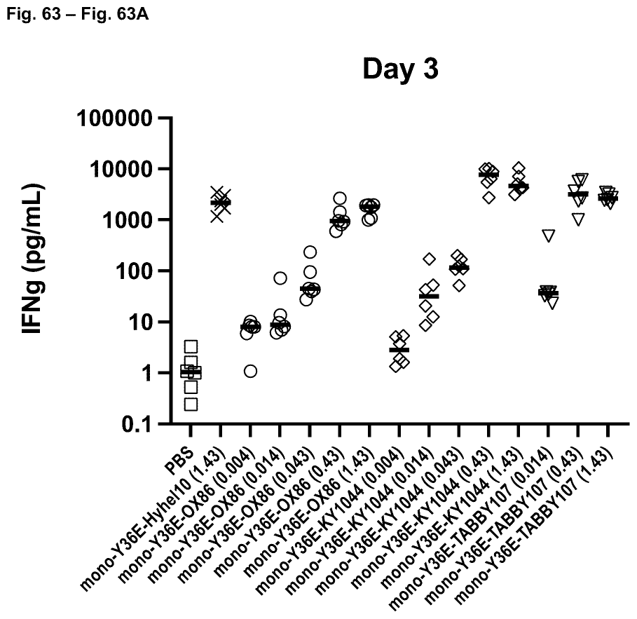

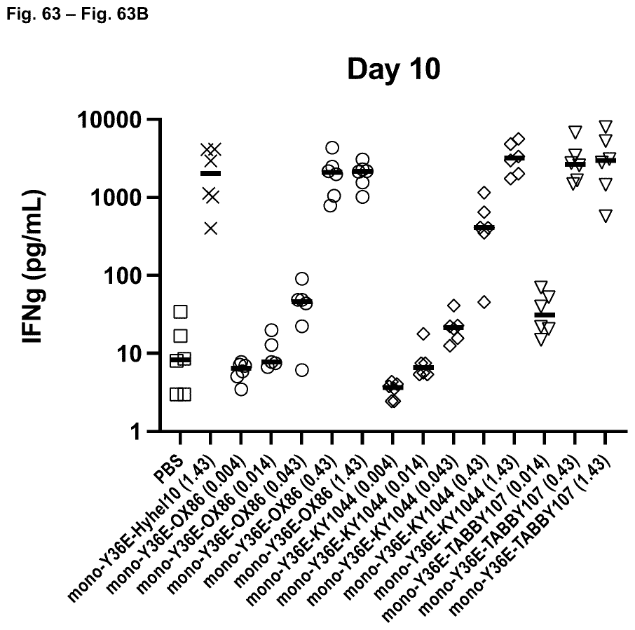

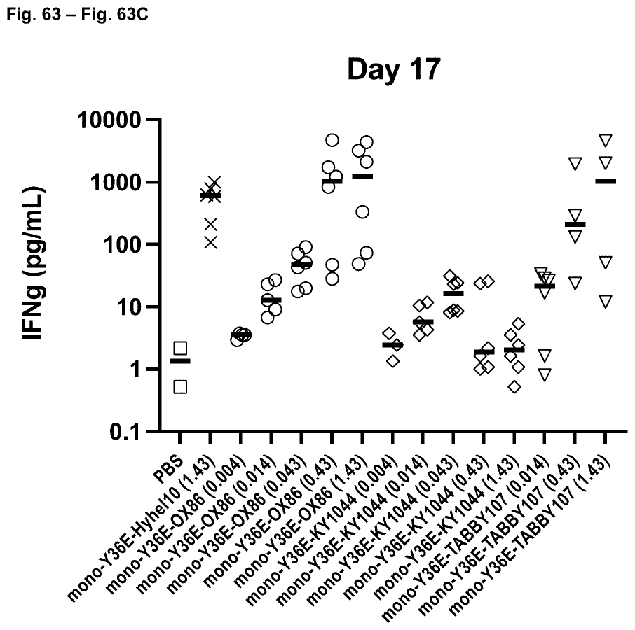

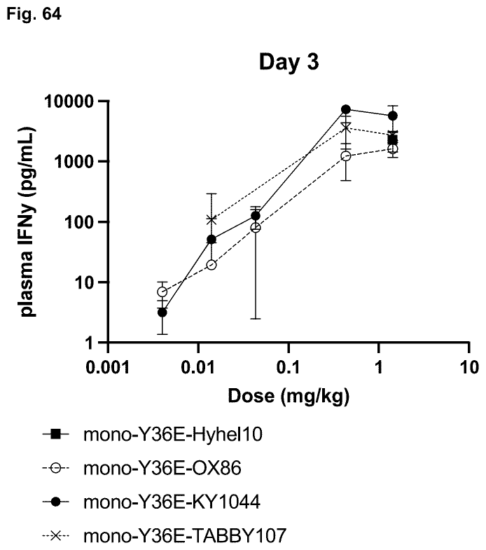

Plasma IFNγ levels were measured three days after each weekly treatment (on days 3, 10 and 17) and are depicted in FIGS. 63 and 64. On day 3 (when plasma IFNγ levels typically peak), each of the IL-12 ABD fusion proteins demonstrated a dose-dependent increase in plasma IFNγ. For mice treated with mono-Y36E-OX86 and mono-Y36E-TABBY107, plasma IFNγ levels were maintained until day 17, indicating these treatments triggered a sustained pharmacodynamic response. This was not the case for mice treated with mono-Y36E-KY1044, which exhibited considerably lower IFNγ on day 17. Commensurate with its improved safety profile, mono-Y36E-OX86 exhibited lower levels of IFNγ on day 3 relative to mono-Y36E-TABBY107 and mono-Y36E-KY1044 across almost all dose levels. Indeed, mono-Y36E-OX86 induced particularly low levels of IFNγ in the plasma on day 3 at doses that resulted in complete responses in 100% of mice (0.043 mg/kg dose, mean IFNγ 78.1 pg/mL). Thus, targeting reduced potency IL-12 variants via ABD specific for OX40 results in a greater improvement in therapeutic index relative to targeting via ABD specific for ICOS or 41BB.

### L. Example 12—OX40 Targeted IL-12-ABD Fusion Proteins as Monotherapy and in Combination with Anti-PD-1 Therapy in a Hard-to-Treat AT3 Model

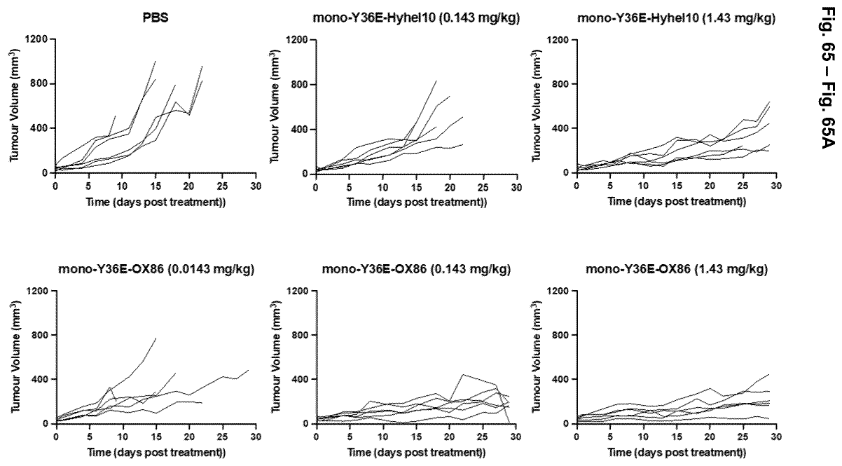

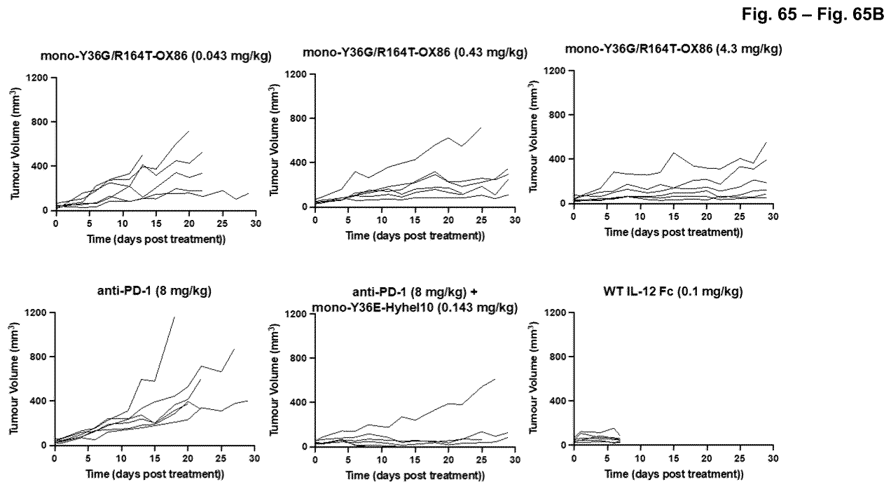

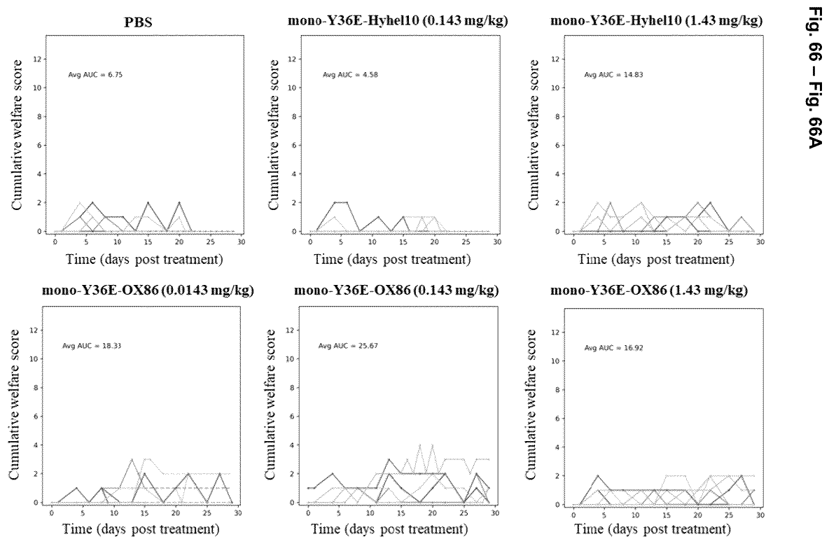

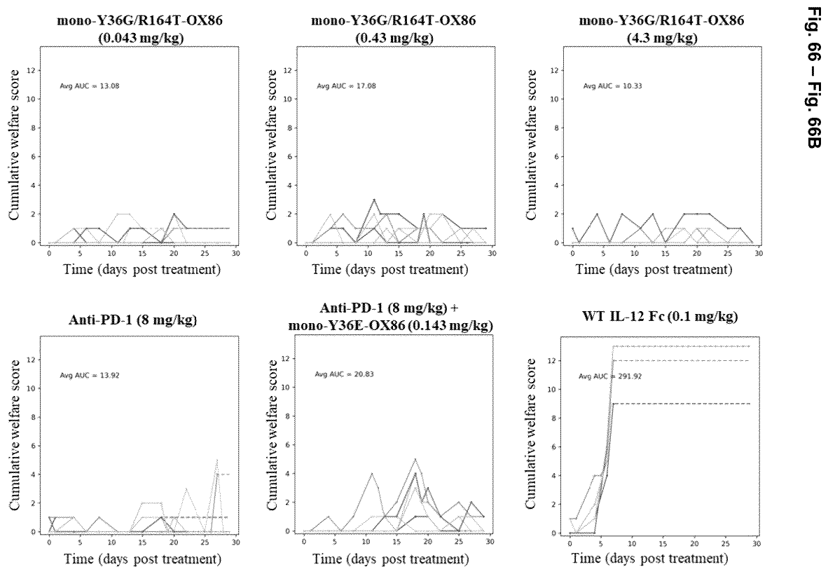

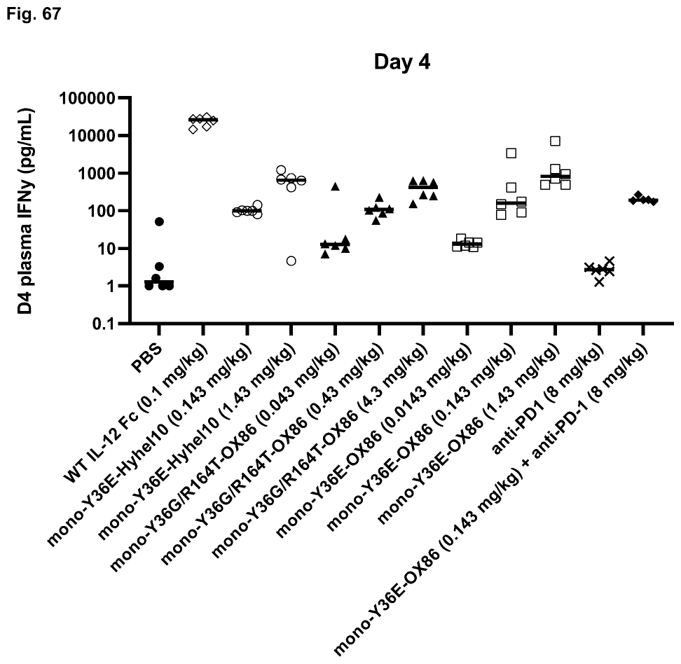

The anti-tumor activity of selected targeted IL-12 ABD fusion proteins were next assessed in C57BL/6 mice that were subcutaneously injected with 5×10⁶ AT3 mammary carcinoma cells. When tumors reached a size of approximately 20 mm² (referred to as day 0), mice were randomized into treatment groups (n=6/group) and treated via IP injections on days 0, 7, and 14 with either PBS, 0.1 mg/kg of WT IL-12 Fc or various concentrations of OX40-targeted IL-12 ABD fusion proteins that harbored either a Y36E single mutation or a Y36G/R164T double mutation within the IL-12 p35 subunit. Additional groups of mice were treated with 8 mg/kg of anti-PD-1 mAb (clone RMP1-14, BioXCell) on days 0, 4, and 8 as a monotherapy, or in mice that had also received treatment with an OX40-targeted IL-12 ABD fusion protein. Tumor measurements were performed three times per week and tumor growth curves are depicted in FIG. 65. Toxicity was assessed using the welfare monitoring system depicted in FIG. 59 and via measurement of plasma IFNγ levels on day 4, the results of which are shown in FIG. 66 and FIG. 67, respectively. Treatment with 0.1 mg/kg WT IL-12 Fc resulted in severe toxicity, with all treated mice being euthanized due to high welfare scores prior to receiving a second treatment dose.

In line with the observed toxicity, this group of mice exhibited extremely high levels of IFNγ (23,752±6,397 pg/mL) in the plasma on day 4. In contrast, treatment with non-targeted or OX40 targeted IL-12 ABD fusion proteins did not result in welfare issues, even at substantially higher doses (up to 1.43 mg/kg for mono-Y36E-Hyhel10 and mono-Y36E-OX40, and up to 4.3 mg/kg of mono-Y36G/R164T-OX86). While mono-Y36E-Hyhel10 significantly reduced tumor growth only at a relatively high dose (1.43 mg/kg), the OX40-targeted IL-12 ABD fusion proteins demonstrated potent anti-tumor activity at doses as low as 0.143 mg/kg (for mono-Y36E-OX86) or 0.43 mg/kg (for mono-Y36G/R164T-OX86). Notably, mice treated with experimentally effective doses of OX40 targeted IL-12 ABD fusion proteins exhibited lower levels of IFNγ in the plasma on day 4 (median of 162 pg/mL for 0.143 mg/kg of mono-Y36E-OX86 and 108 pg/mL for 0.43 mg/kg of mono-Y36G/R164T-OX86) relative to an experimentally effective dose of the non-targeted IL-12 ABD fusion protein (median of 656 μg/mL for 1.43 mg/kg of mono-Y36E-Hyhel10), indicating that OX40 targeting results in a more favorable therapeutic index.

The AT3 model is typically considered refractory to immunotherapy. Indeed, treatment with 8 mg/kg of anti-PD-1 mAb resulted in only a marginal reduction in average tumor volume compared to PBS-treated mice. However, mice treated with a combination of 0.143 mg/kg mono-Y36E-OX86 and anti-PD-1 mAb exhibited strong anti-tumor activity that was beyond that achieved with treatment of either agent alone. Importantly, combination treatment was well tolerated as judged by both welfare score and plasma IFNγ levels relative to treatment with either agent alone. These data demonstrate the potential utility of combining OX40-targeted, reduced potency IL-12 ABD fusion proteins with checkpoint blockade for the treatment of cancer.

### M. Example 13—PK/PD Study of an Exemplary OX40 Targeted IL-12 ABD Fusion Protein

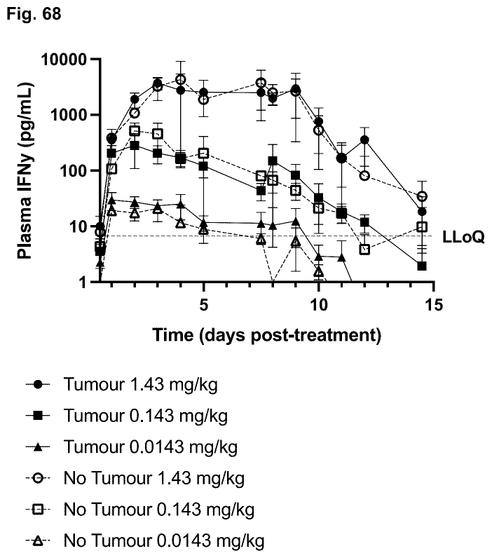

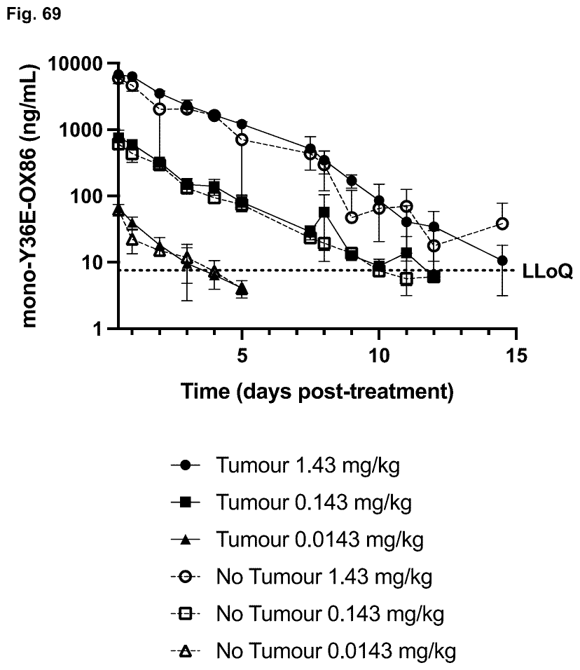

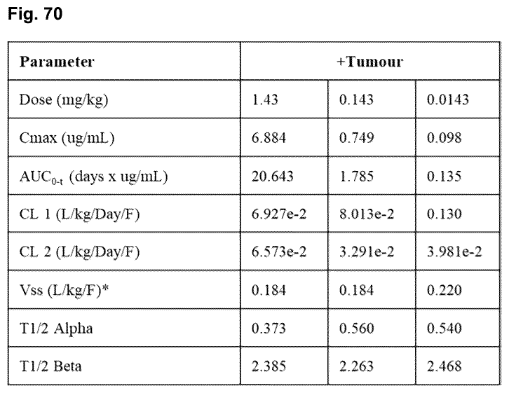

The pharmacokinetic (PK) profile and pharmacodynamic (PD) parameters of mono-Y36E-OX86 was assessed at three different dose levels (0.014, 0.143, and 1.43 mg/kg) in tumor free C57BL/6 mice and C57BL/6 mice that were subcutaneously injected with MC-38 tumors (average tumor size upon treatment was 22 mm²). Mice (n=3 per group) were treated with a single dose of mono-Y36E-OX86 on day 0 and blood was collected at various time points, processed into plasma, and analyzed using an AlphaLISA High Performance mouse IFNγ Detection Kit (Perkin Elmer), the data for which is depicted in FIG. 68. Plasma mono-Y36E-OX86 levels were also determined using a sandwich ELISA that utilized an anti-mouse IL-12 p70 antibody (Unconjugated, BioXCell) as a capture reagent and an anti-mouse IgG1 HRP conjugate (Southern Biotech) for detection. PK profiles and calculated PK parameters (modelled using a 2-compartment extravascular analysis in PK solver) are depicted in FIGS. 69 and 70, respectively.

Overall, mono-Y36E-OX86 exhibited a dose proportional PK profile, indicating that target-mediated clearance did not have a major impact on drug clearance at the doses tested. The half-life of mono-Y36E-OX86 during the terminal phase was approximately 2.4 days, which compares favorably with the 30-hour half-life reported for WT IL-12 Fc (see, e.g., Gutierrez, E. et al. Med (2023): 4, 326-340, the contents of which are herein incorporated by reference in its entirety, with particularity for its discussion of IL-12). A dose proportional increase in plasma IFNγ levels was evident for 7 days (0.0143 mg/kg dose), 12 days (0.143 mg/kg dose) or 14 days (1.43 mg/kg dose), with peak levels occurring between days 2-4 days post treatment.

### N. Example 14—Mouse OX40-Targeted IL-12 ABD Fusion Proteins do not Trigger OX40 Signaling

Agonistic OX40 antibodies have demonstrated robust anti-tumor activity in mouse models through a mechanism that is dependent on FcγR-mediated clustering of OX40 subunits (see, e.g., Yeung, Y. A. et al. Cell Reports (2019): 27, 3117-3123, incorporated by reference in its entirety). While the Fc domain of mono-Y36E-OX86 harbored mutations to silence Fc effector function, it was conceivable that binding to IL-12R could inadvertently lead to clustering of OX40, thereby triggering OX40 signaling. Indeed, a PD-L1/OX40 bispecific antibody has been reported to trigger OX40 signaling in a PD-L1-dependent manner (see, e.g., Liu, J. et al. Mol Cancer Ther (2020): 19, 2564-74, incorporated by reference in its entirety).

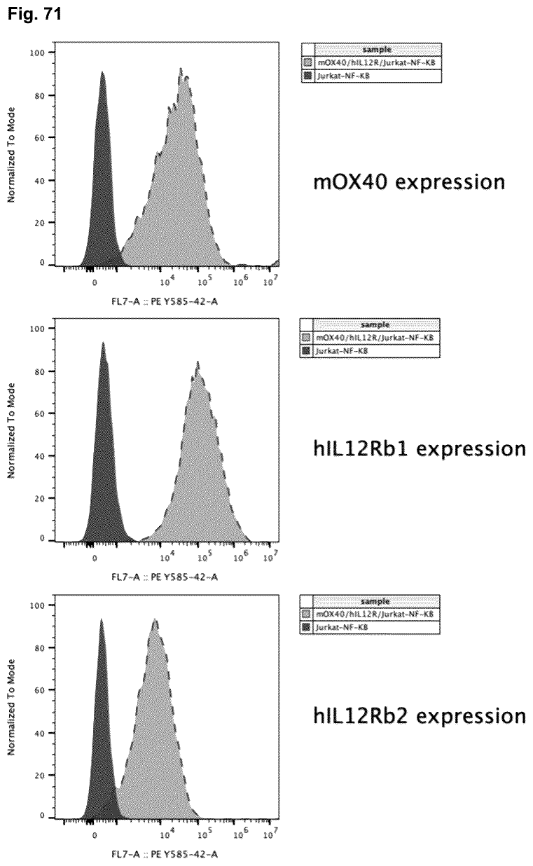

Activation of OX40 by OX40L or agonistic antibodies triggers intracellular activation of the nuclear factor kappa-light-chain-enhancer of activated B cells (NF-κB) signaling pathway. Thus, to investigate whether mono-Y36E-OX86 was able to induce OX40 signaling, a Jurkat reporter cell line was constructed whereby activation of NF-κB resulted in expression of green fluorescent protein (GFP), which could readily be detected using flow cytometry. Jurkat reporter cells were transduced with full-length mouse OX40 and human IL-12R(31/IL-12Rβ2 subunits that lacked cytoplasmic domains (note that mouse IL-12 cross reacts with human IL-12R) to produce the mOX40/hIL-12R/Jurkat-NF-κB reporter cell line. The expression of OX40, IL-12R(31, and IL-12Rβ2 on the mOX40/hIL-12R/Jurkat-NF-κB reporter cells is depicted in FIG. 71.

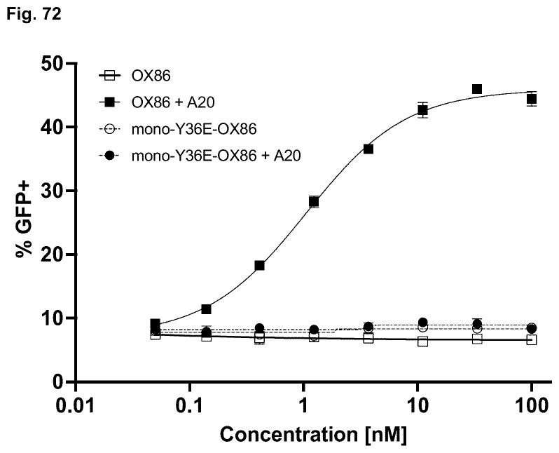

For the assay, 30,000 mOX40/hIL-12R/Jurkat-NF-κB reporter cells were incubated overnight with varying concentrations of bivalent OX86 antibody (mouse IgG2a format) or mono-Y36E-OX86 in the presence or absence of Deep Red-stained A20 mouse B cell lymphoma cells that had been transduced to express the high affinity mouse FcγRI. The following day, cells were washed in MACS buffer and analyzed by flow cytometry, the results of which are depicted in FIG. 72. As expected, the bivalent OX86 antibody in IgG2 format induced GFP expression in mOX40/hIL-12R/Jurkat-NF-κB reporter cells in a dose-dependent manner, but only in the presence of A20 cells. In contrast, mono-Y36E-OX86 did not induce GFP expression in mOX40/hIL-12R/Jurkat-NF-κB reporter cells even in the presence of A20 cells. These data indicates that binding of mono-Y36E-OX86 to IL-12R on the same cells (i.e., in cis) or on adjacent cells (i.e., in trans) does not induce OX40 signaling. Moreover, mono-Y36E-OX86 does not induce OX40 signaling in the presence of cells that express high affinity FcγR. Accordingly, the improved anti-tumor activity observed in mice treated with mono-Y36E-OX86 relative to untargeted mono-Y36E-Hyhel10 was not due to signaling downstream of OX40.

### O. Example 15—Mouse OX40-Targeting Enhances IL-12 Activity on Cells that Express Sufficient Amounts of OX40

While OX40 targeting did not enhance IL-12 activity on activated C57BL/6 mouse cells (see, e.g., Example 5), this could have been due to technical difficulties associated with the induction of high OX40 expression on mouse cells in vitro. To further investigate this, the activity of OX40 targeted IL-12 ABD fusion protein was assessed in T cells derived from OT-II transgenic mice. OT-II transgenic mice express a TCR that pairs with the CD4 coreceptor and is specific for the MHC-II molecule I-A^{b} presenting a peptide encompassing residues 323-339 of chicken ovalbumin (I-A^{b-OVA}). OT-II mice are observed to have a higher proportion of CD4⁺ T cells (relative to CD8⁺ T cells) in their peripheral organs, and these can readily be activated/expanded upon culture with OVA³²³⁻³³⁹ peptide.

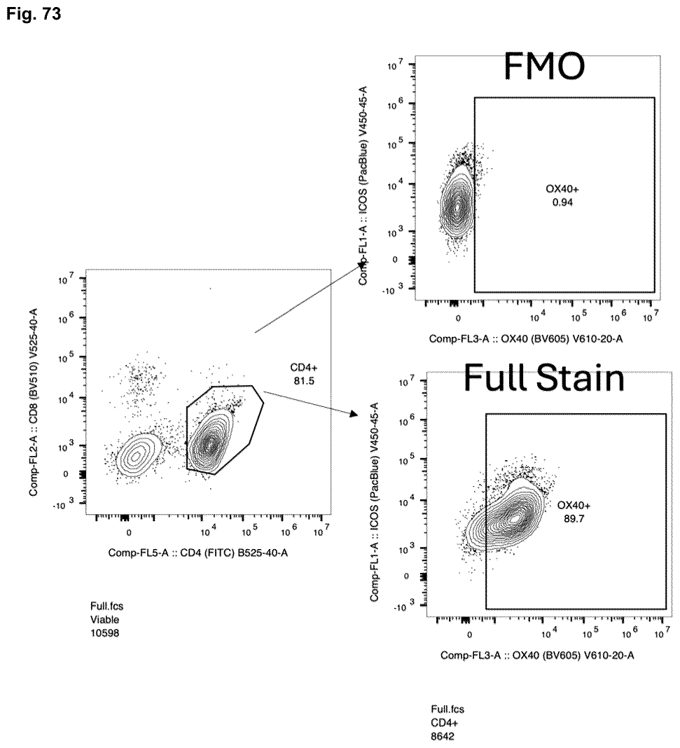

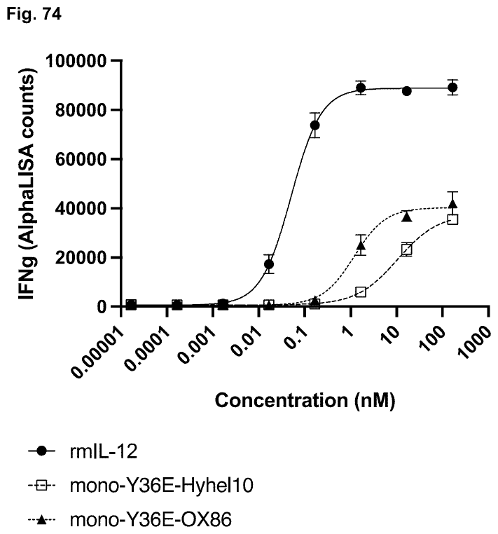

Splenocytes from OT-II mice were resuspended in T-cell media and activated for three days in the presence of 40 nM OVA³²³⁻³³⁹ peptide and 100 U/mL recombinant human IL-2 (Miltenyi Biotec). Following activation, cells were stained with a panel of antibodies including anti-mouse CD134 (BV605, BD Biosciences), anti-mouse CD278 (PB, BioLegend), anti-mouse CD8α, (BV510, BioLegend), anti-mouse CD4 (FITC, BD Biosciences), and the live/dead marker 7AAD, and analyzed by flow cytometry, the results of which are shown in FIG. 73. Following activation, 81.5% of the cells were CD4⁺ T cells, and of these, 89.7% expressed OX40. Purified cells were incubated for 2 days in the presence of 50 U/mL human IL-2 and varying concentrations of commercially available recombinant mouse IL-12 (rmIL-12, BioLegend), OX40-targeted IL-12-ABD fusion protein or non-targeted IL-12-ABD fusion protein. IFNγ within the supernatant were measured using an AlphaLISA High Performance mouse IFNγ Detection Kit (Perkin Elmer), and are depicted in FIG. 74. Here, mono-Y36E-OX86 demonstrated more potent induction of IFNγ relative to the non-targeted mono-Y36E-Hyhel10 control, although under these conditions, mono-Y36E-OX86 was not as potent as recombinant mouse IL-12. These data demonstrate that OX40-targeting can enhance the activity of reduced potency IL-12 on cell populations that express OX40.

### P. Example 16—Binding Affinity of Antibody Clones to Human and Cynomolgus OX40

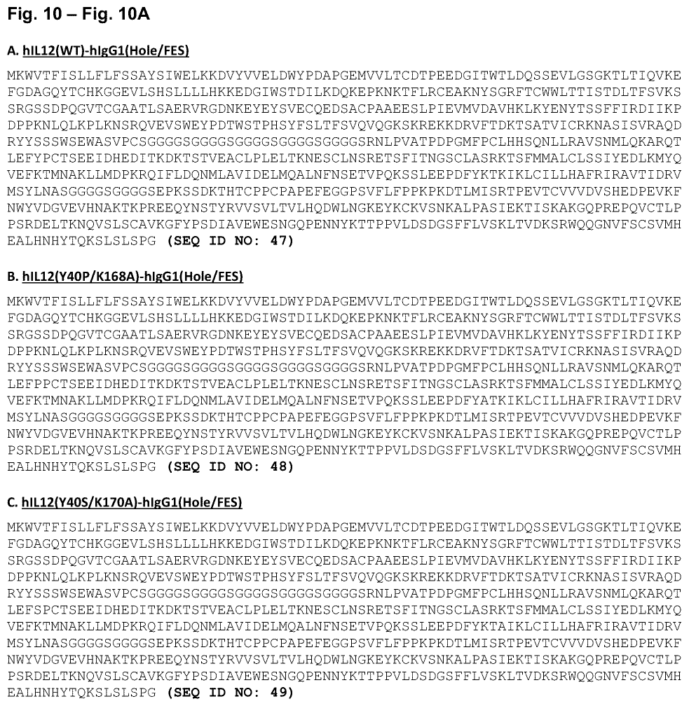

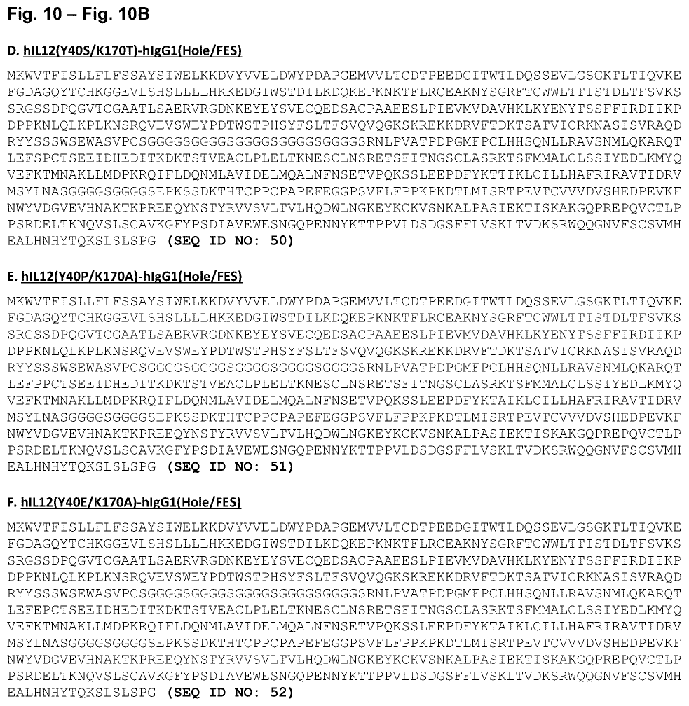

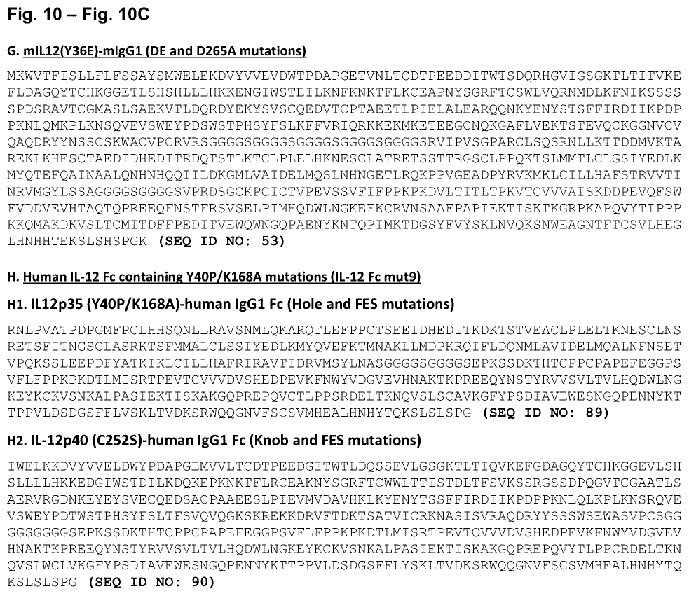

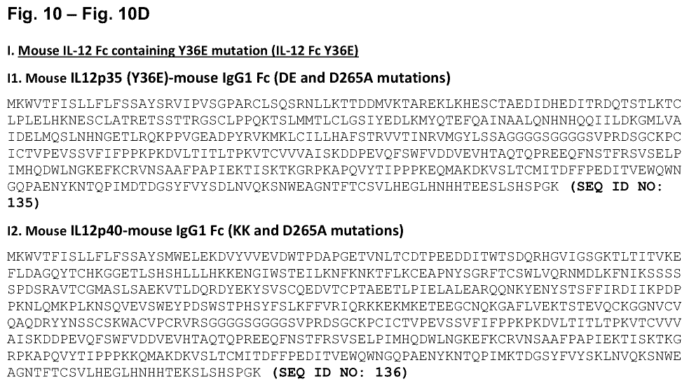

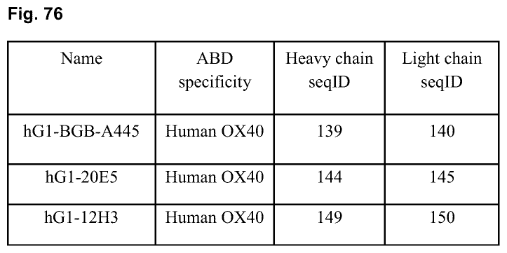

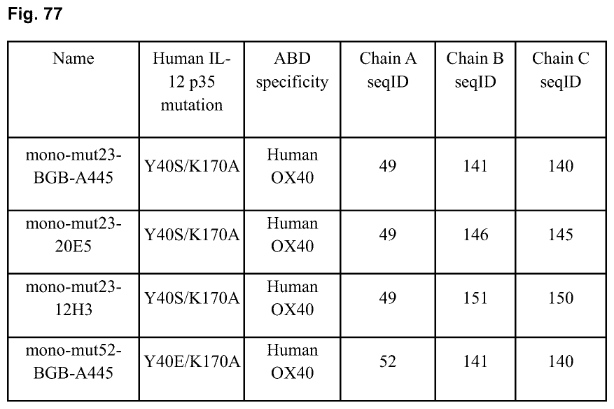

Next, we assessed the binding of three anti-human OX40 clones (BGB-A445, 20E5 and 12H3) to Jurkat-NF-κB reporter cells that has been transduced with either human (hOX40-Jurkat-NF-κB) or cynomolgus monkey OX40 (cOX40-Jurkat-NF-κB). Each of the anti-OX40 clones were expressed as traditional monospecific, bivalent antibodies (human IgG1 isotype) and as bispecific, monovalent IL-12 ABD fusion proteins as described in Example 1 and depicted in FIG. 18A. Sequence information for the full-length bivalent antibodies and OX40-targeted IL-12-ABD fusion proteins are provided in FIGS. 10 and 75-77.

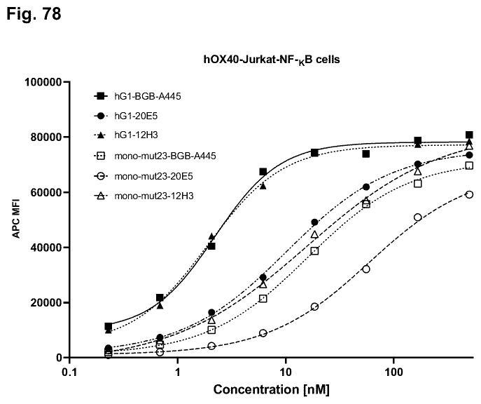

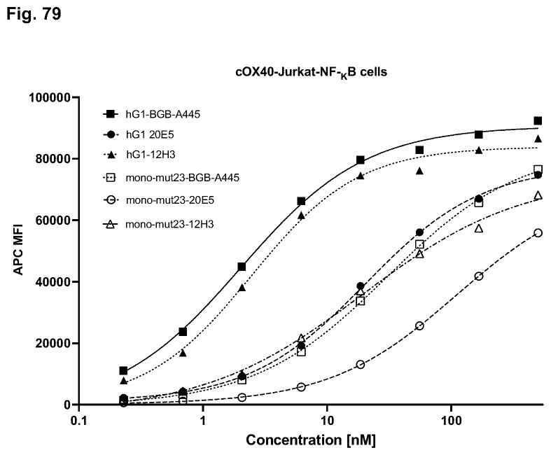

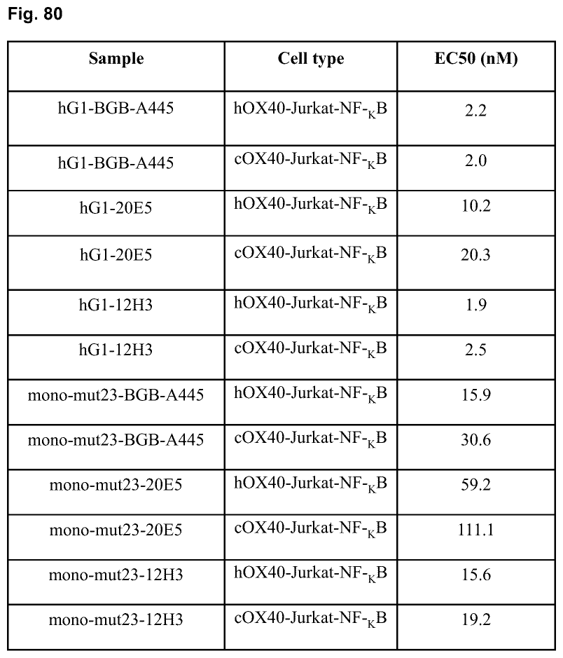

Thirty thousand (30,000) hOX40-Jurkat-NF-κB or cOX40-Jurkat-NF-κB cells were incubated with varying concentrations of full-length bivalent antibody or selected OX40-targeted IL-12-ABD fusion protein for 20 minutes on ice, washed with MACS buffer, and stained with an APC-conjugated polyclonal goat anti-human IgG Fc F(ab′)2 fragment (Jackson Immuno Research, catalogue number 109-136-098). Stained hOX40-Jurkat-NF-κB and cOX40-Jurkat-NF-κB cells were analyzed by flow cytometry using a CytoFLEX (Beckman Coulter), the results of which are depicted in FIGS. 78 and 79, respectively, and summarized in FIG. 80.

When produced in traditional IgG1 bivalent mAb format, each of the clones tested bound in a dose-dependent manner to hOX40-Jurkat-NF-κB cells with EC50 values in the low nM range (2.2 nM for BGB-A445, 10.2 nM for 20E5, and 1.9 nM for 12H3). As expected, IL-12 ABD fusion proteins wherein the ABD comprised BGB-A445, 20E5, or 12H3 also bound to hOX40-Jurkat-NF-κB cells, although with lower apparent affinity (approximately 5- to 8-fold higher EC50 values) due to the monovalent nature of the interaction. A similar binding profile was observed on cOX40-Jurkat-NF-κB cells, with all EC50 values measured being within 2-fold of the values observed on hOX40-Jurkat-NF-κB cells. These data indicate that BGB-A445, 20E5, and 12H3 clones bind to both human and cynomolgus OX40, and thus represent candidates that could be incorporated into an IL-12 ABD fusion protein.

### Q. Example 17—Sequence Optimization of 20E5 Clone

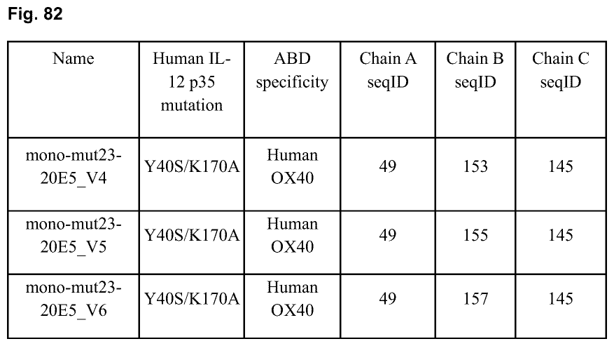

The heavy chain variable region of 20E5 contains an isomerization motif (D₅₆G₅₇) within the HCDR2 loop. To remove this potential manufacturing liability, three IL-12 ABD fusion proteins containing sequence optimized versions of 20E5 were produced, whereby D56 in HCDR2 was replaced with a glutamic acid (mono-mut23-20E5_V4) or alanine residue (mono-mut23-20E5_V5), or G57 was replaced with an alanine residue (mono-mut23-20E5_V6). Sequence information relating to the sequence optimized IL-12 ABD fusion proteins is provided in FIGS. 10, 81, and 82.

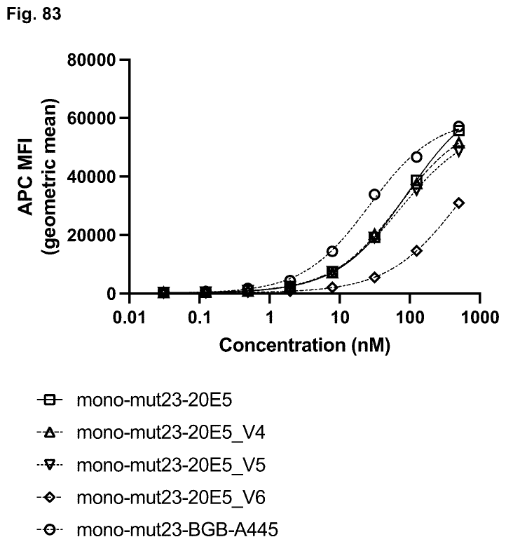

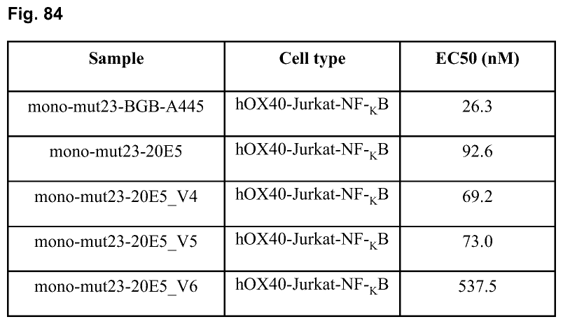

Binding of each of the sequence optimized IL-12 ABD fusion proteins to hOX40-Jurkat-NF-κB cells was performed as described in Example 16, the results of which are depicted in FIG. 83 and summarized in FIG. 84. The results of these experiments indicate that replacement of D56 with glutamic acid or alanine did not alter the binding affinity of the IL-12 ABD fusion protein to human OX40, whereas replacement of G57 with alanine reduced the binding affinity of the IL-12 ABD fusion protein to human OX40.

### R. Example 18—Sequence Optimization of 12H3 Clone

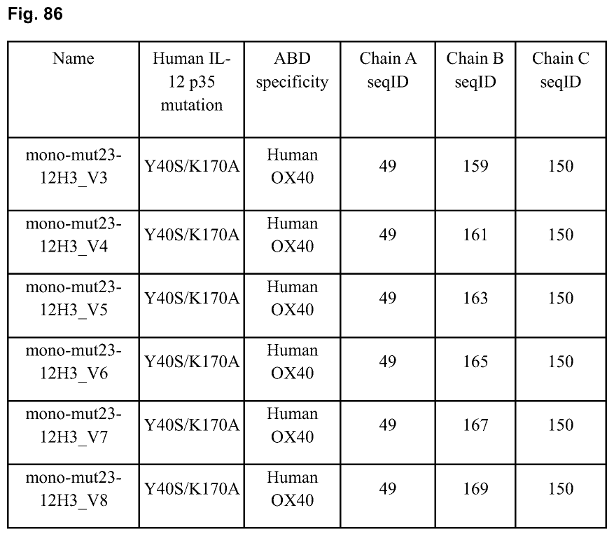

The heavy chain variable region of 12H3 contains a deamidation motif (N₅₅G₅₆) within the HCDR2 loop and a methionine residue (M99) that could represent a site for oxidation. To remove these potential manufacturing liabilities, six IL-12 ABD fusion proteins containing sequence optimized versions of 12H3 were produced, whereby N55 in HCDR2 was replaced with a glutamine (mono-mut23-12H3_V3) or alanine residue (mono-mut23-12H3_V4), G56 was replaced with an alanine residue (mono-mut23-12H3_V5), M99 was replaced with a leucine (mono-mut23-12H3_V6) or valine (mono-mut23-12H3_V7) residue, or a double substitution of N55Q and M99I was introduced (mono-mut23-12H3_V8). Sequence information relating to the sequence optimized IL-12 ABD fusion proteins is provided in FIGS. 10, 85, and 86.

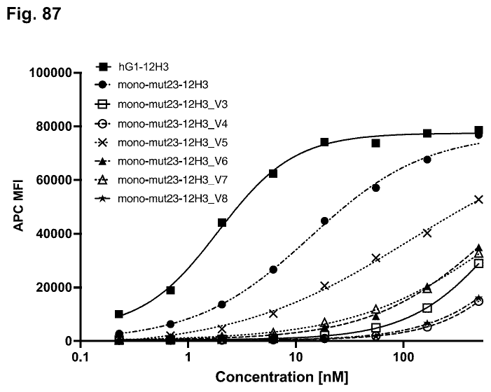

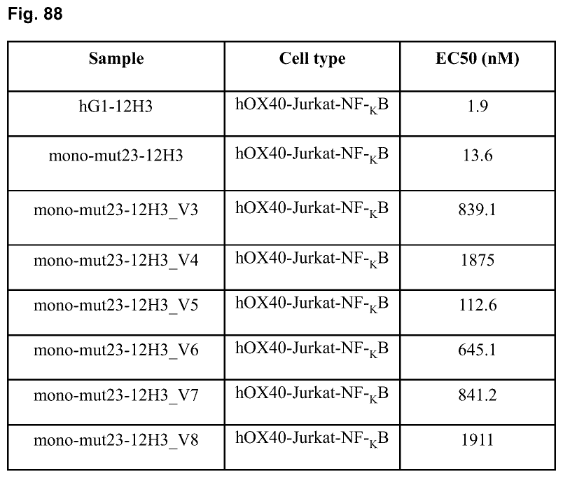

Binding of each of the sequence optimized IL-12 ABD fusion proteins to hOX40-Jurkat-NF-κB cells was performed as described in Example 16, the results of which are depicted in FIG. 87 and summarized in FIG. 88. The results of these experiments indicate that replacement of G56 with alanine resulted in a moderate reduction in apparent affinity (EC50 of 112.6 nM for mono-mut23-12H3_V5 compared to EC50 of 13.6 nM for mono-mut23-12H3), whereas substitution of M99 with leucine resulted in a greater reduction in apparent affinity (EC50 of 645.1 nM for mono-mut23-12H3_V6). All other substitutions tested resulted in very weak binding (EC50>800 nM) to hOX40-Jurkat-NF-κB cells.

### S. Example 19-20E5 and BGB-A445 Clones do not Compete with OX40L for OX40 Binding

OX40 is a co-stimulatory receptor that is expressed on certain immune cell lineages including activated CD4⁺ and CD8⁺ T cells, as well as Treg cells. The natural ligand of OX40, OX40L, is predominantly expressed on activated antigen presenting cells, such as, for example, dendritic cells, B cells, macrophages, and endothelial cells. Given that OX40 may encounter OX40L within the tumor microenvironment, it might be beneficial for an OX40-targeted IL-12 ABD fusion protein to bind to an epitope on OX40 that is different to what is bound by OX40L. By binding to an epitope that is distinct from the OX40L epitope, an IL-12 ABD fusion protein would not have to compete with endogenous OX40L for OX40 binding, and therefore could potentially achieve improved targeting within the tumor microenvironment, relative to an IL-12 ABD fusion protein that competed with OX40L for the OX40 binding site. Indeed, the OX86 clone that constitutes the OX40 binding arm within the mouse OX40-targeted IL-12 ABD fusion protein (for example, mono-Y36E-OX86) does not block mouse OX40L binding (see, e.g., Zhang et al. Cell Reports (2019): 27, 3117-3123, incorporated by reference in its entirety).

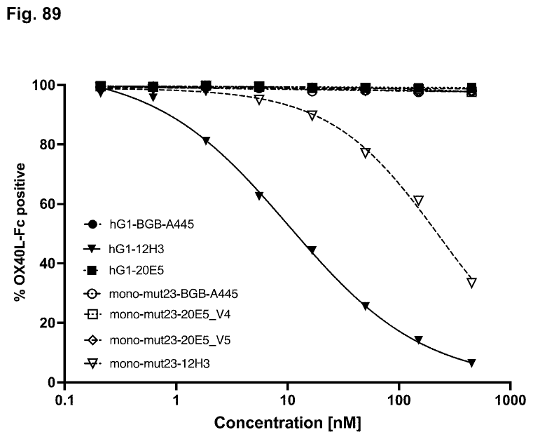

To investigate whether any of BGB-A445, 20E5, or 12H3 competed with OX40L for the OX40 binding site, we assessed the capacity of these clones, either in traditional IgG1 monospecific, bivalent mAb format or as bispecific, monovalent IL-12 ABD fusion proteins, to block binding of OX40L to hOX40-Jurkat-NF-κB cells. For the experiment, 30,000 hOX40-Jurkat-NF-κB cells were pre-incubated with 0-450 nM mAb or IL-12 ABD fusion protein for 10 minutes on ice prior to being stained for an additional 10 minutes on ice with 2.5 nM OX40L-Fc (Sino Biological, catalogue number 13127-H01H) that had been coupled to an A647 fluorophore using a Zenon Human IgG labelling kit (Invitrogen, catalogue number Z25408). Stained hOX40-Jurkat-NF-κB cells were analyzed by flow cytometry, and are depicted as percentage of OX40L-Fc positive cells within the OX40 positive gate (defined using a non-competing anti-OX40 mAb (clone Ber-ACT35, BioLegend catalogue number 350012) in FIG. 89.

Both test articles that contained a binding arm derived from the 12H3 clone inhibited OX40L-Fc binding to hOX40-Jurkat-NF-κB cells in a dose-dependent manner, indicating that 12H3 blocks OX40-OX40L binding. Further, a test article that contained two 12H3 binding arms (hG1-12H3) more potently inhibited OX40L-Fc binding (IC₅₀=10.3 nM) compared to a test article that contained a single 12H3 binding arm (mono-mut23-12H3, IC₅₀=227.2 nM). In contrast, test articles that contained either a single copy or two copies of BGB-A445 or 20E5 binding arm(s) did not interfere with OX40L binding even at concentrations that were well in excess (160-fold greater) of OX40L-Fc. These data indicate that IL-12 ABD fusion proteins that contain a targeting arm comprising BGB-A445, 20E5, 20E5_V4, or 20E5-V5 do not compete with OX40L for OX40 binding.

### T. Example 20—Capacity of OX40 Targeted IL-12 ABD Fusion Proteins to Block OX40L-Induced Signaling

OX40L-induced signaling has the potential to enhance the response of the immune system against cancer cells by promoting T cell survival and differentiation whilst also suppressing the differentiation and activity of immunosuppressive Tregs. Indeed, treatment with exogenous OX40L or agonistic OX40 antibodies have demonstrated anti-tumor activity in pre-clinical models of cancer (see, e.g., Weinberg, A. D. et al. J. Immunology (2000): 164 (4), 2160-2169, incorporated by reference in its entirety). Accordingly, it may be desirable for an IL-12 ABD fusion protein not to block OX40 signaling so as to not dampen the endogenous anti-cancer immune response.

Given that OX40 signaling is a consequence of OX40 receptor clustering, which is induced upon binding of homotrimeric OX40L, it is conceivable that an antibody that does not block OX40L binding could still interfere with OX40 signaling via preventing OX40 clustering. To investigate whether the various anti-human OX40 ABD interfere with OX40L-induced signaling, hOX40-Jurkat-NF-κB cells were incubated overnight with 10 nM human OX40L-Fc (Sino Biological, catalogue number 13127-H01H) in the presence or absence of 10, 50, or 250 nM IL-12 ABD fusion protein, and OX40 signaling was quantified by measuring the percentage of GFP+ cells. After a 1 hour or overnight incubation, OX40 levels were monitored by staining cells with a non-competing anti-human OX40 mAb (clone Ber-ACT35, BioLegend catalogue number 350012).

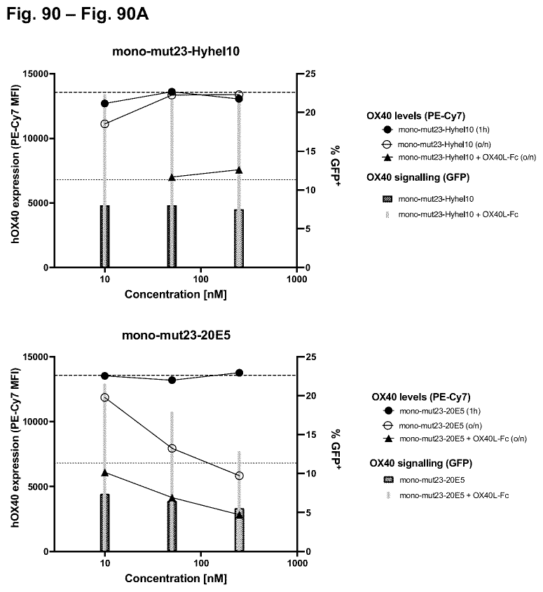

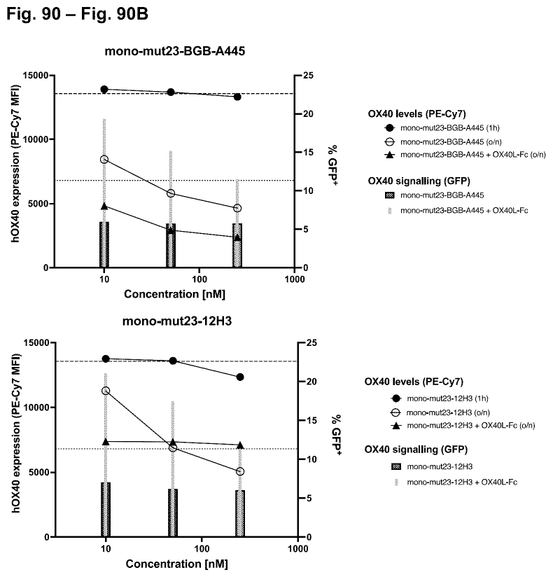

The results of the OX40 signaling assay are shown in FIG. 90. Following overnight incubation, treatment with OX40L-Fc or OX40-targeted IL-12 ABD fusion protein individually resulted in a dose-dependent decrease in OX40 expression, potentially due to receptor internalization. Of note, overnight incubation with a combination of OX40L-Fc and either mono-mut23-BGB-A445 or mono-mut23-20E5 further reduced OX40 expression levels compared to treatment with each test article alone. These observations are in agreement with results shown in Example 19, where it was demonstrated that mono-mut23-BGB-A445 and mono-mut23-20E5 do not block OX40L-Fc binding to OX40. A similar effect was not observed for the OX40L-blocking test article, mono-mut23-12H3. Within the context of this assay, treatment with each of the OX40-targeted IL-12 ABD fusion proteins resulted in a dose-dependent reduction in GFP signaling. However, these results are potentially biased due to the lower OX40 expression on cells that were treated overnight with OX40L-Fc and OX40-targeted IL-12 ABD fusion proteins.

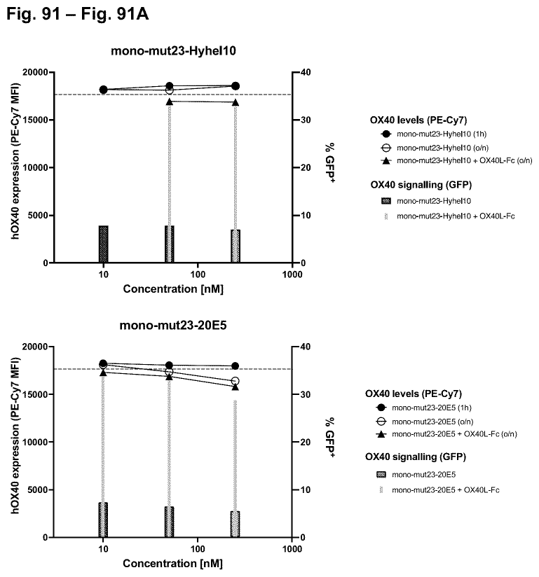

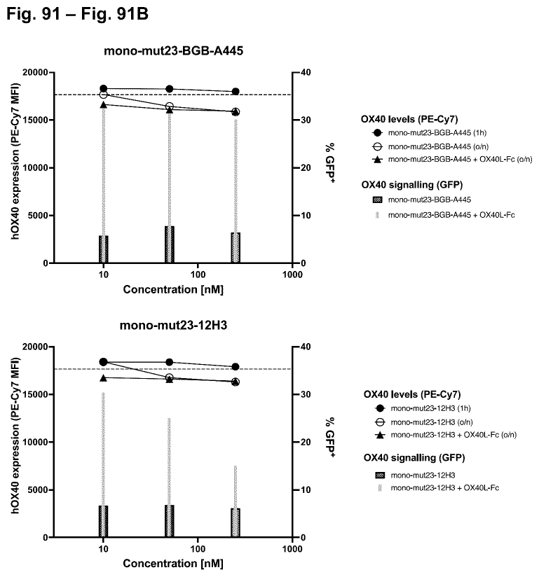

To account for this discrepancy, a gate was applied such that GFP expression was assessed only on cells that expressed high (and equivalent amongst cells treated with different test articles) levels of OX40, the results of which are depicted in FIG. 91. Using this analysis, mono-mut23-12H3 again resulted in a dose-dependent decrease in GFP, as would be expected for a test article known to block OX40L-Fc binding. However, cells treated with mono-mut23-BGB-A445 or mono-mut23-20E5 exhibited similar GFP levels to those treated with the non-targeted IL-12 ABD fusion protein (mono-mut23-Hyhel10), indicating that these test articles do not block OX40L-induced OX40 signaling.

### U. Example 21—Activity of Human OX40 Targeted IL-12 ABD Fusion Proteins on Activated PBMC

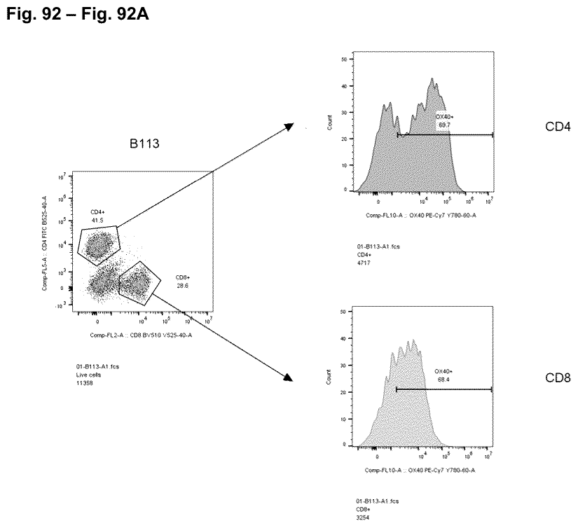

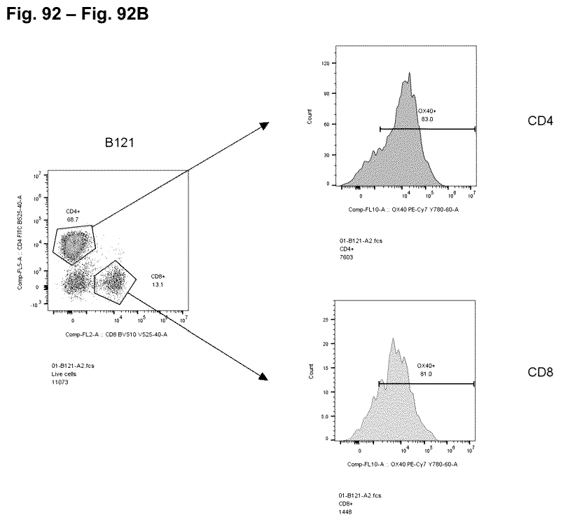

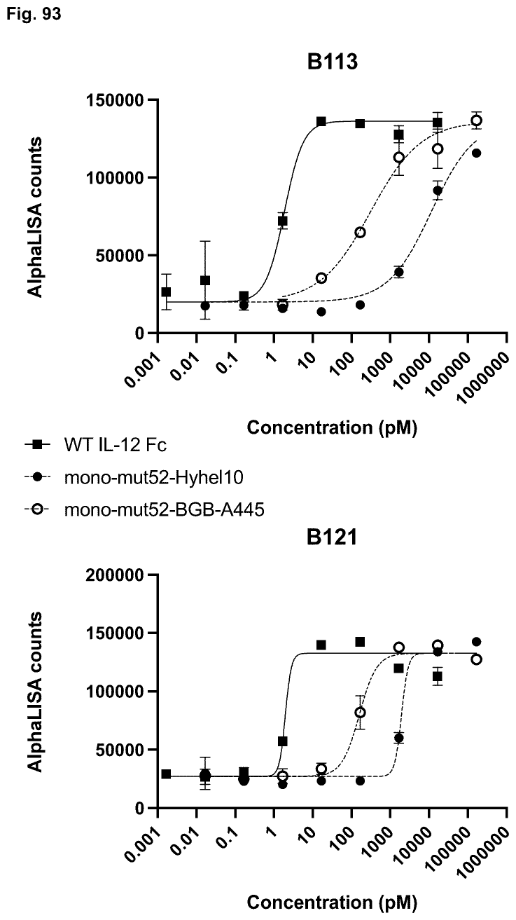

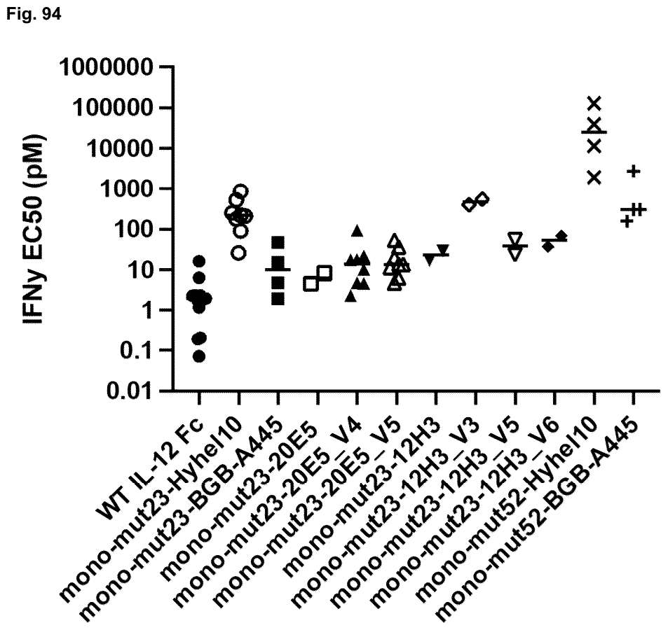

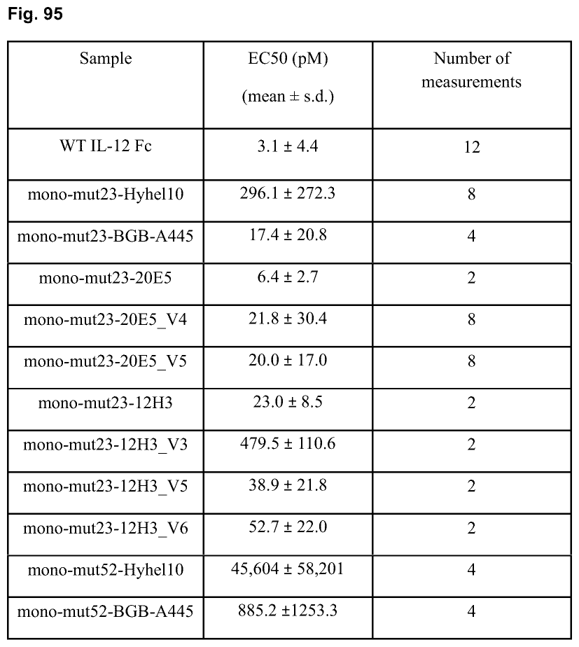

To assess the activity of the human OX40-targeted reduced potency IL-12-ABD fusion proteins, PBMCs from healthy donors were activated for 2 days in T cell media (RPMI 1640 media containing 10% FCS, 1× GlutaMAX, 1% pen/strep, 0.1% b-mercaptoethanol, 10 mM HEPES, 1% non-essential amino acids, 1% sodium pyruvate) containing 600 U/mL IL-2, and 32 ng/mL OKT3. Following activation, the cells were analyzed by flow cytometry, an example of which for two donors (B113 and B121) is shown in FIG. 92. Activated cells typically comprised predominantly CD4+ T cells (41.5% or 68.7% for B113 and B121, respectively), with the remaining cells being CD8+ T cells (28.6% and 13.1%, for B113 and B121, respectively) and non-T cells. The majority of CD4+ and CD8+ T cells (between 68% and 83%) expressed OX40, with a higher median fluorescence intensity being evident on CD4+ T cells. Activated cells were stimulated with WT IL-12 Fc or IL-12 ABD fusion proteins for either 48h or 72 h, and supernatants analyzed using an AlphaLISA High Performance Human IFNγ Detection Kit (Perkin Elmer). Dose response curves showing IFNγ production from donors B113 and B121 are shown in FIG. 93, and a graph of calculated EC50 values from several experiments are shown in FIG. 94 and summarized in FIG. 95. Together, these data demonstrate that fusion to BGB-A445, 20E5, and 12H3 can enhance the activity of reduced potency IL-12 variants on activated PBMC that express OX40. These data also inform as to the relationship between ABD binding affinity and activity of the resulting IL-12 ABD fusion protein. Specifically, ABD's that exhibited monovalent EC50's values for binding to hOX40-Jurkat-NF-κB cells of between 15-113 nM (mono-mut23-BGB-A445, mono-mut23-20E5, mono-mut23-20E5_V4, mono-mut23-20E5_V5, mono-mut23-12H3, mono-mut23-12H3_V5) had similar functional effects on activated PBMCs, whereas those that exhibited substantially reduced affinity for OX40 (for example, mono-mut23-12H3_V3) were functionally more similar to the non-targeted control (mono-mut23-Hyhel10).

### V. Example 22—Activity of Human OX40 Targeted IL-12 ABD Fusion Proteins on Purified Immune Cells Under Different Activation Conditions

Next, the activity of OX40 targeted reduced potency IL-12 ABD fusion proteins was investigated on purified CD4⁺ T cells, CD8⁺ T cells, and NK cells that were subjected to different activation conditions. CD4⁺ T cells, CD8⁺ T cells and NK cells were purified from healthy donor PBMC samples using the following negative selection kits: CD4+ T cell isolation kit (Miltenyi Biotec), EasySep Human CD8+ T cell isolation kit (StemCell Technologies), or human NK cell isolation kit (Miltenyi Biotec), respectively. Purified cells were either treated with WT IL-12 Fc or IL-12 ABD fusion protein either on day 0 (DO), immediately after a two-day activation period (D2), or following activation and an additional five days in culture (D7). For CD4⁺ and CD8⁺ T cells, activation involved culturing in T cell media that contained 100 U/mL IL-2 and a 1:1 bead:cell ratio of CD3/CD28 Dynabeads (Invitrogen), whereas subsequent culture was performed in the presence of the same media without Dynabeads. For NK cells, culturing conditions during and post-activation involved SCGM media containing 10% human serum, 1× GlutaMAX, and 10 ng/mL IL-15.

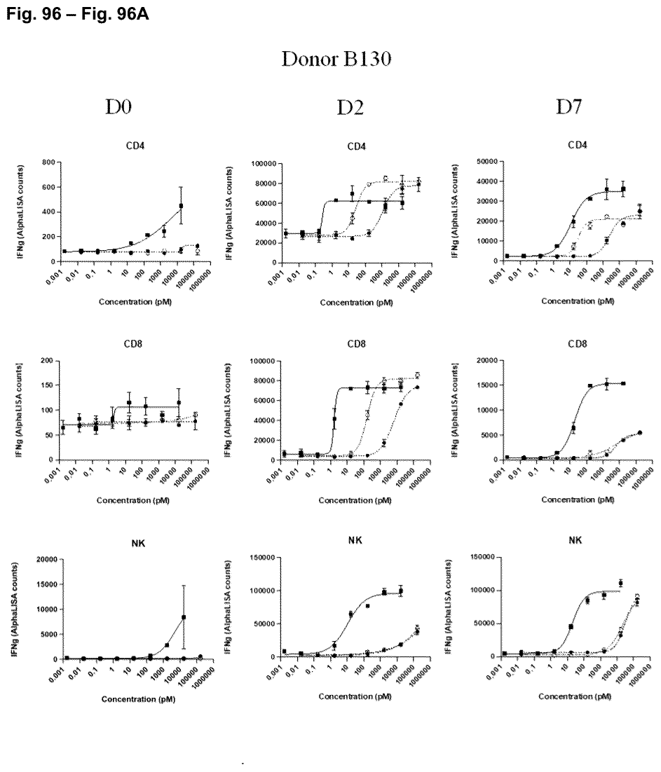

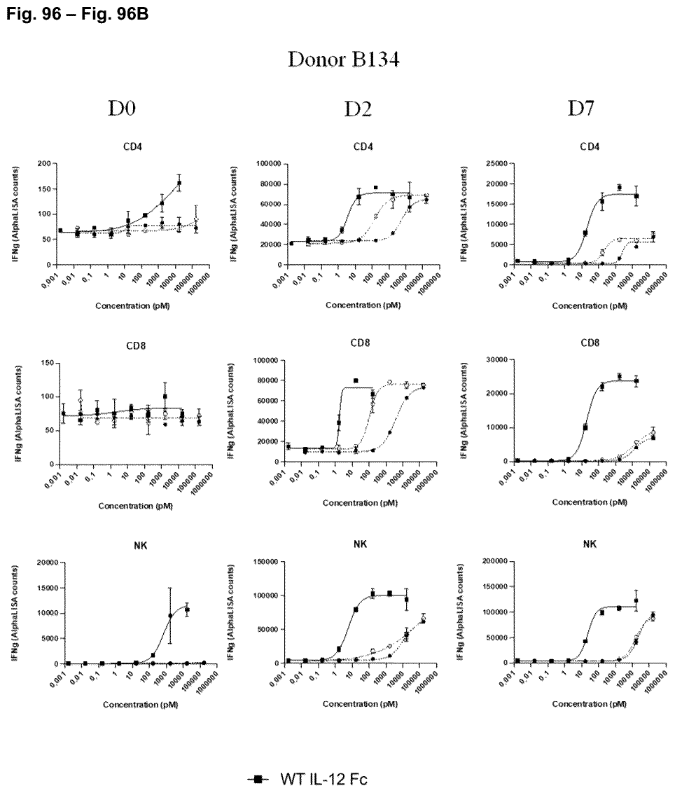

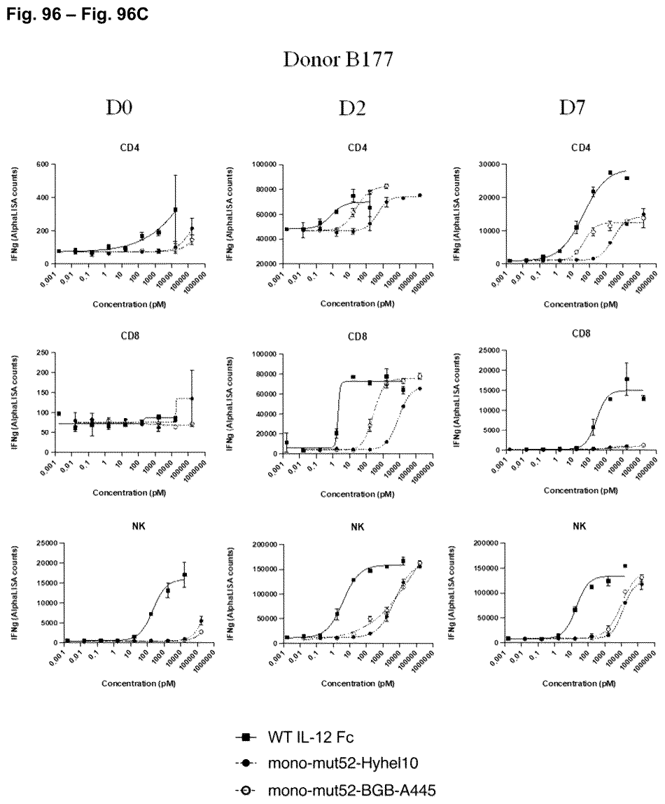

At each time point, cells were treated with WT IL-12 Fc or IL-12 ABD fusion proteins for 2 days in the presence of 50 U/mL IL2 (for T cells) or 1 ng/mL IL-15 (for NK cells). Secreted IFNγ was measured using an AlphaLISA High Performance Human IFNγ Detection Kit (Perkin Elmer), the results of which are depicted in FIG. 96. Immediately prior to treatment, cells were subjected to flow cytometry to assess expression of OX40, IL-12R(31, and IL-12Rβ2, the results of which are depicted in FIG. 97.

Overall, OX40 targeting enhanced the potency of the IL-12 ABD fusion protein (mono-mut52-BGB-A445) on CD4⁺ and CD8⁺ T cells on D2 and CD4⁺ T cells on D7, relative to the non-targeted control (mono-mut52-Hyhel10). In contrast, mono-mut52-BGB-A445 only weakly stimulated NK cells at all time points tested. These trends correlated well with the observed OX40 expression levels on each cell type, which was low or absent on NK cells (at all time points) and CD4⁺ and CD8⁺ T cells on D0, and was transiently induced on CD4⁺ and CD8⁺ cells, where it was expressed at high levels on D2, and diminished (CD4⁺ T cells) or absent (CD8⁺ T cells) on D7.

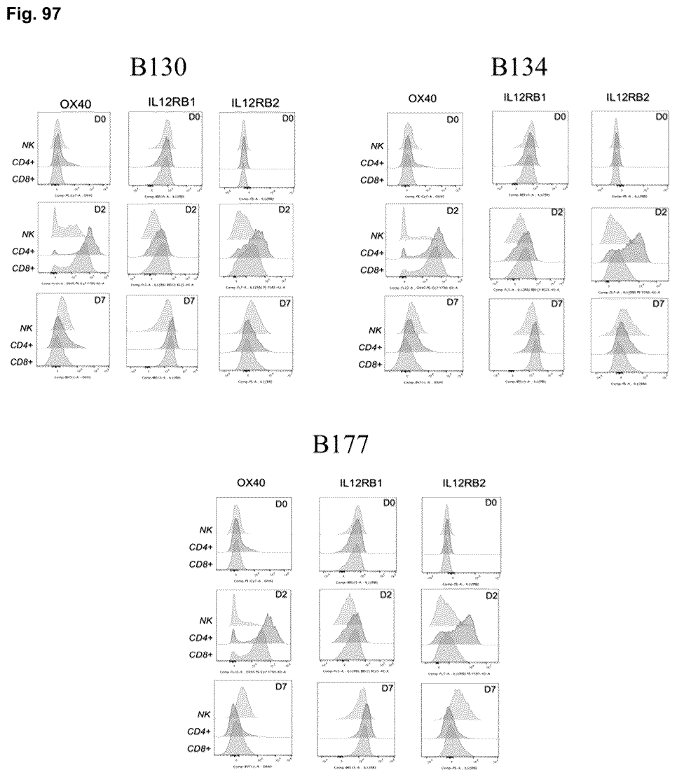

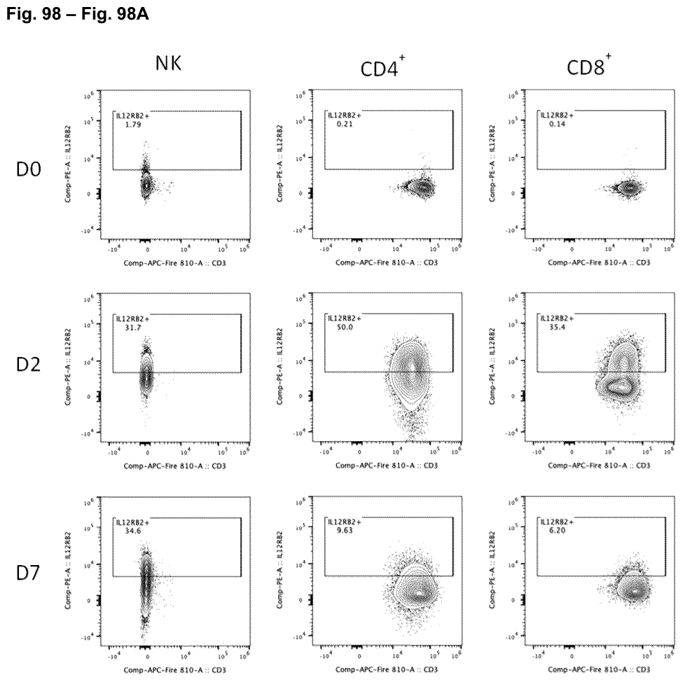

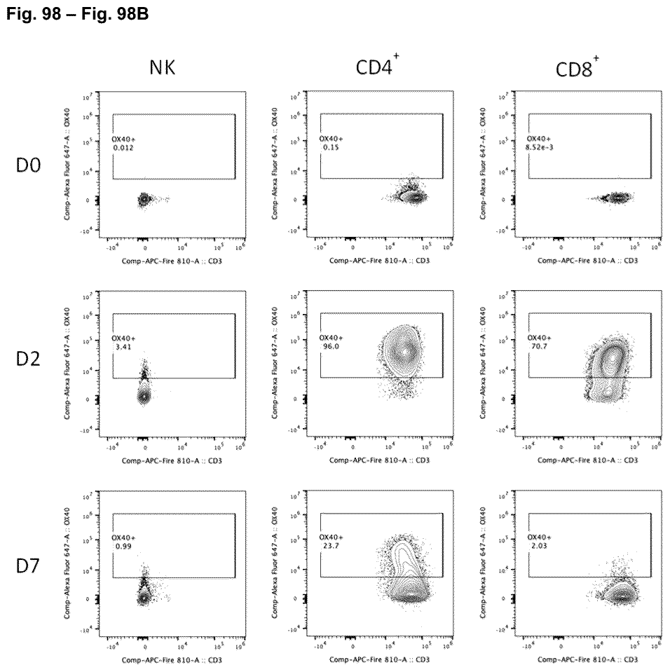

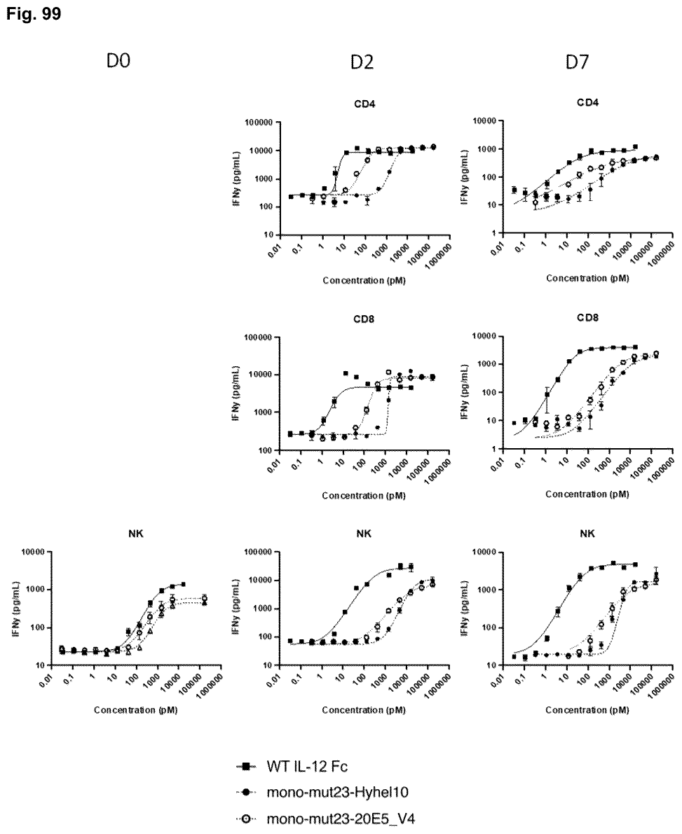

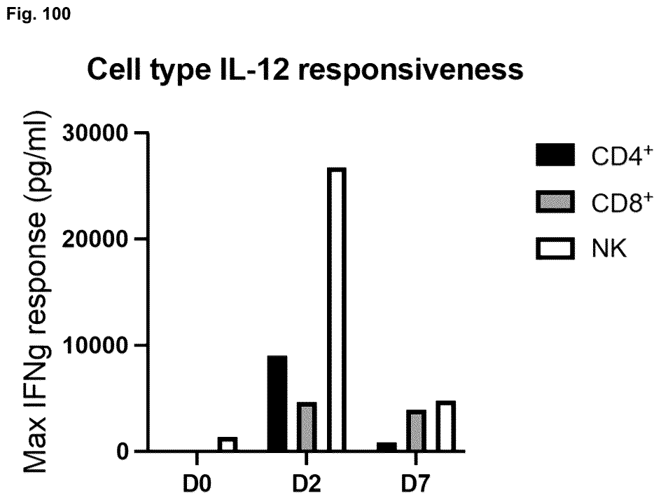

The activity of mono-mut23-20E5_V4 on purified immune cells was assessed in a separate experiment that followed the same experimental workflow as described above. In this case, IFNγ standards were included in the AlphaLISA step to allow for direct quantification of secreted IFNγ levels. The expression of OX40 and IL-12Rβ2 as assessed by flow cytometry prior to treatment with WT IL-12 Fc or IL-12 ABD fusion proteins is depicted in FIG. 98 and IFNγ measurements are depicted in FIG. 99. The IL-12 responsiveness of each cell type under each experimental condition was assessed by plotting the maximum IFNγ response to WT IL-12 Fc, as depicted in FIG. 100.

As shown in FIG. 100, prior to activation (DO), NK cells were the only cell type that produced detectable levels of IFNγ (data is not shown for CD4⁺ and CD8⁺ T cells prior to activation because the measurements were below the lower limit of quantification for the IFNγ standards). All cell types (CD4⁺, CD8⁺ and NK) were most responsive to IL-12 immediately after activation (D2) and their response was generally diminished on D7. Overall, cell type responsiveness to IL-12 correlated well to expression of IL-12Rβ2.

As observed previously, OX40 expression was low on NK cells under all conditions, was highest on early activated CD4⁺ and CD8⁺ cells (D2), and diminished (CD4⁺ T cells) or low (CD8⁺ T cells) on late activated cells (D7). Functionally, mono-mut23-20E5_V4 induced IFNγ more potently than mono-mut23-Hyhel10 in cells that expressed OX40 (CD4⁺ and CD8⁺ cells on D2, and CD4⁺ cells on D7).

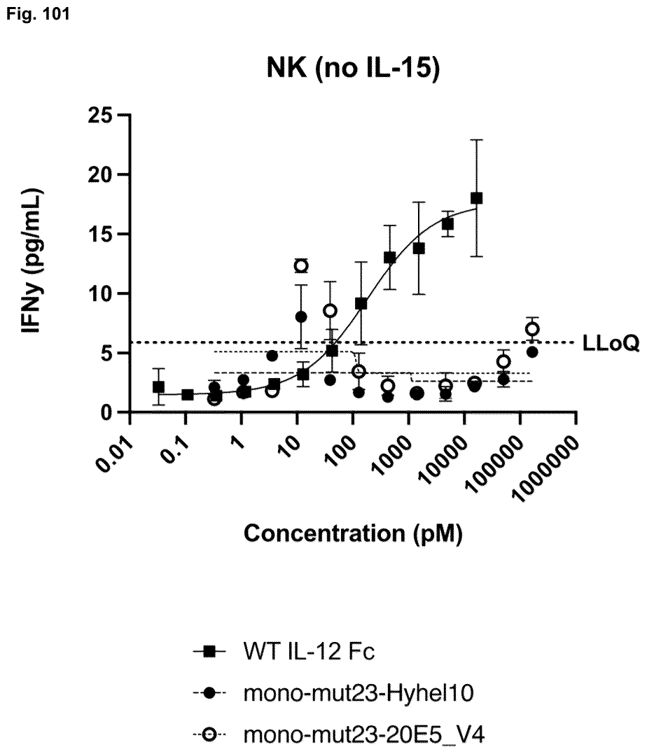

In this experiment, mono-mut23-20E5_V4 did induce measurable levels of IFNγ in non-activated NK cells, albeit at considerably lower levels than in activated T and NK cells. However, this result is unlikely to reflect the responsiveness of naïve NK cells to mono-mut23-20E5_V4 because NK cells were exposed to 1 ng/mL of IL-15 during the 48h duration of the assay, and this can result in upregulation of IL-12R. To investigate the effects of mono-mut23-20E5_V4 in a context that was more closely reflective of naïve NK cells, NK cells were purified from the same PBMC donor, and the assay was performed under the same conditions as described for day 0 but in the absence of IL-15. Results depicting IFNγ measurements are shown in FIG. 101. Under these conditions, only treatment with WT IL-12 Fc (and not mono-mut23-20E5_V4) resulted in levels of IFNγ that were consistently above the lower limit of quantification of the assay. Overall, these data indicate that OX40-targeted low potency IL-12 ABD fusion proteins exhibit preferential activity on cells likely to be located within the tumor environment (for example, OX40 positive activated T and NK cells) relative to cells likely to be located in peripheral organs (for example, OX40 negative non-activated T and NK cells).

### W. Example 23—Activity of Human OX40 Targeted IL-12 ABD Fusion Proteins on Induced Treg Cells

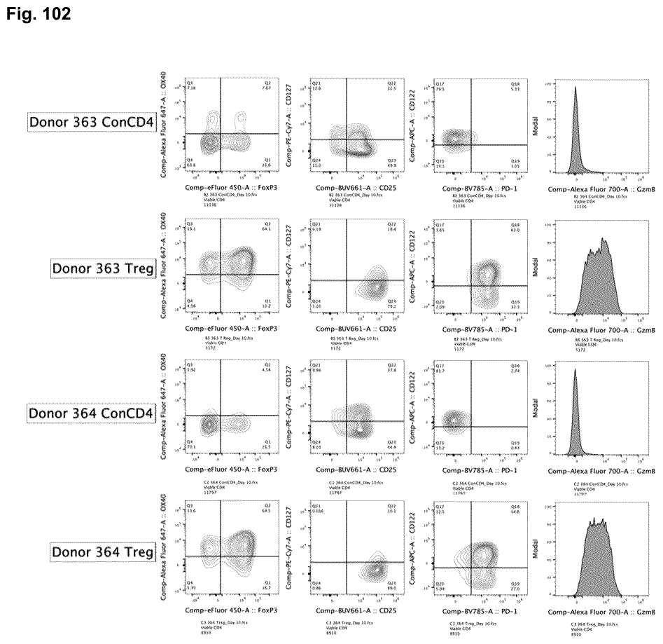

IL-12 has been reported to reverse Treg-mediated suppression of conventional (CD4⁺ FoxP3⁻) T cell proliferation. Thus, the anti-cancer properties of OX40-targeted IL-12 ABD fusion proteins may, at least in part, be due to targeting of IL-12 activity on tumor-infiltrating Treg cells, which express high levels of OX40. To investigate the activity of OX40-targeted IL-12 ABD fusion proteins on Treg cells, CD4+ cells were negatively selected from healthy human donor-derived PBMC using a CD4+ T cell isolation kit (Miltenyi Biotec) and Treg were induced by maintaining cells in T cell media containing a 1:1 bead:cell ratio of CD3/CD28 Dynabeads (Invitrogen), 500 U/mL human IL-2 (Miltenyi Biotec), 5 mg/mL TGF-β (Miltenyi Biotec) and 10 nM retinoic acid. Following this 10-day differentiation period, cells were analyzed by flow cytometry to assess phenotype, the results of which are depicted in FIG. 102. Following differentiation, the cellular phenotype of induced Tregs was markedly different to that of control CD4⁺_{con} T cells that were activated for 3 days in T cell media containing a 1:1 bead:cell ratio of CD3/CD28 Dynabeads (Invitrogen) and 500 U/mL human IL-2 (Miltenyi Biotec) and then maintained in T cells media for a further 7 days. Unlike CD4⁺_{con} T cells, differentiated cells expressed high levels of phenotypic markers that are typically associated with Tregs (including PD-1, FoxP3, CD25 and granzyme B) and low levels of markers typically associated CD4⁺_{con}, T cells (e.g. CD127). Notably, a high proportion of induced Treg cells expressed OX40.

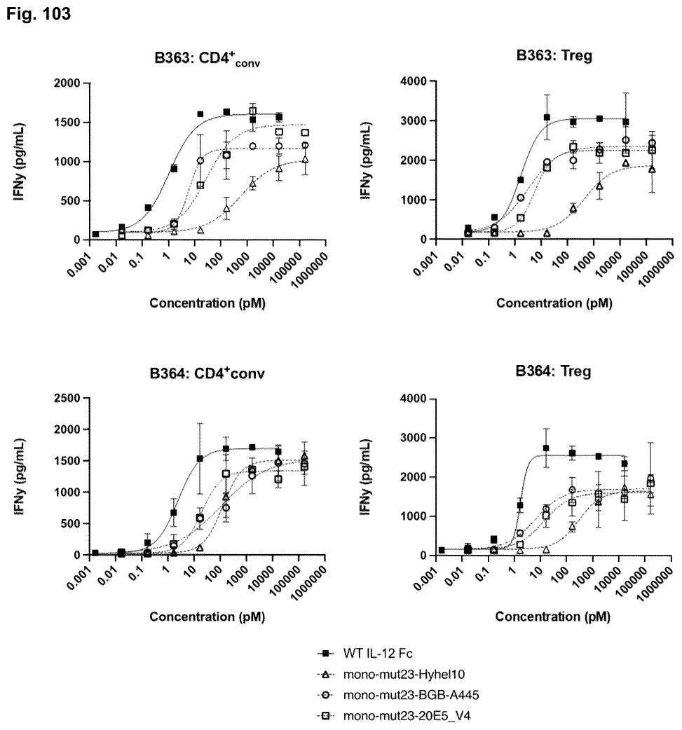

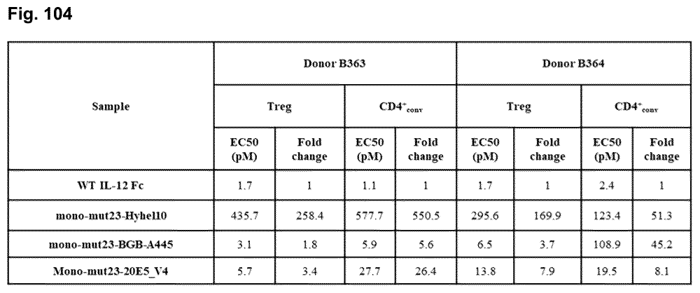

Ten days after their initial purification, cells were treated with WT IL-12 Fc or IL-12 ABD fusion proteins for 3 days and secreted IFNγ was measured using an AlphaLISA High Performance Human IFNγ Detection Kit (Perkin Elmer), the results of which are depicted in FIG. 103 and summarized in FIG. 104. Within this experimental context, both OX40-targeted IL-12 ABD fusion proteins tested exhibited more potent activity on Tregs (EC50 values of 3.1 and 6.5 pM for mono-mut23-BGB-A445 and EC50 values of 5.7 and 13.8 pM for mono-mut23-20E5-V4) than they did on CD4⁺_{con}, T cells (EC50 values of 5.9 and 108.9 pM for mono-mut23-BGB-A445 and EC50 values of 27.7 and 19.5 pM for mono-mut23-20E5-V4), presumably due to the elevated OX40 expression on Treg relative to CD4⁺_{con}, T cells. Thus, OX40-targeting of IL-12 ABD fusion proteins has the capacity to potently enhance IL-12 activity on Treg cells.

### X. Example 24—OX40-Targeted IL-12 ABD Fusion Proteins Enhance IL-12 Potency Only on Cells that Express OX40 and IL-12R in Cis

To investigate whether enhancement of the potency of OX40-targeted IL-12 ABD fusion proteins requires OX40 expression in cis with IL-12R (i.e., on the same cellular membrane as IL-12R), T cells were purified from healthy human donor-derived PBMC using a pan T cell isolation kit (Miltenyi Biotec) and activated for 2 days in T cell media containing a 0.5:1 bead:cell ratio of CD3/CD28 Dynabeads (Invitrogen). Following activation, cells were maintained in T cell media containing 30 U/mL TL-2 for a further 7 days, at which point they were incubated for 30 minutes with either WT IL-12 Fc, a non-targeted reduced potency IL-12 fusion protein (mono-mut52-Hyhel10) or an OX40-targeted IL-12 ABD fusion protein (mono-mut52-BGB-A445) prior to being fixed, permeabilized, and stained with a panel of antibodies that included: anti-human CD8 (BUV737, BD Biosciences), anti-human CD4 (FITC, Miltenyi Biotec), anti-human OX40 (A647, BioLegend), anti-human CD3 (APC Fire-810, BioLegend), and anti-mouse/human STAT4 pY693 (PE, BD Biosciences).

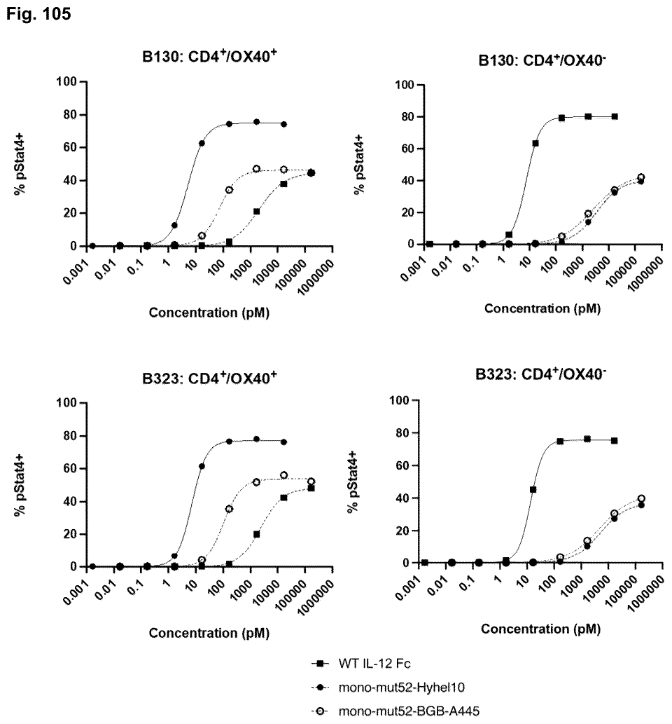

Results depicting pSTAT4 expression (used in this experiment as a readout for cellular activation downstream of IL-12R) in OX40⁺ CD4⁺ cells and in OX40-CD4⁺ cells are shown in FIG. 105. In OX40⁻ cells, the OX40-targeted IL-12 ABD fusion protein (mono-mut52-BGB-A445) induced pSTAT4 to the same degree as the non-targeted control (mono-mut52-Hyhel10). However, in OX40⁺ cells, mono-mut52-BGB-A445 resulted in more potent induction of pSTAT4 than mono-mut52-Hyhel10.

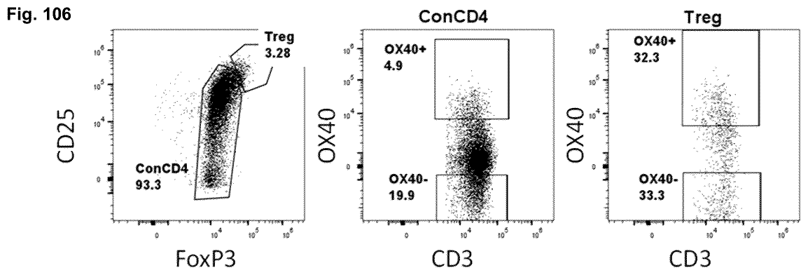

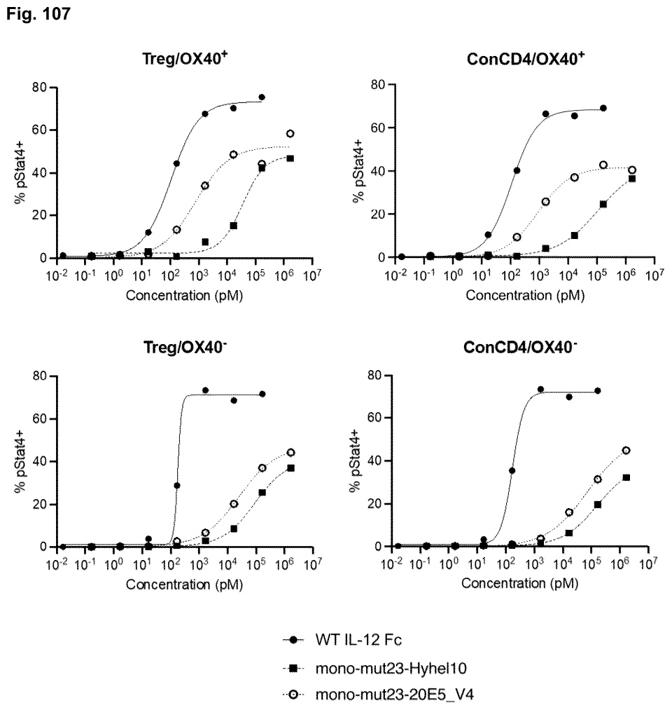

The capacity of mono-mut23-20E5-V4 was assessed in a separate pSTAT4 assay using late activated (D7) purified CD4⁺ T cells that were generated using the same methodology as described in Example 22. Here, anti-human FoxP3 (e450, Bioscience) and anti-human CD25 (BUV661, BD Biosciences) antibodies were included in the staining to allow for discrimination between conventional CD4⁺ cells and Treg cells. The cellular composition, OX40 expression and gates used to define OX40 positive and negative populations are depicted in FIG. 106, and results depicting pSTAT4 expression are shown in FIG. 107. In both Treg and conventional CD4⁺ T cells, mono-mut23-20E5-V4 more potently induced pSTAT4 expression than the non-targeted control (mono-mut23-Hyhel10) specifically in OX40⁺ cells. Overall, these data indicate that OX40-targeted IL-12 ABD fusion proteins enhance the activity of IL-12 only on cells that express OX40 and IL-12R in cis (i.e., within the same membrane).

### Y. Example 25—Human OX40 Targeted IL-12 ABD Fusion Proteins do not Trigger OX40 Signaling

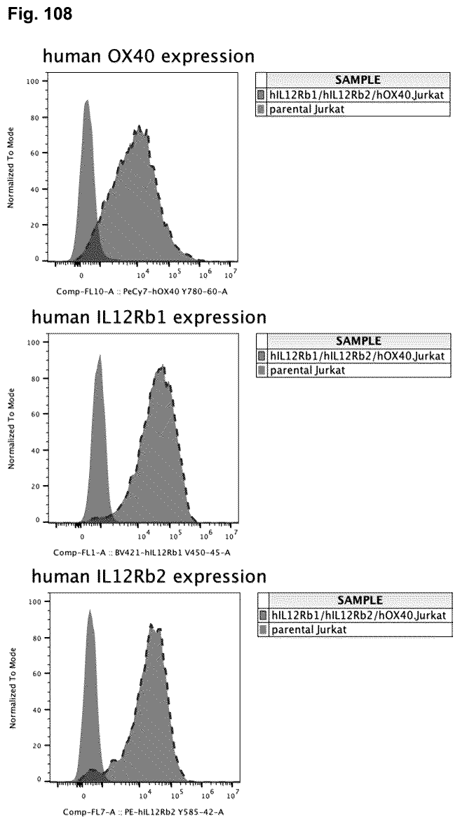

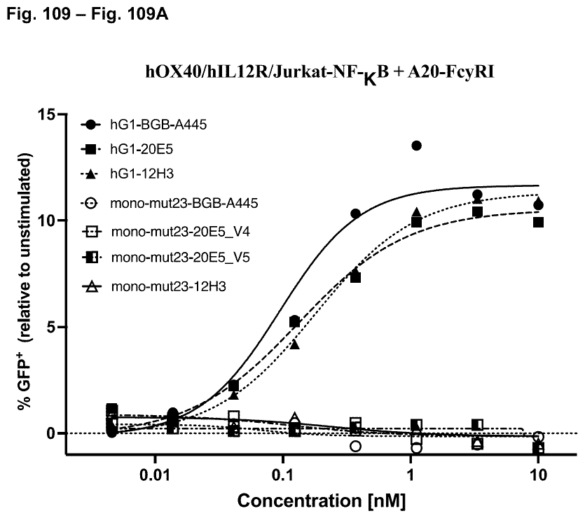

As described in Example 14, mouse OX40-targeted IL-12 ABD fusion proteins do not stimulate OX40 signaling in the presence of IL-12R in cis/trans, or high affinity FcγR in trans. To assess if the human OX40-targeted IL-12 ABD fusion proteins behaved similarly, we performed a similar assay to that described in Example 14, whereby hOX40-Jurkat-NF-κB cells were transduced with human IL-12R(31/IL-12Rβ2 subunits that lacked cytoplasmic domains to produce a hOX40/hIL-12R/Jurkat-NF-κB reporter cell line. The expression of OX40, IL-12Rβ1, and IL-12Rβ2 on these cells is depicted in FIG. 108. hOX40/hIL-12R/Jurkat-NF-κB reporter cells were incubated with anti-human OX40 antibodies either in traditional IgG1 bivalent mAb format or as monovalent IL-12 ABD fusion proteins, and cultured either alone (FIG. 109B) or in the presence of A20 cells that were transduced with human FcγRI (A20-hFcγRI; FIG. 109A). After an overnight incubation, GFP expression in the reporter cells was measured by flow cytometry, the results of which are shown in FIG. 109.

In the presence of A20-hFcγRI cells, each of the traditional IgG1 bivalent mAb clones tested (hG1-BGB-A445, hG1-20E5 and hG1-12H3) induced a dose-dependent expression of GFP in hOX40/hIL-12R/Jurkat-NF-κB reporter cells with EC50 values of 0.09, 0.13 and 0.18 nM, respectively. This induction of OX40 reporter signal was mediated via FcγR clustering, since the same samples did not trigger GFP expression in the absence of A20-hFcγRI cells.

In contrast, none of the monovalent IL-12 ABD fusion proteins containing a BGB-A445, 20E5 or 12H3 binding arm induced GFP expression in hOX40/hIL-12R/Jurkat-NF-κB reporter cells in the presence of A20-hFcγRI cells, indicating that these molecules do not trigger OX40-mediated intracellular signaling upon exposure to IL-12R in cis or trans, or the presence of FcγR in trans (note that the IL-12 ABD fusion proteins contain mutations within the Fc region to prevent FcγR binding).

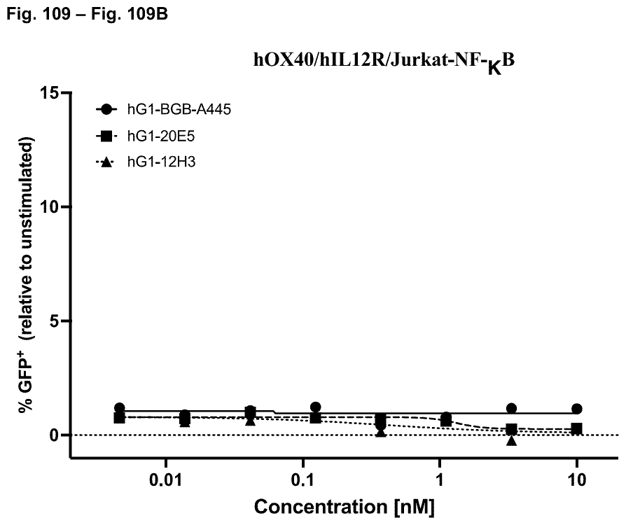

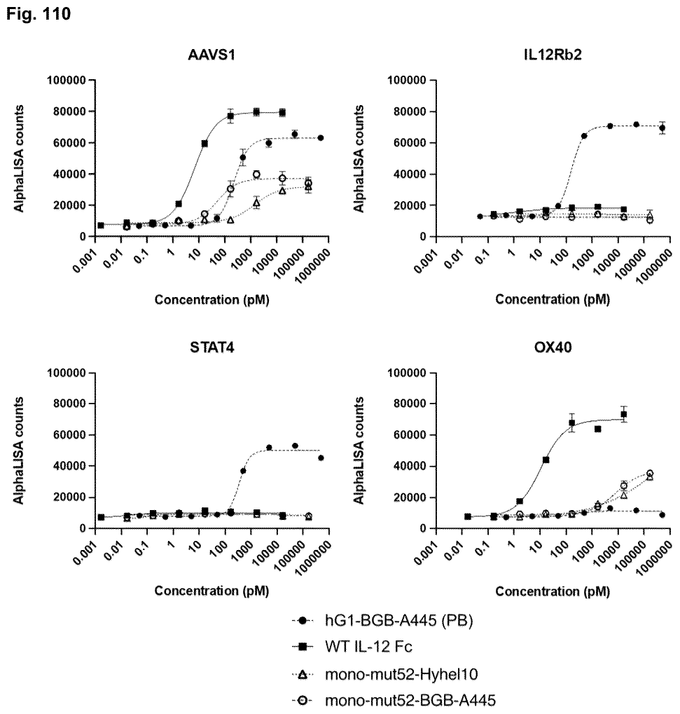

To confirm these findings in a more physiological setting, the experimental setup of the PBMC IFNγ assay was modified to include co-stimulation with plate bound anti-CD3 mAb, such that signaling downstream of OX40 would result in a measurable IFNγ response. To delineate whether secreted IFNγ was a result of IL-12R or OX40-mediated signaling, certain groups of cells were subjected to CRISPR/Cas9-mediated knockout using ribonucleoproteins targeting either OX40 or components that are necessary for IL-12R signaling (IL-12Rβ2 or STAT4). Ribonucleoprotein targeting a gene desert (AAVS1) were used as a negative control. Following activation and gene knockout, cells were washed and stimulated with plate bound anti-CD3 (clone OKT3, eBioscience) in the presence of soluble forms of either WT IL-12 Fc or IL-12 ABD fusion proteins, or incubated with plates upon which OKT3 and hG1-BGB-A445 were co-immobilized. After 3 days, secreted IFNγ was measured using an AlphaLISA High Performance Human IFNγ Detection Kit (Perkin Elmer), the results of which are depicted in FIG. 110.

Stimulation with plate bound anti-OX40 (hG1-BGB-A445 (PB)) resulted in robust IFNγ production in cells that were treated with the negative control guides (AAVS1). IFNγ production in response to plate bound hG1-BGB-A445 was OX40-dependent and IL-12R/STAT4-independent, since it was ablated in OX40 knockout cells, but not IL-12Rβ2 or STAT4 knockout cells. Thus, under these assay conditions, stimulation of OX40 signaling results in a measurable IFNγ response. Under the same conditions, an exemplary OX40-targeted IL-12 ABD fusion protein (mono-mut52-BGB-A445) also resulted in IFNγ production, which it induced more potently (EC50 of 50.0 pM) than a non-targeted reduced potency IL-12 ABD fusion protein (mono-mut52-Hyhel10, EC50 of 1417 pM). The enhanced activity of mono-mut52-BGB-A445 relative to mono-mut52-Hyhel10 was dependent on OX40 expression, since both test articles induced IFNγ to a similar extent in OX40 knockout cells. However, IFNγ production in response to mono-mut52-BGB-A445 was entirely attributed to IL-12R signaling, since it was fully ablated in cells that included knockouts of IL-12R pathway components (IL-12Rβ2 or STAT4). These data indicate that OX40-targeted IL-12 ABD fusion proteins do not trigger OX40 signaling in primary human cells.

### Z. Example 26—OX40 Targeted IL-12 ABD Fusion Proteins Trigger Immunological Memory Responses Against MC38 Tumors

In some circumstances, immunotherapy can induce long lasting durable responses due to the induction of immunological memory. To investigate whether OX40-targeted IL-12 ABD fusion proteins are capable of inducing immunological memory, C57BL/6 mice were implanted with MC38 tumors and treated with a single injection of 0.143 mg/kg mono-Y36E-OX86 (referred to as Day 0), resulting in 5/6 mice exhibiting complete responses by day 17. On day 41, MC38 tumor cells were re-implanted into mice that remained tumor free (n=5) and tumor growth was monitored three times per week. As a control, the same MC38 cells were also implanted into naïve mice (n=5) that had not previously been exposed to MC38 cells or treated with mono-Y36E-OX86. Tumor growth in naïve and previously treated mice is depicted in FIG. 111 (naïve—top panel; previously treated—bottom panel). MC38 tumors grew in an uncontrolled manner in naïve mice. However, no measurable tumors grew on any of the mice that exhibited a complete response with respect to MC38 tumors using mono-Y36E-OX86. These data indicate that treatment with OX40 targeted IL-12 ABD fusion protein protects mice from subsequent tumor challenge, most likely via the induction of long-lasting immunological memory.

### AA. Example 27—an Additional Anti-Human OX40 Antibody with Desirable Characteristics

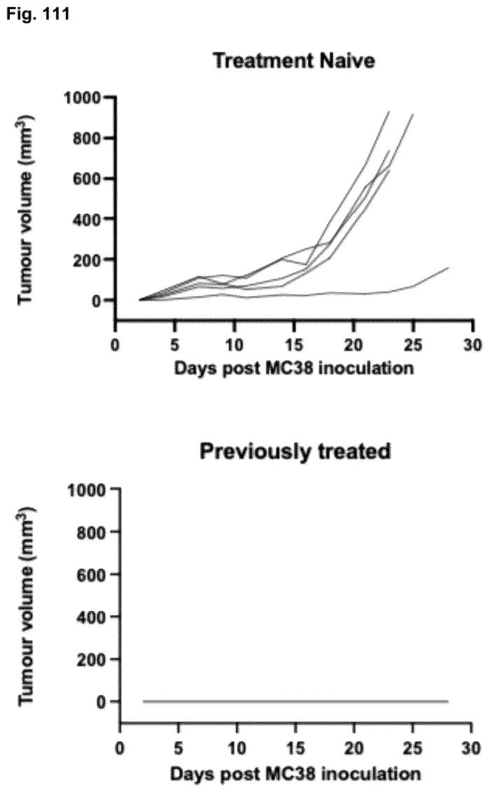

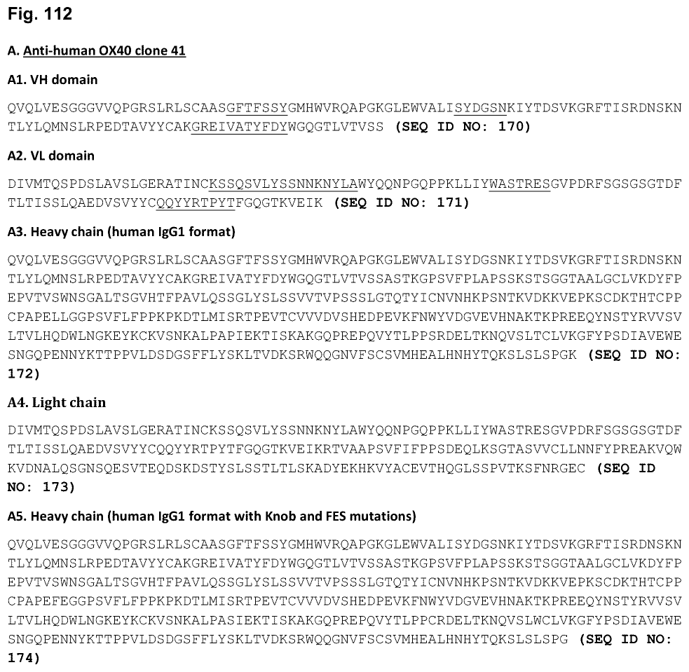

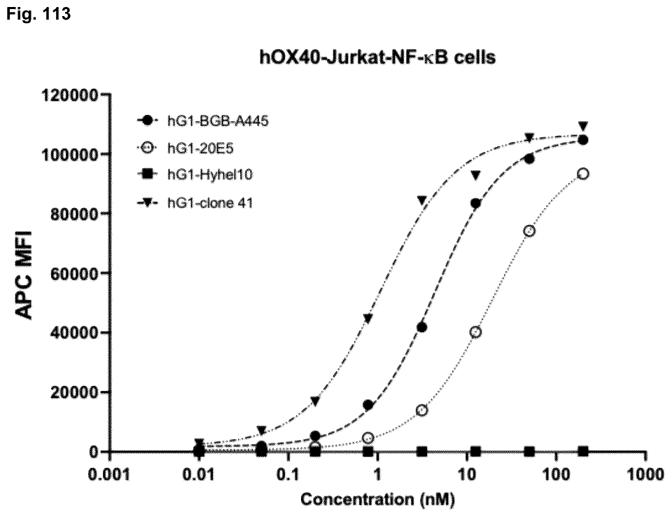

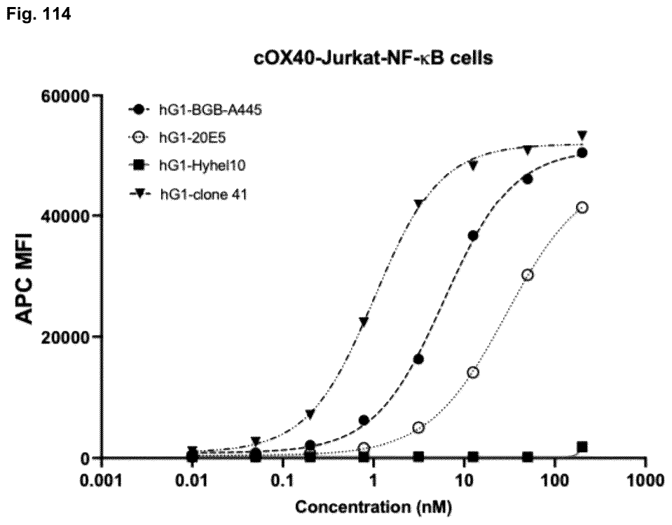

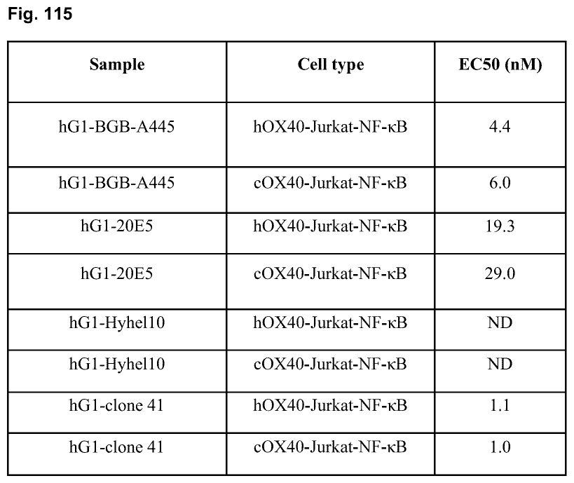

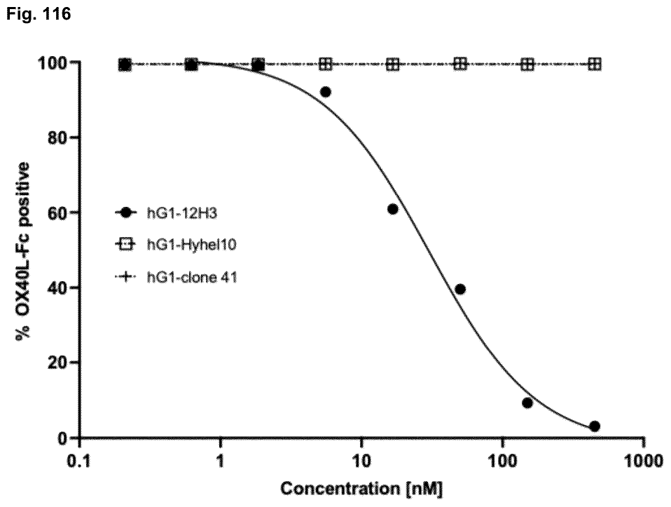

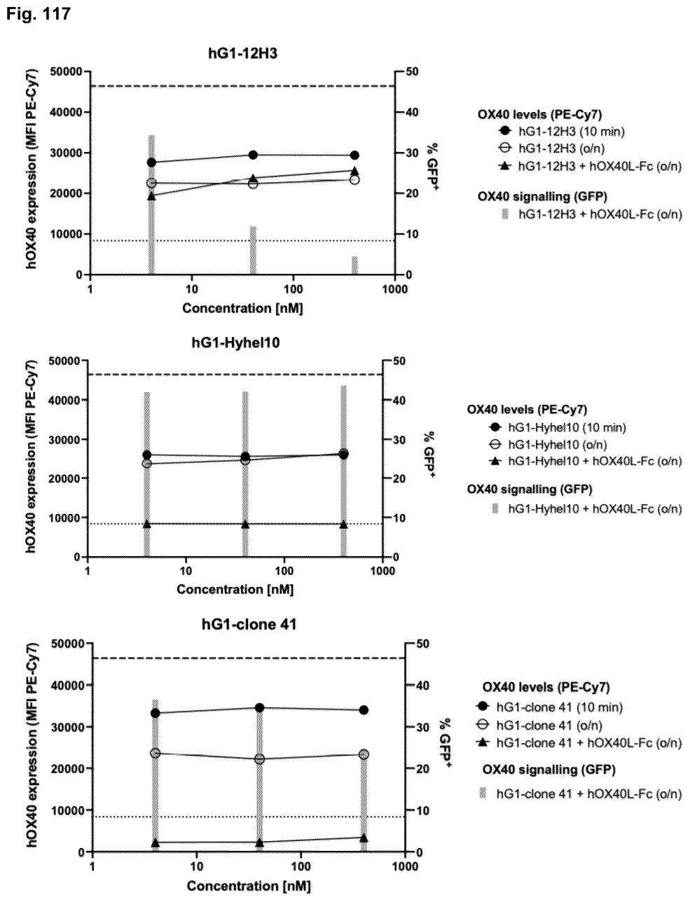

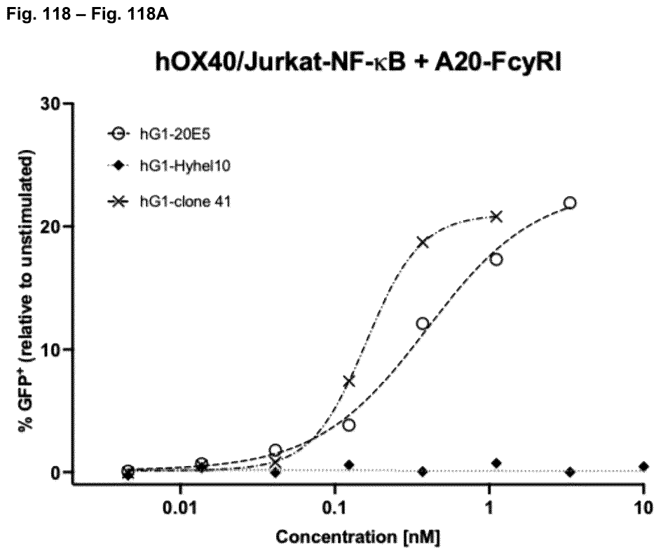

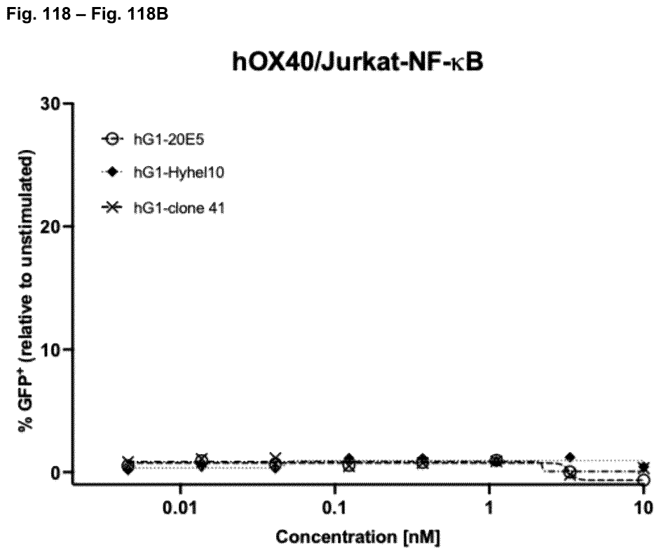

Sequences for the VH and VL domains of an additional anti-human OX40 antibody (clone 41) are provided in FIG. 112 (SEQ ID NOs: 170 and 171, respectively). To investigate whether clone 41 would be suitable for use within IL-12 ABD fusion proteins, this antibody was recombinantly expressed in bivalent human IgG1 format (termed ‘hG1-clone 41’) and subjected to protein A purification. Purified hG1-clone 41 exhibited greater than 98% purity as determined by analytical size exclusion chromatography. hG1-clone 41 was initially tested for binding to human and cynomolgus OX40 using flow cytometry as described in Example 16, the results of which are depicted in FIGS. 113 and 114, respectively, and summarized in FIG. 115. hG1-clone 41 bound with high affinity to both human and cynomolgus OX40, with EC50 values of 1.1 and 1.0 nM, respectively. Next, hG1-clone 41 was assessed for its capacity to block the binding of OX40L to hOX40-Jurkat-NF-κB cells, as described in Example 19. As shown in FIG. 116, hG1-clone 41 did not alter the binding of OX40L-Fc to hOX40-Jurkat-NF-κB cells, indicating that it does not compete with OX40L for the OX40 binding site. The capacity of hG1-clone 41 to block OX40L-induced signaling in hOX40-Jurkat-NF-κB cells was also assessed using a similar experimental setup as described in Example 20, the results of which are depicted in FIG. 117. Here, hG1-clone 41 marginally decreased OX40L-induced OX40 signaling (measured as % of GFP+ cells) compared to the ligand blocking 12H3 antibody (hG1-12H3), indicating that clone 41 does not potently inhibit OX40L-induced signaling. Finally, the capacity of hG1-clone 41 to trigger OX40 signaling was assessed in hOX40-Jurkat-NF-κB cells, as described in Example 25. As shown in FIG. 118, hG1-clone 41 induced dose-dependent expression of GFP in hOX40-Jurkat-NF-κB cells in the presence, but not absence, of A20 cells that expressed hFcγRI. Thus, hG1-clone41 requires FcγR-mediated clustering in order to trigger OX40 signaling. Overall, these data demonstrate that the characteristics of clone 41 corroborate the hypothesis that it is a suitable binding arm for use in OX40-targeted IL-12 ABD fusion proteins described herein.

1. An IL-12 Fc fusion protein, comprising:
(a) a first monomer comprising, from N-terminus to C-terminus: (i) a first IL-12 subunit domain, (ii) a first domain linker, (iii) a second IL-12 subunit domain, (iv) a second domain linker, and (v) a first Fc domain, wherein:
(1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and
(2) the variant IL-12 p35 subunit domain comprises a set of amino acid substitutions selected from the group consisting of: (a) Y40S/K170A, (b) Y40S/K170T, (c) Y40P/K168A, (d) Y40P/K170A, and (e) Y40E/K170A;
(b) a second monomer comprising, from N-terminus to C-terminus: (i) a variable heavy (VH) domain, (ii) a constant heavy (CH1) domain, and (iii) a second Fc domain; and
(c) a third monomer comprising, from N-terminus to C-terminus: (i) a variable light (VL) domain, and (ii) a constant light (CL) domain, wherein the VH domain and VL domain form an antigen binding domain (ABD) that binds a target antigen.

2. The IL-12 Fc fusion protein according to claim 1, wherein the variant IL-12 p35 subunit domain comprises any one of SEQ ID NOs: 17-21.

3. The IL-12 Fc fusion protein according to claim 1, wherein the IL-12 p40 subunit domain comprises SEQ ID NO: 4.

4. The IL-12 Fc fusion protein according to claim 1, wherein the IL-12 p40 subunit domain comprises a variant IL-12 p40 subunit domain.

5. The IL-12 Fc fusion protein according to claim 4, wherein the variant IL-12 p40 subunit domain comprises any one of SEQ ID NOs: 23-25.

6. The IL-12 Fc fusion protein according to claim 5, wherein:
(i) the variant IL-12 p40 subunit domain comprises SEQ ID NO: 23 or SEQ ID NO: 25; and
(ii) the variant IL-12 p35 subunit domain further comprises a C74S substitution mutation.

7. The IL-12 Fc fusion protein according to claim 1, wherein:
(i) the first Fc domain comprises any one of SEQ ID NOs: 26-30; and
(ii) the second Fc domain comprises any one of SEQ ID NOs: 26-30.

8. The IL-12 Fc fusion protein according to claim 7, wherein:
(i) the first Fc domain further comprises a set of substitution mutations comprising T366W/S354C, and the second Fc domain further comprises a set of substitution mutations comprising T366S/L368A/Y407V/Y349C; or
(ii) the first Fc domain further comprises a set of substitution mutations comprising T366S/L368A/Y407V/Y349C, and the second Fc domain further comprises a set of substitution mutations comprising T366W/S354C.

9. The IL-12 Fc fusion protein according to claim 1, wherein the ABD binds a target antigen selected from the group consisting of: (1) human OX40, (2) human LAG3, (3) human PD1, (4) human ICOS, (5) human 41BB, (6) human CCR7, and (7) human CCR8.

10. The IL-12 Fc fusion protein according to claim 9, wherein the target antigen comprises human OX40, and the ABD comprises a VH/VL domain pair selected from the group consisting of: (i) SEQ ID NOs: 152 and 143, respectively, (ii) SEQ ID NOs: 132 and 133, respectively, (iii) SEQ ID NOs: 137 and 138, respectively, (iv) SEQ ID NOs: 142 and 143, respectively, (v) SEQ ID NOs: 147 and 148, respectively, (vi) SEQ ID NOs: 130 and 131, respectively, (vii) SEQ ID NOs: 154 and 143, respectively, (viii) SEQ ID NOs: 156 and 143, respectively, (ix) SEQ ID NOs: 158 and 148, respectively, (x) SEQ ID NOs: 160 and 148, respectively, (xi) SEQ ID NOs: 162 and 148, respectively, (xii) SEQ ID NOs: 164 and 148, respectively, (xiii) SEQ ID NOs: 166 and 148, respectively, (xiv) SEQ ID NOs: 168 and 148, respectively, and (xv) SEQ ID NOs: 170 and 171, respectively, as shown in FIGS. 13, 75, 81, 85, and 112.

11. The IL-12 Fc fusion protein according to claim 9, wherein the target antigen comprises human LAG3, and the ABD comprises a VH/VL domain pair selected from the group consisting of: (i) SEQ ID NOs: 62 and 63, respectively, (ii) SEQ ID NOs: 103 and 104, respectively, (iii) SEQ ID NOs: 105 and 106, respectively, (iv) SEQ ID NOs: 107 and 108, respectively, (v) SEQ ID NOs: 109 and 110, respectively, and (vi) SEQ ID NOs: 111 and 112, respectively.

12. The IL-12 Fc fusion protein according to claim 9, wherein the target antigen comprises human PD1, and the ABD comprises a VH/VL domain pair selected from the group consisting of: (i) SEQ ID NOs: 54 and 55, respectively, (ii) SEQ ID NOs: 93 and 94, respectively, (iii) SEQ ID NOs: 95 and 96, respectively, (iv) SEQ ID NOs: 97 and 98, respectively, (v) SEQ ID NOs: 99 and 100, respectively, and (vi) SEQ ID NOs: 101 and 102, respectively.

13. The IL-12 Fc fusion protein according to claim 9, wherein the target antigen comprises human ICOS, and the ABD comprises a VH/VL domain pair selected from the group consisting of: (i) SEQ ID NOs: 74 and 75, respectively, (ii) SEQ ID NOs: 117 and 118, respectively, (iii) SEQ ID NOs: 119 and 120, respectively, and (iv) SEQ ID NOs: 121 and 122, respectively.

14. The IL-12 Fc fusion protein according to claim 9, wherein the target antigen comprises human 41BB, and the ABD comprises a VH/VL domain pair selected from the group consisting of: (i) SEQ ID NOs: 70 and 71, respectively, (ii) SEQ ID NOs: 123 and 124, respectively, (iii) SEQ ID NOs: 125 and 126, respectively, and (iv) SEQ ID NOs: 127 and 128, respectively.

15. The IL-12 Fc fusion protein according to claim 9, wherein the target antigen comprises human CCR7, and the ABD comprises a VH/VL domain pair selected from the group consisting of: (i) SEQ ID NOs: 78 and 79, respectively.

16. The IL-12 Fc fusion protein according to claim 9, wherein the target antigen comprises human CCR8, and the ABD comprises a VH/VL domain pair selected from the group consisting of: (i) SEQ ID NOs: 82 and 83, respectively, (ii) SEQ ID NOs: 113 and 114, respectively, and (iii) SEQ ID NOs: 115 and 116, respectively.

17. The IL-12 Fc fusion protein according to claim 9, wherein the ABD binds an extracellular domain (ECD) of the target antigen.

18. The IL-12 Fc fusion protein according to claim 1, wherein the first domain linker and/or the second domain linker comprises any one of SEQ ID NOs: 31-40.

19.-152. (canceled)

153. An IL-12 Fc fusion protein, comprising:
(a) a first monomer comprising, from N-terminus to C-terminus: (i) a first IL-12 subunit domain, (ii) a first domain linker, (iii) a second IL-12 subunit domain, and (iv) a first Fc domain, wherein:
(1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and
(2) the variant IL-12 p35 subunit domain comprises a set of amino acid substitutions selected from the group consisting of: (a) Y40S/K170A, (b) Y40S/K170T, (c) Y40P/K168A, (d) Y40P/K170A, and (e) Y40E/K170A; and
(b) a second monomer comprising: (i) an anti-target antigen scFv, wherein the anti-target antigen scFv comprises a VH domain, a second domain linker, and a VL domain, (ii) a third domain linker, and (iii) a second Fc domain, wherein the anti-target antigen scFv is covalently attached to the third domain linker at the N-terminus of the second Fc domain.

154.-284. (canceled)

285. An IL-12 Fc fusion protein, comprising:
(a) a first monomer comprising, from N-terminus to C-terminus: (i) a first IL-12 subunit domain, (ii) a first domain linker, (iii) a second IL-12 subunit domain, and (iv) a first Fc domain, wherein:
(1) the first IL-12 subunit domain comprises an IL-12 p40 subunit domain and the second IL-12 subunit domain comprises a variant IL-12 p35 subunit domain or the first IL-12 subunit domain comprises a variant IL-12 p35 subunit domain and the second IL-12 subunit domain comprises an IL-12 p40 subunit domain, and
(2) the variant IL-12 p35 subunit domain comprises a set of amino acid substitutions selected from the group consisting of: (a) Y40S/K170A, (b) Y40S/K170T, (c) Y40P/K168A, (d) Y40P/K170A, and (e) Y40E/K170A; and
(b) a second monomer comprising, from N-terminus to C-terminus: (i) a VHH domain,
(ii) a second domain linker, and (iii) a second Fc domain, wherein the VHH domain binds a target antigen.

286.-401. (canceled)

402. An IL-12 Fc fusion protein, comprising:
(a) a first monomer;
(b) a second monomer; and
(c) a third monomer, wherein the first monomer, the second monomer, and the third monomer comprise:
(i) SEQ ID NOs: 49, 153, and 145, respectively,
(ii) SEQ ID NOs: 48, 146, and 145, respectively,
(iii) SEQ ID NOs: 48, 151, and 150, respectively,
(iv) SEQ ID NOs: 48, 153, and 145, respectively,
(v) SEQ ID NOs: 48, 155, and 145, respectively,
(vi) SEQ ID NOs: 48, 157, and 145, respectively,
(vii) SEQ ID NOs: 48, 159, and 150, respectively,
(viii) SEQ ID NOs: 48, 161, and 150, respectively,
(ix) SEQ ID NOs: 48, 163, and 150, respectively,
(x) SEQ ID NOs: 48, 165, and 150, respectively,
(xi) SEQ ID NOs: 48, 167, and 150, respectively,
(xii) SEQ ID NOs: 48, 169, and 150, respectively,
(xiii) SEQ ID NOs: 49, 141, and 140, respectively,
(xiv) SEQ ID NOs: 49, 146, and 145, respectively,
(xv) SEQ ID NOs: 49, 151, and 150, respectively,
(xvi) SEQ ID NOs: 48, 141, and 140, respectively,
(xvii) SEQ ID NOs: 49, 155, and 145, respectively,
(xviii) SEQ ID NOs: 49, 157, and 145, respectively,
(xix) SEQ ID NOs: 49, 159, and 150, respectively,
(xx) SEQ ID NOs: 49, 161, and 150, respectively,
(xxi) SEQ ID NOs: 49, 163, and 150, respectively,
(xxii) SEQ ID NOs: 49, 165, and 150, respectively,
(xxiii) SEQ ID NOs: 49, 167, and 150, respectively,
(xxiv) SEQ ID NOs: 49, 169, and 150, respectively,
(xxv) SEQ ID NOs: 50, 141, and 140, respectively,
(xxvi) SEQ ID NOs: 50, 146, and 145, respectively,
(xxvii) SEQ ID NOs: 50, 151, and 150, respectively,
(xxviii) SEQ ID NOs: 50, 153, and 145, respectively,
(xxix) SEQ ID NOs: 50, 155, and 145, respectively,
(xxx) SEQ ID NOs: 50, 157, and 145, respectively,
(xxxi) SEQ ID NOs: 50, 159, and 150, respectively,
(xxxii) SEQ ID NOs: 50, 161, and 150, respectively,
(xxxiii) SEQ ID NOs: 50, 163, and 150, respectively,
(xxxiv) SEQ ID NOs: 50, 165, and 150, respectively,
(xxxv) SEQ ID NOs: 50, 167, and 150, respectively,
(xxxvi) SEQ ID NOs: 50, 169, and 150, respectively,
(xxxvii) SEQ ID NOs: 51, 141, and 140, respectively,
(xxxviii) SEQ ID NOs: 51, 146, and 145, respectively,
(xxxix) SEQ ID NOs: 51, 151, and 150, respectively,
(xl) SEQ ID NOs: 51, 153, and 145, respectively,
(xli) SEQ ID NOs: 51, 155, and 145, respectively,
(xlii) SEQ ID NOs: 51, 157, and 145, respectively,
(xliii) SEQ ID NOs: 51, 159, and 150, respectively,
(xliv) SEQ ID NOs: 51, 161, and 150, respectively,
(xlv) SEQ ID NOs: 51, 163, and 150, respectively,
(xlvi) SEQ ID NOs: 51, 165, and 150, respectively,
(xlvii) SEQ ID NOs: 51, 167, and 150, respectively,
(xlviii) SEQ ID NOs: 51, 169, and 150, respectively,
(xlix) SEQ ID NOs: 52, 141, and 140, respectively,
(l) SEQ ID NOs: 52, 146, and 145, respectively,
(li) SEQ ID NOs: 52, 151, and 150, respectively,
(lii) SEQ ID NOs: 52, 153, and 145, respectively,
(liii) SEQ ID NOs: 52, 155, and 145, respectively,
(liv) SEQ ID NOs: 52, 157, and 145, respectively,
(lv) SEQ ID NOs: 52, 159, and 150, respectively,
(lvi) SEQ ID NOs: 52, 161, and 150, respectively,
(lvii) SEQ ID NOs: 52, 163, and 150, respectively,
(lviii) SEQ ID NOs: 52, 165, and 150, respectively,
(lix) SEQ ID NOs: 52, 167, and 150, respectively,
(lx) SEQ ID NOs: 52, 169, and 150, respectively,
(lxi) SEQ ID NOs: 48, 174, and 173, respectively,
(lxii) SEQ ID NOs: 49, 174, and 173, respectively,
(lxiii) SEQ ID NOs: 50, 174, and 173, respectively,
(lxiv) SEQ ID NOs: 51, 174, and 173, respectively, or
(lxv) SEQ ID NOs: 52, 174, and 173, respectively.

403. A nucleic acid composition comprising one or more nucleic acids encoding the IL-12 Fc fusion protein according to claim 1.

404. An expression vector comprising the one or more nucleic acids of claim 403.

405. A host cell transformed with the expression vector of claim 404.

406. A method of making an IL-12 Fc fusion protein, comprising:
(a) culturing the host cell of claim 405 under conditions wherein the IL-12 Fc fusion protein is expressed; and
(b) isolating and/or purifying the IL-12 Fc fusion protein.

407. A method of treating a cancer in a human subject comprising administering the IL-12 Fc fusion protein according to claim 1 to a human subject in need thereof, wherein:
(i) the IL-12 Fc fusion protein binds to a target cell;
(ii) the target cell expresses: (1) IL-12Rβ2, and (2) human OX40; and
(iii) the target cell comprises: (A) activated CD8+ T cell(s), (B) exhausted CD8+ T cell(s), (C) activated CD4+ T cell(s), (D) exhausted CD4+ T cell(s), (E) activated NK cell(s), (F) exhausted NK cell(s), (G) activated regulatory T cell(s), or (H) exhausted regulatory T cell(s).

408.-419. (canceled)


SEQUENCE LISTING (174 sequences)

SEQ ID NO: 1 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 662 aa, synthetic construct
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 6 - protein, 522 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 7 - protein, 862 aa, synthetic construct
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 8 - protein, 599 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKAN

SEQ ID NO: 9 - protein, 215 aa, synthetic construct
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 10 - protein, 193 aa, synthetic construct
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 11 - protein, 335 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 12 - protein, 313 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 13 - protein, 738 aa, synthetic construct
MDMMGLAGTSKHITFLLLCQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAKIKFLVEPLNQGGRRRLTMQGQSPQLAVPEGCRGRPGAQVKKHLVLVRMLSCRCQAQTSKTVPLGKKLNLSGATYDLNVLAKTRFGRSTIQKWHLPAQELTETRALNVSVGGNMTSMQWAAQAPGTTYCLEWQPWFQHRNHTHCTLIVPEEEDPAKMVTHSWSSKPTLEQEECYRITVFASKNPKNPMLWATVLSSYYFGGNASRAGTPRHVSVRNQTGDSVSVEWTASQLSTCPGVLTQYVVRCEAEDGAWESEWLVPPTKTQVTLDGLRSRVMYKVQVRADTARLPGAWSHPQRFSFEVQISRLSIIFASLGSFASVLLVGSLGYIGLNRAAWHLCPPLPTPCGSTAVEFPGSQGKQAWQWCNPEDFPEVLYPRDALVVEMPGDRGDGTESPQAAPECALDTRRPLETQRQRQVQALSEARRLGLAREDCPRGDLAHVTLPLLLGGVTQGASVLDDLWRTHKTAEPGPPTLGQEA

SEQ ID NO: 14 - protein, 546 aa, synthetic construct
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAKIKFLVEPLNQGGRRRLTMQGQSPQLAVPEGCRGRPGAQVKKHLVLVRMLSCRCQAQTSKTVPLGKKLNLSGATYDLNVLAKTRFGRSTIQKWHLPAQELTETRALNVSVGGNMTSMQWAAQAPGTTYCLEWQPWFQHRNHTHCTLIVPEEEDPAKMVTHSWSSKPTLEQEECYRITVFASKNPKNPMLWATVLSSYYFGGNASRAGTPRHVSVRNQTGDSVSVEWTASQLSTCPGVLTQYVVRCEAEDGAWESEWLVPPTKTQVTLDGLRSRVMYKVQVRADTARLPGAWSHPQRFSFEVQIS

SEQ ID NO: 15 - protein, 874 aa, synthetic construct
MAQTVRECSLALLFLFMWLLIKANIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEEEPVGILDIWYMKQDIDYDRQQISLFWKSLNPSEARGKILHYQVTLQEVTKKTTLQNTTRHTSWTRVIPRTGAWTASVSAANSKGASAPTHINIVDLCGTGLLAPHQVSAKSENMDNILVTWQPPKKADSAVREYIVEWRALQPGSITKFPPHWLRIPPDNMSALISENIKPYICYEIRVHALSESQGGCSSIRGDSKHKAPVSGPHITAITEKKERLFISWTHIPFPEQRGCILHYRIYWKERDSTAQPELCEIQYRRSQNSHPISSLQPRVTYVLWMTAVTAAGESPQGNEREFCPQGKANWKAFVISSICIAIITVGTFSIRYFRQKAFTLLSTLKPQWYSRTIPDPANSTWVKKYPILEEKIQLPTDNLLMAWPTPEEPEPLIIHEVLYHMIPVVRQPYYFKRGQGFQGYSTSKQDAMYIANPQATGTLTAETRQLVNLYKVLESRDPDSKLANLTSPLTVTPVNYLPSHEGYLPSNIEDLSPHEADPTDSFDLEHQHISLSIFASSSLRPLIFGGERLTLDRLKMGYDSLMSNEA

SEQ ID NO: 16 - protein, 614 aa, synthetic construct
NIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEEEPVGILDIWYMKQDIDYDRQQISLFWKSLNPSEARGKILHYQVTLQEVTKKTTLQNTTRHTSWTRVIPRTGAWTASVSAANSKGASAPTHINIVDLCGTGLLAPHQVSAKSENMDNILVTWQPPKKADSAVREYIVEWRALQPGSITKFPPHWLRIPPDNMSALISENIKPYICYEIRVHALSESQGGCSSIRGDSKHKAPVSGPHITAITEKKERLFISWTHIPFPEQRGCILHYRIYWKERDSTAQPELCEIQYRRSQNSHPISSLQPRVTYVLWMTAVTAAGESPQGNEREFCPQGKAN

SEQ ID NO: 17 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNAS

SEQ ID NO: 18 - protein, 197 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 19 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTTIKLCILLHAFRIRAVTIDRVMSYLNAS

SEQ ID NO: 20 - protein, 197 aa, synthetic construct
RNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 21 - protein, 197 aa, synthetic construct
RNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNAS

SEQ ID NO: 22 - protein, 193 aa, synthetic construct
RVIPVSGPARCLSQSRNLLKTTDDMVKTAREKLKHESCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 23 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 24 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCS

SEQ ID NO: 25 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCS

SEQ ID NO: 26 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 231 aa, synthetic construct
EPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 231 aa, synthetic construct
EPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 29 - protein, 231 aa, synthetic construct
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 231 aa, synthetic construct
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 31 - protein, 5 aa, synthetic construct
GGGGS

SEQ ID NO: 32 - protein, 10 aa, synthetic construct
GGGGSGGGGS

SEQ ID NO: 33 - protein, 15 aa, synthetic construct
GGGGSGGGGSGGGGS

SEQ ID NO: 34 - protein, 17 aa, synthetic construct
TRGGGGSGGGGSGGGGS

SEQ ID NO: 35 - protein, 20 aa, synthetic construct
GGGGSGGGGSGGGGSGGGGS

SEQ ID NO: 36 - protein, 25 aa, synthetic construct
GGGGSGGGGSGGGGSGGGGSGGGGS

SEQ ID NO: 37 - protein, 30 aa, synthetic construct
GGGGSGGGGSGGGGSGGGGSGGGGSGGGGS

SEQ ID NO: 38 - protein, 35 aa, synthetic construct
GGGGSGGGGSGGGGSGGGGSGGGGSGGGGSGGGGS

SEQ ID NO: 39 - protein, 40 aa, synthetic construct
GGGGSGGGGSGGGGSGGGGSGGGGSGGGGSGGGGSGGGGS

SEQ ID NO: 40 - protein, 18 aa, synthetic construct
GSTSGSGKPGSGEGSTKG

SEQ ID NO: 41 - protein, 113 aa, synthetic construct
DVQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSA

SEQ ID NO: 42 - protein, 107 aa, synthetic construct
DIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIK

SEQ ID NO: 43 - protein, 460 aa, synthetic construct
MKWVTFISLLFLFSSAYSDVQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 243 aa, synthetic construct
MGILPSPGMPALLSLVSLLSVLLMGCVAEDIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 466 aa, synthetic construct
MGILPSPGMPALLSLVSLLSVLLMGCVAEDVQLQESGPSLVKPSQTLSLTCSVTGDSITSDYWSWIRKFPGNRLEYMGYVSYSGSTYYNPSLKSRISITRDTSKNQYYLDLNSVTTEDTATYYCANWDGDYWGQGTLVTVSAAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 46 - protein, 232 aa, synthetic construct
MKWVTFISLLFLFSSAYSDIVLTQSPATLSVTPGNSVSLSCRASQSIGNNLHWYQQKSHESPRLLIKYASQSISGIPSRFSGSGSGTDFTLSINSVETEDFGMYFCQQSNSWPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 47 - protein, 787 aa, synthetic construct
MKWVTFISLLFLFSSAYSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 48 - protein, 787 aa, synthetic construct
MKWVTFISLLFLFSSAYSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSGGGGSGGGGSRNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 49 - protein, 787 aa, synthetic construct
MKWVTFISLLFLFSSAYSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSGGGGSGGGGSRNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 50 - protein, 787 aa, synthetic construct
MKWVTFISLLFLFSSAYSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSGGGGSGGGGSRNLPVATPDPGMFPCLHHSQNLLRAVSNMLQKARQTLEFSPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTTIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 51 - protein, 787 aa, synthetic construct
MKWVTFISLLFLFSSAYSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSGGGGSGGGGSRNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 52 - protein, 787 aa, synthetic construct
MKWVTFISLLFLFSSAYSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSGGGGSGGGGSRNLPVATPDPGMFPCLHHSQNLLRAVSNMLQKARQTLEFEPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTAIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 53 - protein, 786 aa, synthetic construct
MKWVTFISLLFLFSSAYSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSGGGGSGGGGSRVIPVSGPARCLSQSRNLLKTTDDMVKTAREKLKHESCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSVPRDSGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKKQMAKDKVSLTCMITDFFPEDITVEWQWNGQPAENYKNTQPIMKTDGSYFVYSKLNVQKSNWEAGNTFTCSVLHEGLHNHHTEKSLSHSPGK

SEQ ID NO: 54 - protein, 117 aa, synthetic construct
QVQLVQSGAEVKKPGSSVKVSCKASGYTFTSYWINWVRQAPGQGLEWMGNIYPGSSITNYAQKFQGRVTITADESTSTAYMELSSLRSEDTAVYYCARLTTGTFAYWGQGTLVTVSS

SEQ ID NO: 55 - protein, 113 aa, synthetic construct
DIQMTQSPSSLSASVGDRVTITCKSSQSLWDSGNQKNFLTWYQQKPGKAPKLLIYWTSYRESGVPSRFSGSGSGTDFTLTISSLQPEDFATYYCQNDYFYPLTFGGGTKVEIK

SEQ ID NO: 56 - protein, 464 aa, synthetic construct
MKWVTFISLLFLFSSAYSQVQLVQSGAEVKKPGSSVKVSCKASGYTFTSYWINWVRQAPGQGLEWMGNIYPGSSITNYAQKFQGRVTITADESTSTAYMELSSLRSEDTAVYYCARLTTGTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 249 aa, synthetic construct
MGILPSPGMPALLSLVSLLSVLLMGCVAEDIQMTQSPSSLSASVGDRVTITCKSSQSLWDSGNQKNFLTWYQQKPGKAPKLLIYWTSYRESGVPSRFSGSGSGTDFTLTISSLQPEDFATYYCQNDYFYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 118 aa, synthetic construct
EVQLVESGGGLVKPGGSLELSCAASGFTFSSYWMSWVRQAPEKGLEWVAAISPSGGSTYYADSVKGRFTISRDNAKNTLFLQMTSLRSEDTAMYYCAKESWGAYYDLWGQGTTVTVSS

SEQ ID NO: 59 - protein, 107 aa, synthetic construct
DIVMTQSPSSLSVSAGDKVTMSCRASQGISSWLAWYQQKPWQPPKLLIYKASTLESGVPDRFTGSGSGTDFTLTISSVQAEDLAVYYCQQSYSTPWTFGGGTKLEIK

SEQ ID NO: 60 - protein, 460 aa, synthetic construct
MKWVTFISLLFLFSSAYSEVQLVESGGGLVKPGGSLELSCAASGFTFSSYWMSWVRQAPEKGLEWVAAISPSGGSTYYADSVKGRFTISRDNAKNTLFLQMTSLRSEDTAMYYCAKESWGAYYDLWGQGTTVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSDLNVQKSNWEAGNTFTCSVLHEGLHNHHTEESLSHSPGK

SEQ ID NO: 61 - protein, 232 aa, synthetic construct
MKWVTFISLLFLFSSAYSDIVMTQSPSSLSVSAGDKVTMSCRASQGISSWLAWYQQKPWQPPKLLIYKASTLESGVPDRFTGSGSGTDFTLTISSVQAEDLAVYYCQQSYSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 62 - protein, 120 aa, synthetic construct
QLQLQESGPGLVKPSETLSLTCTVSGGSIYSESYYWGWIRQPPGKGLEWIGSIVYSGYTYYNPSLKSRVTISVDTSKNQFSLKLSSVTAADTAVYYCARVRTWDAAFDIWGQGTMVTVSS

SEQ ID NO: 63 - protein, 108 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQVLELPPWTFGGGTKVEIK

SEQ ID NO: 64 - protein, 462 aa, synthetic construct
MKWVTFISLLFLFSSAYSQLQLQESGPGLVKPSETLSLTCTVSGGSIYSESYYWGWIRQPPGKGLEWIGSIVYSGYTYYNPSLKSRVTISVDTSKNQFSLKLSSVTAADTAVYYCARVRTWDAAFDIWGQGTMVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSDLNVQKSNWEAGNTFTCSVLHEGLHNHHTEESLSHSPGK

SEQ ID NO: 65 - protein, 233 aa, synthetic construct
MKWVTFISLLFLFSSAYSDIQMTQSPSSLSASVGDRVTITCQASQDISNYLNWYQQKPGKAPKLLIYDASNLETGVPSRFSGSGSGTDFTFTISSLQPEDIATYYCQQVLELPPWTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 66 - protein, 117 aa, synthetic construct
QVQLKESGPGLVQPSQTLSLTCTVSGFSLTGYNLHWVRQPPGKGLEWMGRMRYDGDTYYNSVLKSRLSISRDTSKNQVFLKMNSLQTDDTAIYYCTRDGRGDSFDYWGQGVMVTVSS

SEQ ID NO: 67 - protein, 112 aa, synthetic construct
DIVMTQGALPNPVPSGESASITCRSSQSLVYKDGQTYLNWFLQRPGQSPQLLTYWMSTRASGVSDRFSGSGSGTYFTLKISRVRAEDAGVYYCQQVREYPFTFGSGTKLEIK

SEQ ID NO: 68 - protein, 459 aa, synthetic construct
MKWVTFISLLFLFSSAYSQVQLKESGPGLVQPSQTLSLTCTVSGFSLTGYNLHWVRQPPGKGLEWMGRMRYDGDTYYNSVLKSRLSISRDTSKNQVFLKMNSLQTDDTAIYYCTRDGRGDSFDYWGQGVMVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSDLNVQKSNWEAGNTFTCSVLHEGLHNHHTEESLSHSPGK

SEQ ID NO: 69 - protein, 237 aa, synthetic construct
MKWVTFISLLFLFSSAYSDIVMTQGALPNPVPSGESASITCRSSQSLVYKDGQTYLNWFLQRPGQSPQLLTYWMSTRASGVSDRFSGSGSGTYFTLKISRVRAEDAGVYYCQQVREYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 70 - protein, 118 aa, synthetic construct
QVKLVQSGAALVKPGASVKMSCKASDYTFNDYWVSWVKQRHGESLEWIGEIYPNSGATNFNGKFRGKATLTVDNPTSTAYMELSRLTSEDSAIYYCTREYTRDWFAYWGQGTLVTVSS

SEQ ID NO: 71 - protein, 112 aa, synthetic construct
DVVLTQTPSILSATIGQSVSISCRSSQSLLDSDGNTYLYWFLQRPGQSPQRLIYLVSNLGSGVPNRFSGSGSGTDFTLKISGVEAEDLGIYYCMQPTHAPYTFGAGTKLELK

SEQ ID NO: 72 - protein, 460 aa, synthetic construct
MKWVTFISLLFLFSSAYSQVKLVQSGAALVKPGASVKMSCKASDYTFNDYWVSWVKQRHGESLEWIGEIYPNSGATNFNGKFRGKATLTVDNPTSTAYMELSRLTSEDSAIYYCTREYTRDWFAYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSDLNVQKSNWEAGNTFTCSVLHEGLHNHHTEESLSHSPGK

SEQ ID NO: 73 - protein, 237 aa, synthetic construct
MKWVTFISLLFLFSSAYSDVVLTQTPSILSATIGQSVSISCRSSQSLLDSDGNTYLYWFLQRPGQSPQRLIYLVSNLGSGVPNRFSGSGSGTDFTLKISGVEAEDLGIYYCMQPTHAPYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 74 - protein, 124 aa, synthetic construct
EVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSS

SEQ ID NO: 75 - protein, 108 aa, synthetic construct
EIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIK

SEQ ID NO: 76 - protein, 466 aa, synthetic construct
MKWVTFISLLFLFSSAYSEVQLVESGGGVVRPGGSLRLSCVASGVTFDDYGMSWVRQAPGKGLEWVSGINWNGGDTDYSDSVKGRFTISRDNAKNSLYLQMNSLRAEDTALYYCARDFYGSGSYYHVPFDYWGQGILVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSDLNVQKSNWEAGNTFTCSVLHEGLHNHHTEESLSHSPGK

SEQ ID NO: 77 - protein, 233 aa, synthetic construct
MKWVTFISLLFLFSSAYSEIVLTQSPGTLSLSPGERATLSCRASQSVSRSYLAWYQQKRGQAPRLLIYGASSRATGIPDRFSGDGSGTDFTLSISRLEPEDFAVYYCHQYDMSPFTFGPGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 78 - protein, 117 aa, synthetic construct
DVQLQESGPGLVKPSQTLSLTCTVSGYSITSGYSWHWIRQHPGKGLEWMAHIHSSGSTNYNPSLKSRITISRDTSKNQFSLKLSSVTAADTAVYYCARGGVQAFAYWGQGTLVTVSS

SEQ ID NO: 79 - protein, 106 aa, synthetic construct
EIVLTQSPATLSASPGERVTMSCSASSSVIYMHWYQQKPGQAPRRWIYDTSKLASGVPARFSGSGSGTDYTLTISSMEPEDAAVYYCQQWSSNPLTFGQGTKLEIK

SEQ ID NO: 80 - protein, 464 aa, synthetic construct
MKWVTFISLLFLFSSAYSDVQLQESGPGLVKPSQTLSLTCTVSGYSITSGYSWHWIRQHPGKGLEWMAHIHSSGSTNYNPSLKSRITISRDTSKNQFSLKLSSVTAADTAVYYCARGGVQAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 242 aa, synthetic construct
MGILPSPGMPALLSLVSLLSVLLMGCVAEEIVLTQSPATLSASPGERVTMSCSASSSVIYMHWYQQKPGQAPRRWIYDTSKLASGVPARFSGSGSGTDYTLTISSMEPEDAAVYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 116 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSS

SEQ ID NO: 83 - protein, 112 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIK

SEQ ID NO: 84 - protein, 463 aa, synthetic construct
MKWVTFISLLFLFSSAYSQVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 248 aa, synthetic construct
MGILPSPGMPALLSLVSLLSVLLMGCVAEDIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 123 aa, synthetic construct
DVQLVESGGGLVQPGGSLRLSCTVSGSIFSLRTVGWYRQAPGKQRELVATISAGGATDYADSVKGRFTISRDNAKNTVYLQMNSLRPEDTAVYYCYAQDSYKIYKSRYTQDYWGQGTLVTVSS

SEQ ID NO: 87 - protein, 382 aa, synthetic construct
MKWVTFISLLFLFSSAYSDVQLVESGGGLVQPGGSLRLSCTVSGSIFSLRTVGWYRQAPGKQRELVATISAGGATDYADSVKGRFTISRDNAKNTVYLQMNSLRPEDTAVYYCYAQDSYKIYKSRYTQDYWGQGTLVTVSSGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 355 aa, synthetic construct
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 89 - protein, 438 aa, synthetic construct
RNLPVATPDPGMFPCLHHSQNLLRAVSNMLQKARQTLEFPPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYATKIKLCILLHAFRIRAVTIDRVMSYLNASGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 90 - protein, 547 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 22 aa, synthetic construct
MCPARSLLLVATLVLLDHLSLA

SEQ ID NO: 92 - protein, 22 aa, synthetic construct
MCHQQLVISWFSLVFLASPLVA

SEQ ID NO: 93 - protein, 113 aa, synthetic construct [sequence omitted]

SEQ ID NO: 94 - protein, 107 aa, synthetic construct [sequence omitted]

SEQ ID NO: 95 - protein, 120 aa, synthetic construct [sequence omitted]

SEQ ID NO: 96 - protein, 111 aa, synthetic construct [sequence omitted]

SEQ ID NO: 97 - protein, 120 aa, synthetic construct
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 98 - protein, 107 aa, synthetic construct
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 99 - protein, 125 aa, synthetic construct [sequence omitted]

SEQ ID NO: 100 - protein, 112 aa, synthetic construct [sequence omitted]

SEQ ID NO: 101 - protein, 121 aa, synthetic construct
EVQLVESGGGLVQPGGSLRLSCAASGFSFGDFDMRWFRQAPGKGLEWVGTIKSRAYLEATEFAASVEGRFTISRDDAKNSAYLQMNSLRAEDTAVYYCTRDAYSSGLLDYWGQGTLVTVSS

SEQ ID NO: 102 - protein, 107 aa, synthetic construct
DIQMTQSPSSLSASVGDRVTITCRASQGISNYLAWFQQKPGKAPKRLIYAAQIPGSGVPSRFSGSGSGTDFTLTISSLQPEDFATYYCLQHYSYPLTFGGGTKVEIK

SEQ ID NO: 103 - protein, 120 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 104 - protein, 107 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 105 - protein, 122 aa, synthetic construct
QVTLVESGGGVVQPGRSLRLSCAFSGFSLSTSDMGVGWIRQAPGKGLEWVAHIWWDDVKRYNPALKSRFTISRDNSKNTLYLQMNSLRAEDTAVYFCARIEDYGVSYYFDYWGQGTTVTVSS

SEQ ID NO: 106 - protein, 107 aa, synthetic construct
DIQMTQSPSFLSASVGDRVSITCKASQDVSTAVAWYQQKPGKAPKLLIYSASYRYTGVPDRFSGSGSGTDFTLTISSLQPEDFATYYCQQHYSIPLTFGQGTKLEIK

SEQ ID NO: 107 - protein, 125 aa, synthetic construct
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 108 - protein, 107 aa, synthetic construct
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 109 - protein, 123 aa, synthetic construct
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 110 - protein, 107 aa, synthetic construct
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 111 - protein, 119 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 112 - protein, 111 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 113 - protein, 119 aa, synthetic construct
EVQLLESGGGLVQPGGSLRLSCAASGIDLSTYAMGWVRQAPGKGLEWVGLIHRSGRTYYATWAKGRFTISKDSSKNTLYLQMNSLRAEDTAVYYCTRSYPDYSATASIWGQGTTVTVSS

SEQ ID NO: 114 - protein, 111 aa, synthetic construct
DIQVTQSPSSLSASVGDRVTITCQASENIANALAWYQQKPGKPPKFLIYGASNLASGVPSRFSGSGSGTDFTFTISSLQPEDIATYYCQQAYYGNSFVEGTFGGGTKVEIK

SEQ ID NO: 115 - protein, 124 aa, synthetic construct
EVQLLESGGGLVQPGGSLRLSCAASGFSLSNYAMIWVRQAPGKGLEWVSTISLGGYTYYANWAKGRFTISRDSSKTTVYLQMNSLRAEDTAVYFCARARWSTDSAIYTYAFDPWGPGTLVTVSS

SEQ ID NO: 116 - protein, 110 aa, synthetic construct
DYQMTQSPSSLSASVGDRVTITCQASQSISSYLSWYQQKPGKAPKLLIYKASTLASGVPSRFSGSGSGTDFTLTISSLQPEDFATYYCQQGYTSSNIDNIFGGGTKVEIK

SEQ ID NO: 117 - protein, 120 aa, synthetic construct
QVQLVESGGGVVQPGMSLRLSCAASGFTFSTYGMQWVRQAPGKGLEWVTVIWHDGSHKDYADSVKGRFTISRDNSKNTMYLQMNSLRAEDTAVYYCARDRQTGEGYFDFWGQGTLVTVSS

SEQ ID NO: 118 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 119 - protein, 117 aa, synthetic construct
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 120 - protein, 111 aa, synthetic construct
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 121 - protein, 121 aa, synthetic construct
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 122 - protein, 106 aa, synthetic construct
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 123 - protein, 120 aa, synthetic construct
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVS

SEQ ID NO: 124 - protein, 109 aa, synthetic construct [sequence omitted]

SEQ ID NO: 125 - protein, 120 aa, synthetic construct
EVQLLESGGGLVQPGGSLRLSCAASGFTFSYGSMYWVRQAPGKGLEWVSSISSGSGSTYYADSVKGRFTISRDNSKNTLYLQMNSLRAEDTAVYYCARSSYYGSYYSIDYWGQGTLVTVS

SEQ ID NO: 126 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQYYDNLPTFGQGTKLEIK

SEQ ID NO: 127 - protein, 122 aa, synthetic construct
EVQLVESGGGLVQPGGSLRLSCAASGFSFSANYYPCWVRQAPGKGLEWIGCIYGGSSDITYDANWTKGRFTISRDNSKNTVYLQMNSLRAEDTAVYYCARSAWYSGWGGDLWGQGTLVTVSS

SEQ ID NO: 128 - protein, 109 aa, synthetic construct
DIQMTQSPSSLSASVGDRVTITCQASQSISNRLAWYQQKPGKAPKLLIYSASTLASGVPSRFSGSGSGTDFTLTISSLQPEDFATYYCQSTYYGNDGNAFGTGTKVTVL

SEQ ID NO: 129 - protein, 126 aa, synthetic construct
QVQLVESGGGVVQPGRSLRLSCAASGSTFSIVAMGWYRQAPGKQRELVASIITGDGDTNYADSVKGRFTISRDNSKNTMYLQMNSLKPEDTAVYYCYARTGYGSSWLMGHEYDYWGQGTQVTVSSL

SEQ ID NO: 130 - protein, 123 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDHASSSWYTTHLDLWGRGTLVTVSS

SEQ ID NO: 131 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQSANYPYTFGGGTKVEIK

SEQ ID NO: 132 - protein, 120 aa, synthetic construct
QVQLVQSGAEVKKPGSSVKVSCKASGYKFTSYIIHWVRQAPGQGLEWMGYINPYNEGTRYNQKFQGRVTLTADKSTSTAYMELSSLRSEDTAVYYCARGYYGSSYAMDYWGQGTTVTVSS

SEQ ID NO: 133 - protein, 107 aa, synthetic construct
DIQMTQSPSSLSASVGDRVTITCRASQGISNYLNWYQQKPDGAIKLLIYDASTLYSGVPSRFSGSGSGTDFTLTISSLQPEDFATYYCQQYSKLPYTFGGGTKVEIK

SEQ ID NO: 134 - protein, 378 aa, synthetic construct
MKWVTFISLLFLFSSAYSDVQLVESGGGLVQPGGSLRLSCTVSGSIFSLRTVGWYRQAPGKQRELVATISAGGATDYADSVKGRFTISRDNAKNTVYLQMNSLRPEDTAVYYCYAQDSYKIYKSRYTQDYWGQGTLVTVSSGGGGSGGGGSVPRDSGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSDLNVQKSNWEAGNTFTCSVLHEGLHNHHTEESLSHSPGK

SEQ ID NO: 135 - protein, 448 aa, synthetic construct
MKWVTFISLLFLFSSAYSRVIPVSGPARCLSQSRNLLKTTDDMVKTAREKLKHESCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSVPRDSGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSDLNVQKSNWEAGNTFTCSVLHEGLHNHHTEESLSHSPGK

SEQ ID NO: 136 - protein, 568 aa, synthetic construct
MKWVTFISLLFLFSSAYSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSVPRDSGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKKQMAKDKVSLTCMITDFFPEDITVEWQWNGQPAENYKNTQPIMKTDGSYFVYSKLNVQKSNWEAGNTFTCSVLHEGLHNHHTEKSLSHSPGK

SEQ ID NO: 137 - protein, 120 aa, Synthetic construct; identical to SEQ ID NO: 132

SEQ ID NO: 138 - protein, 107 aa, Synthetic construct; identical to SEQ ID NO: 133

SEQ ID NO: 139 - protein, 450 aa, Synthetic construct
  VARIANT 396: note = X is Threonine or Alanine
QVQLVQSGAEVKKPGSSVKVSCKASGYKFTSYIIHWVRQAPGQGLEWMGYINPYNEGTRYNQKFQGRVTLTADKSTSTAYMELSSLRSEDTAVYYCARGYYGSSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKXTPPVLDSDGSFFLYSKLTVDKSRWQQGNVFSCSVMHEALHNHYTQKSLSLSPGK

SEQ ID NO: 140 - protein, 214 aa, Synthetic construct
DIQMTQSPSSLSASVGDRVTITCRASQGISNYLNWYQQKPDGAIKLLIYDASTLYSGVPSRFSGSGSGTDFTLTISSLQPEDFATYYCQQYSKLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 449 aa, Synthetic construct
QVQLVQSGAEVKKPGSSVKVSCKASGYKFTSYIIHWVRQAPGQGLEWMGYINPYNEGTRYNQKFQGRVTLTADKSTSTAYMELSSLRSEDTAVYYCARGYYGSSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 119 aa, Synthetic construct
QVQLVQSGAEVKKPGASVKVSCKASGYTFTSYVMHWVRQAPGQRLEWIGYINPYNDGTKYNEKFKGRATLTSDKSASTAYMELSSLRSEDTAVYYCANYYGSSLSMDYWGQGTLVTVSS

SEQ ID NO: 143 - protein, 107 aa, Synthetic construct
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPWTFGQGTKVEIK

SEQ ID NO: 144 - protein, 449 aa, Synthetic construct
  VARIANT 395: note = X is Threonine or Alanine
QVQLVQSGAEVKKPGASVKVSCKASGYTFTSYVMHWVRQAPGQRLEWIGYINPYNDGTKYNEKFKGRATLTSDKSASTAYMELSSLRSEDTAVYYCANYYGSSLSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKXTPPVLDSDGSFFLYSKLTVDKSRWQQGNVFSCSVMHEALHNHYTQKSLSLSPGK

SEQ ID NO: 145 - protein, 214 aa, synthetic construct
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 448 aa, Synthetic construct
QVQLVQSGAEVKKPGASVKVSCKASGYTFTSYVMHWVRQAPGQRLEWIGYINPYNDGTKYNEKFKGRATLTSDKSASTAYMELSSLRSEDTAVYYCANYYGSSLSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 121 aa, Synthetic construct
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARMGYHGPHLDFDVWGQGTTVTVSS

SEQ ID NO: 148 - protein, 107 aa, Synthetic construct
DIQMTQSPSSLSASVGDRVTITCKASQDVGAAVAWYQQKPGKAPKLLIYWASTRHTGVPSRFSGSGSGTDFTLTISSLQPEDFATYYCQQYINYPLTFGGGTKVEIK

SEQ ID NO: 149 - protein, 451 aa, Synthetic construct
  VARIANT 397: note = X is Threonine or Alanine
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARMGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKXTPPVLDSDGSFFLYSKLTVDKSRWQQGNVFSCSVMHEALHNHYTQKSLSLSPGK

SEQ ID NO: 150 - protein, 214 aa, Synthetic construct
DIQMTQSPSSLSASVGDRVTITCKASQDVGAAVAWYQQKPGKAPKLLIYWASTRHTGVPSRFSGSGSGTDFTLTISSLQPEDFATYYCQQYINYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 450 aa, Synthetic construct
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARMGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 119 aa, Synthetic construct
QVQLVQSGAEVKKPGASVKVSCKASGYTFTSYVMHWVRQAPGQRLEWIGYINPYNEGTKYNEKFKGRATLTSDKSASTAYMELSSLRSEDTAVYYCANYYGSSLSMDYWGQGTLVTVSS

SEQ ID NO: 153 - protein, 448 aa, Synthetic construct
QVQLVQSGAEVKKPGASVKVSCKASGYTFTSYVMHWVRQAPGQRLEWIGYINPYNEGTKYNEKFKGRATLTSDKSASTAYMELSSLRSEDTAVYYCANYYGSSLSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 119 aa, Synthetic construct
QVQLVQSGAEVKKPGASVKVSCKASGYTFTSYVMHWVRQAPGQRLEWIGYINPYNAGTKYNEKFKGRATLTSDKSASTAYMELSSLRSEDTAVYYCANYYGSSLSMDYWGQGTLVTVSS

SEQ ID NO: 155 - protein, 448 aa, Synthetic construct
QVQLVQSGAEVKKPGASVKVSCKASGYTFTSYVMHWVRQAPGQRLEWIGYINPYNAGTKYNEKFKGRATLTSDKSASTAYMELSSLRSEDTAVYYCANYYGSSLSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 119 aa, Synthetic construct
QVQLVQSGAEVKKPGASVKVSCKASGYTFTSYVMHWVRQAPGQRLEWIGYINPYNDATKYNEKFKGRATLTSDKSASTAYMELSSLRSEDTAVYYCANYYGSSLSMDYWGQGTLVTVSS

SEQ ID NO: 157 - protein, 448 aa, Synthetic construct
QVQLVQSGAEVKKPGASVKVSCKASGYTFTSYVMHWVRQAPGQRLEWIGYINPYNDATKYNEKFKGRATLTSDKSASTAYMELSSLRSEDTAVYYCANYYGSSLSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - protein, 121 aa, Synthetic construct
QVQLVQSGAEVKKPGSSVKVSCKASGYTFKDYTMHWVRQAPGQGLEWIGGIYPNQGGSTYNQNFKDRVTLTADKSTSTAYMELSSLRSEDTAVYYCARMGYHGPHLDFDVWGQGTTVTVSS

SEQ ID NO: 159 - protein, 450 aa, Synthetic construct
QVQLVQSGAEVKKPGSSVKVSCKASGYTFKDYTMHWVRQAPGQGLEWIGGIYPNQGGSTYNQNFKDRVTLTADKSTSTAYMELSSLRSEDTAVYYCARMGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 121 aa, Synthetic construct
QVQLVQSGAEVKKPGSSVKVSCKASGYTFKDYTMHWVRQAPGQGLEWIGGIYPNAGGSTYNQNFKDRVTLTADKSTSTAYMELSSLRSEDTAVYYCARMGYHGPHLDFDVWGQGTTVTVSS

SEQ ID NO: 161 - protein, 450 aa, Synthetic construct
QVQLVQSGAEVKKPGSSVKVSCKASGYTFKDYTMHWVRQAPGQGLEWIGGIYPNAGGSTYNQNFKDRVTLTADKSTSTAYMELSSLRSEDTAVYYCARMGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 121 aa, Synthetic construct
QVQLVQSGAEVKKPGSSVKVSCKASGYTFKDYTMHWVRQAPGQGLEWIGGIYPNNAGSTYNQNFKDRVTLTADKSTSTAYMELSSLRSEDTAVYYCARMGYHGPHLDFDVWGQGTTVTVSS

SEQ ID NO: 163 - protein, 450 aa, Synthetic construct
QVQLVQSGAEVKKPGSSVKVSCKASGYTFKDYTMHWVRQAPGQGLEWIGGIYPNNAGSTYNQNFKDRVTLTADKSTSTAYMELSSLRSEDTAVYYCARMGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 121 aa, Synthetic construct
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARLGYHGPHLDFDVWGQGTTVTVSS

SEQ ID NO: 165 - protein, 450 aa, Synthetic construct
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARLGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 121 aa, Synthetic construct
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARIGYHGPHLDFDVWGQGTTVTVSS

SEQ ID NO: 167 - protein, 450 aa, Synthetic construct
QVQLVQSGAEVKKPGSSVKVSCKASGYTFKDYTMHWVRQAPGQGLEWIGGIYPNNGGSTYNQNFKDRVTLTADKSTSTAYMELSSLRSEDTAVYYCARIGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 168 - protein, 121 aa, Synthetic construct
QVQLVQSGAEVKKPGSSVKVSCKASGYTFKDYTMHWVRQAPGQGLEWIGGIYPNQGGSTYNQNFKDRVTLTADKSTSTAYMELSSLRSEDTAVYYCARIGYHGPHLDFDVWGQGTTVTVSS

SEQ ID NO: 169 - protein, 450 aa, Synthetic construct
QVQLVQSGAEVKKPGSSVKVSCKASGYTFKDYTMHWVRQAPGQGLEWIGGIYPNQGGSTYNQNFKDRVTLTADKSTSTAYMELSSLRSEDTAVYYCARIGYHGPHLDFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 170 - protein, 120 aa, Synthetic construct
QVQLVESGGGVVQPGRSLRLSCAASGFTFSSYGMHWVRQAPGKGLEWVALISYDGSNKIYTDSVKGRFTISRDNSKNTLYLQMNSLRPEDTAVYYCAKGREIVATYFDYWGQGTLVTVSS

SEQ ID NO: 171 - protein, 113 aa, Synthetic construct
DIVMTQSPDSLAVSLGERATINCKSSQSVLYSSNNKNYLAWYQQNPGQPPKLLIYWASTRESGVPDRFSGSGSGTDFTLTISSLQAEDVSVYYCQQYYRTPYTFGQGTKVEIK

SEQ ID NO: 172 - protein, 450 aa, Synthetic construct
QVQLVESGGGVVQPGRSLRLSCAASGFTFSSYGMHWVRQAPGKGLEWVALISYDGSNKIYTDSVKGRFTISRDNSKNTLYLQMNSLRPEDTAVYYCAKGREIVATYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 220 aa, Synthetic construct
DIVMTQSPDSLAVSLGERATINCKSSQSVLYSSNNKNYLAWYQQNPGQPPKLLIYWASTRESGVPDRFSGSGSGTDFTLTISSLQAEDVSVYYCQQYYRTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 449 aa, Synthetic construct
QVQLVESGGGVVQPGRSLRLSCAASGFTFSSYGMHWVRQAPGKGLEWVALISYDGSNKIYTDSVKGRFTISRDNSKNTLYLQMNSLRPEDTAVYYCAKGREIVATYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG